SEQUENCE LISTING (4 sequences)

SEQ ID NO: 1 - DNA, 20 bp, Artificial Sequence
  Synthetic polynucleotide
actcctacgggaggcagcag

SEQ ID NO: 2 - DNA, 17 bp, Artificial Sequence
  Synthetic polynucleotide
attaccgcggctgctgg

SEQ ID NO: 3 - DNA, 27 bp, Artificial Sequence
  Synthetic polynucleotide
aagatgacccagatcatgtttgagacc

SEQ ID NO: 4 - DNA, 20 bp, Artificial Sequence
  Synthetic polynucleotide
agccagtccagacgcaggat

(19) United States
(12) Patent Application Publication
(10) Pub. No.: US 2020/0332344 A1
(43) Pub. Date: Oct. 22, 2020

(54) TREATING AND DETECTING DYSBIOSIS

(71) Applicant: THE REGENTS OF THE UNIVERSITY OF CALIFORNIA, Oakland, CA (US)

(72) Inventor: Susan LYNCH, Piedmont, CA (US)

(21) Appl. No.: 16/609,161

(22) PCT Filed: May 11, 2018
(86) PCT No.: PCT/US18/32437

§ 371 Date: Oct. 28, 2019

Related U.S. Application Data
(60) Provisional application No. 62/505,799, filed on May 12, 2017.

(51) Int. Cl. C12Q 1/689; A61K 35/747; A61K 39/395; A61K 45/06; A61P 11/06

(52) U.S. Cl. CPC C12Q 1/689; A61K 35/747; C12Q 2600/158; A61K 45/06; A61P 11/06; A61K 39/3955

(57) ABSTRACT

Provided herein are, inter alia, methods, compositions, and systems, for detecting and treating dysbiosis such as nasal and sinus dysbiosis. In aspects, included herein are methods, compositions, and systems for detecting asthma, chronic rhinosinusitis, and infections (such as rhinovirus infections), and methods of treating such disorders. Also provided are methods, compositions, and systems for detecting whether a subject has an increased risk of asthma, an infection, asthma exacerbation, chronic rhinosinusitis, or nasal polyposis, as well as methods of treating at-risk subjects. Methods, compositions, and systems for monitoring subjects diagnosted as having a disease or risk as disclosed herein are also included.

### CROSS-REFERENCE TO RELATED APPLICATION

This application claims the benefit of priority to U.S. Provisional Application No. 62/505,799, filed May 12, 2017, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant nos. AI114271, P01 A1089473 and U01 HL098964 awarded by the National Institutes of Health. The government has certain rights in the invention.

### INCORPORATION-BY-REFERENCE OF SEQUENCE LISTING

The content of the text file named “048536-601001WO_SequenceListing.txt”, which was created on May 11, 2018, and is 1,010 bytes in size, is hereby incorporated by reference in its entirety.

### BACKGROUND

The microbiome may co-vary with host health status [Huang et al. J Allergy Clin Immunol 2011, 127(2):372-381 e371-373; Morgan et al. Genome biology 2012, 13(9):R79; and Yatsunenko et al. Nature 2012, 486(7402):222-227]. Microbes overtly colonize the upper respiratory mucosal surface of healthy subjects [Teo et al. Cell host & microbe 2015, 17(5):704-715; Abreu et al. Science translational medicine 2012, 4(151):151ra124], with lower bacterial burden and diversity observed in the lower airways [Charlson et al. Am J Respir Crit Care Med 2011, 184(8):957-963].

### BRIEF SUMMARY

Provided herein are, inter alia, methods, compositions, and systems, for detecting and treating dysbiosis such as nasal and sinus dysbiosis. Included are methods, compositions, and systems for detecting a nasal or sinus microbiome in a subject who has asthma. In aspects, provided herein are methods, compositions, and systems for detecting asthma, chronic rhinosinusitis, and infections (such as rhinovirus infections), and methods of treating such disorders. Also provided are methods, compositions, and systems for detecting whether a subject has an increased risk of asthma, an infection, asthma exacerbation, chronic rhinosinusitis, or nasal polyposis, as well as methods of treating at-risk subjects. Methods, compositions, and systems for monitoring subjects diagnosted as having a disease or risk as disclosed herein are also included.

In an aspect, a method of detecting a nasal or sinus microbiome in a subject who has asthma is provided. In embodiments, the method includes detecting bacteria, or a proportion of bacteria, in a biological sample from the subject that are in 1 of or any combination of the following taxa: Moraxella, Staphylococcaceae, Corynebacterium, Corynebacteriaceae, Staphylococcus, Staphylococcaceae, Streptococcus, Streptococcaceae, Pseudomonadaceae, Haemophilus, Fusobacteriaceae, Pasteruellaceae, Prevotellaceae, Fusobacteriaceae, Neisseriaceae, and/or Enterobacteriaceae.

In an aspect, a method of detecting nasal dysbiosis in a subject is provided. In embodiments, the method includes detecting a plurality of microorganisms in a biological sample from the subject.

In an aspect, a method of detecting sinus dysbiosis in a subject is provided. In embodiments, the method includes detecting a plurality of microorganisms in a biological sample from the subject.

In an aspect, a method of detecting whether a subject who has asthma has an increased risk of asthma exacerbation compared to a general population of subjects who have asthma. In embodiments, the method includes detecting a plurality of microorganisms in a biological sample from the subject.

In an aspect, a method of detecting whether a subject who has asthma has an increased risk of rhinovirus infection compared to a general population of subjects who have asthma. In embodiments, the method includes detecting a plurality of microorganisms in a biological sample from the subject.

In an aspect, a method of detecting whether a subject has chronic rhinosinusitis is provided. In embodiments, the method includes detecting a plurality of microorganisms in a biological sample from the subject.

In an aspect, a method of detecting whether a subject is at risk of developing chronic rhinosinusitis is provided. In embodiments, the method includes detecting a plurality of microorganisms in a biological sample from the subject.

In an aspect, method of detecting whether a subject who has chronic rhinosinusitis has an increased risk of nasal polyposis compared to a general population of subjects who have chronic rhinosinusitis is provided. In embodiments, the method includes detecting a plurality of microorganisms in a biological sample from the subject.

In an aspect, a method of detecting a nasal or sinus microbiome in a subject who has asthma is provided. In embodiments, the method includes (a) obtaining a biological sample from the subject; and (b) detecting bacteria, or a proportion of bacteria, in the biological sample that are in 1 of or any combination of the following taxa: Moraxella, Staphylococcaceae, Corynebacterium, Corynebacteriaceae, Staphylococcus, Staphylococcaceae, Streptococcus, Streptococcaceae, Pseudomonadaceae, Haemophilus, Fusobacteriaceae, Pasteruellaceae, Prevotellaceae, Fusobacteriaceae, Neisseriaceae, and/or Enterobacteriaceae.

In an aspect, a method of detecting nasal dysbiosis in a subject is provided. In embodiments, the method includes (a) obtaining a biological sample from the subject; and (b) detecting a plurality of microorganisms in the biological sample.

In an aspect, a method of detecting sinus dysbiosis in a subject is provided. In embodiments, the method includes (a) obtaining a biological sample from the subject; and (b) detecting a plurality of microorganisms in the biological sample.

In an aspect, a method of detecting whether a subject who has asthma has an increased risk of asthma exacerbation compared to a general population of subjects who have asthma. In embodiments, the method includes (a) obtaining a biological sample from the subject; and (b) detecting a plurality of microorganisms in the biological sample.

In an aspect, a method of detecting whether a subject who has asthma has an increased risk of rhinovirus infection compared to a general population of subjects who have asthma. In embodiments, the method includes (a) obtaining a biological sample from the subject; and (b) detecting a plurality of microorganisms in the biological sample.

In an aspect, a method of detecting whether a subject has chronic rhinosinusitis is provided. In embodiments, the method includes (a) obtaining a biological sample from the subject; and (b) detecting a plurality of microorganisms in the biological sample.

In an aspect, a method of detecting whether a subject is at risk of developing chronic rhinosinusitis is provided. In embodiments, the method includes (a) obtaining a biological sample from the subject; and (b) detecting a plurality of microorganisms in the biological sample.

In an aspect, method of detecting whether a subject who has chronic rhinosinusitis has an increased risk of nasal polyposis compared to a general population of subjects who have chronic rhinosinusitis is provided. In embodiments, the method includes (a) obtaining a biological sample from the subject; and (b) detecting a plurality of microorganisms in the biological sample.

In an aspect, a method of treating or preventing asthma, an asthma exacerbation, or a rhinovirus infection in a subject in need thereof is provided. In embodiments, the method includes detecting nasal dysbiosis in the subject, and administering to the subject an effective amount of an antibiotic compound.

In an aspect, a method of treating or preventing asthma, an asthma exacerbation, or a rhinovirus infection in a subject in need thereof is provided. In embodiments, the method includes administering to the subject an effective amount of an antibiotic compound, wherein the subject has been identified as having an increased risk of asthma exacerbation or rhinovirus infection compared to a general population of subjects who have asthma.

In an aspect, a method of treating or preventing asthma, an asthma exacerbation, or a rhinovirus infection in a subject in need thereof is provided. In embodiments, the method includes administering to the subject an effective amount of an antibiotic compound.

In an aspect, a method of treating or preventing chronic rhinosinusitis or nasal polyposis in a subject in need thereof is provided. In embodiments, the method includes detecting sinus dysbiosis in the subject, and administering to the subject an effective amount of an antibiotic compound.

In an aspect, a method of treating or preventing chronic rhinosinusitis or nasal polyposis in a subject in need thereof is provided. In embodiments, the method includes administering to the subject an effective amount of an antibiotic compound, wherein the subject has been identified as having of chronic rhinosinusitis or nasal polyposis or at risk of chronic rhinosinusitis or nasal polyposis.

In an aspect, a method of treating or preventing chronic rhinosinusitis or nasal polyposis in a subject in need thereof is provided. In embodiments, the method includes administering to the subject an effective amount of an antibiotic compound.

In an aspect, a method of treating or preventing chronic rhinosinusitis or nasal polyposis in a subject in need thereof is provided. In embodiments, the method includes administering to the subject an effective amount of an anti-IL-5 compound.

In an aspect, a method of treating or preventing asthma, asthma exacerbation, or a rhinovirus infection in a subject in need thereof is provided. In embodiments, the method includes detecting nasal dysbiosis in the subject, and administering to the subject an effective amount of at least one bacterium.

In an aspect, a method of treating or preventing asthma, an asthma exacerbation, or a rhinovirus infection in a subject in need thereof is provided. In embodiments, the method includes administering to the subject an effective amount of at least one bacterium, wherein the subject has been identified as having an increased risk of asthma exacerbation or rhinovirus infection compared to a general population of subjects who have asthma.

In an aspect, a method of treating or preventing asthma, an asthma exacerbation, or a rhinovirus infection in a subject in need thereof is provided. In embodiments, the method includes administering to the subject an effective amount of at least one bacterium.

In an aspect, a method of treating or preventing chronic rhinosinusitis or nasal polyposis in a subject in need thereof is provided. In embodiments, the method includes detecting sinus dysbiosis in the subject, and administering to the subject an effective amount of at least one bacterium.

In an aspect, a method of treating or preventing chronic rhinosinusitis or nasal polyposis in a subject in need thereof is provided. In embodiments, the method includes administering to the subject an effective amount of at least one bacterium, wherein the subject has been identified as having of chronic rhinosinusitis or nasal polyposis or at risk of chronic rhinosinusitis or nasal polyposis.

In an aspect, a method of treating or preventing chronic rhinosinusitis or nasal polyposis in a subject in need thereof is provided. In embodiments, the method includes administering to the subject an effective amount of at least one bacterium.

In an aspect, a method of reducing the amount of Corynebacterium sp. bacteria or Pseudomonas sp. bacteria in the sinus microbiome of a subject is provided. In embodiments, the method comprises administering to the subject an effective amount of a Lactobacillus sakei bacterium. In embodiments, the subject has rhinosinusitis (e.g., chronic rhinosinusitis) or nasal polyposis. In embodiments, there is an increased proportion or amount of Corynebacterium sp. bacteria or Pseudomonas sp. bacteria in the sinus microbiome of the subject compared to a standard control.

In an aspect, a method of treating or preventing an infection of Corynebacterium sp. bacteria or Pseudomonas sp. bacteria in the sinus of a subject in need thereof is provided. In embodiments, the method comprises administering to the subject an effective amount of a Lactobacillus sakei bacterium.

In an aspect, a method of treating or preventing chronic rhinosinusitis or nasal polyposis in a subject in need thereof is provided. In embodiments, the method includes detecting an increased proportion or amount of Corynebacterium sp. bacteria or Pseudomonas sp. bacteria in the sinus microbiome of the subject compared to a standard control, and administering to the subject an effective amount of a Lactobacillus sakei bacterium.

In an aspect, a method of treating or preventing acute sinusitis in a subject in need thereof is provided. In embodiments, the method comprises administering to the subject an effective amount of a Lactobacillus sakei bacterium.

In an aspect, a method of increasing bacterial divsersity in the sinus of a subject in need thereof is provided. In embodiments, the method comprises administering to the subject an effective amount of a Lactobacillus sakei bacterium.

In an aspect, a method of treating or preventing chronic rhinosinusitis or nasal polyposis in a subject in need thereof is provided. In embodiments, the method includes administering to the subject an effective amount of a Lactobacillus sakei bacterium, wherein the subject has been identified as having an increased proportion or amount of Corynebacterium sp. bacteria or Pseudomonas sp. bacteria in the sinus microbiome of the subject compared to a standard control. The “proportion” of a bacterial type (e.g., family, genus, species, or other taxon) in a population of bacterial cells is the percentage of the total number of bacterial cells that are of the bacterial type.

In an aspect, a method of treating or preventing chronic rhinosinusitis or nasal polyposis in a subject in need thereof is provided. In embodiments, the method includes administering to the subject an effective amount of a Lactobacillus sakei bacterium, wherein the subject has an increased proportion or amount of Corynebacterium sp. bacteria or Pseudomonas sp. bacteria in the sinus microbiome of the subject compared to a standard control.

In an aspect, a method of treating or preventing dysbiosis in a subject in need thereof is provided. In embodiments, the method includes administering to the subject an effective amount of a Lactobacillus sakei bacterium.

In an aspect, a composition that includes an isolated Lactobacillus sakei bacterium and a pharmaceutically acceptable excipient is provided.

### BRIEF DESCRIPTION OF THE DRAWINGS

FIG. 1. Multiple taxa from specific bacterial genera are associated with exacerbation and RV. Specific bacterial heat-map indicating taxa that exhibit significantly higher (black) or lower (gray) relative abundance in participants with an exacerbation (vs non-exacerbation); RV (vs non-RV samples); RV-A infection, RV-B infection, or RV-C infection (vs non-RV samples), using Poisson, Negative Binomial or Zero-Inflated Negative Binomial mixed-effects models. Bacterial taxa are ordered according to their position in the phylogenetic tree generated during the OTU-picking process; OTUs closer together are more genetically similar than those further apart. Significance was defined as a false discovery q-value <0.1; sign of the coefficient was used to determine directionality of the relationship.

FIGS. 2A-B. Compositionally distinct nasal bacterial community states exist in children with asthma. FIG. 2A: The majority of asthmatic nasal samples are dominated by taxa belonging to either Moraxella or Staphylococcaceae, with smaller proportions of samples dominated by Streptococcus, Alloiococcus, Corynebacterium, Haemophilus or other genera. FIG. 2B: Moraxella and Haemophilus-dominated communities exhibit significantly lower bacterial diversity compared to all other microbiota states.

FIG. 3. Staphylococcaceae and Moraxella-dominated nasal community states exhibit the greatest temporal stability in children with asthma. Heat map indicates the frequency of transition from a community state at time point 1 (x-axis) to a community state in the subsequent sample (Time point 2; y-axis). The proportion of transitions is provided within each square and color coded. The diagonal represents sample transitions that remained in the same state for subsequent samples. Data generated from longitudinally collected sample transitions (n=2,709) from all participants (n=413).

FIG. 4. Age and ECP concentration at randomization relates to the dominant genus of the first sample collected from each individual. Numbers below boxplots indicate the total number of samples included for each community state. Significant differences across community states (q<0.05) are indicated with *.

FIGS. 5A-5B. Temporal stability does not associate differentially between children who experience an exacerbation and those who do not. FIG. 5A: Calculated distance between an individuals' first sample and all other subsequent samples, stratified by exacerbation status. FIG. 5B: Average between-sample distance for each individual, stratified by exacerbation status.

FIGS. 6A-6B. Transition probabilities are consistent over different methods of reducing potential bias due to uneven sampling. Frequency of inter- or intra-community state transitions across samples collected (A) between 7 and 13 days apart (n=1536 transitions from N=390 participants), and (B) the first three samples (n=826 transitions from N=413 participants) collected from each child with asthma. The color intensity reflects the frequency with which community states transition to the same or a distinct state.

FIG. 7. Study design and microbiome sample collection for the Preventive Omalizumab and Step-Up Therapy for Severe Fall Exacerbations (PROSE) study, 2012-2013.

FIGS. 8A-8D. CRS patients (NonCF-CRS and CF-CRS) exhibit similar total bacterial burden compared with healthy subjects, however their microbiota exhibit significantly reduced richness, evenness and diversity. Comparative analyses of sinus microbiota FIG. 8A: bacterial burden; FIG. 8B: richness (FDR p=0.006 [nonCF-CRS(−)Asthma v. healthy], p=0.0015 [nonCF-CRS(+)Asthma v. healthy] and p=0.0015 [CF-CRS v. healthy]; permutation t-test); FIG. 8C: Pielou's evenness (FDR p=0.132 [nonCF-CRS(−)Asthma], FDR p=0.015 [nonCF-CRS(+)Asthma v. healthy], and p=0.003 [CF-CRS v. healthy]; permutation t-test), and FIG. 8D: Faith's phylogenetic diversity (FDR p=0.007 [nonCF-CRS(−)Asthma v. healthy], FDR p=0.003 [nonCF-CRS(+)Asthma v. healthy], and p=0.003 [CF-CRS v. healthy]; permutation t-test), indices using V4 16S rRNA amplicon sequencing of healthy, nonCF-CRS and CF-CRS patients. Values represent the median+/−1.5 IQR.

FIGS. 9A-9E. Dirichlet Multinomial Mixtures modeling identifies microbial states that explain a large portion of variation in microbiota composition. FIG. 9A: Distribution of co-morbidities (CF or physician-diagnosed asthma) significantly differ across microbiota states. DSI was represented by CRS patients and 9/10 of the healthy controls. DSII was enriched for CRS+CF and CRS+A patients, whereas DSIII was comprosed primarily of CRS patients without concomitant lower airway disease, and one healthy control subject (Chi-squared; p=0.0007); FIGS. 9C-9E: Three-model testing of differential taxon abundance indicates that each Dirichlet State (DS) is associated with enrichment for specific taxa and co-colonizers and depletion of microbiota associated with healthy individuals (ZINB; p<0.05, q<0.10).

FIGS. 10A-10D. Variation in predicted metagenomes associated with each Dirichlet state. FIG. 10A: DSII is significantly functionally depleted, measured by total unique KEGG pathways compared to healthy individuals (permutational t-test, FDR p=0.0025); FIG. 10B: Tryptophan metabolism is enriched in DSII [Pseudomonadaceae-defined, (Negative Binomial p=0.021, q=0.059)] and DSIII(b) [Staphylococcaceae-defined (Negative Binomial p=0.005, q=0.012)] FIG. 10C: The two-component response system virulence pathway is enriched in DSII [Pseudomonadaceae-defined, (Negative Binomial p=0.0002, q=0.002)] and DSIII(b) [Staphylococcaceae-defined (Negative Binomial p=0.0002, q=0.002)] FIG. 10D: PPAR-gamma signaling pathway is enriched in DSIII(a) [Corynebacteriaceae-defined (Negative Binomial p=0.003, q=0.0175)].

FIGS. 11A-11C. Microbial states confer a differential risk for polyposis and are significantly associated with distinct profiles of host immune response. FIG. 11A: Multivariate permutation (PERMANOVA) testing of presence or absence of polyps at time of surgery does not indicate a significant relationship at the whole-community level (p=0.152, 2.5% variation explained) FIG. 11B: Patients with DSIII(a) have a significantly increased risk for polyposis (88.9% of patients have polyps; Fisher's Exact p=0.032, RR=2.159 compared to DSI). FIG. 11C: Heatmap of Z score-normalized mean fold change (2-AACt) for each gene examined indicates that immune responses distinct from that of healthy subjects are evident in CRS patients; samples are grouped by DS and healthy individuals (* indicates Kruskal Wallis p<0.05, q<0.15; ** indicates Kruskal Wallis p<0.05, q<0.05; DS vs. non-CRS).

FIGS. 12A-12B. FIG. 12A: Lund MacKay scores associated with disease state. No differences were observed between CF-CRS patients and CRS patients with or without asthma; FIG. 12B: CF-CRS patients are significantly younger than non-CF CRS patients with asthma (ANOVA, Tukey's p=0.041), however, no differences in age were observed for pairwise comparisons between the other groups (p>0.05, Tukey's post hoc test).

FIGS. 13A-13B. FIG. 13A: Laplace model fit demonstrates three distinct Dirichlet multinomial mixtures groups. FIG. 13B: Reciprocal relationship between Corynebacteriaceae and Staphylococcaceae.

FIG. 14. Expression levels of all host immune genes measured by QPCR (* indicates Kruskal Wallis p<0.05, q<0.15; ** indicates Kruskal Wallis p<0.05, q<0.05; DS vs. non-CRS).

### DETAILED DESCRIPTION

### I. Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “isolated”, when applied to a bacterium, refers to a bacterium that has been (1) separated from at least some of the components with which it was associated when initially produced (whether in nature or in an experimental setting), and/or (2) produced, prepared, purified, and/or manufactured by the hand of man, e.g. using artificial culture conditions such as (but not limited to) culturing on a plate and/or in a fermenter. Isolated bacteria include those bacteria that are cultured, even if such cultures are not monocultures. In embodiments, isolated bacteria are in a monoculture. Isolated bacteria may be separated from at least about 10%, about 20%, about 30%, about 40%, about 50%, about 60%, about 70%, about 80%, about 90%, or more of the other components with which they were initially associated (e.g., by weight). In embodiments, isolated bacteria are more than about 80%, about 85%, about 90%, about 91%, about 92%, about 93%, about 94%, about 95%, about 96%, about 97%, about 98%, about 99%, or more than about 99% pure (e.g., by weight). In embodiments, a bacterial population provided herein includes isolated bacteria. In embodiments, a composition provided herein includes isolated bacteria. In embodiments, the bacteria that are administered are isolated bacteria.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Patient” or “subject in need thereof” refers to a living member of the animal kingdom suffering from or that may suffer from the indicated disorder. In embodiments, the subject is a member of a species that includes individuals who naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human. In embodiments, the subject is a non-mammalian animal such as a turkey, a duck, or a chicken. In embodiments, a subject is a living organism suffering from or prone to a disease or condition that can be treated by administration of a composition or pharmaceutical composition as provided herein.

As used herein, a “symptom” of a disease includes any clinical or laboratory manifestation associated with the disease, and is not limited to what a subject can feel or observe.

As used herein the term “dysbiosis” means a difference in the microbiota compared to a general or healthy population. The term “nasal dysbiosis” means a difference in the nasal microbiota compared to a general or healthy population. In embodiments, dysbiosis includes a difference in nasal microbiota commensal species diversity compared to a general or healthy population. In embodiments, dysbiosis includes a decrease of beneficial microorganisms and/or increase of pathobionts (pathogenic or potentially pathogenic microorganisms) and/or decrease of overall microbiota species diversity. Many factors can harm the beneficial members of the nasal microbiota leading to dysbiosis, including (but not limited to) infection, antibiotic use, psychological and physical stress, radiation, and dietary changes. In embodiments, the dysbiosis includes a reduced amount (absolute number or proportion of the total microbial population) of bacterial or fungal cells of a species or genus (e.g., 5%, 10%, 15%, 20%, 25%, 30%, 35%, 40%, 45%, 50%, 55%, 60%, 65%, 70%, 75%, 80%, 85%, 90%, 95% or more lower) compared to a healthy subject (e.g., a corresponding subject who does not have asthma or an infection, and who has not been administered an antibiotic within about 1, 2, 3, 4, 5,or 6 months, and/or compared to a general or healthy population). In embodiments, the dysbiosis includes an increased amount (absolute number or proportion of the total microbial population) of bacterial or fungal cells within a species or genus (e.g., 5%, 10%, 15%, 20%, 25%, 30%, 35%, 40%, 45%, 50%, 55%, 60%, 65%, 70%, 75%, 80%, 85%, 90%, 95% or more higher) compared to a healthy subject (e.g., a corresponding subject who does not have asthma or an infection, and who has not been administered an antibiotic within about 1, 2, 3, 4, 5, or 6 months, and/or compared to a general or healthy population). In embodiments, a subject who has asthma or who has received an antibiotic within about 1, 2, 3, 4, 5, 6, 7, 8, 9, or 10 weeks is deemed to have nasal dysbiosis. In embodiments, antibiotic administration (e.g., systemically, such as by intravenous injection or orally) is causing or has caused a major alteration in the normal microbiota. Thus, as used herein, the term “antibiotic-induced dysbiosis” refers to dysbiosis caused by or following the administration of an antibiotic. In embodiments, an antibiotic is administered, but the subject has dysbiosis at the time of administration.

As used herein the term “sinus dysbiosis” means a difference in the sinus microbiota compared to a general or healthy population. In embodiments, dysbiosis includes a difference in sinus microbiota commensal species diversity compared to a general or healthy population. In embodiments, dysbiosis includes a decrease of beneficial microorganisms and/or increase of pathobionts (pathogenic or potentially pathogenic microorganisms) and/or decrease of overall microbiota species diversity. Many factors can harm the beneficial members of the sinus microbiota leading to dysbiosis, including (but not limited to) infection, antibiotic use, psychological and physical stress, radiation, and dietary changes. In embodiments, the dysbiosis includes a reduced amount (absolute number or proportion of the total microbial population) of bacterial or fungal cells of a species or genus (e.g., 5%, 10%, 15%, 20%, 25%, 30%, 35%, 40%, 45%, 50%, 55%, 60%, 65%, 70%, 75%, 80%, 85%, 90%, 95% or more lower) compared to a healthy subject (e.g., a corresponding subject who does not have rhinosinusitis (such as chronic rhinosinusitis) or an infection, and who has not been administered an antibiotic within about 1, 2, 3, 4, 5, or 6 months, and/or compared to a general or healthy population). In embodiments, the dysbiosis includes an increased amount (absolute number or proportion of the total microbial population) of bacterial or fungal cells within a species or genus (e.g., 5%, 10%, 15%, 20%, 25%, 30%, 35%, 40%, 45%, 50%, 55%, 60%, 65%, 70%, 75%, 80%, 85%, 90%, 95% or more higher) compared to a healthy subject (e.g., a corresponding subject who does not have rhinosinusitis (such as chronic rhinosinusitis) or an infection, and who has not been administered an antibiotic within about 1, 2, 3, 4, 5, or 6 months, and/or compared to a general or healthy population). In embodiments, a subject who includes a chronic rhinosinusitis infection or who has received an antibiotic within about 1, 2, 3, 4, 5, 6, 7, 8, 9, or 10 weeks is deemed to include sinus dysbiosis. In embodiments, antibiotic administration (e.g., systemically, such as by intravenous injection or orally) is causing or has caused a major alteration in the normal microbiota. Thus, as used herein, the term “antibiotic-induced dysbiosis” refers to dysbiosis caused by or following the administration of an antibiotic.

Non-limiting examples of nasal dysbiosis are described in the examples provided herein. In some embodiments, a subject with nasal dysbiosis has the a microbiome profile as set forth in Example 1.

Non-limiting examples of sinus dysbiosis are described in the examples provided herein. In some embodiments, a subject with sinus dysbiosis has the a microbiome profile as set forth in Example 2.

Non-limiting examples of sinus dysbiosis are also described in Cope et al. (2017) “Compositionally and functionally distinct sinus microbiota in chronic rhinosinusitis patients have immunological and clinically divergent consequences” Microbiome 5:53, PMID: 28494786, PMCID: PMC5427582 (hereinafter “Cope et al. 2017”), the entire content of which (including all supplemental information and data) is incorporated herein by reference. In some embodiments, a subject with dysbiosis has the DSI, DSII, DSIII(a), or DSIII(b) microbiome profile as set forth in Cope et al. 2017.

A “control” or “standard control” refers to a sample, measurement, or value that serves as a reference, usually a known reference, for comparison to a test sample, measurement, or value. For example, a test sample can be taken from a patient suspected of having a given disease (e.g. dysbiosis, asthma, rhinosinusitis, chronic rhinosinusitis, nasal polyposis, or other disease, such as an infection, e.g., a rhinovirus infection) and compared to a known normal (non-diseased) individual (e.g. a standard control subject). A standard control can also represent an average measurement or value gathered from a population of similar individuals (e.g. standard control subjects) that do not have a given disease (e.g. standard control population), e.g., healthy individuals with a similar medical background, same age, weight, etc. In embodiments, a standard control is a proportion, level, or amount (e.g., an average proportion, level, or amount) in a general or healthy population of subjects. In embodiments, a standard control is a proportion, level, or amount (e.g., an average proportion, level, or amount) in a general population of subjects. In embodiments, a standard control is a proportion, level, or amount (e.g., an average proportion, level, or amount) in a healthy population of subjects. In embodiments, a general population of subjects is a general population of subjects in a geographical area (such as a country or continent, e.g., Asia, Australia, Africa, North America, South America, or Europe). In embodiments, a general population of subjects is a general population of subjects in (e.g., that self-identify as being within) an ethnic group such as caucasian (e.g., white), African, of African descent (e.g., African American), Native American, Asian, or of Asian descent. In embodiments, a general population of subjects is a general population of subjects without a disease such as asthma, a rhinovirus infection, rhinosinusitis (e.g., chronic rhinosinusitis), or nasal polyposis. In embodiments, a general population of subjects is a general population of subjects with a disease such as asthma, a rhinovirus infection, rhinosinusitis (e.g., chronic rhinosinusitis), or nasal polyposis. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. microbiome, RNA levels, protein levels, specific cell types, specific bodily fluids, specific tissues, metabolites, etc.).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The term “diagnosis” refers to a determination or relative probability that a disease (e.g. dysbiosis, asthma, rhinosinusitis, chronic rhinosinusitis, nasal polyposis, or other disease, such as an infection, e.g., a rhinovirus infection infection) is present in the subject. In embodiments, a subject is diagnosed with a disease when the disease has been detected (e.g., with an assay) in a subject. Similarly, the term “prognosis” refers to a relative probability that a certain future outcome may occur in the subject with respect to a disease state. For example, in the context of the present disclosure, prognosis can refer to the likelihood that an individual will develop a disease (e.g. dysbiosis, asthma, rhinosinusitis, chronic rhinosinusitis, nasal polyposis, or other disease, such as an infection, e.g., a rhinovirus infection infection), or the likely severity of the disease (e.g., duration of disease). The terms are not intended to be absolute, as will be appreciated by any one of skill in the field of medical diagnostics.

“Biological sample” or “sample” refers to materials obtained from or derived from a subject or patient. In embodiments, a biological sample is or includes a bodily fluid such as nasal discharge or mucus. In embodiments, a biological sample is or includes a wash, such as a saline wash, e.g., a nasal saline wash. In embodiments, the biological sample is or includes mucus. In embodiments, the mucus is sinus mucus (e.g., mucus obtained or collected from the surface of a sinus). In embodiments, the biological sample is a sinus brushing including mucus from the surface of a sinus. In embodiments, a biological sample is or includes sputum, phlegm, saliva, or mucus. In embodiments, a biological sample is or includes blood, serum, or plasma. In embodiments, a biological samples is or includes blood, a blood fraction, or product (e.g., serum, plasma, platelets, red blood cells, and the like). In embodiments, a biological sample is or includes tissue, such as nasal or sinus tissue. In embodiments, a sample is obtained from a eukaryotic organism, such as a mammal such as a primate e.g., chimpanzee or human; cow; dog; cat; a rodent, e.g., guinea pig, rat, or mouse; rabbit; or a bird; reptile; or fish. In embodiments, a biological sample includes sections of tissues such as biopsy and autopsy samples, and frozen sections taken for histological purposes.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein the abbreviation “sp.” for species means at least one species (e.g., 1, 2, 3, 4, 5, or more species) of the indicated genus. The abbreviation “spp.” for species means 2 or more species (e.g., 2, 3, 4, 5, 6, 7, 8, 9, 10 or more) of the indicated genus. In embodiments, methods and compositions provided herein include a single species within an indicated genus or indicated genera, or 2 or more (e.g., a plurality including more than 2) species within an indicated genus or indicated genera. In embodiments, 1, 2, 3, 4, 5, or more or all or the indicated species is or are isolated. In embodiments, the indicated species are administered together. In embodiments, each of the indicated species is present in a single composition that includes each of the species. In embodiments, each of the species is administered concurrently, e.g., within about 1, 2, 3, 4, 5, 6, 7, 8, 9, 10, 30, or 60, 1-5, 1-10, 1-30, 1-60, or 5-15 seconds or minutes of each other.

The phrase “stringent hybridization conditions” refers to conditions under which a primer or probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium).

Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

In embodiments, nucleic acids that do not hybridize to each other under stringent conditions are still considdered substantially identical if the polypeptides which they encode are substantially identical. This occurs, for example, when a copy of a nucleic acid is created using the maximum codon degeneracy permitted by the genetic code. In embodiments, the nucleic acids hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

In embodiments, detecting includes an assay. In embodiments, the assay is an analytic procedure to qualitatively assess or quantitatively measure the presence, amount, or functional activity of an entity, element, or feature (e.g., a compound, a level of gene expression, a bacterial type or taxon, or a bacterial population such as in a microbiome). In embodiments, assaying the level of a compound (such as a protein, an mRNA molecule, or a metabolite) includes using an analytic procedure (such as an in vitro procedure) to qualitatively assess or quantitatively measure the presence or amount of the compound.

In this disclosure, “comprises,” “comprising,” “containing,” and “having” and the like can have the meaning ascribed to them in U.S. Patent law and can mean “includes,” “including,” and the like. Thus, the transitional term “comprising,” which is synonymous with “including,” “containing,” or “characterized by,” is inclusive or open-ended and does not exclude additional, unrecited features, integers, steps, operations, elements, and/or components. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. By contrast, the transitional phrase “consisting of” excludes any feature, integer, element, step, operation, component, and/or ingredient not specified.

As used herein, the term “about” in the context of a numerical value or range means±10% of the numerical value or range recited or claimed, unless the context requires a more limited range.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

### II. Methods of Detecting

In an aspect, a method of detecting nasal dysbiosis in a subject is provided. In embodiments, the method includes detecting a plurality of microorganisms in a biological sample from the subject.

In an aspect, a method of detecting a nasal or sinus microbiome in a subject who has asthma is provided. In embodiments, the method includes detecting bacteria, or a proportion of bacteria, in a biological sample from the subject that are in 1 of or any combination of the following taxa: Moraxella, Staphylococcaceae, Corynebacterium, Corynebacteriaceae, Staphylococcus, Staphylococcaceae, Streptococcus, Streptococcaceae, Pseudomonadaceae, Haemophilus, Fusobacteriaceae, Pasteruellaceae, Prevotellaceae, Fusobacteriaceae, Neisseriaceae, and/or Enterobacteriaceae.

In an aspect, a method of detecting sinus dysbiosis in a subject is provided. In embodiments, the method includes detecting a plurality of microorganisms in a biological sample from the subject.

In embodiments, distinct pathogenic microbiota exist in patients with a disease or disorder, e.g., asthma, nasal polyposis, chronic rhinosinusitis, infection, and dysbiosis. In embodiments, distinct microbiota states induce distinct but reproducible immune dysfunction and are associated with significant differences in clinical responses. In embodiments, this approach is used to stratify patients in cohorts of patients with distinct infectious diseases, risks, diagnoses, and/or prognoses.

In embodiments, methods provided herein provide for the detection or understanding of patient heterogeneity. In embodiments, methods included herein tailor therapy to the specific microbiota dysbiosis and immune dysfunction presented by the patient.

In an aspect, a precision medicine application is provided. In embodiments, patient samples are tested to identify the specific pathogenic microbiota and therapy is tailored based on test results.

Current approaches to stratify patients fail to consider the pathogenic microbiota that is responsible for the observed immune dysfunction. Approaches provided herein permit identification of patient microbial endotypes whose treatment either stand alone or as an adjuvant to existing therapy will improve patient outcomes. In embodiments, methods provided herein provide relatively inexpensive technology, relatively rapid turn-around, opportunity for precision medicine.

In embodiments, a shotgun metagenomics and/or transcriptomic approach is used to identify, characterize, detect, or determine a metagenome. In embodiments, a shotgun metagenomics and/or transcriptomic approach is used to identify, characterize, detect, or determine viral and fungal taxa in a subject. In embodiments, metagenomics, in parallel with metabolomics and transcriptomics is used to stratify subjects based on their microbiomes.

In embodiments, by examining microbial population (e.g., characterizing a microbiome), a diverse immune profile that exists within a patient population is identified, characterized, detected, or determined. In embodiments, the microbial and immunological features described herein inform strategies for tailored therapy in a patient population.

In an aspect, a method of detecting whether a subject who has asthma has an increased risk of asthma exacerbation compared to a general population of subjects who have asthma. In embodiments, the method includes detecting a plurality of microorganisms in a biological sample from the subject.

In an aspect, a method of detecting whether a subject who has asthma has an increased risk of rhinovirus infection compared to a general population of subjects who have asthma. In embodiments, the method includes detecting a plurality of microorganisms in a biological sample from the subject.

In embodiments, the method includes obtaining the biological sample from the subject. In embodiments, obtaining the biological sample from the subject comprises collecting the biological sample directly from the subject. In embodiments, obtaining the biological sample from the subject comprises receiving a biological sample that has been collected (e.g, directly) from the subject (e.g., by another actor, such as a clinical professional such as a nurse, medic, or doctor). In embodiments, the biological sample has been submitted by the subject (e.g., by mail or currior).

In embodiments, the biological sample is a nasal saline wash.

In embodiments, the biological sample is a bodily fluid.

In embodiments, the bodily fluid is nasal mucus or discharge.

In embodiments, the microorganisms are bacterial microorganisms.

In embodiments, the subject is 1-20 years old (e.g., about or at least 1, 2, 3, 4, 5, 6, 7, 8, 9, 10, 11, 12, 13, 14, 15, 16, 17, 18, 19, or 20 years old, or 1-5, 5-10, 10-15, 15-20, 3-6, 6-9, or 6-18 years old). In embodiments, the subject is 6-18 years old.

In embodiments, a monoclonal antibody targeting the high-affinity receptor binding site on human immunoglobulin (Ig)E was previously administered to the subject. In embodiments, omalizumab was previously administered to the subject.

In embodiments, the biological sample is obtained in June, July, August, or September.

In embodiments, the rhinovirus infection is a rhinovirus A infection or a rhinovirus B infection.

In embodiments, detecting a plurality of microorganisms in the biological sample includes characterizing a microbiome in the biological sample.

In embodiments, characterizing the microbiome in the biological sample includes determining the number and/or identity of bacterial taxa represented by bacteria in the biological sample. In embodiments, the bacterial taxa include bacterial families, genera, and/or species. In embodiments, the bacterial taxa comprise, consist essentially of, or consist of bacterial families. In embodiments, the bacterial taxa are bacterial families. In embodiments, the bacterial taxa comprise, consist essentially of, or consist of bacterial genera. In embodiments, the bacterial taxa are bacterial genera. In embodiments, the bacterial taxa comprise, consist essentially of, or consist of bacterial species. In embodiments, the bacterial taxa are bacterial species. In embodiments, the bacterial taxa are bacterial families and/or genera.

In embodiments, characterizing the microbiome in the biological sample includes detecting the proportion of bacteria in the biological sample that are within 1 of or any combination of 2, 3, 4, 5, 6, 7, 8, 9, or 10 of the following taxa: Moraxella, Staphylococcaceae, Corynebacterium, Staphylococcus, Streptococcus, and/or Haemophilus.

In embodiments, detecting a plurality of microorganisms in the biological sample includes amplifying and sequencing 16S rRNA genes, or portions thereof, of microorganisms in the sample.

In embodiments, detecting a plurality of microorganisms in the biological sample includes amplifying and sequencing the V4 region of 16S rRNA genes of microorganisms in the sample.

In embodiments, the method includes detecting the proportion of bacteria in the biological sample that are within 1 of or any combination of 2, 3, 4, 5, 6, 7, 8, 9, or 10 of the following taxa: Moraxella, Staphylococcaceae, Corynebacterium, Staphylococcus, Streptococcus, and/or Haemophilus.

In embodiments, the method includes detecting the proportion of bacteria in the biological sample that are within 1 of or any combination of 2, 3, 4, 5, 6, 7, 8, 9, or 10 of the following families: Corynebacteriaceae, Staphylococcaceae, Pseudomonadaceae, Streptococcaceae, Fusobacteriaceae, Pasteruellaceae, Prevotellaceae, Fusobacteriaceae, Neisseriaceae, and/or Enterobacteriaceae.

In embodiments, the method includes detecting whether the biological sample has an increased proportion of bacteria in the Moraxella genus of bacteria compared to a general population of subjects who have asthma. In embodiments, the method includes detecting whether the biological sample has an increased proportion of bacteria in the Neisseria genus of bacteria compared to a general population of subjects who have asthma. In embodiments, the method includes detecting whether the biological sample has a decreased proportion of bacteria in the Staphylococcaceae family or Corynebacterium genus of bacteria compared to a general population of subjects who have asthma. In embodiments, the method includes detecting whether the biological sample has a decreased proportion of bacteria in the Corynebacterium genus of bacteria compared to a general population of subjects who have asthma. In embodiments, the method includes detecting whether the biological sample has decreased proportion of bacteria in the Haemophilus genus of bacteria compared to a general population of subjects who have asthma. In embodiments, the method includes detecting whether the biological sample has decreased proportion of bacteria in the Staphylococcaceae family of bacteria compared to a general population of subjects who have asthma. In embodiments, the method includes detecting whether the biological sample has an increased proportion of bacteria in the Staphylococcus genus or Corynebacterium genus of bacteria compared to a general population of subjects who have asthma. In embodiments, the method includes detecting whether the biological sample has an increased proportion of bacteria in the Streptococcus genus of bacteria compared to a general population of subjects who have asthma. In embodiments, the method includes detecting whether the biological sample has an increased proportion of bacteria in the Haemophilus genus of bacteria compared to a general population of subjects who have asthma.

In embodiments, the method includes detecting whether the subject has an increased proportion of nasal microbiome bacteria in the Haemophilus genus of bacteria compared to a general population of subjects who have asthma. In embodiments, the method includes detecting whether the subject has an increased proportion of nasal microbiome bacteria in the Streptococcus genus of bacteria compared to a general population of subjects who have asthma. In embodiments, the method includes detecting whether the subject has an increased proportion of nasal microbiome bacteria in the Moraxella genus of bacteria compared to a general population of subjects who have asthma. In embodiments, the method includes detecting whether the subject has an increased proportion of nasal microbiome bacteria in the Neisseria genus of bacteria compared to a general population of subjects who have asthma. In embodiments, the method includes detecting whether the subject has a decreased proportion of nasal microbiome bacteria in the Staphyloccocaceae family of bacteria compared to a general population of subjects who have asthma.

In embodiments, the method includes detecting whether the biological sample has an increased proportion of bacteria in the Prevotellaceae and Fusobacteriaceae families of bacteria compared to a general or healthy population of subjects. In embodiments, the method includes detecting whether the biological sample has an increased proportion of bacteria in the Pseudomonas genus of bacteria compared to a general or healthy population of subjects. In embodiments, the method includes detecting whether the biological sample has an increased proportion of bacteria in the Streptococcus, Porphyromonas, Tannerella, Treponema, Bacteroides, Dialister, and Akkermansia taxa of bacteria compared to a general or healthy population of subjects. In embodiments, the method includes detecting whether the biological sample has an increased proportion of bacteria in the Prevotellaceae family of bacteria compared to a general or healthy population of subjects, wherein the increased proportion is a proportion of at least about 10% or 20%. In embodiments, the method includes detecting whether the biological sample has an increased proportion of bacteria in the Fusobacteriaceae family of bacteria compared to a general or healthy population of subjects, wherein the increased proportion is a proportion of at least about 5% or 10%. In embodiments, the method includes detecting whether at least 10% of the bacteria in the biological sample are in the Prevotellaceae family of bacteria, and at least 5% of the bacteria in the biological sample are in the Fusobacteriaceae family of bacteria. In embodiments, the method includes detecting whether the biological sample has an increased proportion of bacteria in the Pseudomonadaceae, Fusobacteriaceae, Staphylococcaceae, and Enterobacteriaceae families of bacteria compared to a general or healthy population of subjects. In embodiments, the method includes detecting whether the biological sample has an increased proportion of bacteria in the Pseudomonadaceae family of bacteria compared to a general or healthy population of subjects, wherein the increased proportion is a proportion of at least about 20%, 30%, 40%, or 50%. In embodiments, the method includes detecting whether the biological sample has an increased proportion of bacteria in the Fusobacteriaceae family of bacteria compared to a general or healthy population of subjects, wherein the increased proportion is a proportion of at least about 5% or 10%. In embodiments, the method includes detecting whether the biological sample has an increased proportion of bacteria in the Staphylococcaceae family of bacteria compared to a general or healthy population of subjects, wherein the increased proportion is a proportion of at least about 10% or 20%. In embodiments, the method includes detecting whether the biological sample has an increased proportion of bacteria in the Enterobacteriaceae family of bacteria compared to a general or healthy population of subjects, wherein the increased proportion is a proportion of at least about 5%. In embodiments, the method includes detecting whether at least 20% of the bacteria in the biological sample are in the Pseudomonadaceae family of bacteria, at least 5% of the bacteria in the biological sample are in the Fusobacteriaceae family of bacteria, at least 10% of the bacteria in the biological sample are in the Staphylococcaceae family of bacteria, and at least 5% of the bacteria in the biological sample are in the Enterobacteriaceae family of bacteria. In embodiments, the method includes detecting whether the biological sample has an increased proportion of bacteria in the Sphingomonas genus of bacteria compared to a general or healthy population of subjects. In embodiments, the method includes detecting whether the biological sample has an increased proportion of bacteria in the Corynebacteriaceae and Staphylococcaceae families of bacteria compared to a general or healthy population of subjects. In embodiments, the method includes detecting whether the biological sample has an increased proportion of bacteria in the Corynebacteriaceae family of bacteria compared to a general or healthy population of subjects, wherein the increased proportion is a proportion of at least about 50%. In embodiments, the method includes detecting whether the biological sample has an increased proportion of bacteria in the Staphylococcaceae family of bacteria compared to a general or healthy population of subjects, wherein the increased proportion is a proportion of at least about 10%. In embodiments, the method includes detecting whether at least 50% of the bacteria in the biological sample are in the Corynebacteriaceae family of bacteria, and at least 10% of the bacteria in the biological sample are in the Staphylococcaceae family of bacteria. In embodiments, the method includes detecting whether the biological sample has an increased proportion of bacteria in the Enterobacteriaceae and Staphylococcaceae families of bacteria compared to a general or healthy population of subjects. In embodiments, the method includes detecting whether the biological sample has an increased proportion of bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects. In embodiments, the method includes detecting whether the biological sample has an increased proportion of bacteria in the Enterobacteriaceae family of bacteria compared to a general or healthy population of subjects, wherein the increased proportion is a proportion of at least about 5%. In embodiments, the method includes detecting whether the biological sample has an increased proportion of bacteria in the Staphylococcaceae family of bacteria compared to a general or healthy population of subjects, wherein the increased proportion is a proportion of at least about 10%, 20%, 30%, 40%, or 50%. In embodiments, the method includes detecting whether at least 5% of the bacteria in the biological sample are in the Enterobacteriaceae family of bacteria, and at least 30% of the bacteria in the biological sample are in the Staphylococcaceae family of bacteria.

In embodiments, the method further includes determining the expression level of at least one gene in the biological sample. In embodiments, the at least one gene is any 1 of or any combination of 2, 3, 4, 5, 6, 7, 8, 9, 10, 11, 12, 13, 14, 15, 16, 17, 18, or 19 of any combination of Occludin, Claudin 2, MUCSAC, IL-4, IL-5, IL-6, IL-8, IL-25, IL-17A, IL-10, IL-1β, IL-33, CCL11, TSLP, TNF-α, ARG1, TGFβ1, CLCA1, and/or IFN-γ.

In embodiments, the method includes detecting whether the subject has increased expression any 1 of or 2, 3, 4, or 5 of any combination of IL-1β, IL-6, IL-10, IL-5, and IFN-γ compared to a general or healthy population of subjects. In embodiments, the method includes detecting whether the biological sample has an increased proportion of bacteria in the Pseudomonas genus of bacteria compared to a general or healthy population of subjects. In embodiments, the method includes detecting whether has an increased proportion of sinus microbiome bacteria in the Prevotellaceae and Fusobacteriaceae families of bacteria compared to a general or healthy population of subjects. In embodiments, the method includes detecting whether has an increased proportion of sinus microbiome bacteria in the Streptococcus, Porphyromonas, Tannerella, Treponema, Bacteroides, Dialister, and Akkermansia taxa of bacteria compared to a general or healthy population of subjects. In embodiments, the method includes detecting whether has an increased proportion of sinus microbiome bacteria in the Prevotellaceae family of bacteria compared to a general or healthy population of subjects, wherein the increased proportion is a proportion of at least about 10% or 20%. In embodiments, the method includes detecting whether has an increased proportion of sinus microbiome bacteria in the Fusobacteriaceae family of bacteria compared to a general or healthy population of subjects, wherein the increased proportion is a proportion of at least about 5% or 10%. In embodiments, the method includes detecting whether has an increased proportion of sinus microbiome bacteria in the Pseudomonadaceae, Fusobacteriaceae, Staphylococcaceae, and Enterobacteriaceae families of bacteria compared to a general or healthy population of subjects. In embodiments, the method includes detecting whether has an increased proportion of sinus microbiome bacteria in the Pseudomonadaceae family of bacteria compared to a general or healthy population of subjects, wherein the increased proportion is a proportion of at least about 20%, 30%, 40%, or 50%. In embodiments, the method includes detecting whether has an increased proportion of sinus microbiome bacteria in the Fusobacteriaceae family of bacteria compared to a general or healthy population of subjects, wherein the increased proportion is a proportion of at least about 5% or 10%. In embodiments, the method includes detecting whether has an increased proportion of sinus microbiome bacteria in the Staphylococcaceae family of bacteria compared to a general or healthy population of subjects, wherein the increased proportion is a proportion of at least about 10% or 20%. In embodiments, the method includes detecting whether has an increased proportion of sinus microbiome bacteria in the Enterobacteriaceae family of bacteria compared to a general or healthy population of subjects, wherein the increased proportion is a proportion of at least about 5%. In embodiments, the method includes detecting whether has an increased proportion of sinus microbiome bacteria in the Sphingomonas genus of bacteria compared to a general or healthy population of subjects. In embodiments, the method includes detecting whether has an increased proportion of sinus microbiome bacteria in the Corynebacteriaceae and Staphylococcaceae families of bacteria compared to a general or healthy population of subjects. In embodiments, the method includes detecting whether has an increased proportion of sinus microbiome bacteria in the Corynebacteriaceae family of bacteria compared to a general or healthy population of subjects, wherein the increased proportion is a proportion of at least about 50%. In embodiments, the method includes detecting whether has an increased proportion of sinus microbiome bacteria in the Staphylococcaceae family of bacteria compared to a general or healthy population of subjects, wherein the increased proportion is a proportion of at least about 10%. In embodiments, the method includes detecting whether has an increased proportion of sinus microbiome bacteria in the Enterobacteriaceae and Staphylococcaceae families of bacteria compared to a general or healthy population of subjects. In embodiments, the method includes detecting whether has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects. In embodiments, the method includes detecting whether has an increased proportion of sinus microbiome bacteria in the Enterobacteriaceae family of bacteria compared to a general or healthy population of subjects, wherein the increased proportion is a proportion of at least about 5%. In embodiments, the method includes detecting whether has an increased proportion of sinus microbiome bacteria in the Staphylococcaceae family of bacteria compared to a general or healthy population of subjects, wherein the increased proportion is a proportion of at least about 10%, 20%, 30%, 40%, or 50%.

In embodiments, detecting whether the subject has increased IL-5 or IFN-γ expression compared to a general or healthy population of subjects. In embodiments, detecting whether the biological sample has an increased proportion of bacteria in the Pseudomonas genus of bacteria compared to a general or healthy population of subjects. In embodiments, detecting whether has an increased proportion of sinus microbiome bacteria in the Enterobacteriaceae and Staphylococcaceae families of bacteria compared to a general or healthy population of subjects. In embodiments, detecting whether has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects. In embodiments, detecting whether has an increased proportion of sinus microbiome bacteria in the Enterobacteriaceae family of bacteria compared to a general or healthy population of subjects, wherein the increased proportion is a proportion of at least about 5%. In embodiments, detecting whether has an increased proportion of sinus microbiome bacteria in the Staphylococcaceae family of bacteria compared to a general or healthy population of subjects, wherein the increased proportion is a proportion of at least about 10%, 20%, 30%, 40%, or 50%.

In embodiments, the microorganisms are bacterial microorganisms, and wherein detecting a plurality of microorganisms in the biological sample includes detecting bacteria, or a proportion of bacteria, that are in 1, 2, 3, 4, 5, or 6 of the following taxa: Moraxella, Staphylococcaceae, Corynebacterium, Staphylococcus, Streptococcus, and/or Haemophilus.

In embodiments, detecting a plurality of microorganisms in the biological sample includes detecting whether the biological sample has an increased proportion of bacteria in the Moraxella genus of bacteria compared to standard control (such as the proportion in a general population of subjects who have asthma). In embodiments, detecting a plurality of microorganisms in the biological sample includes detecting whether the biological sample has an increased proportion of bacteria in the Neisseria genus of bacteria compared to a standard control (such as the proportion in general population of subjects who have asthma). In embodiments, detecting a plurality of microorganisms in the biological sample includes detecting whether the biological sample has a decreased proportion of bacteria in the Staphylococcaceae family or Corynebacterium genus of bacteria compared to a standard control (such as the proportion in a general population of subjects who have asthma). In embodiments, detecting a plurality of microorganisms in the biological sample includes detecting whether the biological sample has a decreased proportion of bacteria in the Corynebacterium genus of bacteria compared to a standard control (such as the proportion in a general population of subjects who have asthma). In embodiments, detecting a plurality of microorganisms in the biological sample includes detecting whether the biological sample has decreased proportion of bacteria in the Haemophilus genus of bacteria compared to a standard control (such as the proportion in a general population of subjects who have asthma). In embodiments, detecting a plurality of microorganisms in the biological sample includes detecting whether the biological sample has decreased proportion of bacteria in the Staphylococcaceae family of bacteria compared to a standard control (such as the proportion in a general population of subjects who have asthma). In embodiments, detecting a plurality of microorganisms in the biological sample includes detecting whether the biological sample has an increased proportion of bacteria in the Staphylococcus genus or Corynebacterium genus of bacteria compared to a standard control (such as the proportion in a general population of subjects who have asthma). In embodiments, detecting a plurality of microorganisms in the biological sample includes detecting whether the biological sample has an increased proportion of bacteria in the Streptococcus genus of bacteria compared to a standard control (such as the proportion in a general population of subjects who have asthma). In embodiments, detecting a plurality of microorganisms in the biological sample includes detecting whether the biological sample has an increased proportion of bacteria in the Haemophilus genus of bacteria compared to a standard control (such as the proportion in a general population of subjects who have asthma. In embodiments, detecting a plurality of microorganisms in the biological sample includes detecting whether: (a) the biological sample has an increased proportion of bacteria in the Moraxella genus of bacteria compared to a general population of subjects who have asthma; (b) the biological sample has an increased proportion of bacteria in the Neisseria genus of bacteria compared to a general population of subjects who have asthma; (c) the biological sample has a decreased proportion of bacteria in the Staphylococcaceae family or Corynebacterium genus of bacteria compared to a general population of subjects who have asthma; (d) the biological sample has a decreased proportion of bacteria in the Corynebacterium genus of bacteria compared to a general population of subjects who have asthma; (e) the biological sample has decreased proportion of bacteria in the Haemophilus genus of bacteria compared to a general population of subjects who have asthma; (f) the biological sample has decreased proportion of bacteria in the Staphylococcaceae family of bacteria compared to a general population of subjects who have asthma; (g) the biological sample has an increased proportion of bacteria in the Staphylococcus genus or Corynebacterium genus of bacteria compared to a general population of subjects who have asthma; (h) the biological sample has an increased proportion of bacteria in the Streptococcus genus of bacteria compared to a general population of subjects who have asthma; and/or (i) the biological sample has an increased proportion of bacteria in the Haemophilus genus of bacteria compared to a general population of subjects who have asthma.

In embodiments, a method provided herein includes identifying a subject as having an increased risk of asthma exacerbation if the subject has an increased proportion of nasal microbiome bacteria in the Moraxella genus of bacteria compared to a standard control (such as the proportion in a general population of subjects who have asthma). In embodiments, a method provided herein includes identifying a subject as having an increased risk of asthma exacerbation if the subject has an increased proportion of nasal microbiome bacteria in the Moraxella genus of bacteria compared to a standard control (such as the proportion in a general population of subjects who have asthma). In embodiments, a method provided herein includes identifying a subject as having an increased risk of asthma exacerbation if the subject has an increased proportion of nasal microbiome bacteria in the Neisseria genus of bacteria compared to a standard control (such as the proportion in a general population of subjects who have asthma). In embodiments, a method provided herein includes identifying a subject as having an increased risk of asthma exacerbation if the subject has an increased proportion of nasal microbiome bacteria in the Staphylococcaceae family or Corynebacterium genus of bacteria compared to a standard control (such as the proportion in a general population of subjects who have asthma). In embodiments, a method provided herein includes identifying a subject as having an increased risk of asthma exacerbation if the subject has an increased proportion of nasal microbiome bacteria in the Corynebacterium genus of bacteria compared to a standard control (such as the proportion in a general population of subjects who have asthma). In embodiments, a method provided herein includes identifying a subject as having an increased risk of asthma exacerbation if the subject has an increased proportion of nasal microbiome bacteria in the Haemophilus genus of bacteria compared to a standard control (such as the proportion in a general population of subjects who have asthma). In embodiments, a method provided herein includes identifying a subject as having an increased risk of asthma exacerbation if the subject has an increased proportion of nasal microbiome bacteria in the Staphylococcaceae family of bacteria compared to a standard control (such as the proportion in a general population of subjects who have asthma). In embodiments, a method provided herein includes identifying a subject as having an increased risk of asthma exacerbation if the subject has an increased proportion of nasal microbiome bacteria in the Staphylococcus genus or Corynebacterium genus of bacteria compared to a standard control (such as the proportion in a general population of subjects who have asthma). In embodiments, a method provided herein includes identifying a subject as having an increased risk of asthma exacerbation if the subject (a) has an increased proportion of nasal microbiome bacteria in the Moraxella genus of bacteria compared to a general population of subjects who have asthma; (b) has an increased proportion of nasal microbiome bacteria in the Moraxella genus of bacteria compared to a general population of subjects who have asthma; (c) has an increased proportion of nasal microbiome bacteria in the Neisseria genus of bacteria compared to a general population of subjects who have asthma; (d) has an increased proportion of nasal microbiome bacteria in the Staphylococcaceae family or Corynebacterium genus of bacteria compared to a general population of subjects who have asthma; (e) has an increased proportion of nasal microbiome bacteria in the Corynebacterium genus of bacteria compared to a general population of subjects who have asthma; (f) has an increased proportion of nasal microbiome bacteria in the Haemophilus genus of bacteria compared to a general population of subjects who have asthma; (g) has an increased proportion of nasal microbiome bacteria in the Staphylococcaceae family of bacteria compared to a general population of subjects who have asthma; and/or (h) has an increased proportion of nasal microbiome bacteria in the Staphylococcus genus or Corynebacterium genus of bacteria compared to a general population of subjects who have asthma.

In embodiments, a method provided herein includes identifying a subject as having an increased risk of rhinovirus infection if the subject has an increased proportion of nasal microbiome bacteria in the Haemophilus genus of bacteria compared to a standard control (such as the proportion in a general population of subjects who have asthma). In embodiments, a method provided herein includes identifying a subject as having an increased risk of rhinovirus infection if the subject has an increased proportion of nasal microbiome bacteria in the Streptococcus genus of bacteria compared to a standard control (such as the proportion in a general population of subjects who have asthma. In embodiments, a method provided herein includes identifying a subject as having an increased risk of rhinovirus infection if the subject has an increased proportion of nasal microbiome bacteria in the Moraxella genus of bacteria compared to a standard control (such as the proportion in a general population of subjects who have asthma). In embodiments, a method provided herein includes identifying a subject as having an increased risk of rhinovirus infection if the subject has an increased proportion of nasal microbiome bacteria in the Neisseria genus of bacteria compared to a standard control (such as the proportion in a general population of subjects who have asthma). In embodiments, a method provided herein includes identifying a subject as having an increased risk of rhinovirus infection if the subject has a decreased proportion of nasal microbiome bacteria in the Staphyloccocaceae family of bacteria compared to a standard control (such as the proportion in a general population of subjects who have asthma). In embodiments, a method provided herein includes identifying a subject as having an increased risk of rhinovirus infection if the subject (a) has an increased proportion of nasal microbiome bacteria in the Haemophilus genus of bacteria compared to a general population of subjects who have asthma; (b) has an increased proportion of nasal microbiome bacteria in the Streptococcus genus of bacteria compared to a general population of subjects who have asthma; (c) has an increased proportion of nasal microbiome bacteria in the Moraxella genus of bacteria compared to a general population of subjects who have asthma; (d) has an increased proportion of nasal microbiome bacteria in the Neisseria genus of bacteria compared to a general population of subjects who have asthma; and/or (e) has a decreased proportion of nasal microbiome bacteria in the Staphyloccocaceae family of bacteria compared to a general population of subjects who have asthma.

In an aspect, a method of detecting whether a subject has chronic rhinosinusitis is provided. In embodiments, the method includes detecting a plurality of microorganisms in a biological sample from the subject.

In an aspect, a method of detecting whether a subject is at risk of developing chronic rhinosinusitis is provided. In embodiments, the method includes detecting a plurality of microorganisms in a biological sample from the subject.

In an aspect, method of detecting whether a subject who has chronic rhinosinusitis has an increased risk of nasal polyposis compared to a general population of subjects who have chronic rhinosinusitis is provided. In embodiments, the method includes detecting a plurality of microorganisms in a biological sample from the subject.

In embodiments, the method includes obtaining the biological sample from the subject. In embodiments, obtaining the biological sample from the subject comprises collecting the biological sample directly from the subject. In embodiments, obtaining the biological sample from the subject comprises receiving a biological sample that has been collected (e.g, directly) from the subject (e.g., by another actor, such as a clinical professional such as a nurse, medic, or doctor). In embodiments, the biological sample has been submitted by the subject (e.g., by mail or currior).

In embodiments, the biological sample is sinus mucus.

In embodiments, the biological sample is a sinus brushing that includes mucus from the surface of a sinus.

In embodiments, the microorganisms are bacterial microorganisms.

In embodiments, detecting a plurality of microorganisms in the biological sample includes characterizing the microbiome in the biological sample.

In embodiments, characterizing the microbiome in the biological sample includes detecting the number and/or identity of bacterial taxa represented by bacteria in the biological sample.

In embodiments, the bacterial taxa include bacterial families, genera, and/or species. In embodiments, the bacterial taxa comprise, consist essentially of, or consist of bacterial families. In embodiments, the bacterial taxa are bacterial families. In embodiments, the bacterial taxa comprise, consist essentially of, or consist of bacterial genera. In embodiments, the bacterial taxa are bacterial genera. In embodiments, the bacterial taxa comprise, consist essentially of, or consist of bacterial species. In embodiments, the bacterial taxa are bacterial species. In embodiments, the bacterial taxa are bacterial families and/or genera.

In embodiments, characterizing the microbiome in the biological sample includes detecting the proportion of bacteria in the biological sample that are within 1 of or any combination of 2, 3, 4, 5, 6, 7, 8, 9, or 10 of the following families: Corynebacteriaceae, Staphylococcaceae, Pseudomonadaceae, Streptococcaceae, Fusobacteriaceae, Pasteruellaceae, Prevotellaceae, Fusobacteriaceae, Neisseriaceae, and/or Enterobacteriaceae.

In embodiments, detecting a plurality of microorganisms in the biological sample includes amplifying and sequencing 16S rRNA genes of microorganisms in the sample.

In embodiments, detecting a plurality of microorganisms in the biological sample includes amplifying and sequencing the V4 region of 16S rRNA genes of microorganisms in the sample.

In embodiments, the microorganisms are bacterial microorganisms, and wherein detecting a plurality of microorganisms in the biological sample includes detecting bacteria, or a proportion of bacteria, that are in 1 of or any combination of 2, 3, 4, 5, or 6 of the following families: Corynebacteriaceae, Staphylococcaceae, Pseudomonadaceae, Streptococcaceae, Fusobacteriaceae, Pasteruellaceae, Prevotellaceae, Fusobacteriaceae, Neisseriaceae, and/or Enterobacteriaceae.

In embodiments, detecting a plurality of microorganisms in the biological sample includes detecting whether the biological sample has an increased proportion of bacteria in the Prevotellaceae and Fusobacteriaceae families of bacteria compared to a standard control (such as the proportion in a general or healthy population of subjects). In embodiments, detecting a plurality of microorganisms in the biological sample includes detecting whether the biological sample has an increased proportion of bacteria in the Streptococcus, Streptococcus, Porphyromonas, Tannerella, Treponema, Bacteroides, Dialister, and Akkermansia taxa of bacteria compared to a standard control (such as the proportion in a general or healthy population of subjects). In embodiments, detecting a plurality of microorganisms in the biological sample includes detecting whether the biological sample has an increased proportion of bacteria in the Prevotellaceae family of bacteria compared to a standard control (such as the proportion in a general or healthy population of subjects). In embodiments, the increased proportion is a proportion of at least about 10% or 20%. In embodiments, detecting a plurality of microorganisms in the biological sample includes detecting whether the biological sample has an increased proportion of bacteria in the Fusobacteriaceae family of bacteria compared to a standard control (such as the proportion in a general or healthy population of subjects). In embodiments, the increased proportion is a proportion of at least about 5% or 10%. In embodiments, detecting a plurality of microorganisms in the biological sample includes detecting whether at least 10% of the bacteria in the biological sample are in the Prevotellaceae family of bacteria, and at least 5% of the bacteria in the biological sample are in the Fusobacteriaceae family of bacteria. In embodiments, detecting a plurality of microorganisms in the biological sample includes detecting whether the biological sample has an increased proportion of bacteria in the Pseudomonadaceae, Fusobacteriaceae, Staphylococcaceae, and Enterobacteriaceae families of bacteria compared to a standard control (such as the proportion in a general or healthy population of subjects). In embodiments, detecting a plurality of microorganisms in the biological sample includes detecting whether the biological sample has an increased proportion of bacteria in the Pseudomonadaceae family of bacteria compared to a standard control (such as the proportion in a general or healthy population of subjects). In embodiments, the increased proportion is a proportion of at least about 20%, 30%, 40%, or 50%. In embodiments, detecting a plurality of microorganisms in the biological sample includes detecting whether the biological sample has an increased proportion of bacteria in the Fusobacteriaceae family of bacteria compared to a standard control (such as the proportion in a general or healthy population of subjects). In embodiments, the increased proportion is a proportion of at least about 5% or 10%. In embodiments, detecting a plurality of microorganisms in the biological sample includes detecting whether the biological sample has an increased proportion of bacteria in the Staphylococcaceae family of bacteria compared to a standard control (such as a general or healthy population of subjects). In embodiments, the increased proportion is a proportion of at least about 10% or 20%. In embodiments, detecting a plurality of microorganisms in the biological sample includes detecting whether the biological sample has an increased proportion of bacteria in the Enterobacteriaceae family of bacteria compared to a standard control (such as the proportion in a general or healthy population of subjects). In embodiments, the increased proportion is a proportion of at least about 5%. In embodiments, detecting a plurality of microorganisms in the biological sample includes detecting whether at least 20% of the bacteria in the biological sample are in the Pseudomonadaceae family of bacteria, at least 5% of the bacteria in the biological sample are in the Fusobacteriaceae family of bacteria, at least 10% of the bacteria in the biological sample are in the Staphylococcaceae family of bacteria, and at least 5% of the bacteria in the biological sample are in the Enterobacteriaceae family of bacteria. In embodiments, detecting a plurality of microorganisms in the biological sample includes detecting whether the biological sample has an increased proportion of bacteria in the Sphingomonas genus of bacteria compared to a standard control (such as the proportion in a general or healthy population of subjects). In embodiments, detecting a plurality of microorganisms in the biological sample includes detecting whether the biological sample has an increased proportion of bacteria in the Corynebacteriaceae and Staphylococcaceae families of bacteria compared to a standard control (such as the proportion in a general or healthy population of subjects). In embodiments, detecting a plurality of microorganisms in the biological sample includes detecting whether the biological sample has an increased proportion of bacteria in the Corynebacteriaceae family of bacteria compared to a standard control (such as the level in a general or healthy population of subjects). In embodiments, the increased proportion is a proportion of at least about 50%. In embodiments, detecting a plurality of microorganisms in the biological sample includes detecting whether the biological sample has an increased proportion of bacteria in the Staphylococcaceae family of bacteria compared to a standard control (such as a general or healthy population of subjects). In embodiments, the increased proportion is a proportion of at least about 10%. In embodiments, detecting a plurality of microorganisms in the biological sample includes detecting whether at least 50% of the bacteria in the biological sample are in the Corynebacteriaceae family of bacteria, and at least 10% of the bacteria in the biological sample are in the Staphylococcaceae family of bacteria. In embodiments, detecting a plurality of microorganisms in the biological sample includes detecting whether the biological sample has an increased proportion of bacteria in the Enterobacteriaceae and Staphylococcaceae families of bacteria compared to a standard control (such as the proportion in a general or healthy population of subjects). In embodiments, detecting a plurality of microorganisms in the biological sample includes detecting whether the biological sample has an increased proportion of bacteria in the Actinobacteria, Bifidobacterium, Haemophilus, Enterobacteriaceae, Pseudomonadaceae, Sphingomonadaceae, Selenomonas and Streptophyta taxa of bacteria compared to a standard control (such as the proportion in a general or healthy population of subjects. In embodiments, detecting a plurality of microorganisms in the biological sample includes detecting whether the biological sample has an increased proportion of bacteria in the Enterobacteriaceae family of bacteria compared to a standard control (such as the proportion in a general or healthy population of subjects). In embodiments, the increased proportion is a proportion of at least about 5%. In embodiments, detecting a plurality of microorganisms in the biological sample includes detecting whether the biological sample has an increased proportion of bacteria in the Staphylococcaceae family of bacteria compared to a standard control (such as the proportion in a general or healthy population of subjects). In embodiments, the increased proportion is a proportion of at least about 10%, 20%, 30%, 40%, or 50%. In embodiments, detecting a plurality of microorganisms in the biological sample includes detecting whether at least 5% of the bacteria in the biological sample are in the Enterobacteriaceae family of bacteria, and at least 30% of the bacteria in the biological sample are in the Staphylococcaceae family of bacteria. In embodiments, detecting a plurality of microorganisms in the biological sample includes detecting whether the biological sample has an increased proportion of bacteria in the Pseudomonas genus of bacteria compared to a standard control (such as the proportion in a general or healthy population of subjects). In embodiments, detecting a plurality of microorganisms in the biological sample includes detecting whether (a) the biological sample has an increased proportion of bacteria in the Prevotellaceae and Fusobacteriaceae families of bacteria compared to a general or healthy population of subjects; (b) the biological sample has an increased proportion of bacteria in the Streptococcus, Streptococcus, Porphyromonas, Tannerella, Treponema, Bacteroides, Dialister, and Akkermansia taxa of bacteria compared to a general or healthy population of subjects; (c) the biological sample has an increased proportion of bacteria in the Prevotellaceae family of bacteria compared to a general or healthy population of subjects, wherein the increased proportion is a proportion of at least about 10% or 20%; (c) the biological sample has an increased proportion of bacteria in the Fusobacteriaceae family of bacteria compared to a general or healthy population of subjects, wherein the increased proportion is a proportion of at least about 5% or 10%; (d) at least 10% of the bacteria in the biological sample are in the Prevotellaceae family of bacteria, and at least 5% of the bacteria in the biological sample are in the Fusobacteriaceae family of bacteria; (e) the biological sample has an increased proportion of bacteria in the Pseudomonadaceae, Fusobacteriaceae, Staphylococcaceae, and Enterobacteriaceae families of bacteria compared to a general or healthy population of subjects; (f) the biological sample has an increased proportion of bacteria in the Pseudomonadaceae family of bacteria compared to a general or healthy population of subjects, wherein the increased proportion is a proportion of at least about 20%, 30%, 40%, or 50%; (g) the biological sample has an increased proportion of bacteria in the Fusobacteriaceae family of bacteria compared to a general or healthy population of subjects, wherein the increased proportion is a proportion of at least about 5% or 10%; (h) the biological sample has an increased proportion of bacteria in the Staphylococcaceae family of bacteria compared to a general or healthy population of subjects, wherein the increased proportion is a proportion of at least about 10% or 20%; (i) the biological sample has an increased proportion of bacteria in the Enterobacteriaceae family of bacteria compared to a general or healthy population of subjects, wherein the increased proportion is a proportion of at least about 5%; (j) at least 20% of the bacteria in the biological sample are in the Pseudomonadaceae family of bacteria, at least 5% of the bacteria in the biological sample are in the Fusobacteriaceae family of bacteria, at least 10% of the bacteria in the biological sample are in the Staphylococcaceae family of bacteria, and at least 5% of the bacteria in the biological sample are in the Enterobacteriaceae family of bacteria; (k) the biological sample has an increased proportion of bacteria in the Sphingomonas genus of bacteria compared to a general or healthy population of subjects; (1) the biological sample has an increased proportion of bacteria in the Corynebacteriaceae and Staphylococcaceae families of bacteria compared to a general or healthy population of subjects; (m) the biological sample has an increased proportion of bacteria in the Corynebacteriaceae family of bacteria compared to a general or healthy population of subjects, wherein the increased proportion is a proportion of at least about 50%; (n) the biological sample has an increased proportion of bacteria in the Staphylococcaceae family of bacteria compared to a general or healthy population of subjects, wherein the increased proportion is a proportion of at least about 10%; (o) at least 50% of the bacteria in the biological sample are in the Corynebacteriaceae family of bacteria, and at least 10% of the bacteria in the biological sample are in the Staphylococcaceae family of bacteria; (p) the biological sample has an increased proportion of bacteria in the Enterobacteriaceae and Staphylococcaceae families of bacteria compared to a general or healthy population of subjects; (q) the biological sample has an increased proportion of bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects; (r) the biological sample has an increased proportion of bacteria in the Enterobacteriaceae family of bacteria compared to a general or healthy population of subjects, wherein the increased proportion is a proportion of at least about 5%; (s) the biological sample has an increased proportion of bacteria in the Staphylococcaceae family of bacteria compared to a general or healthy population of subjects, wherein the increased proportion is a proportion of at least about 10%, 20%, 30%, 40%, or 50%; (t) at least 5% of the bacteria in the biological sample are in the Enterobacteriaceae family of bacteria, and at least 30% of the bacteria in the biological sample are in the Staphylococcaceae family of bacteria; and/or (u) the biological sample has an increased proportion of bacteria in the Pseudomonas genus of bacteria compared to a general or healthy population of subjects.

In embodiments, a method herein includes determining the expression level of at least one gene in the biological sample.

In embodiments, the at least one gene is 1 of or any combination of 2, 3, 4, 5, 6, 7, 8, 9, 10, 11, 12, 13, 14, 15, 16, 17, 18, or 19 of any combination of Occludin, Claudin 2, Mucin 5AC (MUC5AC), Interleukin-4 (IL-4), Interleukin-5 (IL-5), Interleukin-6 (IL-6), Interleukin-8 (IL-8), Interleukin-25 (IL-25), Interleukin-17A (IL-17A), Interleukin-10 (IL-10), Interleukin-1β (IL-1β), Interleukin-33 (IL-33), C-C motif chemokine 11 (CCL11), Thymic stromal lymphopoietin (TSLP), Tumor necrosis factor alpha (TNF-α), Arginase 1 (ARG1), Transforming growth factor beta 1 (TGFβ1), Chloride channel accessory 1 (CLCA1), and/or Interferon gamma (IFN-γ).

In embodiments, a method provided herein includes identifying the subject as having or at risk of developing chronic rhinosinusitis if the subject has increased expression of 1 of or 2, 3, 4, or 5 of any combination of IL-1β, IL-6, IL-10, IL-5, and IFN-γ compared to a standard control (such as the level of expression in a general or healthy population of subjects). In embodiments, a method provided herein includes identifying the subject as having or at risk of developing chronic rhinosinusitis if the subject has an increased proportion of sinus microbiome bacteria in the Prevotellaceae and Fusobacteriaceae families of bacteria compared to a standard control (such as the propotion in a general or healthy population of subjects). In embodiments, a method provided herein includes identifying the subject as having or at risk of developing chronic rhinosinusitis if the subject has an increased proportion of sinus microbiome bacteria in the Streptococcus, Streptococcus, Porphyromonas, Tannerella, Treponema, Bacteroides, Dialister, and Akkermansia taxa of bacteria compared to a standard control (such as the proportion in a general or healthy population of subjects. In embodiments, a method provided herein includes identifying the subject as having or at risk of developing chronic rhinosinusitis if the subject has an increased proportion of sinus microbiome bacteria in the Prevotellaceae family of bacteria compared to a standard control (such as the proportion in a general or healthy population of subjects). In embodiments, the increased proportion is a proportion of at least about 10% or 20%. In embodiments, a method provided herein includes identifying the subject as having or at risk of developing chronic rhinosinusitis if the subject has an increased proportion of sinus microbiome bacteria in the Fusobacteriaceae family of bacteria compared to a standard control (such as the proportion in a general or healthy population of subjects). In embodiments, the increased proportion is a proportion of at least about 5% or 10%. In embodiments, a method provided herein includes identifying the subject as having or at risk of developing chronic rhinosinusitis if the subject has an increased proportion of sinus microbiome bacteria in the Pseudomonadaceae, Fusobacteriaceae, Staphylococcaceae, and Enterobacteriaceae families of bacteria compared to a standard control (such as the proportion in a general or healthy population of subjects). In embodiments, a method provided herein includes identifying the subject as having or at risk of developing chronic rhinosinusitis if the subject has an increased proportion of sinus microbiome bacteria in the Pseudomonadaceae family of bacteria compared to a standard control (such as the proportion in a general or healthy population of subjects). In embodiments, the increased proportion is a proportion of at least about 20%, 30%, 40%, or 50%. In embodiments, a method provided herein includes identifying the subject as having or at risk of developing chronic rhinosinusitis if the subject has an increased proportion of sinus microbiome bacteria in the Fusobacteriaceae family of bacteria compared to a standard control (such as a general or healthy population of subjects). In embodiments, the increased proportion is a proportion of at least about 5% or 10%. In embodiments, a method provided herein includes identifying the subject as having or at risk of developing chronic rhinosinusitis if the subject has an increased proportion of sinus microbiome bacteria in the Staphylococcaceae family of bacteria compared to a standard control (such as the proportion in a general or healthy population of subjects). In embodiments, the increased proportion is a proportion of at least about 10% or 20%. In embodiments, a method provided herein includes identifying the subject as having or at risk of developing chronic rhinosinusitis if the subject has an increased proportion of sinus microbiome bacteria in the Enterobacteriaceae family of bacteria compared to a standard control (such as the proportion in a general or healthy population of subjects). In embodiments, the increased proportion is a proportion of at least about 5%. In embodiments, a method provided herein includes identifying the subject as having or at risk of developing chronic rhinosinusitis if the subject has an increased proportion of sinus microbiome bacteria in the Sphingomonas genus of bacteria compared to a standard control (such as the proportion in a general or healthy population of subjects). In embodiments, a method provided herein includes identifying the subject as having or at risk of developing chronic rhinosinusitis if the subject has an increased proportion of sinus microbiome bacteria in the Corynebacteriaceae and Staphylococcaceae families of bacteria compared to a standard control (such as the proportion in a general or healthy population of subjects). In embodiments, a method provided herein includes identifying the subject as having or at risk of developing chronic rhinosinusitis if the subject has an increased proportion of sinus microbiome bacteria in the Corynebacteriaceae family of bacteria compared to standard control (such as the proportion in a general or healthy population of subjects). In embodiments, the increased proportion is a proportion of at least about 50%. In embodiments, a method provided herein includes identifying the subject as having or at risk of developing chronic rhinosinusitis if the subject has an increased proportion of sinus microbiome bacteria in the Staphylococcaceae family of bacteria compared to a standard control (such as the proportion in a general or healthy population of subjects). In embodiments, the increased proportion is a proportion of at least about 10%. In embodiments, a method provided herein includes identifying the subject as having or at risk of developing chronic rhinosinusitis if the subject has an increased proportion of sinus microbiome bacteria in the Enterobacteriaceae and Staphylococcaceae families of bacteria compared to a standard control (such as the proportion in a general or healthy population of subjects). In embodiments, a method provided herein includes identifying the subject as having or at risk of developing chronic rhinosinusitis if the subject has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a standard control (such as the proportion in a general or healthy population of subjects). In embodiments, a method provided herein includes identifying the subject as having or at risk of developing chronic rhinosinusitis if the subject has an increased proportion of sinus microbiome bacteria in the Enterobacteriaceae family of bacteria compared to a standard control (such as a general or healthy population of subjects). In embodiments, the increased proportion is a proportion of at least about 5%. In embodiments, a method provided herein includes identifying the subject as having or at risk of developing chronic rhinosinusitis if the subject has an increased proportion of sinus microbiome bacteria in the Staphylococcaceae family of bacteria compared to a standard control (such as the proportion in a general or healthy population of subjects). In embodiments, the increased proportion is a proportion of at least about 10%, 20%, 30%, 40%, or 50%. In embodiments, a method provided herein includes identifying the subject as having or at risk of developing chronic rhinosinusitis if the subject has an increased proportion of bacteria in the Pseudomonas genus of bacteria compared to a standard control (such as the proportion in a general or healthy population of subjects). In embodiments, a method provided herein includes identifying the subject as having or at risk of developing chronic rhinosinusitis if the subject (a) has increased expression of 1, 2, 3, 4, or 5 of any combination of IL-113, IL-6, IL-10, IL-5, and IFN-γ compared to a general or healthy population of subjects; (b) has an increased proportion of sinus microbiome bacteria in the Prevotellaceae and Fusobacteriaceae families of bacteria compared to a general or healthy population of subjects; (c) has an increased proportion of sinus microbiome bacteria in the Streptococcus, Streptococcus, Porphyromonas, Tannerella, Treponema, Bacteroides, Dialister, and Akkermansia taxa of bacteria compared to a general or healthy population of subjects; (d) has an increased proportion of sinus microbiome bacteria in the Prevotellaceae family of bacteria compared to a general or healthy population of subjects, wherein the increased proportion is a proportion of at least about 10% or 20%; (e) has an increased proportion of sinus microbiome bacteria in the Fusobacteriaceae family of bacteria compared to a general or healthy population of subjects, wherein the increased proportion is a proportion of at least about 5% or 10%; (f) has an increased proportion of sinus microbiome bacteria in the Pseudomonadaceae, Fusobacteriaceae, Staphylococcaceae, and Enterobacteriaceae families of bacteria compared to a general or healthy population of subjects; (g) has an increased proportion of sinus microbiome bacteria in the Pseudomonadaceae family of bacteria compared to a general or healthy population of subjects, wherein the increased proportion is a proportion of at least about 20%, 30%, 40%, or 50%; (h) has an increased proportion of sinus microbiome bacteria in the Fusobacteriaceae family of bacteria compared to a general or healthy population of subjects, wherein the increased proportion is a proportion of at least about 5% or 10%; (i) has an increased proportion of sinus microbiome bacteria in the Staphylococcaceae family of bacteria compared to a general or healthy population of subjects, wherein the increased proportion is a proportion of at least about 10% or 20%; (j) has an increased proportion of sinus microbiome bacteria in the Enterobacteriaceae family of bacteria compared to a general or healthy population of subjects, wherein the increased proportion is a proportion of at least about 5%; (k) has an increased proportion of sinus microbiome bacteria in the Sphingomonas genus of bacteria compared to a general or healthy population of subjects; (1) has an increased proportion of sinus microbiome bacteria in the Corynebacteriaceae and Staphylococcaceae families of bacteria compared to a general or healthy population of subjects; (m) has an increased proportion of sinus microbiome bacteria in the Corynebacteriaceae family of bacteria compared to a general or healthy population of subjects, wherein the increased proportion is a proportion of at least about 50%; (n) has an increased proportion of sinus microbiome bacteria in the Staphylococcaceae family of bacteria compared to a general or healthy population of subjects, wherein the increased proportion is a proportion of at least about 10%; (o) has an increased proportion of sinus microbiome bacteria in the Enterobacteriaceae and Staphylococcaceae families of bacteria compared to a general or healthy population of subjects; (p) has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects; (q) has an increased proportion of sinus microbiome bacteria in the Enterobacteriaceae family of bacteria compared to a general or healthy population of subjects, wherein the increased proportion is a proportion of at least about 5%; (r) has an increased proportion of sinus microbiome bacteria in the Staphylococcaceae family of bacteria compared to a general or healthy population of subjects, wherein the increased proportion is a proportion of at least about 10%, 20%, 30%, 40%, or 50%; and/or (s) the biological sample has an increased proportion of bacteria in the Pseudomonas genus of bacteria compared to a general or healthy population of subjects.

In embodiments, a method provided herein includes identifying a subject as having or at risk of developing nasal polyposis if the subject has increased IL-5 or IFN-γ expression compared to a standard control (such as the level of expression in a general or healthy population of subjects). In embodiments, a method provided herein includes identifying a subject as having or at risk of developing nasal polyposis if the subject has an increased proportion of sinus microbiome bacteria in the Enterobacteriaceae and Staphylococcaceae families of bacteria compared to a standard control (such as the proportion in a general or healthy population of subjects). In embodiments, a method provided herein includes identifying a subject as having or at risk of developing nasal polyposis if the subject has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a standard control (such as the proportion in a general or healthy population of subjects). In embodiments, a method provided herein includes identifying a subject as having or at risk of developing nasal polyposis if the subject has an increased proportion of sinus microbiome bacteria in the Enterobacteriaceae family of bacteria compared to a standard control (such as the proportion in a general or healthy population of subjects). In embodiments, the increased proportion is a proportion of at least about 5%. In embodiments, a method provided herein includes identifying a subject as having or at risk of developing nasal polyposis if the subject has an increased proportion of sinus microbiome bacteria in the Staphylococcaceae family of bacteria compared to a standard control (such as the proportion in a general or healthy population of subjects). In embodiments, the increased proportion is a proportion of at least about 10%, 20%, 30%, 40%, or 50%. In embodiments, a method provided herein includes identifying a subject as having or at risk of developing nasal polyposis if the subject has an increased proportion of bacteria in the Pseudomonas genus of bacteria compared to a standard control (such as the proportion in a general or healthy population of subjects). In embodiments, a method provided herein includes identifying a subject as having or at risk of developing nasal polyposis if the subject (a) has increased IL-5 or IFN-γ expression compared to a general or healthy population of subjects; (b) has an increased proportion of sinus microbiome bacteria in the Enterobacteriaceae and Staphylococcaceae families of bacteria compared to a general or healthy population of subjects; (c) has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects; (d) has an increased proportion of sinus microbiome bacteria in the Enterobacteriaceae family of bacteria compared to a general or healthy population of subjects, wherein the increased proportion is a proportion of at least about 5%; (e) has an increased proportion of sinus microbiome bacteria in the Staphylococcaceae family of bacteria compared to a general or healthy population of subjects, wherein the increased proportion is a proportion of at least about 10%, 20%, 30%, 40%, or 50%; and/or (f) the biological sample has an increased proportion of bacteria in the Pseudomonas genus of bacteria compared to a general or healthy population of subjects.

In embodiments, bacteria may be differentiated at, e.g., the family level, the genus level, the species, level, the sub-species level, the strain level or by any other taxonomic method described herein and otherwise known in the art.

In embodiments, a biological sample is a bodily fluid obtained by filtration and/or centrifugation. For example, the biological sample may be a filtrate of e.g., a nasal wash, mucus, nasal discharge, a sinus brushing, sputum, phlegm, or saliva, or the supernatant of a centrifuged a nasal wash, mucus, nasal discharge, a sinus brushing, sputum, phlegm, or saliva. In embodiments, a filtrate is centrifuged. In embodiments a supernatant is filtered. In embodiments, centrifugation is used to increase the passage of a fluid through a filter. Non-limiting examples of filters include filters that restrict any molecule greater than, e.g., 50, 100, 200, 300, 400, 500, 50-500, 50-100, 100-500 nm in diameter (or average diameter), or greater than 0.5, 1, 1.5, 2, 2.5, 5, 10, 15, 25, 50, 100, or 200 microns in diameter (e.g., average diameter). In embodiments, a filter has pores of about 50, 100, 200, 300, 400, 500, 50-500, 50-100, 100-500 nm in diameter or about 0.5, 1, 1.5, 2, 2.5, 5, 10, 15, 25, 50, 100, or 200 microns in diameter.

In embodiments, detecting a compound (e.g., a metabolite) and/or the expression level thereof includes Ultrahigh Performance Liquid Chromatography-Tandem Mass Spectrometry (UPLC-MS/MS), Gas Chromatography-Mass Spectrometry (GC-MS), high performance liquid chromatography (HPLC), gas chromatography, liquid chromatography, Mass spectrometry (MS), inductively coupled plasma-mass spectrometry (ICP-MS), accelerator mass spectrometry (AMS), thermal ionization-mass spectrometry (TIMS) and spark source mass spectrometry (SSMS), matrix-assisted laser desorption/ionization (MALDI), and/or MALDI-TOF.

In embodiments, detecting the expression level of a protein includes assaying the level of the compound (e.g., with high-performance liquid chromatography (HPLC), liquid chromatography-mass spectrometry (LC/MS), an enzyme-linked immunosorbent assay (ELISA), protein immunoprecipitation, immunoelectrophoresis, protein immunostaining, and/or Western blot) or the level of mRNA that encodes the protein. In embodiments, detecting the expression level of a compound (e.g., a protein) includes lysing a cell. In embodiments, detecting the expression level of a compound includes a polymerase chain reaction (e.g., reverse transcriptase polymerase chain reaction), RNA sequencing, microarray analysis, immunohistochemistry, or flow cytometry.

In an aspect, provided herein is a kit or system for performing a diagnostic method disclosed herein. In embodiments, the kit or system includes one or more primers, probes, or antibodies specific for a protein or any combination of any one of the proteins mentioned herein. In embodiments, the kit or system includes one or more primers or probes specific for the mRNA of a protein or any combination of any one of the proteins mentioned herein. In embodiments, the kit or system includes one or more primers or probes specific for one or more of any combination of the bacterial species, genera, families or other taxa, disclosed herein. In embodmients, the one or more probes or primers hybridize the 16S rRNA gene of one or more bacterial taxa disclosed herein under stringent hybridization conditions.

### III. Methods of Treatment and Monitoring

In an aspect, a method of treating or preventing asthma, an asthma exacerbation, or a rhinovirus infection in a subject in need thereof is provided. In embodiments, the subject has an increased risk of asthma exacerbation or rhinovirus infection compared to a general population of subjects who have asthma. In embodiments, the subject has been identified as having an asthma or a rhinovirus infection according to a method provided herein. In embodiments, the subject has been identified as having an increased risk of asthma, an asthma exacerbation, or a rhinovirus infection according to a method provided herein.

In an aspect, a method of monitoring asthma, a rhinovirus infection or an increased risk of asthma, an asthma exacerbation, or a rhinovirus infection is provided herein. In embodiments, the subject has an increased risk of asthma exacerbation or rhinovirus infection compared to a general population of subjects who have asthma. In embodiments, the subject has been identified as having an asthma or a rhinovirus infection according to a method provided herein. In embodiments, the subject has been identified as having an increased risk of asthma, an asthma exacerbation, or a rhinovirus infection according to a method provided herein.

In an aspect, a method of treating or preventing rhinosinusitis (e.g. chronic rhinosinusitis), or nasal polyposis, in a subject in need thereof is provided. In embodiments, the subject has rhinosinusitis (e.g. chronic rhinosinusitis). In embodiments, the subject has an increased risk of chronic rhinosinusitis compared to a healthy or general population (e.g., a general population of subjects who have rhinosinusitis). In embodiments, the subject has an increased risk of nasal polyposis compared to a general population of subjects who have chronic rhinosinusitis. In embodiments, the subject has been identified as having rhinosinusitis (e.g. chronic rhinosinusitis) or nasal polyposis according to a method provided herein. In embodiments, the subject has been identified as having rhinosinusitis (e.g. chronic rhinosinusitis) or nasal polyposis according to a method provided herein.

In an aspect, a method of monitoring rhinosinusitis (e.g. chronic rhinosinusitis) or nasal polyposis or an increased risk of rhinosinusitis (e.g. chronic rhinosinusitis) or nasal polyposis is provided herein. In embodiments, the subject has been identified as having rhinosinusitis (e.g. chronic rhinosinusitis) or nasal polyposis according to a method provided herein. In embodiments, the subject has been identified as having rhinosinusitis (e.g. chronic rhinosinusitis) or nasal polyposis according to a method provided herein.

In an aspect, a method of treating or preventing asthma, an asthma exacerbation, or a rhinovirus infection in a subject in need thereof is provided. In embodiments, the method includes detecting nasal dysbiosis in the subject, and administering to the subject an effective amount of an antibiotic compound.

In an aspect, a method of treating or preventing asthma, an asthma exacerbation, or a rhinovirus infection in a subject in need thereof is provided. In embodiments, the method includes administering to the subject an effective amount of an antibiotic compound, wherein the subject has been identified as having an increased risk of asthma exacerbation or rhinovirus infection compared to a general population of subjects who have asthma.

In an aspect, a method of treating or preventing asthma, an asthma exacerbation, or a rhinovirus infection in a subject in need thereof is provided. In embodiments, the method includes administering to the subject an effective amount of an antibiotic compound.

In an aspect, a method of treating or preventing chronic rhinosinusitis or nasal polyposis in a subject in need thereof is provided. In embodiments, the method includes detecting sinus dysbiosis in the subject, and administering to the subject an effective amount of an antibiotic compound.

In an aspect, a method of treating or preventing chronic rhinosinusitis or nasal polyposis in a subject in need thereof is provided. In embodiments, the method includes administering to the subject an effective amount of an antibiotic compound, wherein the subject has been identified as having of chronic rhinosinusitis or nasal polyposis or at risk of chronic rhinosinusitis or nasal polyposis.

In an aspect, a method of treating or preventing chronic rhinosinusitis or nasal polyposis in a subject in need thereof is provided. In embodiments, the method includes administering to the subject an effective amount of an antibiotic compound.

In embodiments, the subject has nasal dysbiosis or sinus dysbiosis. In embodiments, the subject has nasal dysbiosis. In embodiments, the subject has sinus dysbiosis.

In embodiments, the nasal dysbiosis or sinus dysbiosis includes an increased proportion or amount of Staphylococcus sp. bacteria compared to a standard control.

In embodiments, the nasal dysbiosis or sinus dysbiosis includes an increased proportion or amount of Corynebacterium sp. bacteria compared to a standard control.

In embodiments, the nasal dysbiosis or sinus dysbiosis includes an increased proportion or amount of Pseudomonas sp. bacteria compared to a standard control.

In embodiments, the nasal dysbiosis or sinus dysbiosis includes an increased proportion or amount of Streptococcus sp. bacteria compared to a standard control.

In embodiments, the nasal dysbiosis or sinus dysbiosis includes an increased proportion or amount of Prevotella sp. bacteria compared to a standard control.

In embodiments, the antibiotic compound is a beta-lactam, a cephalosporin, a lincosamide, a macrolide, a tetracycline, a sulfa drug (e.g., compound), or mupirocin.

In embodiments, the antibiotic compound is oxacillin, flucloxacillin, cefazolin, cephalothin, cephalexin, erythromycin, doxycycline, or minocycline.

In embodiments, the mupirocin is administered in a cream.

In embodiments, the subject has nasal dysbiosis including an increased proportion or amount of Staphylococcus sp. bacteria compared to a standard control.

In embodiments, the antibiotic compound is erythromycin, penicillin G, clarithromycin, bactrim DS, ciprofloxacin, vancomycin, daptomycin, or linezolid.

In embodiments, the subject has nasal dysbiosis including an increased proportion or amount of Corynebacterium sp. bacteria compared to a standard control.

In embodiments, the antibiotic compound is a penicillin, a +/−beta-lactam inhibitor, a cephalosporin, a monobactam, a fluoroquinolone, a carbapenem, an aminoglycoside, or a polymixin.

Non-limiting examples of beta-lactam antibiotics include penicillin derivatives (penams), cephalosporins (cephems), monobactams, and carbapenems, penicillin G, penicillin V, methicillin; oxacillin, nafcillin, ampicillin, amoxicillin, and carbenicillin. Non-limiting examples of lincosamides include lincomycin, clindamycin, and pirlimycin. Non-limiting examples of macrolides include azithromycin, clarithromycin, erythromycin, fidaxomicin, and telithromycin. Non-limitng examples of tetracyclines include tetracycline, chlortetracycline, oxytetracycline, demeclocycline, lymecycline, meclocycline, methacycline, minocycline, and rolitetracycline. Non-limiting examples of sulfa drugs include co-trimoxazole, sulfadiazine, sulfamethoxazole, trimethoprim-sulfamethoxazole, trimethoprim, sulfasalazine, and sulfisoxazole. Non-limiting examples of cephalosporins include ceftobiprole, ceftaroline, ceftolozane, cefclidine, cefepime, cefluprenam, cefoselis, cefozopran, cefpirome, and cefquinome. Non-limiting examples of monobactams include aztreonam, tigemonam, nocardicin A, and tabtoxin. Non-limiting examples of fluoroquinolones include ciprofloxacin, garenoxacin, gatifloxacin, gemifloxacin, levofloxacin, and moxifloxacin. Non-limiting examples of carbapenems include imipenem, meropenem, ertapenem, and doripenem. Non-limiting examples of aminoglycosides include kanamycin A, amikacin, tobramycin, dibekacin, gentamicin, sisomicin, netilmicin, neomycins B, C, paromomycin, and streptomycin. Non-limiting examples of polymixins include mattacin, polymyxin B, and colistin.

In embodiments, the number or proportion of Staphylococcus bacteria is reduced using a B-lactam (such as oxacillin or flucloxacillin, a first generation cephalosporin (such as cefazolin, cephalothin or cephalexin), a lincosamide (such as clindamycin or lincomycin), a macrolide (such as erythromycin), a tetracycline (such as doxycycline or minocycline), a sulfa drug, or mupirocin cream (e.g., for nose infections).

In embodiments, the number or proportion of Corynebacterium bacteria is reduced using erythromycin, penicillin G, clarithromycin, bactrim DS, ciprofloxacin, vancomycin, daptomycin, or linezolid.

In embodiments, the number or proportion of Pseudomonas bacteria is reduced using a penicillin, a cephalosporin (+/−beta-lactam inhibitor), a monobactam, a fluoroquinolone, a carbapenem, an aminoglycosides, or polymixin (e.g., in cases of antibiotic resistant strains).

In embodiments, the number or proportion of Streptococcus bacteria is reduced using a penicillin or a cephalosporin.

In embodiments, the number or proportion of Prevotella bacteria is reduced using metronidazole, amoxycillin/clavulanate, a ureidopenicilin, a carbapenem, a cephalosporin, clindamycin, or chloramphenicol.

In embodiments, the subject has sinus dysbiosis including an increased proportion or amount of Pseudomonas sp. bacteria compared to a standard control.

In embodiments, the subject has sinus dysbiosis including an increased proportion or amount of Pseudomonas sp. bacteria compared to a standard control, wherein the Pseudomonas sp. bacteria include an antibiotic resistant strain, and wherein the antibiotic is a polymixin.

In embodiments, the antibiotic is a penicillin or a cephalosporin.

In embodiments, the subject has nasal dysbiosis including an increased proportion or amount of Streptococcus sp. bacteria compared to a standard control.

In embodiments, the antibiotic is metronidazole, amoxicillin, clavulanate, amoxicillin and clavulanate, a ureidopenicilin, a carbapenem, a cephalosporin, clindamycin, or chloramphenicol.

In embodiments, the subject has sinus dysbiosis including an increased proportion or amount of Prevotella sp. bacteria compared to a standard control.

In embodiments, a method provided herein further includes administering at least one bacterium to the subject.

In embodiments, the at least one bacterium includes a Lactobacillus sakei bacterium. In embodiments, Lactobacillus sakei has efficacy against Pseudomonas (e.g., P. aeruginosa) and Corynebacterium (e.g., C. tuberculostearicum).

In embodiments, a method provided herein further includes administering an anti-IL-5 compound to the subject.

In an aspect, a method of treating or preventing chronic rhinosinusitis or nasal polyposis in a subject in need thereof is provided. In embodiments, the method includes administering to the subject an effective amount of an anti-IL-5 compound.

In embodiments, the anti-IL-5 compound is an anti-IL-5 antibody.

In embodiments, the anti-IL-5 antibody is reslizumab or mepolizumab.

In embodiments, the anti-IL-5 compound is an anti-IL-5 receptor antibody.

In embodiments, the anti-IL-5 receptor antibody is benralizumab.

In embodiments, the method includes administering at least one bacterium to the subject.

In embodiments, the at least one bacterium includes a Lactobacillus sakei bacterium.

In an aspect, a method of treating or preventing asthma, asthma exacerbation, or a rhinovirus infection in a subject in need thereof is provided. In embodiments, the method includes detecting nasal dysbiosis in the subject, and administering to the subject an effective amount of at least one bacterium.

In an aspect, a method of treating or preventing asthma, an asthma exacerbation, or a rhinovirus infection in a subject in need thereof is provided. In embodiments, the method includes administering to the subject an effective amount of at least one bacterium, wherein the subject has been identified as having an increased risk of asthma exacerbation or rhinovirus infection compared to a general population of subjects who have asthma.

In an aspect, a method of treating or preventing asthma, an asthma exacerbation, or a rhinovirus infection in a subject in need thereof is provided. In embodiments, the method includes administering to the subject an effective amount of at least one bacterium.

In an aspect, a method of treating or preventing chronic rhinosinusitis or nasal polyposis in a subject in need thereof is provided. In embodiments, the method includes detecting sinus dysbiosis in the subject, and administering to the subject an effective amount of at least one bacterium.

In an aspect, a method of treating or preventing chronic rhinosinusitis or nasal polyposis in a subject in need thereof is provided. In embodiments, the method includes administering to the subject an effective amount of at least one bacterium, wherein the subject has been identified as having of chronic rhinosinusitis or nasal polyposis or at risk of chronic rhinosinusitis or nasal polyposis.

In an aspect, a method of treating or preventing chronic rhinosinusitis or nasal polyposis in a subject in need thereof is provided. In embodiments, the method includes administering to the subject an effective amount of at least one bacterium.

In embodiments, the subject has nasal dysbiosis or sinus dysbiosis.

In embodiments, the nasal dysbiosis or sinus dysbiosis includes an increased proportion or amount of Corynebacterium sp. (e.g., C. tuberculostearicum) bacteria compared to a standard control.

In embodiments, the nasal dysbiosis or sinus dysbiosis includes an increased proportion or amount of Pseudomonas sp. (e.g., P. aeruginosa) bacteria compared to a standard control.

In embodiments, the at least one bacterium is Lactobacillus sakei.

In an aspect, a method of reducing the amount of Corynebacterium sp. (e.g., C. tuberculostearicum) bacteria or Pseudomonas sp. (e.g., P. aeruginosa) bacteria in the sinus microbiome of a subject is provided. In embodiments, the method comprises administering to the subject an effective amount of a Lactobacillus sakei bacterium. In embodiments, the subject has rhinosinusitis (e.g., chronic rhinosinusitis) or nasal polyposis. In embodiments, there is an increased proportion or amount of Corynebacterium sp. bacteria (e.g., C. tuberculostearicum) or Pseudomonas sp. (e.g., P. aeruginosa) bacteria in the sinus microbiome of the subject compared to a standard control.

In an aspect, a method of treating or preventing an infection of Corynebacterium sp. (e.g., C. tuberculostearicum) bacteria or Pseudomonas sp. (e.g., P. aeruginosa) bacteria in the sinus of a subject in need thereof is provided. In embodiments, the method comprises administering to the subject an effective amount of a Lactobacillus sakei bacterium.

In an aspect, a method of treating or preventing acute sinusitis in a subject in need thereof is provided. In embodiments, the method comprises administering to the subject an effective amount of a Lactobacillus sakei bacterium.

In an aspect, a method of increasing bacterial divsersity in the sinus of a subject in need thereof is provided. In embodiments, the method comprises administering to the subject an effective amount of a Lactobacillus sakei bacterium.

In an aspect, a method of treating or preventing chronic rhinosinusitis or nasal polyposis in a subject in need thereof is provided. In embodiments, the method includes detecting an increased proportion or amount of Corynebacterium sp. (e.g., C. tuberculostearicum) bacteria or Pseudomonas sp. (e.g., P. aeruginosa) bacteria in the sinus microbiome of the subject compared to a standard control, and administering to the subject an effective amount of a Lactobacillus sakei bacterium.

In an aspect, a method of treating or preventing chronic rhinosinusitis or nasal polyposis in a subject in need thereof is provided. In embodiments, the method includes administering to the subject an effective amount of a Lactobacillus sakei bacterium, wherein the subject has been identified as having an increased proportion or amount of Corynebacterium sp. (e.g., C. tuberculostearicum) bacteria or Pseudomonas sp. (e.g., P. aeruginosa) bacteria in the sinus microbiome of the subject compared to a standard control.

In an aspect, a method of treating or preventing chronic rhinosinusitis or nasal polyposis in a subject in need thereof is provided. In embodiments, the method includes administering to the subject an effective amount of a Lactobacillus sakei bacterium, wherein the subject has an increased proportion or amount of Corynebacterium sp. (e.g., C. tuberculostearicum) bacteria or Pseudomonas sp. (e.g., P. aeruginosa) bacteria in the sinus microbiome of the subject compared to a standard control.

In an aspect, a method of treating or preventing dysbiosis in a subject in need thereof is provided. In embodiments, the method includes administering to the subject an effective amount of a Lactobacillus sakei bacterium.

In embodiments, a method provided herein includes administering at least one bacterium to a subject. In embodiments, the at least one bacterium (such as a Lactobacillus sakei bacterium) is administered before another active agent (such as an antibiotic or an anti-IL-5 compound). In embodiments, the at least one bacterium (such as a Lactobacillus sakei bacterium) is administered concurrently with another active agent (such as an antibiotic or an anti-IL-5 compound). In embodiments, the at least one bacterium (such as a Lactobacillus sakei bacterium) is administered in a composition that further includes another active agent (such as an antibiotic or an anti-IL-5 compound). In embodiments, the at least one bacterium (such as a Lactobacillus sakei bacterium) is administered after another active agent (such as an antibiotic or an anti-IL-5 compound). In embodiments, the antibiotic is bacteriostatic or bactericidal to Staphylococcus bacteria, Corynebacterium bacteria, Pseudomonas bacteria, and/or Prevotella bacteria. In embodiments, the antibiotic is bacteriostatic or bactericidal to Corynebacterium bacteria and/or Pseudomonas bacteria. In embodiments, the antibiotic is not bacteriostatic or bactericidal to Lactobacillus sakei bacteria. In embodiments, Lactobacillus sakei bacteria are resistant to the antibiotic. In embodiments, the antibiotic is administered followed by the at least one bacterium (such as a Lactobacillus sakei bacterium). In embodiments, the at least one bacterium (such as a Lactobacillus sakei bacterium) is administered about 1, 2, 3, 4, 5, 6, 7, 8, 9, 10, 11, 12, 13, 14, or 15 days after the antibiotic. In embodiments, the at least one bacterium (such as a Lactobacillus sakei bacterium) is administered within about 1, 2, 3, 4, 5, 6, 7, 8, 9, 10, 11, 12, 13, 14, or 15 days of the antibiotic. In embodiments, the at least one bacterium (such as a Lactobacillus sakei bacterium) is administered within 1-10, 1-7, 1-3, 1-4, or 5-15 days after the antibiotic. In embodiments, the antibody is administered for about 1, 2, 3, 4, 5, 6, 7, 8, 9, 10, 11, 12, 13, 14, or 15 days, and then the at least one bacterium (such as a Lactobacillus sakei bacterium) is administered about 1, 2, 3, 4, 5, 6, 7, 8, 9, 10, 11, 12, 13, 14, or 15 days after the last dose of the antibiotic. In embodiments, the antibody is administered for about 1, 2, 3, 4, 5, 6, 7, 8, 9, 10, 11, 12, 13, 14, or 15 days, and then the at least one bacterium (such as a Lactobacillus sakei bacterium) is administered within 1-10, 1-7, 1-3, 1-4, or 5-15 days after the last dose of the antibiotic.

In embodiments, the at least one bacterium (such as a Lactobacillus sakei bacterium) is administered without an antibiotic. In embodiments, the the at least one bacterium (such as a Lactobacillus sakei bacterium) is administered without an anti-IL-5 compound. In embodiments, the at least one bacterium (such as a Lactobacillus sakei bacterium) is administered without another active agent.

In embodiments, the isolated Lactobacillus sakei bacterium is administered nasally. In embodments, the isolated Lactobacillus sakei bacterium is administered in a nasal spray.

In an aspect, a composition including an isolated Lactobacillus sakei bacterium and a pharmaceutically acceptable excipient is provided.

In embodiments, the pharmaceutically acceptable excipient is suitable for nasal administration.

In embodiments, the composition is a capsule, a tablet, a suspension, a suppository, a powder, a cream, an oil, an oil-in-water emulsion, a water-in-oil emulsion, or an aqueous solution.

In embodiments, the composition is in the form of a powder, a solid, a semi-solid, or a liquid.

In embodiments, the composition further includes an anti-IL-5 compound.

In embodiments, a compound or bacterium is administered orally. In embodiments, a compound or bacterium is administered nasally. In embodiments, a composition that includes a compound (such as an anti-IL-5 compound or an antibiotic) and/or a bacterium is administered into one or more nostrils of a subject. In embodiments, a composition that includes a compound (such as an anti-IL-5 compound or an antibiotic) and/or a bacterium is sprayed into one or more nostrils of a subject. In embodiments, a composition that includes a compound (such as an anti-IL-5 compound or an antibiotic) and/or a bacterium is inhaled into one or more nostrils of a subject.

In embodiments, a subject is administered an effective amount of one or more compounds and/or bacterial cells (e.g., therapeutic compounds). The terms effective amount and effective dosage are used interchangeably. The term effective amount is defined as any amount necessary to produce a desired physiologic response (e.g., reduction of dysbiosis, asthma, rhinosinusitis, chronic rhinosinusitis, nasal polyposis, or other disease, such as an infection, e.g., a rhinovirus infection infection). In embodiments, an effective amount is an amount sufficient to accomplish a stated purpose (e.g. achieve the effect for which it is administered, treat a disease, kill pathogenic cells, reduce one or more symptoms of a disease or condition such as asthma, nasal polyposis, chronic rhinosinusitis, infection or dysbiosis). An example of an “effective amount” is an amount sufficient to contribute to the treatment, prevention, or reduction of a symptom or symptoms of a disease, which could also be referred to as a “therapeutically effective amount.” Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

For prophylactic use, a therapeutically effective amount of an antibiotic, an anti-IL-5 compound (i.e., an IL-5 inhibitor), or a bacterium described herein is administered to a subject prior to or during early onset of (e.g., upon initial signs and symptoms of) dysbiosis, asthma, rhinosinusitis, chronic rhinosinusitis, nasal polyposis, or other disease, such as an infection, e.g., a rhinovirus infection infection. In embodiments, therapeutic treatment involves administering to a subject a therapeutically effective amount of an agent described herein after diagnosis or development of disease. Thus, in embodiments, a method of treating a disease (e.g., an inflammatory disease, an infection, or dysbiosis) in a subject in need thereof is provided.

The terms “subject,” “patient,” “individual,” etc. are not intended to be limiting and can be generally interchanged. In embodiments, an individual described as a “patient” does not necessarily have a given disease, but may, e.g., be merely seeking medical advice.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. In embodiments, treatment can refer to a 10%, 20%, 30%, 40%, 50%, 60%, 70%, 80%, 90%, or 100% reduction in the severity of an established disease, condition, or symptom of the disease or condition (e.g., asthma, nasal polyposis, chronic rhinosinusitis, infection or dysbiosis). For example, a method for treating a disease is considered to be a treatment if there is a 10% reduction in one or more symptoms of the disease in a subject as compared to a control. In embodiments, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

Regardless of how the compositions are formulated, the dosage required will depend on the route of administration, the nature of the formulation, the nature of the subject's condition, e.g., immaturity of the immune system or a gastrointestinal disorder, the subject's size, weight, surface area, age, and sex, other drugs being administered, and the judgment of the attending clinicians.

Administrations can be single or multiple (e.g., 2- or 3-, 4-, 6-, 8-, 10-, 20-, 50-, 100-, 150-, or more fold). The duration of treatment with any composition provided herein can be any length of time from as short as one day to as long as the life span of the host (e.g., many years). For example, a composition can be administered 1, 2, 3, 4, 5, 6, or 7 times a week (for, for example, 4 weeks to many months or years); once a month (for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The compositions may also be administered in conjunction with other therapeutic agents. Other therapeutic agents will vary according to the particular disorder, but can include, for example, dysbiosis or an infection. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of a disease, pathology, or condition, or their symptoms. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase (e.g., improvement of function, such as nasal or sinus function) or decrease (e.g., reduction of a symptom) of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

In embodiments, a compound is administered in a composition that includes a pharmaceutically acceptable excipient. “Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

In embodiments, a composition includes one or more species or strains of bacteria. In embodiments, the bacteria comprise, consist essentially of, or consist of Lactobacillus sakei bacteria. In embodiments, a composition includes less than about 20, 19, 18, 17, 16, 15, 14, 13, 12, 11, 10, 9, 8, 7, 6, 5, 4, 3, or 2 different species of bacteria. In embodiments, the composition includes less than about 20 different species of bacteria. In embodiments, the composition includes less than 20 different species of bacteria. In embodiments, the composition includes less than about 15 different species of bacteria. In embodiments, the composition includes less than 15 different species of bacteria. In embodiments, the composition includes less than about 10 different species of bacteria. In embodiments, the composition includes less than 10 different species of bacteria. In embodiments, the composition includes less than about 9 different species of bacteria. In embodiments, the composition includes less than 9 different species of bacteria. In embodiments, the composition includes less than about 8 different species of bacteria. In embodiments, the composition includes less than 8 different species of bacteria. In embodiments, the composition includes less than about 7 different species of bacteria. In embodiments, the composition includes less than 7 different species of bacteria. In embodiments, the composition includes less than about 6 different species of bacteria. In embodiments, the composition includes less than 6 different species of bacteria. In embodiments, the composition includes less than about 5 different species of bacteria. In embodiments, the composition includes less than 5 different species of bacteria. In embodiments, the composition includes less than about 4 different species of bacteria. In embodiments, the composition includes less than 4 different species of bacteria. In embodiments, the composition includes less than about 3 different species of bacteria. In embodiments, the composition includes less than 3 different species of bacteria. In embodiments, the composition includes less than about 2 different species of bacteria. In embodiments, the composition includes less than 2 different species of bacteria.

The compositions that include a compound or bacterium can be formulated in a unit dosage form, each dosage containing, for example, from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of

In embodiments, a composition provided herein may be administered orally or nasally and include live microorganisms (e.g., comprising, consisting essentially of, or consisting of Lactobacillus sakei bacterial cells) from 10³ to 10′⁵ colony forming units (cfu)/g. In embodiments, the composition includes 10⁴ to 10′⁵ cfu/g. In embodiments, the composition includes 10⁵ to 10′⁵ cfu/g. In embodiments, the composition includes 10⁶ to 10′⁵ cfu/g. In embodiments, the composition includes 10⁷ to 10′⁵ cfu/g. In embodiments, the composition includes 10⁸ to 10′⁵ cfu/g. In embodiments, the composition includes 10⁹ to 10¹⁵ cfu/g. In embodiments, the composition includes 10¹⁰ to 10¹⁵ cfu/g. In embodiments, the composition includes 10¹¹ to 10¹⁵ cfu/g. In embodiments, the composition includes 10¹² to 10¹⁵ cfu/g. In embodiments, the composition includes 10¹³ to 10¹⁵ cfu/g. In embodiments, the composition includes 10¹⁴ to 10¹⁵ cfu/g. In embodiments, the composition includes from 10³ to 10¹⁵ cfu. In embodiments, the composition includes 10⁴ to 10¹⁵ cfu. In embodiments, the composition includes 10⁵ to 10¹⁵ cfu. In embodiments, the composition includes 10⁶ to 10¹⁵ cfu. In embodiments, the composition includes 10⁷ to 10¹⁵ cfu. In embodiments, the composition includes 10⁸ to 10¹⁵ cfu. In embodiments, the composition includes 10⁹ to 10¹⁵ cfu. In embodiments, the composition includes 10¹⁰ to 10¹⁵ cfu. In embodiments, the composition includes 10¹¹ to 10¹⁵ cfu. In embodiments, the composition includes 10¹² to 10¹⁵ cfu. In embodiments, the composition includes 10¹³ to 10¹⁵ cfu. In embodiments, the composition includes 10¹⁴ to 10¹⁵ cfu.

In embodiments, a composition provided herein may be administered orally or nasally and include live microorganisms (e.g., comprising, consisting essentially of, or consisting of Lactobacillus sakei bacterial cells) from 10³ to 10¹⁴ colony forming units (cfu)/g. In embodiments, the composition includes 10⁴ to 10¹⁴ cfu/g. In embodiments, the composition includes 10⁵ to 10¹⁴ cfu/g. In embodiments, the composition includes 10⁶ to 10⁷ cfu/g. In embodiments, the composition includes 10⁷ to 10¹⁴ cfu/g. In embodiments, the composition includes 10⁸ to 10¹¹ cfu/g. In embodiments, the composition includes 10⁹ to 10¹⁴ cfu/g. In embodiments, the composition includes 10¹⁰ to 10¹⁴ cfu/g. In embodiments, the composition includes 10¹¹ to 10¹⁴ cfu/g. In embodiments, the composition includes 10¹² to 10¹⁴ cfu/g. In embodiments, the composition includes 10¹³ to 10¹⁴ cfu/g. In embodiments, the composition includes from 10³ to 10¹⁴ cfu. In embodiments, the composition includes 10⁴ to 10¹⁴ cfu. In embodiments, the composition includes 10⁵ to 10¹⁴ cfu. In embodiments, the composition includes 10⁶ to 10¹⁴ cfu. In embodiments, the composition includes 10⁷ to 10¹⁴ cfu. In embodiments, the composition includes 10⁸ to 10¹¹ cfu. In embodiments, the composition includes 10⁹ to 10¹⁴ cfu. In embodiments, the composition includes 10¹⁰ to 10¹⁴ cfu. In embodiments, the composition includes 10¹¹ to 10¹⁴ cfu. In embodiments, the composition includes 10¹² to 10¹⁴ cfu. In embodiments, the composition includes 10¹³ to 10¹⁴ cfu.

In embodiments, a composition provided herein may be administered orally or nasally and include live microorganisms (e.g., comprising, consisting essentially of, or consisting of Lactobacillus sakei bacterial cells) from 10³ to 10¹³ colony forming units (cfu)/g. In embodiments, the composition includes 10⁴ to 10¹³ cfu/g. In embodiments, the composition includes 10⁵ to 10¹³ cfu/g. In embodiments, the composition includes 10⁶ to 10¹³ cfu/g. In embodiments, the composition includes 10⁷ to 10′³ cfu/g. In embodiments, the composition includes 10⁸ to 10¹³ cfu/g. In embodiments, the composition includes 10⁹ to 10¹³ cfu/g. In embodiments, the composition includes 10¹⁰ to 10¹³ cfu/g. In embodiments, the composition includes 10¹¹ to 10¹³ cfu/g. In embodiments, the composition includes 10¹² to 10¹³ cfu/g. In embodiments, the composition includes from 10³ to 10¹³ cfu. In embodiments, the composition includes 10⁴ to 10¹³ cfu. In embodiments, the composition includes 10⁵ to 10¹³ cfu. In embodiments, the composition includes 10⁶ to 10¹³ cfu. In embodiments, the composition includes 10⁷ to 10¹³ cfu. In embodiments, the composition includes 10⁸ to 10¹³ cfu. In embodiments, the composition includes 10⁹ to 10¹³ cfu. In embodiments, the composition includes 10¹⁰ to 10¹³ cfu. In embodiments, the composition includes 10¹¹ to 10¹³ cfu. In embodiments, the composition includes 10¹² to 10¹³ cfu.

In embodiments, a composition provided herein may be administered orally or nasally and include live microorganisms (e.g., comprising, consisting essentially of, or consisting of Lactobacillus sakei bacterial cells) from 10³ to 10¹² colony forming units (cfu)/g. In embodiments, the composition includes 10⁴ to 10¹² cfu/g. In embodiments, the composition includes 10⁵ to 10¹² cfu/g. In embodiments, the composition includes 10⁶ to 10¹² cfu/g. In embodiments, the composition includes 10⁷ to 10¹² cfu/g. In embodiments, the composition includes 10⁸ to 10¹² cfu/g. In embodiments, the composition includes 10⁹ to 10¹² cfu/g. In embodiments, the composition includes 10¹⁰ to 10¹² cfu/g. In embodiments, the composition includes 10¹¹ to 10¹² cfu/g. In embodiments, the composition includes from 10³ to 10¹² cfu. In embodiments, the composition includes 10⁴ to 10¹² cfu/g. In embodiments, the composition includes 10⁵ to 10¹² cfu. In embodiments, the composition includes 10⁶ to 10¹² cfu. In embodiments, the composition includes 10⁷ to 10¹² cfu. In embodiments, the composition includes 10⁸ to 10¹² cfu. In embodiments, the composition includes 10⁹ to 10¹² cfu. In embodiments, the composition includes 10¹⁰ to 10¹² cfu. In embodiments, the composition includes 10¹¹ to 10¹² cfu.

In embodiments, a composition provided herein may be administered orally or nasally and include live microorganisms from 10³ to 10¹¹ colony forming units (cfu)/g. In embodiments, the composition includes 10⁴ to 10¹¹ cfu/g. In embodiments, the composition includes 10⁵ to 10¹¹ cfu/g. In embodiments, the composition includes 10⁶ to 10¹¹ cfu/g. In embodiments, the composition includes 10⁷ to 10¹¹ cfu/g. In embodiments, the composition includes 10⁸ to 10¹¹ cfu/g. In embodiments, the composition includes 10⁹ to 10¹¹ cfu/g. In embodiments, the composition includes from 10³ to 10¹¹ cfu. In embodiments, the composition includes 10⁴ to 10¹¹ cfu. In embodiments, the composition includes 10⁵ to 10¹¹ cfu. In embodiments, the composition includes 10⁶ to 10¹¹ cfu. In embodiments, the composition includes 10⁷ to 10¹¹ cfu. In embodiments, the composition includes 10⁸ to 10¹¹ cfu. In embodiments, the composition includes 10⁹ to 10¹¹ cfu.

In embodiments, a composition provided herein may be administered orally nasally and include live microorganisms (e.g., comprising, consisting essentially of, or consisting of Lactobacillus sakei bacterial cells) from 10³ to 10¹⁰ colony forming units (cfu)/g. In embodiments, the composition includes 10⁴ to 10¹⁰ cfu/g. In embodiments, the composition includes 10⁵ to 10¹⁰ cfu/g. In embodiments, the composition includes 10⁶ to 10¹⁰ cfu/g. In embodiments, the composition includes 10⁷ to 10¹⁰ cfu/g. In embodiments, the composition includes 10⁸ to 10¹⁰ cfu/g. In embodiments, the composition includes 10⁹ to 10¹⁰ cfu/g. In embodiments, the composition includes from 10³ to 10¹⁰ cfu. In embodiments, the composition includes 10⁴ to 10¹⁰ cfu. In embodiments, the composition includes 10⁵ to 10¹⁰ cfu. In embodiments, the composition includes 10⁶ to 10¹⁰ cfu. In embodiments, the composition includes 10⁷ to 10¹⁰ cfu. In embodiments, the composition includes 10⁸ to 10¹⁰ cfu. In embodiments, the composition includes 10⁹ to 10¹⁰ cfu.

In embodiments, a composition provided herein may be administered orally or nasally and include live microorganisms (e.g., comprising, consisting essentially of, or consisting of Lactobacillus sakei bacterial cells) from 10³ to 10⁹ colony forming units (cfu)/g. In embodiments, the composition includes 10⁴ to 10⁹ cfu/g. In embodiments, the composition includes 10⁵ to 10⁹ cfu/g. In embodiments, the composition includes 10⁶ to 10⁹ cfu/g. In embodiments, the composition includes 10⁷ to 10⁹ cfu/g. In embodiments, the composition includes 10⁸ to 10⁹ cfu/g. In embodiments, the composition includes from 10³ to 10⁹ cfu. In embodiments, the composition includes 10⁴ to 10⁹ cfu. In embodiments, the composition includes 10⁵ to 10⁹ cfu. In embodiments, the composition includes 10⁶ to 10⁹ cfu. In embodiments, the composition includes 10⁷ to 10⁹ cfu. In embodiments, the composition includes 10⁸ to 10⁹ cfu.

In embodiments, a composition provided herein may be administered orally or nasally and include live microorganisms (e.g., comprising, consisting essentially of, or consisting of Lactobacillus sakei bacterial cells) from 10³ to 10⁸ colony forming units (cfu)/g. In embodiments, the composition includes 10⁴ to 10⁸ cfu/g. In embodiments, the composition includes 10⁵ to 10⁸ cfu/g. In embodiments, the composition includes 10⁶ to 10⁸ cfu/g. In embodiments, the composition includes 10⁷ to 10⁸ cfu/g. In embodiments, the composition includes from 10³ to 10⁸ cfu. In embodiments, the composition includes 10⁴ to 10⁸ cfu. In embodiments, the composition includes 10⁵ to 10⁸ cfu. In embodiments, the composition includes 10⁶ to 10⁸ cfu. In embodiments, the composition includes 10⁷ to 10⁸ cfu.

In embodiments, a composition provided herein may be administered orally or nasally and include live microorganisms (e.g., comprising, consisting essentially of, or consisting of Lactobacillus sakei bacterial cells) from 10³ to 10⁷ colony forming units (cfu)/g. In embodiments, the composition includes 10⁴ to 10⁷ cfu/g. In embodiments, the composition includes 10⁵ to 10⁷ cfu/g. In embodiments, the composition includes 10⁶ to 10⁷ cfu/g. In embodiments, the composition includes from 10³ to 10⁷ cfu. In embodiments, the composition includes 10⁴ to 10⁷ cfu. In embodiments, the composition includes 10⁵ to 10⁷ cfu. In embodiments, the composition includes 10⁶ to 10⁷ cfu.

It is understood that the amount of colony forming units (cfu)/g and cfu as provided herein may refer to the amount of each bacterial species strain administered (individually) or the total cfu/g or cfu for a bacterial population.

The proportion or concentration of the compositions of the invention in a pharmaceutical composition can vary depending upon a number of factors including dosage, chemical characteristics (e.g., hydrophobicity), and the route of administration. For example, the defined microbial composition can be provided in a capsule containing from about 0.005 mg to about 1000 mg for oral administration. Alternatively or in addition, the dosage can be expressed as cfu or cfu/g of bacteria (e.g., of dry weight when expressed as cfu/g) as described above. In embodiments, the dosage may vary, but can range from the equivalent of about 10² to about 10¹⁵ cfu/g, e.g., 1×10² cfu/g, 5×10² cfu/g, 1×10³ cfu/g, 5×10³ cfu/g, 1×10⁴ cfu/g, 5×10⁴ cfu/g, 1×10⁵ cfu/g, 5×10⁵ cfu/g, 1×10⁶ cfu/g, 5×10⁶ cfu/g, 1×10⁷ cfu/g, 5×10⁷ cfu/g, 1×10⁸ cfu/g, 5×10⁸ cfu/g, 1×10⁹ cfu/g, 5×10⁹ cfu/g, 1×10¹⁰ cfu/g, 5×10¹⁰ cfu/g, 1×10¹¹ cfu/g, 5×10¹¹ cfu/g, or 1×10¹² cfu/g of dry weight.

### EMBODIMENTS

Embodiments include P1 to P94 following.

### Embodiment P1

A method of detecting whether a subject who has asthma has an increased risk of asthma exacerbation or rhinovirus infection compared to a general population of subjects who have asthma, comprising:

(a) obtaining a biological sample from the subject; and

(b) detecting a plurality of microorganisms in the biological sample.

### Embodiment P2

The method of embodiment P1, wherein the biological sample is a nasal saline wash.

### Embodiment P3

The method of embodiment P1, wherein the biological sample is a bodily fluid.

### Embodiment P4

The method of embodiment P1, wherein the bodily fluid is nasal mucus or discharge.

### Embodiment P5

The method of any one of embodiments P1-P4, wherein the microorganisms are bacterial microorganisms.

### Embodiment P6

The method of any one of embodiments P1-P5, wherein the subject is 6-18 years old.

### Embodiment P7

The method of any one of embodiments P1-P6, wherein omalizumab was previously administered to the subject.

### Embodiment P8

The method of any one of embodiments P1-P7, wherein the biological sample is obtained in June, July, August, or September.

### Embodiment P9

The method of any one of embodiments P1-P8, wherein the rhinovirus infection is a rhinovirus A infection or a rhinovirus B infection.

### Embodiment P10

The method of any one of embodiments P1-P9, wherein detecting a plurality of microorganisms in the biological sample comprises characterizing a microbiome in the biological sample.

### Embodiment P11

The method of any one of embodiments P1-P10, wherein characterizing the microbiome in the biological sample comprises determining the number and/or identity of bacterial taxa represented by bacteria in the biological sample.

### Embodiment P12

The method of embodiment P11, wherein the bacterial taxa are bacterial genera.

### Embodiment P13

The method of embodiment P11, wherein the bacterial taxa are bacterial families.

### Embodiment P14

The method of embodiment P11, wherein the bacterial taxa are bacterial families and/or genera.

### Embodiment P15

The method of any one of embodiments P1-P14, wherein characterizing the microbiome in the biological sample comprises detecting the proportion of bacteria in the biological sample that are within 1 of or any combination of 2, 3, 4, 5, 6, 7, 8, 9, or 10 of the following taxa: Moraxella, Staphylococcaceae, Corynebacterium, Staphylococcus, Streptococcus, and/or Haemophilus.

### Embodiment P16

The method of any one of embodiments P1-P15, wherein detecting a plurality of microorganisms in the biological sample comprises amplifying and sequencing 16S rRNA genes, or portions thereof, of microorganisms in the sample.

### Embodiment P17

The method of embodiment P16, wherein detecting a plurality of microorganisms in the biological sample comprises amplifying and sequencing the V4 region of 16S rRNA genes of microorganisms in the sample.

### Embodiment P18

The method of any one of embodiments P1-P17, wherein the microorganisms are bacterial microorganisms, and wherein detecting a plurality of microorganisms in the biological sample comprises detecting bacteria, or a proportion of bacteria, that are in 1, 2, 3, 4, 5, or 6 of the following taxa: Moraxella, Staphylococcaceae, Corynebacterium, Staphylococcus, Streptococcus, and/or Haemophilus.

### Embodiment P19

The method of embodiment P18, wherein detecting a plurality of microorganisms in the biological sample comprises detecting whether:
(a) the biological sample has an increased proportion of bacteria in the Moraxella genus of bacteria compared to a general population of subjects who have asthma;
(b) the biological sample has an increased proportion of bacteria in the Neisseria genus of bacteria compared to a general population of subjects who have asthma;
(c) the biological sample has a decreased proportion of bacteria in the Staphylococcaceae family or Corynebacterium genus of bacteria compared to a general population of subjects who have asthma;
(d) the biological sample has a decreased proportion of bacteria in the Corynebacterium genus of bacteria compared to a general population of subjects who have asthma;
(e) the biological sample has decreased proportion of bacteria in the Haemophilus genus of bacteria compared to a general population of subjects who have asthma;
(f) the biological sample has decreased proportion of bacteria in the Staphylococcaceae family of bacteria compared to a general population of subjects who have asthma;
(g) the biological sample has an increased proportion of bacteria in the Staphylococcus genus or
Corynebacterium genus of bacteria compared to a general population of subjects who have asthma;
(h) the biological sample has an increased proportion of bacteria in the Streptococcus genus of bacteria compared to a general population of subjects who have asthma; and/or
(i) the biological sample has an increased proportion of bacteria in the Haemophilus genus of bacteria compared to a general population of subjects who have asthma.

### Embodiment P20

The method of any one of embodiments P1-P19, further comprising identifying the subject as having an increased risk of asthma exacerbation if the subject:
(a) has an increased proportion of nasal microbiome bacteria in the Moraxella genus of bacteria compared to a general population of subjects who have asthma;
(b) has an increased proportion of nasal microbiome bacteria in the Moraxella genus of bacteria compared to a general population of subjects who have asthma;
(c) has an increased proportion of nasal microbiome bacteria in the Neisseria genus of bacteria compared to a general population of subjects who have asthma;
(d) has an increased proportion of nasal microbiome bacteria in the Staphylococcaceae family or
Corynebacterium genus of bacteria compared to a general population of subjects who have asthma;
(e) has an increased proportion of nasal microbiome bacteria in the Corynebacterium genus of bacteria compared to a general population of subjects who have asthma;
(f) has an increased proportion of nasal microbiome bacteria in the Haemophilus genus of bacteria compared to a general population of subjects who have asthma;
(g) has an increased proportion of nasal microbiome bacteria in the Staphylococcaceae family of bacteria compared to a general population of subjects who have asthma; and/or
(h) has an increased proportion of nasal microbiome bacteria in the Staphylococcus genus or Corynebacterium genus of bacteria compared to a general population of subjects who have asthma.

### Embodiment P21

The method of any one of embodiments P1-P20, further comprising identifying the subject as having an increased risk of rhinovirus infection if the subject:
(a) has an increased proportion of nasal microbiome bacteria in the Haemophilus genus of bacteria compared to a general population of subjects who have asthma;
(b) has an increased proportion of nasal microbiome bacteria in the Streptococcus genus of bacteria compared to a general population of subjects who have asthma;
(c) has an increased proportion of nasal microbiome bacteria in the Moraxella genus of bacteria compared to a general population of subjects who have asthma;
(d) has an increased proportion of nasal microbiome bacteria in the Neisseria genus of bacteria compared to a general population of subjects who have asthma; and/or
(e) has a decreased proportion of nasal microbiome bacteria in the Staphyloccocaceae family of bacteria compared to a general population of subjects who have asthma.

### Embodiment P22

A method of detecting whether a subject has or is at risk of developing chronic rhinosinusitis, comprising:

(a) obtaining a biological sample from the subject; and

(b) detecting a plurality of microorganisms in the biological sample.

### Embodiment P23

The method of embodiment P22, wherein the biological sample is sinus mucus.

### Embodiment P24

The method of embodiment P22, wherein the biological sample is a sinus brushing comprising mucus from the surface of a sinus.

### Embodiment P25

The method of any one of embodiments P22-P24, wherein the microorganisms are bacterial microorganisms.

### Embodiment P26

The method of any one of embodiments P22-P25, wherein detecting a plurality of microorganisms in the biological sample comprises characterizing the microbiome in the biological sample.

### Embodiment P27

The method of any one of embodiments P22-P26, wherein characterizing the microbiome in the biological sample comprises detecting the number and/or identity of bacterial taxa represented by bacteria in the biological sample.

### Embodiment P28

The method of embodiment P27, wherein the bacterial taxa are bacterial genera.

### Embodiment P29

The method of embodiment P27, wherein the bacterial taxa are bacterial families.

### Embodiment P30

The method of embodiment P27, wherein the bacterial taxa are bacterial families and/or genera.

### Embodiment P31

The method of any one of embodiments P22-P30, wherein characterizing the microbiome in the biological sample comprises detecting the proportion of bacteria in the biological sample that are within 1 of or any combination of 2, 3, 4, 5, 6, 7, 8, 9, or 10 of the following families: Corynebacteriaceae, Staphylococcaceae, Pseudomonadaceae, Streptococcaceae, Fusobacteriaceae, Pasteruellaceae, Prevotellaceae, Fusobacteriaceae, Neisseriaceae, and/or Enterobacteriaceae.

### Embodiment P32

The method of any one of embodiments P22-P30, wherein detecting a plurality of microorganisms in the biological sample comprises amplifying and sequencing 16S rRNA genes of microorganisms in the sample.

### Embodiment P33

The method of embodiment P32, wherein detecting a plurality of microorganisms in the biological sample comprises amplifying and sequencing the V4 region of 16S rRNA genes of microorganisms in the sample.

### Embodiment P34

The method of any one of embodiments P22-P33, wherein the microorganisms are bacterial microorganisms, and wherein detecting a plurality of microorganisms in the biological sample comprises detecting bacteria, or a proportion of bacteria, that are in 1 of or any combination of 2, 3, 4, 5, or 6 of the following families: Corynebacteriaceae, Staphylococcaceae, Pseudomonadaceae, Streptococcaceae, Fusobacteriaceae, Pasteruellaceae, Prevotellaceae, Fusobacteriaceae, Neisseriaceae, and/or Enterobacteriaceae.

### Embodiment P35

The method of embodiment P34, wherein detecting a plurality of microorganisms in the biological sample comprises detecting whether:
(a) the biological sample has an increased proportion of bacteria in the Prevotellaceae and Fusobacteriaceae families of bacteria compared to a general or healthy population of subjects;
(b) the biological sample has an increased proportion of bacteria in the Pseudomonas genus of bacteria compared to a general or healthy population of subjects;
(c) the biological sample has an increased proportion of bacteria in the Streptococcus, Porphyromonas, Tannerella, Treponema, Bacteroides, Dialister, and Akkermansia taxa of bacteria compared to a general or healthy population of subjects;
(d) the biological sample has an increased proportion of bacteria in the Prevotellaceae family of bacteria compared to a general or healthy population of subjects, wherein the increased proportion is a proportion of at least about 10% or 20%;
(e) the biological sample has an increased proportion of bacteria in the Fusobacteriaceae family of bacteria compared to a general or healthy population of subjects, wherein the increased proportion is a proportion of at least about 5% or 10%;
(f) at least 10% of the bacteria in the biological sample are in the Prevotellaceae family of bacteria, and at least 5% of the bacteria in the biological sample are in the Fusobacteriaceae family of bacteria;
(g) the biological sample has an increased proportion of bacteria in the Pseudomonadaceae, Fusobacteriaceae, Staphylococcaceae, and Enterobacteriaceae families of bacteria compared to a general or healthy population of subjects;
(h) the biological sample has an increased proportion of bacteria in the Pseudomonadaceae family of bacteria compared to a general or healthy population of subjects, wherein the increased proportion is a proportion of at least about 20%, 30%, 40%, or 50%;
(i) the biological sample has an increased proportion of bacteria in the Fusobacteriaceae family of bacteria compared to a general or healthy population of subjects, wherein the increased proportion is a proportion of at least about 5% or 10%;
(j) the biological sample has an increased proportion of bacteria in the Staphylococcaceae family of bacteria compared to a general or healthy population of subjects, wherein the increased proportion is a proportion of at least about 10% or 20%;
(k) the biological sample has an increased proportion of bacteria in the Enterobacteriaceae family of bacteria compared to a general or healthy population of subjects, wherein the increased proportion is a proportion of at least about 5%;
(l) at least 20% of the bacteria in the biological sample are in the Pseudomonadaceae family of bacteria, at least 5% of the bacteria in the biological sample are in the Fusobacteriaceae family of bacteria, at least 10% of the bacteria in the biological sample are in the Staphylococcaceae family of bacteria, and at least 5% of the bacteria in the biological sample are in the Enterobacteriaceae family of bacteria;
(m) the biological sample has an increased proportion of bacteria in the Sphingomonas genus of bacteria compared to a general or healthy population of subjects;
(n) the biological sample has an increased proportion of bacteria in the Corynebacteriaceae and Staphylococcaceae families of bacteria compared to a general or healthy population of subjects;
(o) the biological sample has an increased proportion of bacteria in the Corynebacteriaceae family of bacteria compared to a general or healthy population of subjects, wherein the increased proportion is a proportion of at least about 50%;
(p) the biological sample has an increased proportion of bacteria in the Staphylococcaceae family of bacteria compared to a general or healthy population of subjects, wherein the increased proportion is a proportion of at least about 10%;
(q) at least 50% of the bacteria in the biological sample are in the Corynebacteriaceae family of bacteria, and at least 10% of the bacteria in the biological sample are in the Staphylococcaceae family of bacteria;
(r) the biological sample has an increased proportion of bacteria in the Enterobacteriaceae and Staphylococcaceae families of bacteria compared to a general or healthy population of subjects;
(s) the biological sample has an increased proportion of bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects;
(t) the biological sample has an increased proportion of bacteria in the Enterobacteriaceae family of bacteria compared to a general or healthy population of subjects, wherein the increased proportion is a proportion of at least about 5%;
(u) the biological sample has an increased proportion of bacteria in the Staphylococcaceae family of bacteria compared to a general or healthy population of subjects, wherein the increased proportion is a proportion of at least about 10%, 20%, 30%, 40%, or 50%; and/or (v) at least 5% of the bacteria in the biological sample are in the Enterobacteriaceae family of bacteria, and at least 30% of the bacteria in the biological sample are in the Staphylococcaceae family of bacteria.

### Embodiment P36

The method of any one of embodiments P22-P35, further comprising determining the expression level of at least one gene in the biological sample.

### Embodiment P37

The method of embodiment P36, wherein the at least one gene is 1 of or any combination of 2, 3, 4, 5, 6, 7, 8, 9, 10, 11, 12, 13, 14, 15, 16, 17, 18, or 19 of any combination of Occludin, Claudin 2, MUCSAC, IL-4, IL-5, IL-6, IL-8, IL-25, IL-17A, IL-10, IL-1β, IL-33, CCL11, TSLP, TNF-α, ARG1, TGFβ1, CLCA1, and/or IFN-γ.

### Embodiment P38

The method of any one of embodiments P22-P37, further comprising identifying the subject as having or at risk of developing chronic rhinosinusitis if the subject:
(a) has increased expression of 1, 2, 3, 4, or 5 of any combination of IL-113, IL-6, IL-10, IL-5, and IFN-γ compared to a general or healthy population of subjects;
(b) the biological sample has an increased proportion of bacteria in the Pseudomonas genus of bacteria compared to a general or healthy population of subjects;
(c) has an increased proportion of sinus microbiome bacteria in the Prevotellaceae and Fusobacteriaceae families of bacteria compared to a general or healthy population of subjects;
(d) has an increased proportion of sinus microbiome bacteria in the Streptococcus, Porphyromonas, Tannerella, Treponema, Bacteroides, Dialister, and Akkermansia taxa of bacteria compared to a general or healthy population of subjects;
(e) has an increased proportion of sinus microbiome bacteria in the Prevotellaceae family of bacteria compared to a general or healthy population of subjects, wherein the increased proportion is a proportion of at least about 10% or 20%;
(f) has an increased proportion of sinus microbiome bacteria in the Fusobacteriaceae family of bacteria compared to a general or healthy population of subjects, wherein the increased proportion is a proportion of at least about 5% or 10%;
(g) has an increased proportion of sinus microbiome bacteria in the Pseudomonadaceae, Fusobacteriaceae, Staphylococcaceae, and Enterobacteriaceae families of bacteria compared to a general or healthy population of subjects;
(h) has an increased proportion of sinus microbiome bacteria in the Pseudomonadaceae family of bacteria compared to a general or healthy population of subjects, wherein the increased proportion is a proportion of at least about 20%, 30%, 40%, or 50%;
(i) has an increased proportion of sinus microbiome bacteria in the Fusobacteriaceae family of bacteria compared to a general or healthy population of subjects, wherein the increased proportion is a proportion of at least about 5% or 10%;
(j) has an increased proportion of sinus microbiome bacteria in the Staphylococcaceae family of bacteria compared to a general or healthy population of subjects, wherein the increased proportion is a proportion of at least about 10% or 20%;
(k) has an increased proportion of sinus microbiome bacteria in the Enterobacteriaceae family of bacteria compared to a general or healthy population of subjects, wherein the increased proportion is a proportion of at least about 5%;
(l) has an increased proportion of sinus microbiome bacteria in the Sphingomonas genus of bacteria compared to a general or healthy population of subjects;
(m) has an increased proportion of sinus microbiome bacteria in the Corynebacteriaceae and Staphylococcaceae families of bacteria compared to a general or healthy population of subjects;
(n) has an increased proportion of sinus microbiome bacteria in the Corynebacteriaceae family of bacteria compared to a general or healthy population of subjects, wherein the increased proportion is a proportion of at least about 50%;
(o) has an increased proportion of sinus microbiome bacteria in the Staphylococcaceae family of bacteria compared to a general or healthy population of subjects, wherein the increased proportion is a proportion of at least about 10%;
(p) has an increased proportion of sinus microbiome bacteria in the Enterobacteriaceae and Staphylococcaceae families of bacteria compared to a general or healthy population of subjects;
(q) has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects;
(r) has an increased proportion of sinus microbiome bacteria in the Enterobacteriaceae family of bacteria compared to a general or healthy population of subjects, wherein the increased proportion is a proportion of at least about 5%; and/or
(s) has an increased proportion of sinus microbiome bacteria in the Staphylococcaceae family of bacteria compared to a general or healthy population of subjects, wherein the increased proportion is a proportion of at least about 10%, 20%, 30%, 40%, or 50%.

### Embodiment P39

A method of detecting whether a subject who has chronic rhinosinusitis has an increased risk of nasal polyposis compared to a general population of subjects who have chronic rhinosinusitis comprising:

(a) obtaining a biological sample from the subject; and

(b) detecting a plurality of microorganisms in the biological sample.

### Embodiment P40

The method of embodiment P39, further comprising identifying the subject as having or at risk of developing nasal polyposis if the subject:
(a) has increased IL-5 or IFN-γ expression compared to a general or healthy population of subjects;
(b) the biological sample has an increased proportion of bacteria in the Pseudomonas genus of bacteria compared to a general or healthy population of subjects;
(c) has an increased proportion of sinus microbiome bacteria in the Enterobacteriaceae and Staphylococcaceae families of bacteria compared to a general or healthy population of subjects;
(d) has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects;
(e) has an increased proportion of sinus microbiome bacteria in the Enterobacteriaceae family of bacteria compared to a general or healthy population of subjects, wherein the increased proportion is a proportion of at least about 5%; and/or
(f) has an increased proportion of sinus microbiome bacteria in the Staphylococcaceae family of bacteria compared to a general or healthy population of subjects, wherein the increased proportion is a proportion of at least about 10%, 20%, 30%, 40%, or 50%.

### Embodiment P41

A method of treating or preventing asthma, an asthma exacerbation, or a rhinovirus infection in a subject in need thereof, comprising detecting nasal dysbiosis in the subject, and administering to the subject an effective amount of an antibiotic compound.

### Embodiment P42

A method of treating or preventing asthma, an asthma exacerbation, or a rhinovirus infection in a subject in need thereof, comprising administering to the subject an effective amount of an antibiotic compound, wherein the subject has been identified as having an increased risk of asthma exacerbation or rhinovirus infection compared to a general population of subjects who have asthma.

### Embodiment P43

A method of treating or preventing asthma, an asthma exacerbation, or a rhinovirus infection in a subject in need thereof, comprising administering to the subject an effective amount of an antibiotic compound.

### Embodiment P44

A method of treating or preventing chronic rhinosinusitis or nasal polyposis in a subject in need thereof, comprising detecting sinus dysbiosis in the subject, and administering to the subject an effective amount of an antibiotic compound.

### Embodiment P45

A method of treating or preventing chronic rhinosinusitis or nasal polyposis in a subject in need thereof, comprising administering to the subject an effective amount of an antibiotic compound, wherein the subject has been identified as having of chronic rhinosinusitis or nasal polyposis or at risk of chronic rhinosinusitis or nasal polyposis.

### Embodiment P46

A method of treating or preventing chronic rhinosinusitis or nasal polyposis in a subject in need thereof, comprising administering to the subject an effective amount of an antibiotic compound.

### Embodiment P47

The method of any one of embodiments P41-P46, wherein the subject has nasal dysbiosis or sinus dysbiosis.

### Embodiment P48

The method of embodiment P47, wherein the nasal dysbiosis or sinus dysbiosis comprises an increased proportion or amount of Staphylococcus sp. bacteria compared to a standard control.

### Embodiment P49

The method of embodiment P47, wherein the nasal dysbiosis or sinus dysbiosis comprises an increased proportion or amount of Corynebacterium sp. bacteria compared to a standard control.

### Embodiment P50

The method of embodiment P47, wherein the nasal dysbiosis or sinus dysbiosis comprises an increased proportion or amount of Pseudomonas sp. bacteria compared to a standard control.

### Embodiment P51

The method of embodiment P47, wherein the nasal dysbiosis or sinus dysbiosis comprises an increased proportion or amount of Streptococcus sp. bacteria compared to a standard control.

### Embodiment P52

The method of embodiment P47, wherein the nasal dysbiosis or sinus dysbiosis comprises an increased proportion or amount of Prevotella sp. bacteria compared to a standard control.

### Embodiment P53

The method of any one of embodiments P41-P52, wherein the antibiotic compound is a B-lactam, a cephalosporin, a lincosamide, a macrolide, a tetracycline, a sulfa compound, or mupirocin.

### Embodiment P54

The method of embodiment P53, wherein the antibiotic compound is oxacillin, flucloxacillin, cefazolin, cephalothin, cephalexin, erythromycin, doxycycline, or minocycline.

### Embodiment P55

The method of embodiment P53, wherein the mupirocin is administered in a cream

### Embodiment P56

The method of any one of embodiments P53-P55, wherein the subject has nasal dysbiosis comprising an increased proportion or amount of Staphylococcus sp. bacteria compared to a standard control.

### Embodiment P57

The method of any one of embodiments P41-P52, wherein the antibiotic compound is erythromycin, penicillin G, clarithromycin, bactrim DS, ciprofloxacin, vancomycin, daptomycin, or linezolid.

### Embodiment P58

The method of embodiment P57, wherein the subject has nasal dysbiosis comprising an increased proportion or amount of Corynebacterium sp. bacteria compared to a standard control.

### Embodiment P59

The method of any one of embodiments P41-P52, wherein the antibiotic compound is a penicillin, a +/−beta-lactam inhibitor, a cephalosporin, a monobactam, a fluoroquinolone, a carbapenem, an aminoglycoside, or a polymixin.

### Embodiment P60

The method of embodiment P59, wherein the subject has sinus dysbiosis comprising an increased proportion or amount of Pseudomonas sp. bacteria compared to a standard control.

### Embodiment P61

The method of embodiment P60, wherein the subject has sinus dysbiosis comprising an increased proportion or amount of Pseudomonas sp. bacteria compared to a standard control, wherein the Pseudomonas sp. bacteria comprise an antibiotic resistant strain, and wherein the antibiotic is a polymixin.

### Embodiment P62

The method of any one of embodiments P41-P52, wherein the antibiotic is a penicillin or a cephalosporin.

### Embodiment P63

The method of embodiment P62, wherein the subject has nasal dysbiosis comprising an increased proportion or amount of Streptococcus sp. bacteria compared to a standard control.

### Embodiment P64

The method of any one of embodiments P41-P52, wherein the antibiotic is metronidazole, amoxicillin, clavulanate, amoxicillin and clavulanate, a ureidopenicilin, a carbapenem, a cephalosporin, clindamycin, or chloramphenicol.

### Embodiment P65

The method of embodiment P64, wherein the subject has sinus dysbiosis comprising an increased proportion or amount of Prevotella sp. bacteria compared to a standard control.

### Embodiment P66

The method of any one of embodiments P41-P65, further comprising administering at least one bacterium to the subject.

### Embodiment P67

The method of embodiment P66, wherein the at least one bacterium comprises a Lactobacillus sakei bacterium.

### Embodiment P68

The method of any one of embodiments P41-P67, further comprising administering an anti-IL-5 compound to the subject.

### Embodiment P69

A method of treating or preventing chronic rhinosinusitis or nasal polyposis in a subject in need thereof, comprising administering to the subject an effective amount of an anti-IL-5 compound.

### Embodiment P70

The method of embodiment P69, wherein the anti-IL-5 compound is an anti-IL-5 antibody.

### Embodiment P71

The method of embodiment P70, wherein the anti-IL-5 antibody is reslizumab or mepolizumab.

### Embodiment P72

The method of embodiment P69, wherein the anti-IL-5 compound is an anti-IL-5 receptor antibody.

### Embodiment P73

The method of embodiment P72, wherein the anti-IL-5 receptor antibody is benralizumab.

### Embodiment P74

The method of any one of embodiments P69-P73, further comprising administering at least one bacterium to the subject.

### Embodiment P75

The method of embodiment P74, wherein the at least one bacterium comprises a Lactobacillus sakei bacterium.

### Embodiment P76

A method of treating or preventing asthma, asthma exacerbation, or a rhinovirus infection in a subject in need thereof, comprising detecting nasal dysbiosis in the subject, and administering to the subject an effective amount of at least one bacterium.

### Embodiment P77

A method of treating or preventing asthma, an asthma exacerbation, or a rhinovirus infection in a subject in need thereof, comprising administering to the subject an effective amount of at least one bacterium, wherein the subject has been identified as having an increased risk of asthma exacerbation or rhinovirus infection compared to a general population of subjects who have asthma.

### Embodiment P78

A method of treating or preventing asthma, an asthma exacerbation, or a rhinovirus infection in a subject in need thereof, comprising administering to the subject an effective amount of at least one bacterium.

### Embodiment P79

A method of treating or preventing chronic rhinosinusitis or nasal polyposis in a subject in need thereof, comprising detecting sinus dysbiosis in the subject, and administering to the subject an effective amount of at least one bacterium.

### Embodiment P80

A method of treating or preventing chronic rhinosinusitis or nasal polyposis in a subject in need thereof, comprising administering to the subject an effective amount of at least one bacterium, wherein the subject has been identified as having of chronic rhinosinusitis or nasal polyposis or at risk of chronic rhinosinusitis or nasal polyposis.

### Embodiment P81

A method of treating or preventing chronic rhinosinusitis or nasal polyposis in a subject in need thereof, comprising administering to the subject an effective amount of at least one bacterium.

### Embodiment P82

The method of embodiment P81, wherein the subject has nasal dysbiosis or sinus dysbiosis.

### Embodiment P83

The method of embodiment P82, wherein the nasal dysbiosis or sinus dysbiosis comprises an increased proportion or amount of Corynebacterium sp. bacteria compared to a standard control.

### Embodiment P84

The method of embodiment P83, wherein the nasal dysbiosis or sinus dysbiosis comprises an increased proportion or amount of Pseudomonas sp. bacteria compared to a standard control.

### Embodiment P85

The method of any one of embodiments P76-P84, wherein the at least one bacterium is Lactobacillus sakei.

### Embodiment P86

A method of treating or preventing chronic rhinosinusitis or nasal polyposis in a subject in need thereof, comprising detecting an increased proportion or amount of Corynebacterium sp. bacteria or Pseudomonas sp. bacteria in the sinus microbiome of the subject compared to a standard control, and administering to the subject an effective amount of a Lactobacillus sakei bacterium.

### Embodiment P87

A method of treating or preventing chronic rhinosinusitis or nasal polyposis in a subject in need thereof, comprising administering to the subject an effective amount of a Lactobacillus sakei bacterium, wherein the subject has been identified as having an increased proportion or amount of Corynebacterium sp. bacteria or Pseudomonas sp. bacteria in the sinus microbiome of the subject compared to a standard control.

### Embodiment P88

A method of treating or preventing chronic rhinosinusitis or nasal polyposis in a subject in need thereof, comprising administering to the subject an effective amount of a Lactobacillus sakei bacterium, wherein the subject has an increased proportion or amount of Corynebacterium sp. bacteria or Pseudomonas sp. bacteria in the sinus microbiome of the subject compared to a standard control.

### Embodiment P89

A method of treating or preventing dysbiosis in a subject in need thereof, the method comprising administering to the subject an effective amount of a Lactobacillus sakei bacterium.

### Embodiment P90

A composition comprising an isolated Lactobacillus sakei bacterium and a pharmaceutically acceptable excipient.

### Embodiment P91

The composition of embodiment P90, wherein the pharmaceutically acceptable excipient is suitable for nasal administration.

### Embodiment P92

The composition of embodiment P90 or P91, which is a capsule, a tablet, a suspension, a suppository, a powder, a cream, an oil, an oil-in-water emulsion, a water-in-oil emulsion, or an aqueous solution.

### Embodiment P93

The composition of embodiment P90 or P91, which is in the form of a powder, a solid, a semi-solid, or a liquid.

### Embodiment P94

The composition of embodiment P90 or P91, further comprising an anti-IL-5 compound.

Additional embodiments include Embodiments 1 to 75 following:

### Embodiment 1

A method of detecting a nasal or sinus microbiome in a subject who has asthma, the method comprising detecting bacteria, or a proportion of bacteria, in a biological sample from the subject that are in 1 of or any combination of the following taxa: Moraxella, Staphylococcaceae, Corynebacterium, Corynebacteriaceae, Staphylococcus, Staphylococcaceae, Streptococcus, Streptococcaceae, P seudomonadaceae, Haemophilus, Fusobacteriaceae, Pasteruellaceae, Prevotellaceae, Fusobacteriaceae, Neisseriaceae, and/or Enterobacteriaceae.

### Embodiment 2

The method of Embodiment 1, wherein detecting the nasal or sinus microbiome comprises amplifying and sequencing 16S rRNA genes, or portions thereof, of microorganisms in the biological sample.

### Embodiment 3

The method of Embodiment 1 or 2, wherein detecting nasal or sinus microbiome comprises amplifying and sequencing the V4 region of 16S rRNA genes of microorganisms in the biological sample.

### Embodiment 4

The method of any one of Embodiments 1-3, comprising detecting the proportion of bacteria in the biological sample that are within 1 of or any combination of 2, 3, 4, 5, 6, 7, 8, 9, or 10 of the following taxa: Moraxella, Staphylococcaceae, Corynebacterium, Staphylococcus, Streptococcus, and/or Haemophilus.

### Embodiment 5

The method of any one of Embodiments 1-4, comprising detecting the proportion of bacteria in the biological sample that are within 1 of or any combination of 2, 3, 4, 5, 6, 7, 8, 9, or 10 of the following families: Corynebacteriaceae, Staphylococcaceae, Pseudomonadaceae, Streptococcaceae, Fusobacteriaceae, Pasteruellaceae, Prevotellaceae, Fusobacteriaceae, Neisseriaceae, and/or Enterobacteriaceae.

### Embodiment 6

The method of any one of Embodiments 1-5, comprising detecting whether:
(a) the biological sample has an increased proportion of bacteria in the Moraxella genus of bacteria compared to a general population of subjects who have asthma;
(b) the biological sample has an increased proportion of bacteria in the Neisseria genus of bacteria compared to a general population of subjects who have asthma;
(c) the biological sample has a decreased proportion of bacteria in the Staphylococcaceae family or Corynebacterium genus of bacteria compared to a general population of subjects who have asthma;
(d) the biological sample has a decreased proportion of bacteria in the Corynebacterium genus of bacteria compared to a general population of subjects who have asthma;
(e) the biological sample has decreased proportion of bacteria in the Haemophilus genus of bacteria compared to a general population of subjects who have asthma;
(f) the biological sample has decreased proportion of bacteria in the Staphylococcaceae family of bacteria compared to a general population of subjects who have asthma;
(g) the biological sample has an increased proportion of bacteria in the Staphylococcus genus or
Corynebacterium genus of bacteria compared to a general population of subjects who have asthma;
(h) the biological sample has an increased proportion of bacteria in the Streptococcus genus of bacteria compared to a general population of subjects who have asthma; and/or
(i) the biological sample has an increased proportion of bacteria in the Haemophilus genus of bacteria compared to a general population of subjects who have asthma.

### Embodiment 7

The method of any one of Embodiments 1-5, comprising detecting whether the subject:
(a) has an increased proportion of nasal microbiome bacteria in the Moraxella genus of bacteria compared to a general population of subjects who have asthma;
(b) has an increased proportion of nasal microbiome bacteria in the Moraxella genus of bacteria compared to a general population of subjects who have asthma;
(c) has an increased proportion of nasal microbiome bacteria in the Neisseria genus of bacteria compared to a general population of subjects who have asthma;
(d) has an increased proportion of nasal microbiome bacteria in the Staphylococcaceae family or
Corynebacterium genus of bacteria compared to a general population of subjects who have asthma;
(e) has an increased proportion of nasal microbiome bacteria in the Corynebacterium genus of bacteria compared to a general population of subjects who have asthma;
(f) has an increased proportion of nasal microbiome bacteria in the Haemophilus genus of bacteria compared to a general population of subjects who have asthma;
(g) has an increased proportion of nasal microbiome bacteria in the Staphylococcaceae family of bacteria compared to a general population of subjects who have asthma; and/or
(h) has an increased proportion of nasal microbiome bacteria in the Staphylococcus genus or Corynebacterium genus of bacteria compared to a general population of subjects who have asthma.

### Embodiment 8

The method of any one of Embodiments 1-5, comprising detecting whether the subject:
(a) has an increased proportion of nasal microbiome bacteria in the Haemophilus genus of bacteria compared to a general population of subjects who have asthma;
(b) has an increased proportion of nasal microbiome bacteria in the Streptococcus genus of bacteria compared to a general population of subjects who have asthma;
(c) has an increased proportion of nasal microbiome bacteria in the Moraxella genus of bacteria compared to a general population of subjects who have asthma;
(d) has an increased proportion of nasal microbiome bacteria in the Neisseria genus of bacteria compared to a general population of subjects who have asthma; and/or
(e) has a decreased proportion of nasal microbiome bacteria in the Staphyloccocaceae family of bacteria compared to a general population of subjects who have asthma.

### Embodiment 9

The method of any one of Embodiments 1-5, comprising detecting whether:
(a) the biological sample has an increased proportion of bacteria in the Prevotellaceae and Fusobacteriaceae families of bacteria compared to a general or healthy population of subjects;
(b) the biological sample has an increased proportion of bacteria in the Pseudomonas genus of bacteria compared to a general or healthy population of subjects;
(c) the biological sample has an increased proportion of bacteria in the Streptococcus, Porphyromonas, Tannerella, Treponema, Bacteroides, Dialister, and Akkermansia taxa of bacteria compared to a general or healthy population of subjects;
(d) the biological sample has an increased proportion of bacteria in the Prevotellaceae family of bacteria compared to a general or healthy population of subjects, wherein the increased proportion is a proportion of at least about 10% or 20%;
(e) the biological sample has an increased proportion of bacteria in the Fusobacteriaceae family of bacteria compared to a general or healthy population of subjects, wherein the increased proportion is a proportion of at least about 5% or 10%;
(f) at least 10% of the bacteria in the biological sample are in the Prevotellaceae family of bacteria, and at least 5% of the bacteria in the biological sample are in the Fusobacteriaceae family of bacteria;
(g) the biological sample has an increased proportion of bacteria in the Pseudomonadaceae, Fusobacteriaceae, Staphylococcaceae, and Enterobacteriaceae families of bacteria compared to a general or healthy population of subjects;
(h) the biological sample has an increased proportion of bacteria in the Pseudomonadaceae family of bacteria compared to a general or healthy population of subjects, wherein the increased proportion is a proportion of at least about 20%, 30%, 40%, or 50%;
(i) the biological sample has an increased proportion of bacteria in the Fusobacteriaceae family of bacteria compared to a general or healthy population of subjects, wherein the increased proportion is a proportion of at least about 5% or 10%;
(j) the biological sample has an increased proportion of bacteria in the Staphylococcaceae family of bacteria compared to a general or healthy population of subjects, wherein the increased proportion is a proportion of at least about 10% or 20%;
(k) the biological sample has an increased proportion of bacteria in the Enterobacteriaceae family of bacteria compared to a general or healthy population of subjects, wherein the increased proportion is a proportion of at least about 5%;
(l) at least 20% of the bacteria in the biological sample are in the Pseudomonadaceae family of bacteria, at least 5% of the bacteria in the biological sample are in the Fusobacteriaceae family of bacteria, at least 10% of the bacteria in the biological sample are in the Staphylococcaceae family of bacteria, and at least 5% of the bacteria in the biological sample are in the Enterobacteriaceae family of bacteria;
(m) the biological sample has an increased proportion of bacteria in the Sphingomonas genus of bacteria compared to a general or healthy population of subjects;
(n) the biological sample has an increased proportion of bacteria in the Corynebacteriaceae and Staphylococcaceae families of bacteria compared to a general or healthy population of subjects;
(o) the biological sample has an increased proportion of bacteria in the Corynebacteriaceae family of bacteria compared to a general or healthy population of subjects, wherein the increased proportion is a proportion of at least about 50%;
(p) the biological sample has an increased proportion of bacteria in the Staphylococcaceae family of bacteria compared to a general or healthy population of subjects, wherein the increased proportion is a proportion of at least about 10%;
(q) at least 50% of the bacteria in the biological sample are in the Corynebacteriaceae family of bacteria, and at least 10% of the bacteria in the biological sample are in the Staphylococcaceae family of bacteria;
(r) the biological sample has an increased proportion of bacteria in the Enterobacteriaceae and Staphylococcaceae families of bacteria compared to a general or healthy population of subjects;
(s) the biological sample has an increased proportion of bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects;
(t) the biological sample has an increased proportion of bacteria in the Enterobacteriaceae family of bacteria compared to a general or healthy population of subjects, wherein the increased proportion is a proportion of at least about 5%;
(u) the biological sample has an increased proportion of bacteria in the Staphylococcaceae family of bacteria compared to a general or healthy population of subjects, wherein the increased proportion is a proportion of at least about 10%, 20%, 30%, 40%, or 50%; and/or (v) at least 5% of the bacteria in the biological sample are in the Enterobacteriaceae family of bacteria, and at least 30% of the bacteria in the biological sample are in the Staphylococcaceae family of bacteria.

### Embodiment 10

The method of any one of Embodiments 1-9, further comprising determining the expression level of at least one gene in the biological sample.

### Embodiment 11

The method of Embodiment 10, wherein the at least one gene is any 1 of or any combination of 2, 3, 4, 5, 6, 7, 8, 9, 10, 11, 12, 13, 14, 15, 16, 17, 18, or 19 of any combination of Occludin, Claudin 2, MUCSAC, IL-4, IL-5, IL-6, IL-8, IL-25, IL-17A, IL-10, IL-1β, IL-33, CCL11, TSLP, TNF-α, ARG1, TGFβ1, CLCA1, and/or IFN-γ.

### Embodiment 12

The method of any one of Embodiments 1-11, comprising detecting whether the subject:
(a) has increased expression of any 1 of or 2, 3, 4, or 5 of any combination of IL-1β, IL-6, IL-10, IL-5, and IFN-γ compared to a general or healthy population of subjects;
(b) the biological sample has an increased proportion of bacteria in the Pseudomonas genus of bacteria compared to a general or healthy population of subjects;
(c) has an increased proportion of sinus microbiome bacteria in the Prevotellaceae and Fusobacteriaceae families of bacteria compared to a general or healthy population of subjects;
(d) has an increased proportion of sinus microbiome bacteria in the Streptococcus, Porphyromonas, Tannerella, Treponema, Bacteroides, Dialister, and Akkermansia taxa of bacteria compared to a general or healthy population of subjects;
(e) has an increased proportion of sinus microbiome bacteria in the Prevotellaceae family of bacteria compared to a general or healthy population of subjects, wherein the increased proportion is a proportion of at least about 10% or 20%;
(f) has an increased proportion of sinus microbiome bacteria in the Fusobacteriaceae family of bacteria compared to a general or healthy population of subjects, wherein the increased proportion is a proportion of at least about 5% or 10%;
(g) has an increased proportion of sinus microbiome bacteria in the Pseudomonadaceae,
Fusobacteriaceae, Staphylococcaceae, and Enterobacteriaceae families of bacteria compared to a general or healthy population of subjects;
(h) has an increased proportion of sinus microbiome bacteria in the Pseudomonadaceae family of bacteria compared to a general or healthy population of subjects, wherein the increased proportion is a proportion of at least about 20%, 30%, 40%, or 50%;
(i) has an increased proportion of sinus microbiome bacteria in the Fusobacteriaceae family of bacteria compared to a general or healthy population of subjects, wherein the increased proportion is a proportion of at least about 5% or 10%;
(j) has an increased proportion of sinus microbiome bacteria in the Staphylococcaceae family of bacteria compared to a general or healthy population of subjects, wherein the increased proportion is a proportion of at least about 10% or 20%;
(k) has an increased proportion of sinus microbiome bacteria in the Enterobacteriaceae family of bacteria compared to a general or healthy population of subjects, wherein the increased proportion is a proportion of at least about 5%;
(l) has an increased proportion of sinus microbiome bacteria in the Sphingomonas genus of bacteria compared to a general or healthy population of subjects;
(m) has an increased proportion of sinus microbiome bacteria in the Corynebacteriaceae and Staphylococcaceae families of bacteria compared to a general or healthy population of subjects;
(n) has an increased proportion of sinus microbiome bacteria in the Corynebacteriaceae family of bacteria compared to a general or healthy population of subjects, wherein the increased proportion is a proportion of at least about 50%;
(o) has an increased proportion of sinus microbiome bacteria in the Staphylococcaceae family of bacteria compared to a general or healthy population of subjects, wherein the increased proportion is a proportion of at least about 10%;
(p) has an increased proportion of sinus microbiome bacteria in the Enterobacteriaceae and Staphylococcaceae families of bacteria compared to a general or healthy population of subjects;
(q) has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects;
(r) has an increased proportion of sinus microbiome bacteria in the Enterobacteriaceae family of bacteria compared to a general or healthy population of subjects, wherein the increased proportion is a proportion of at least about 5%; and/or
(s) has an increased proportion of sinus microbiome bacteria in the Staphylococcaceae family of bacteria compared to a general or healthy population of subjects, wherein the increased proportion is a proportion of at least about 10%, 20%, 30%, 40%, or 50%.

### Embodiment 13

The method of any one of Embodiments 1-11, comprising detecting whether the subject:
(a) has increased IL-5 or IFN-γ expression compared to a general or healthy population of subjects;
(b) the biological sample has an increased proportion of bacteria in the Pseudomonas genus of bacteria compared to a general or healthy population of subjects;
(c) has an increased proportion of sinus microbiome bacteria in the Enterobacteriaceae and Staphylococcaceae families of bacteria compared to a general or healthy population of subjects;
(d) has an increased proportion of sinus microbiome bacteria in the Actinobacteria, Bifidobacterium, Haemophilus, Enterobacteriaceae, Pseudomonadaceae, Sphingomonadaceae, Selenomonas and Streptophyta taxa of bacteria compared to a general or healthy population of subjects;
(e) has an increased proportion of sinus microbiome bacteria in the Enterobacteriaceae family of bacteria compared to a general or healthy population of subjects, wherein the increased proportion is a proportion of at least about 5%; and/or
(f) has an increased proportion of sinus microbiome bacteria in the Staphylococcaceae family of bacteria compared to a general or healthy population of subjects, wherein the increased proportion is a proportion of at least about 10%, 20%, 30%, 40%, or 50%.

### Embodiment 14

The method of any one of Embodiments 1-13, wherein the biological sample is a nasal saline wash.

### Embodiment 15

The method of any one of Embodiments 1-13, wherein the biological sample is a bodily fluid.

### Embodiment 16

The method of any one of Embodiments 1-13, wherein the bodily fluid is nasal mucus or discharge.

### Embodiment 17

The method of any one of Embodiments 1-13, wherein the biological sample is sinus mucus.

### Embodiment 18

The method of any one of Embodiments 1-13, wherein the biological sample is a sinus brushing comprising mucus from the surface of a sinus.

### Embodiment 19

The method of any one of Embodiments 1-13, wherein the subject is 6-18 years old.

### Embodiment 20

The method of any one of Embodiments 1-13, wherein omalizumab was previously administered to the subject.

### Embodiment 21

The method of any one of Embodiments 1-13, wherein the biological sample is obtained in June, July, August, or September.

### Embodiment 22

A method of treating or preventing asthma, an asthma exacerbation, or a rhinovirus infection in a subject in need thereof, comprising detecting nasal dysbiosis in the subject, and administering to the subject an effective amount of an antibiotic compound.

### Embodiment 23

A method of treating or preventing asthma, an asthma exacerbation, or a rhinovirus infection in a subject in need thereof, comprising administering to the subject an effective amount of an antibiotic compound, wherein the subject has been identified as having an increased risk of asthma exacerbation or rhinovirus infection compared to a general population of subjects who have asthma.

### Embodiment 24

A method of treating or preventing asthma, an asthma exacerbation, or a rhinovirus infection in a subject in need thereof, comprising administering to the subject an effective amount of an antibiotic compound.

### Embodiment 25

A method of treating or preventing chronic rhinosinusitis or nasal polyposis in a subject in need thereof, comprising detecting sinus dysbiosis in the subject, and administering to the subject an effective amount of an antibiotic compound.

### Embodiment 26

A method of treating or preventing chronic rhinosinusitis or nasal polyposis in a subject in need thereof, comprising administering to the subject an effective amount of an antibiotic compound, wherein the subject has been identified as having of chronic rhinosinusitis or nasal polyposis or at risk of chronic rhinosinusitis or nasal polyposis.

### Embodiment 27

A method of treating or preventing chronic rhinosinusitis or nasal polyposis in a subject in need thereof, comprising administering to the subject an effective amount of an antibiotic compound.

### Embodiment 28

The method of any one of Embodiments 22-27, wherein the subject has nasal dysbiosis or sinus dysbiosis.

### Embodiment 29

The method of Embodiment 28, wherein the nasal dysbiosis or sinus dysbiosis comprises an increased proportion or amount of Staphylococcus sp. bacteria compared to a standard control.

### Embodiment 30

The method of Embodiment 28 or 29, wherein the nasal dysbiosis or sinus dysbiosis comprises an increased proportion or amount of Corynebacterium sp. bacteria compared to a standard control.

### Embodiment 31

The method of any one of Embodiments 28-30, wherein the nasal dysbiosis or sinus dysbiosis comprises an increased proportion or amount of Pseudomonas sp. bacteria compared to a standard control.

### Embodiment 32

The method of any one of Embodiments 28-31, wherein the nasal dysbiosis or sinus dysbiosis comprises an increased proportion or amount of Streptococcus sp. bacteria compared to a standard control.

### Embodiment 33

The method of any one of Embodiments 28-32, wherein the nasal dysbiosis or sinus dysbiosis comprises an increased proportion or amount of Prevotella sp. bacteria compared to a standard control.

### Embodiment 34

The method of any one of Embodiments 22-33, wherein the antibiotic compound is a B-lactam, a cephalosporin, a lincosamide, a macrolide, a tetracycline, a sulfa compound, or mupirocin.

### Embodiment 35

The method of any one of Embodiments 22-34, wherein the antibiotic compound is oxacillin, flucloxacillin, cefazolin, cephalothin, cephalexin, erythromycin, doxycycline, or minocycline.

### Embodiment 36

The method of Embodiment 34, wherein the mupirocin is administered in a cream.

### Embodiment 37

The method of Embodiment 34, wherein the subject has nasal dysbiosis comprising an increased proportion or amount of Staphylococcus sp. bacteria compared to a standard control.

### Embodiment 38

The method any one of Embodiments 22-37, wherein the antibiotic compound is erythromycin, penicillin G, clarithromycin, bactrim DS, ciprofloxacin, vancomycin, daptomycin, or linezolid.

### Embodiment 39

The method of Embodiment 38, wherein the subject has nasal dysbiosis comprising an increased proportion or amount of Corynebacterium sp. bacteria compared to a standard control.

### Embodiment 40

The method of any one of Embodiments 22-39, wherein the antibiotic compound is a penicillin, a +/−beta-lactam inhibitor, a cephalosporin, a monobactam, a fluoroquinolone, a carbapenem, an aminoglycoside, or a polymixin.

### Embodiment 41

The method of Embodiment 40, wherein the subject has sinus dysbiosis comprising an increased proportion or amount of Pseudomonas sp. bacteria compared to a standard control.

### Embodiment 42

The method of Embodiment 41, wherein the subject has sinus dysbiosis comprising an increased proportion or amount of Pseudomonas sp. bacteria compared to a standard control, wherein the Pseudomonas sp. bacteria comprise an antibiotic resistant strain, and wherein the antibiotic is a polymixin.

### Embodiment 43

The method of any one of Embodiments 22-42, wherein the antibiotic is a penicillin or a cephalosporin

### Embodiment 44

The method of Embodiment 43, wherein the subject has nasal dysbiosis comprising an increased proportion or amount of Streptococcus sp. bacteria compared to a standard control.

### Embodiment 45

The method of any one of Embodiments 22-44, wherein the antibiotic is metronidazole, amoxicillin, clavulanate, amoxicillin and clavulanate, a ureidopenicilin, a carbapenem, a cephalosporin, clindamycin, or chloramphenicol.

### Embodiment 46

The method of Embodiiment 45, wherein the subject has sinus dysbiosis comprising an increased proportion or amount of Prevotella sp. bacteria compared to a standard control.

### Embodiment 47

The method of any one of Embodiments 22-46, further comprising administering at least one bacterium to the subject.

### Embodiment 48

The method of Embodiment 47, wherein the at least one bacterium comprises a Lactobacillus sakei bacterium.

### Embodiment 48

The method of any one of Embodiments 22-48, further comprising administering an anti-IL-5 compound to the subject.

### Embodiment 50

A method of treating or preventing chronic rhinosinusitis or nasal polyposis in a subject in need thereof, comprising administering to the subject an effective amount of an anti-IL-5 compound.

### Embodiment 51

The method of Embodiment 50, wherein the anti-IL-5 compound is an anti-IL-5 antibody.

### Embodiment 52

The method of Embodiment 51, wherein the anti-IL-5 antibody is reslizumab or mepolizumab.

### Embodiment 53

The method of Embodiment 50, wherein the anti-IL-5 compound is an anti-IL-5 receptor antibody.

### Embodiment 54

The method of Embodiment 53, wherein the anti-IL-5 receptor antibody is benralizumab.

### Embodiment 55

The method of any one of Embodiments 50-54, further comprising administering at least one bacterium to the subject.

### Embodiment 56

The method of Embodiment 55, wherein the at least one bacterium comprises a Lactobacillus sakei bacterium.

### Embodiment 57

A method of treating or preventing asthma, asthma exacerbation, or a rhinovirus infection in a subject in need thereof, comprising detecting nasal dysbiosis in the subject, and administering to the subject an effective amount of at least one bacterium.

### Embodiment 58

A method of treating or preventing asthma, an asthma exacerbation, or a rhinovirus infection in a subject in need thereof, comprising administering to the subject an effective amount of at least one bacterium, wherein the subject has been identified as having an increased risk of asthma exacerbation or rhinovirus infection compared to a general population of subjects who have asthma.

### Embodiment 59

A method of treating or preventing asthma, an asthma exacerbation, or a rhinovirus infection in a subject in need thereof, comprising administering to the subject an effective amount of at least one bacterium.

### Embodiment 60

A method of treating or preventing chronic rhinosinusitis or nasal polyposis in a subject in need thereof, comprising detecting sinus dysbiosis in the subject, and administering to the subject an effective amount of at least one bacterium.

### Embodiment 61

A method of treating or preventing chronic rhinosinusitis or nasal polyposis in a subject in need thereof, comprising administering to the subject an effective amount of at least one bacterium, wherein the subject has been identified as having of chronic rhinosinusitis or nasal polyposis or at risk of chronic rhinosinusitis or nasal polyposis.

### Embodiment 62

A method of treating or preventing chronic rhinosinusitis or nasal polyposis in a subject in need thereof, comprising administering to the subject an effective amount of at least one bacterium.

### Embodiment 63

The method of Embodiment 62, wherein the subject has nasal dysbiosis or sinus dysbiosis.

### Embodiment 64

The method of Embodiment 63, wherein the nasal dysbiosis or sinus dysbiosis comprises an increased proportion or amount of Corynebacterium sp. bacteria compared to a standard control.

### Embodiment 65

The method of Embodiment 63 or 64, wherein the nasal dysbiosis or sinus dysbiosis comprises an increased proportion or amount of Pseudomonas sp. bacteria compared to a standard control.

### Embodiment 66

The method of any one of Embodiments 57-65, wherein the at least one bacterium is Lactobacillus sakei.

### Embodiment 67

A method of treating or preventing chronic rhinosinusitis or nasal polyposis in a subject in need thereof, comprising detecting an increased proportion or amount of Corynebacterium sp. bacteria or Pseudomonas sp. bacteria in the sinus microbiome of the subject compared to a standard control, and administering to the subject an effective amount of a Lactobacillus sakei bacterium.

### Embodiment 68

A method of treating or preventing chronic rhinosinusitis or nasal polyposis in a subject in need thereof, comprising administering to the subject an effective amount of a Lactobacillus sakei bacterium, wherein the subject has been identified as having an increased proportion or amount of Corynebacterium sp. bacteria or Pseudomonas sp. bacteria in the sinus microbiome of the subject compared to a standard control.

### Embodiment 69

A method of treating or preventing chronic rhinosinusitis or nasal polyposis in a subject in need thereof, comprising administering to the subject an effective amount of a Lactobacillus sakei bacterium, wherein the subject has an increased proportion or amount of Corynebacterium sp. bacteria or Pseudomonas sp. bacteria in the sinus microbiome of the subject compared to a standard control.

### Embodiment 70

A method of treating or preventing dysbiosis in a subject in need thereof, the method comprising administering to the subject an effective amount of a Lactobacillus sakei bacterium.

### Embodiment 71

A composition comprising an isolated Lactobacillus sakei bacterium and a pharmaceutically acceptable excipient.

### Embodiment 72

The composition of Embodiment 71, wherein the pharmaceutically acceptable excipient is suitable for nasal administration.

### Embodiment 73

The composition of Embodiment 71 or 72, which is a capsule, a tablet, a suspension, a suppository, a powder, a cream, an oil, an oil-in-water emulsion, a water-in-oil emulsion, or an aqueous solution.

### Embodiment 74

The composition of any one of Embodiments 71-73, which is in the form of a powder, a solid, a semi-solid, or a liquid.

### Embodiment 75

The composition of any one of Embodiments 71-74, further comprising an anti-IL-5 compound.

### EXAMPLES

The following examples are offered to illustrate, but not to limit the claimed invention.

### Example 1. Distinct Upper Airway Bacterial Microbiota Differentially Relate to Rhinovirus Infection and Airway Exacerbation in Pediatric Asthma

Summary.

Taxonomically distinct microbiota states exist in the upper airways of children with asthma, with specific states which are neither modified by inhaled corticosteroid boost nor omalizumab therapy, relating to distinct associations with asthma exacerbation and rhinovirus infection.

Abstract.

Compositionally distinct infant upper airway bacterial microbiota have previously been shown to differentially associate with risk of acute respiratory infection, severity of inflammatory symptoms, and asthma development in childhood. Without being bound by any scientific theory, it was hypothesized that discrete bacterial microbiota exist in the upper airways of children with asthma, and differentially relate to exacerbation and rhinovirus infection. Here, a large (n=413 participants ages 6-17, n=3,122 samples) longitudinal study of nasal wash samples from children with asthma obtained post-randomization over a 3-month period during the Preventative Omalizumab or Step-up Therapy for Severe Fall Exacerbations (PROSE) placebo-controlled trial, underwent 16S rRNA biomarker sequencing and analyses. Though no significant relationship between treatment group and bacterial beta-diversity was identified (p>0.05), factors such as rhinovirus infection (RV; R²=0.0069, p=0.01), age (R²=0.029, p=0.001), study site (R²=0.39; p=0.001) and, in particular, dominant bacterial taxon (R²=0.64; p=0.001) co-varied with nasal microbiota composition. Multiple Moraxella taxa were enriched in participants who experienced asthma exacerbations, while Staphylococcaceae and Corynebacterium were characteristically enriched in those who did not. The majority (90%) of samples belonged to one of six microbiota compositional states, each dominated by Moraxella, Staphylococcaceae, Corynebacterium, Alloiococcus, Haemophilus, or Streptococcus. Moraxella- and Staphylococcaceae-associated states were most common, exhibited temporal stability, and displayed opposing risks for exacerbation (RR=1.75, q=<0.001 and RR=0.72, q=0.04 respectively). Hence, Omalizumab appears to act downstream or distinctly from the upper airway mucosal microbiota to reduce asthma exacerbations. Specific upper airway microbiota may contribute to increased exacerbation and/or RV-induced asthma pathology in children.

Recent advances in culture-independent microbial detection have revealed the diversity of microbes that co-exist on the mucosal surfaces of the healthy upper airways, and that the composition of these mixed species microbiota is related to respiratory health status(1, 2). The Childhood Asthma Study (CAS), a prospective cohort of infants (n=234), examined nasopharyngeal bacterial and viral communities over the first year of life, including samples collected during periods with and without acute respiratory infection(3). Within this cohort, six compositionally distinct bacterial states, each dominated by Moraxella, Staphylococcaceae, Corynebacterium, Streptococcus, Alloiococcus or Haemophilus were identified. Infant nasal microbiota dominated by Moraxella, Streptococcus or Haemophilus were associated with significantly elevated risk of virus-associated acute respiratory infection (3). Moreover, early-life colonization with Streptococcus was associated with increased risk of lower airway infection and subsequent asthma development in childhood. Independently, Bisgaard and colleagues, using culture-based approaches, demonstrated that detection of S. pneumoniae, M catarrhalis or H. influenzae in nasopharyngeal samples obtained at 1 month of age was linked to increased risk for asthma at 5 years of age(4). Hence, early-life upper airway microbiota composition is related to the risk of infection and subsequent lower airway disease development.

A more recent study of 47 currently healthy children (aged 49-84 months), revealed that increased nasopharyngeal (NP) bacterial diversity was positively correlated with the time to develop an upper respiratory infection (URI), and NP microbiota diversity was diminished in children who experienced more frequent URIs (P≤0.05). Furthermore, children who had previously experienced an acute sinus infection exhibited significant relative enrichment of Moraxella, presumably as result of the perturbation elicited by the infection and/or the associated antimicrobial treatment. In addition nasopharyngeal enrichment of Moraxella in healthy (non-URI) baseline samples was predictive of subsequent acute sinusitis events in the 1-year clinical follow-up period(5). Using Q-PCR to specifically quantify Moraxella catarrhalis, Streptococcus pneumoniae and Haemophilus influenzae, Kloepfer and colleagues demonstrated that detection of S. pneumoniae was associated with increased cold symptoms and moderate asthma exacerbations, particularly when co-detected with rhinovirus (RV). M. catarrhalis and RV increased the likelihood of experiencing cold symptoms, asthma symptoms, or both compared with detection of RV alone(6).

These findings indicate that microbiota composition and the presence of specific bacterial genera in the upper airways strongly and reproducibly relate to respiratory infection risk, particularly in relation to viral infection. Without being bound by any scientific theory, given that viral respiratory infection is a risk factor for asthma exacerbation(7), it was hypothesized that specific patterns of microbiota composition also exist in the upper airways of children with asthma, and are related to risk of viral infection and asthma exacerbation. While studies of the asthma-associated microbiota have concentrated on the lower airways, interactions with respiratory viruses are likely to begin in the upper airway and it was hypothesized that these interactions influence subsequent events including the probability of asthma exacerbation.

The present study represents the largest investigation of the upper airway microbiota undertaken to date. It leverages a sizeable number (n=3,122) of longitudinal nasal wash samples collected from children (n=413) enrolled in the Inner City Asthma Consortium (ICAC) Preventive Omalizumab or Step-Up Therapy for Severe Fall Exacerbations (PROSE) study in which Omalizumab therapy significantly reduced the incidence of Fall exacerbations (defined as a need for physician prescribed steroids for asthma symptoms). Bacterial microbiota profiles generated from these samples were examined for their association with asthma exacerbation and RV. While administration of anti-inflammatory therapies are efficacious for prevention of fall exacerbations of asthma(8), the findings herein indicate that they do not profoundly influence the composition of the upper airway microbiota. Moreover, the existence of similar microbiota states in the pediatric asthma population to those previously described in the CAS study, demonstrate their temporal stability over the study observation period, and show that specific upper airway bacterial taxa and their associated co-colonizers increase the risk for asthma exacerbation and RV in this population. The data suggest that the upper airway microbiota may enhance susceptibility to exacerbation and viral respiratory infection in children with asthma, indicating that strategies to manipulate or modify these microbiota may decrease risk in this population.

Results

Of a total of n=3,840 available nasal wash samples from 513 participants were received; n=3,122 samples producing high-quality sequence data and representing at least three or more repeated measures per participant (N=413) were included in the analyses. Community alpha diversity indices (Chao-1 richness, Pielou's evenness and Faith's phylogenetic diversity) were calculated and relationships with primary clinical or viral outcomes [exacerbation (E), Rhinovirus infection (RV), or number of colds] during the 3-month post-randomization observation period were assessed across the entire cohort, and within each treatment group (placebo, boost ICS or omalizumab). Significant reductions in microbiota richness were observed in exacerbation versus non-exacerbation samples, only within the placebo group, and specifically in the step 2-4 subjects (Table 1). Likewise, significant reductions in community alpha diversity were observed in placebo-treated subjects who experienced RV and colds, though it was the more severely ill, step 5 group, that accounted for these observations (Table 1). This data indicates that reduced upper airway bacterial richness is associated with exacerbation in moderately ill, and RV and colds in more severely ill children with asthma.

TABLE 1
Relationship Between Nasal Microbiota Alpha Diversity and Exacerbation, Human Rhinovirus Infection and Number of Colds.

| | | | | | | | | Faith's Phylogenetic | | |
|---|---|---|---|---|---|---|---|---|---|---|
| | | Richness | | | Evenness | | | Diversity | | |
| | | E^{c} | NE^{d} | | E | NE | | E | NE | |
| Exacerbation | n | (mean ± | (mean ± | P- | (mean ± | (mean ± | P- | (mean ± | (mean ± | P- |
| (Participant) | (N^{a}/Y^{b}) | SE^{e}) | SE) | value | SE) | SE) | value | SE) | SE) | value |
| Overall | 2624/498 | 209.3 ± 6.7 | 218.5 ± 2.7 | 0.17 | 0.51 ± 0.01 | 0.51 ± 0.01 | 0.57 | 11.83 ± 0.41 | 12.37 ± 0.16 | 0.18 |
| Placebo | 454/145 | 203.2 ± 12.4 | 233.6 ± 6.4 | 0.02 | 0.51 ± 0.02 | 0.53 ± 0.01 | 0.48 | 11.53 ± 0.71 | 13.28 ± 0.37 | 0.02 |
| Step 2-4 | 253/41 | 194.7 ± 12.4 | 248.3 ± 7.4 | <0.01 | 0.50 ± 0.03 | 0.54 ± 0.02 | 0.15 | 11.03 ± 0.78 | 13.81 ± 0.47 | <0.01 |
| Step 5 | 198/104 | 224.4 ± 25.2 | 221.8 ± 10 | 0.92 | 0.54 ± 0.05 | 0.52 ± 0.02 | 0.62 | 12.82 ± 1.4 | 12.84 ± 0.56 | 0.99 |
| ICS | 737/139 | 218.1 ± 12.2 | 211.2 ± 4.7 | 0.57 | 0.51 ± 0.02 | 0.51 ± 0.01 | 0.93 | 12.5 ± 0.78 | 12.20 ± 0.30 | 0.70 |
| Xolair | 1470/221 | 208.7 ± 10.4 | 217.62 ± 3.8 | 0.39 | 0.50 ± 0.02 | 0.51 ± 0.01 | 0.61 | 11.7 ± 0.62 | 12.18 ± 0.22 | 0.41 |
| Step 2-4 | 701/70 | 198.1 ± 16.8 | 221.1 ± 5.0 | 0.17 | 0.48 ± 0.03 | 0.52 ± 0.01 | 0.15 | 11.01 ± 1.03 | 12.48 ± 0.31 | 0.18 |
| Step 5 | 747/145 | 213.5 ± 13.7 | 214.5 ± 5.6 | 0.94 | 0.51 ± 0.02 | 0.50 ± 0.01 | 0.58 | 11.93 ± 0.78 | 11.91 ± 0.32 | 0.98 |

| RV^{f} Infection | n | | | P- | | | P- | | | P- |
|---|---|---|---|---|---|---|---|---|---|---|
| (Sample) | (RV−^{g}/RV^{h}) | RV− | RV | value | RV− | RV | value | RV− | RV | value |
| Overall | 1883/1239 | 216.2 ± 2.7 | 218.3 ± 2.9 | 0.48 | 0.51 ± 0.01 | 0.51 ± 0.01 | 0.43 | 12.27 ± 0.16 | 12.30 ± 0.15 | 0.83 |
| Placebo | 304/295 | 231.7 ± 6.5 | 219.2 ± 6.5 | 0.06 | 0.52 ± 0.01 | 0.53 ± 0.01 | 0.59 | 12.99 ± 0.37 | 12.62 ± 0.34 | 0.27 |
| Step 2-4 | 157/137 | 217.3 ± 9.6 | 226.3 ± 10 | 0.35 | 0.53 ± 0.02 | 0.52 ± 0.02 | 0.70 | 12.70 ± 0.50 | 12.95 ± 0.56 | 0.62 |
| Step 5 | 145/157 | 220.6 ± 8.9 | 237.7 ± 8.4 | 0.06 | 0.53 ± 0.01 | 0.53 ± 0.02 | 0.70 | 12.55 ± 0.45 | 13.04 ± 0.49 | 0.27 |
| ICS | 551/325 | 206.6 ± 4.8 | 222.3 ± 5.7 | 0.01 | 0.50 ± 0.01 | 0.52 ± 0.01 | 0.26 | 12.13 ± 0.30 | 12.46 ± 0.30 | 0.28 |
| Xolair | 1049/642 | 216.5 ± 3.8 | 216.3 ± 4.0 | 0.97 | 0.51 ± 0.01 | 0.50 ± 0.01 | 0.02 | 12.12 ± 0.22 | 12.12 ± 0.20 | 0.98 |
| Step 2-4 | 461/310 | 224.0 ± 5.7 | 216.0 ± 5.3 | 0.16 | 0.51 ± 0.01 | 0.52 ± 0.01 | 0.28 | 12.52 ± 0.30 | 12.26 ± 0.32 | 0.38 |
| Step 5 | 575/317 | 209.0 ± 5.6 | 217.1 ± 5.4 | 0.15 | 0.49 ± 0.01 | 0.51 ± 0.01 | 0.02 | 11.74 ± 0.28 | 12.00 ± 0.31 | 0.35 |

| Number of | | | | | | | |
|---|---|---|---|---|---|---|---|
| Colds | N | Regression | | Regression | | Regression | |
| (Participant) | [N/Y (range)] | (beta coefficient) | P-value | (beta coefficient) | P-value | (beta coefficient) | P-value |
| Overall | 122/291 (1-6) | −1.136 | 0.53 | 0.0040 | 0.21 | −0.008 | 0.94 |
| Placebo | 16/63 (1-5) | −5.791 | 0.14 | 0.0070 | 0.33 | −0.238 | 0.30 |
| Step 2-4 | | 1.617 | 0.79 | 0.0178 | 0.09 | 0.157 | 0.65 |
| Step 5 | | −13.76 | <0.01 | −0.0037 | 0.72 | −0.706 | 0.02 |
| ICS | 33/80 (1-6) | 2.071 | 0.53 | 0.0080 | 0.17 | 0.100 | 0.64 |
| Xolair | 73/148 (1-6) | −1.468 | 0.58 | −0.0007 | 0.86 | −0.014 | 0.93 |
| Step 2-4 | 263/508 (1-5) | 2.52 | 0.49 | 0.009 | 0.13 | 0.339 | 0.13 |
| Step 5 | 264/628 (1-6) | −4.63 | 0.22 | −0.009 | 0.12 | −0.293 | 0.18 |

^{a}N: No;
^{b}Y: Yes;
^{c}E: Exacerbation (exacerbation in outcome period);
^{d}NE: Non-exacerbation (no exacerbation in outcome period);
^{e}SE: Standard error;
^{f}RV: Rhinovirus;
^{g}RV−: Rhinovirus negative sample;
^{h}RV: Rhinovirus infected sample.
Bold and underline font indicates statistically significant findings at p < 0.05.

Clinical and Viral Factors are Related to Bacterial Community Variation.

An expanded range of clinical, demographic and microbiological factors measured during the 3-month post-randomization observation period (Table 2) were assessed for their relationship with nasal microbiota composition (beta-diversity). At baseline (first post-randomization sample received from each participant in the study) nasal beta-diversity (calculated using Weighted UniFrac and PERMANOVA) was significantly associated with the dominant taxon, study site, age, log eosinophil cationic protein concentration in nasal secretions, total number of RV events and sample-specific RV; treatment group and gender also trended towards significance (Table 2). These observations were largely supported by bootstrapped analysis (using an iterative process of including a randomly selected single sample per participant in PERMANOVA analysis), with the exception of exacerbations, which became significantly related to nasal bacterial beta-diversity (bootstrapped PERMANOVA; R²=0.0074, p=0.045). This latter observation appears to be due to the increased number of exacerbation-associated observations included in the bootstrapped analysis (n=10 exacerbations in baseline vs n=96 across all samples collected).

TABLE 2
Relationships Between Microbiological, Clinical and Viral Events and Nasal Microbiota Composition

| | R² | P-value | R² | R² | P-value | P-value |
|---|---|---|---|---|---|---|
| Variable | (baseline) | (baseline) | (bootstrapped^{a}) | (min, max) | (bootstrapped) | (min, max) |
| Dominant Genus | 0.6372 | 0.001 | 0.6264 | 0.586, 0.664 | 0.001 | 0.001, 0.001 |
| Dominant Family | 0.628 | 0.001 | 0.6158 | 0.576, 0.655 | 0.001 | 0.001, 0.001 |
| Study Site | 0.039 | 0.001 | 0.0259 | 0.015, 0.039 | 0.064 | 0.001, 0.658 |
| Age at Randomization^{b} | 0.0287 | 0.001 | NA^{f} | NA | NA | NA |
| Treatment Group | 0.0081 | 0.078 | 0.0064 | 0.002, 0.015 | 0.287 | 0.002, 0.967 |
| Log(ECP^{c}), Randomization^{b} | 0.0076 | 0.013 | NA | NA | NA | NA |
| Rhinovirus Infection (RV) | 0.0069 | 0.011 | 0.0059 | 0.001, 0.018 | 0.121 | 0.001, 0.858 |
| Total number of RV | 0.005 | 0.029 | 0.0068 | 0.002, 0.016 | 0.06 | 0.001, 0.598 |
| Gender | 0.0049 | 0.066 | 0.0024 | 0, 0.008 | 0.499 | 0.011, 0.994 |
| Exacerbation (Participant) | 0.0041 | 0.13 | 0.0074 | 0.002, 0.016 | 0.045 | 0.001, 0.430 |
| Treatment Step | 0.0028 | 0.364 | 0.0016 | 0, 0.006 | 0.673 | 0.492, 1.000 |
| Active Exacerbation Sample | 0.0025 | 0.394 | 0.0025 | 0, 0.009 | 0.472 | 0.004, 0.995 |
| Number of Colds | 0.0023 | 0.478 | 0.035 | 0, 0.010 | 0.310 | 0.001, 0.980 |
| FEV1^{d}/FVC^{e}, Randomization^{b} | 0.0011 | 0.873 | NA | NA | NA | NA |

^{a}Bootstrapped: A single sample per individual is used to calculate PERMANOVA R2 and p-value, repeating this process 500 times; resulting mean R2 and p-values, as well as range of values are provided.
^{b}Bootstrapped analyses are not presented as this measure occurs only at randomization.
^{c}ECP: Eosinophil Cationic Protein.
^{d}FEV1: Forced Expiratory Volume 1.
^{e}FVC: Forced Vital Capacity.
^{f}NA: Not applicable.

Distinct Taxa Exhibit Differential Associations with Asthma-Related Events.

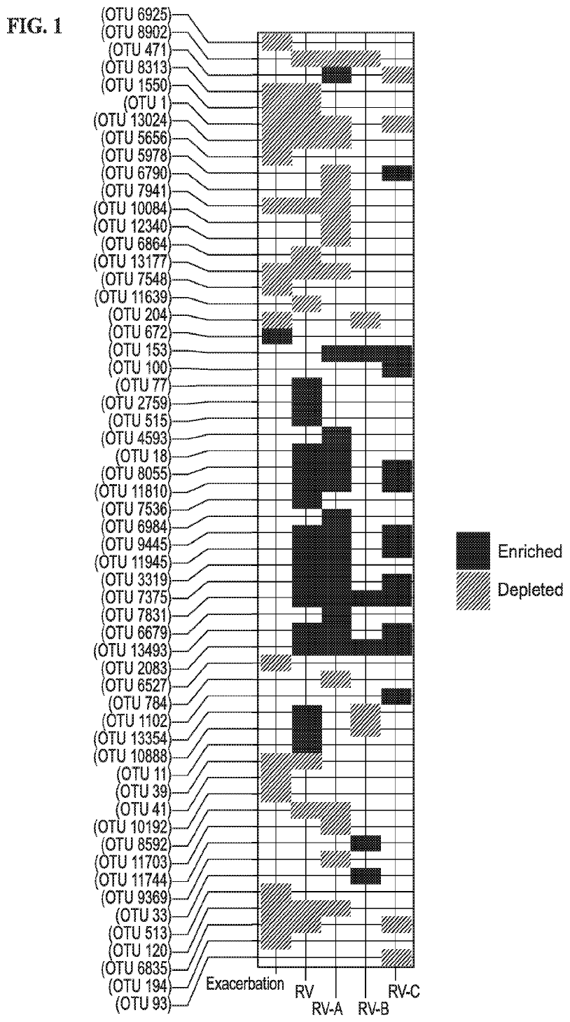

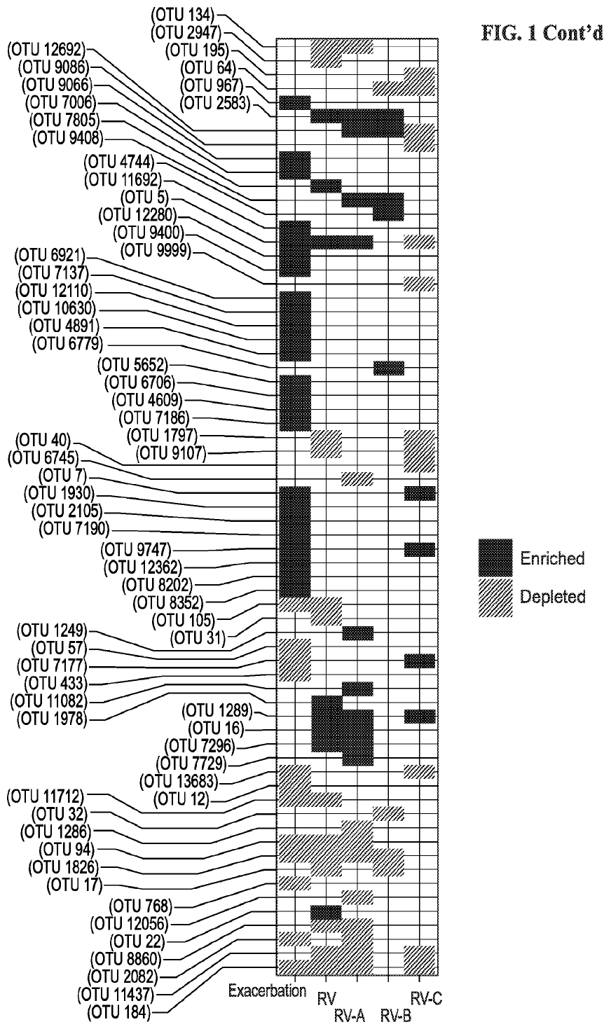

Taxon relative abundance comparisons were made across participant groups stratified based on exacerbation (yes/no; at the participant level), RV (yes/no; at the sample level), and the specific RV strain detected within a sample [RV-A, RV-B, or RV-C; each compared to RV-negative (RV-) uninfected samples]. Multiple OTUs exhibited significantly different relative abundance in more than one comparison, and taxonomic trends were apparent (FIG. 1; Table 17). Consistently, multiple Moraxella and Neisseria taxa were enriched in the nasal microbiota of children who experienced exacerbations, with several of these Moraxella taxa also exhibiting positive relationships with RV, particularly RV-A and RV-B. Conversely, several Staphylococcus and Corynebacterium taxa were enriched in participants who either did not exacerbate or had RV-samples. Interestingly, though not associated with exacerbation, several distinct Streptococcus taxa were significantly relatively enriched in RV samples, specifically in participants who were RV-A or RV-C positive. A similar observation was made for Haemophilus, which was relatively enriched in RV samples, but not associated with exacerbation (FIG. 1). This indicates that specific bacterial genera consistently co-associate with asthma exacerbation and rhinovirus detection in this population that may differentially potentiate the pathogenicity of respiratory viral infection.

Exacerbation and RV-Associated Taxa Co-Associate in Networks.

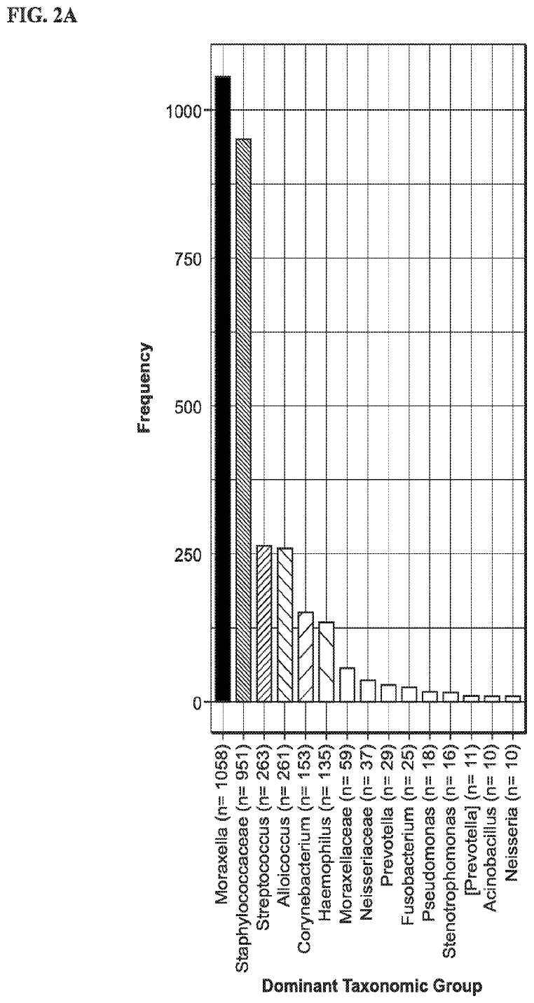

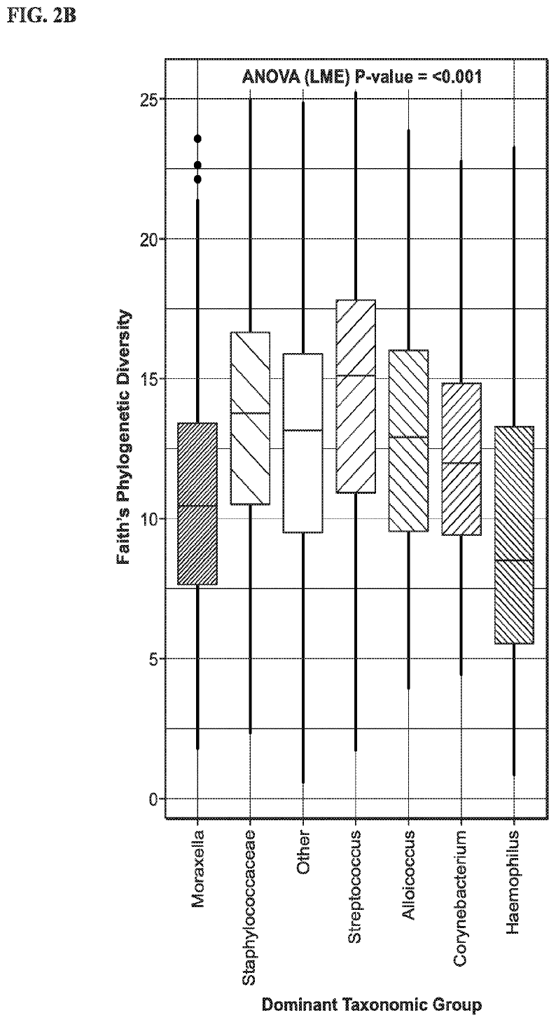

While assessing relationships with bacterial beta-diversity, it was noted that the dominant bacterial taxon detected, explained a large proportion of variability in microbiota composition (Table 2). This suggested that the dominant taxon within a nasal microbiota co-associates with a relatively distinct group of microbes that presumably contribute to overall microbiome function and host interactions. Notably, the majority of samples in these children with asthma were dominated by Moraxella (33.9%) or Staphylococaceae (30.4%), with the remaining samples dominated by Streptococcus (8.4%), Alloiococcus (8.4%), Corynebacterium (4.9%), Haemophilus (4.3%) or other taxa (9.7%; FIG. 2A). A comparison of Faith's phylogenetic diversity across these distinct bacterial compositional states indicated that significant differences existed (ANOVA, LME; p<0.05; FIG. 2B). Specifically, Moraxella and Haemophilus-dominated communities exhibited significantly lower diversity compared to all other groups (post-hoc Tukey method; all p-values <0.05). The taxa characteristically dominating each of the six most common community states were also amongst those significantly enriched or depleted in participants who experienced asthma exacerbations or RV. In addition, these dominant taxa formed distinct co-association networks with other taxa implicated in asthma exacerbation or RV. Specifically, Moraxella was found to primarily co-associate with other Moraxella taxa including several that were enriched in participants who experienced exacerbations (Table 17). In contrast the dominant taxa in each of the other nasal microbiota states, evidenced co-association networks consisting of both phylogenetically related and distinct taxa, which again involved a number of those taxa enriched in non-exacerbation and non-RV samples. This suggests that exacerbation or RV risk in children with asthma who possess specific upper airway microbiota states housing these bacterial networks, may be related not just to the activities of the dominant organism, but to the combined actions of the co-associated bacterial network. Taken together, these data indicate that distinct and relatively conserved patterns of microbial co-association (microbiota states) exist in the upper airways of these children and that multiple members of these communities may play a role in exacerbation and RV outcomes.

Based on these observations it was rationalized that rather than individual taxa, these bacterial community states co-vary with clinical and viral features of children with asthma. Indeed, when samples were stratified based on microbiota state, children at reduced risk of exacerbation possessed nasal microbiota characteristically dominated by Corynebacterium (RR=0.40, q=0.02), Haemophilus (RR=0.38, q=0.02), Staphylococcaceae (RR=0.72, q=0.04; Table 3). In contrast, children with microbiota dominated by Moraxella exhibited increased risk of exacerbation (RR=1.75, q=<0.01). Consistent with a reduced exacerbation risk, Staphyloccocaceae-dominated communities were also associated with a reduced risk of RV (RR=0.77, q=<0.01), specifically RV-A and RV-C (RR=0.74, q=0.02; RR=0.69, q=0.04 respectively). Of note, though risk for exacerbation was not increased in children with a Streptococcus-dominated microbiota, this group exhibited a significantly higher relative risk of RV (RR=1.52, q=<0.01) and trended towards increased detection of RV-A and RV-C(RR=1.64, q=0.02; RR=1.66, q=0.04 respectively). These findings were robust despite adjustment for participants' age at randomization, which did not alter the relative risk by more than ten percent.

TABLE 3
Microbiota States Differentially Associate with Exacerbation, Viral Outcomes and Clinical Events

| | Alloiococcus | | Corynebacterium | | Haemophilus | | Moraxella | | Staphylococcaceae | | Streptococcus | | Other | |
|---|---|---|---|---|---|---|---|---|---|---|---|---|---|---|
| | RR^{a} | Q^{b} | RR | Q | RR | Q | RR | Q | RR | Q | RR | Q | RR | Q |
| Age, randomization^{c} | 1.01 | 0.90 | 1.35 | <0.01 | 1.07 | 0.72 | 0.84 | <0.01 | 1.05 | 0.34 | 0.96 | 0.72 | 0.98 | 0.72 |
| FEV1/FVC, randomization^{c} | 1.01 | 0.66 | 0.97 | 0.66 | 1.02 | 0.68 | 0.99 | 0.68 | 1.01 | 0.68 | 1.02 | 0.66 | 0.98 | 0.66 |
| Log(ECP), randomization^{c} | 0.94 | 0.78 | 0.58 | 0.22 | 0.65 | 0.50 | 1.75 | 0.02 | 0.84 | 0.50 | 0.90 | 0.78 | 1.11 | 0.78 |
| Exacerbation (Participant)^{d} | 0.74 | 0.26 | 0.40 | 0.02 | 0.38 | 0.02 | 1.75 | <0.01 | 0.72 | 0.04 | 1.04 | 0.88 | 1.19 | 0.39 |
| Any Rhinovirus^{d} | 0.92 | 0.63 | 0.97 | 0.87 | 1.33 | 0.28 | 1.12 | 0.25 | 0.77 | <0.01 | 1.52 | <0.01 | 0.85 | 0.31 |
| Rhinovirus-A Infection (RV-A) | 0.83 | 0.46 | 0.88 | 0.65 | 1.95 | 0.02 | 1.10 | 0.47 | 0.74 | 0.02 | 1.64 | 0.02 | 0.72 | 0.18 |
| vs. RV−^{d} | | | | | | | | | | | | | | |
| Rhinovirus-B Infection (RV-B) | 1.17 | 0.57 | 0.85 | 0.61 | 0.88 | 0.71 | 1.22 | 0.28 | 0.84 | 0.30 | 1.30 | 0.39 | 0.71 | 0.28 |
| vs. RV−^{d} | | | | | | | | | | | | | | |
| Rhinovirus-C Infection (RV-C) | 1.00 | 0.99 | 1.21 | 0.88 | 1.15 | 0.88 | 1.06 | 0.88 | 0.69 | 0.04 | 1.66 | 0.04 | 1.06 | 0.88 |
| vs. RV−^{d} | | | | | | | | | | | | | | |
| Multiple-RV vs. RV−^{d} | 0.16 | 0.49 | 0.68 | 0.60 | 0.37 | 0.54 | 1.10 | 0.75 | 1.05 | 0.85 | 1.59 | 0.54 | 1.65 | 0.49 |
| Treatment Group^{d} | | | | | | | | | | | | | | |
| ICS vs. Placebo | 1.25 | 0.33 | 0.42 | 0.01 | 1.44 | 0.35 | 1.33 | 0.06 | 1.17 | 0.52 | 0.91 | 0.01 | 0.53 | 0.40 |
| Xolair vs. Placebo | 0.94 | 0.77 | 0.80 | 0.36 | 2.15 | 0.03 | 1.25 | 0.10 | 0.66 | 0.57 | 1.08 | 0.03 | 0.68 | 0.02 |
| Number of Viral Infections, | 1.04 | 0.49 | 0.90 | 0.14 | 0.99 | 0.99 | 1.11 | <0.01 | 0.88 | <0.01 | 1.06 | 0.35 | 1.00 | 0.99 |
| post-randomization^{d} | | | | | | | | | | | | | | |
| Number of Colds, | 1.09 | 0.35 | 1.02 | 0.83 | 1.03 | 0.83 | 0.97 | 0.59 | 0.92 | 0.07 | 1.14 | 0.07 | 1.05 | 0.42 |
| post-randomization^{d} | | | | | | | | | | | | | | |

^{a}Relative Risks;
^{b}Q-values are p-values adjusted for multiple comparisons using a Benjamini-Hochberg False Discovery Rate across community states for each covariate of interest;
^{c}A11 variables measured at randomization were associated solely with baseline community states, Generalized Linear Models (GLM) using the initial sample were used with a binomial outcome;
^{d}Results use all data from samples collected from an individual throughout the outcome period, Generalized Estimating Equations (GEE) using all longitudinal samples were used with a binomial outcome, and an exchangeable correlation structure.
Significant differences between dominant microbiota groups for each variable are emphasized with bold and underline font and reflect significance of q < 0.05.

TABLE 4
Participant and Sample Characteristics for the
Preventive Omalizumab or Step-Up Therapy for
Severe Fall Exacerbations (PROSE) study.

| | Individuals | | Samples | |
|---|---|---|---|---|
| | N | % | | |
| Variable | (Mean) | (SD) | n | % |
| Treatment Group | | | | |
| ICS | 113 | 0.27 | 863 | 0.28 |
| Placebo | 79 | 0.19 | 596 | 0.19 |
| Xolair | 221 | 0.54 | 1663 | 0.53 |
| Treatment Step at Randomization | | | | |
| Step 2-4 | 252 | 0.61 | 1928 | 0.62 |
| Step 5 | 161 | 0.39 | 1194 | 0.38 |
| Study Site | | | | |
| Boston | 51 | 0.12 | 401 | 0.13 |
| Chicago | 43 | 0.10 | 353 | 0.11 |
| Cincinnati | 49 | 0.12 | 365 | 0.12 |
| Dallas | 40 | 0.10 | 329 | 0.11 |
| Denver | 68 | 0.16 | 489 | 0.16 |
| Detroit | 41 | 0.10 | 281 | 0.09 |
| New York | 47 | 0.11 | 364 | 0.12 |
| Washington D.C. | 74 | 0.18 | 540 | 0.17 |
| Exacerbation During the Outcome | | | | |
| Period | | | | |
| None | 346 | 0.84 | 2624 | 0.84 |
| One or More | 67 | 0.16 | 498 | 0.16 |
| Number of Viral Infections | (3.48) | (1.75) | | |
| Rhinovirus Infection | | | | |
| Yes | | | 1883 | 0.60 |
| No | | | 1239 | 0.40 |
| Rhinovirus Species | | | | |
| None | | | 1883 | 0.60 |
| A | | | 386 | 0.12 |
| B | | | 449 | 0.14 |
| C | | | 335 | 0.11 |
| M | | | 69 | 0.02 |
| Sex | | | | |
| Female | 155 | 0.38 | 1158 | 0.37 |
| Male | 258 | 0.62 | 1964 | 0.63 |
| Race | | | | |
| Black (non-Hispanic) | 239 | 0.58 | 1749 | 0.56 |
| Hispanic | 141 | 0.34 | 1126 | 0.36 |
| Other/Mixed | 26 | 0.06 | 201 | 0.06 |
| White (non-Hispanic) | 7 | 0.02 | 46 | 0.01 |
| Age at Randomization | | | | |
| 6-10 years | 242 | 0.59 | 1852 | 0.59 |
| 10-14 years | 128 | 0.31 | 957 | 0.31 |
| 14-18 years | 43 | 0.1 | 313 | 0.1 |
| Age at Randomization, | (10.25) | (2.97) | | |
| Overall | | | | |
| Income above 15^{th} Percentile | | | | |
| No | 186 | 0.45 | 1398 | 0.45 |
| Yes | 223 | 0.55 | 1690 | 0.55 |

Microbiota Community States are Associated with Distinct Clinical Feature.

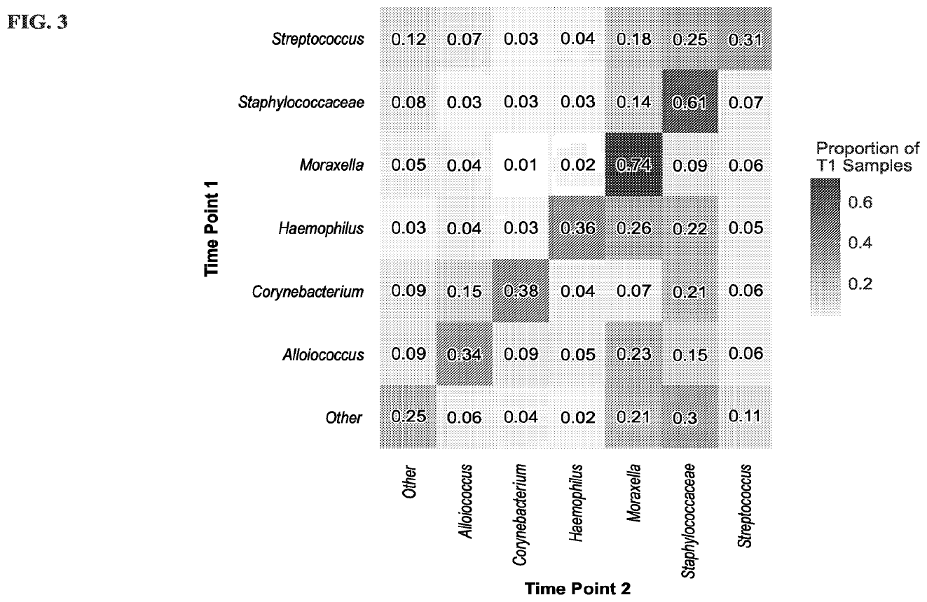

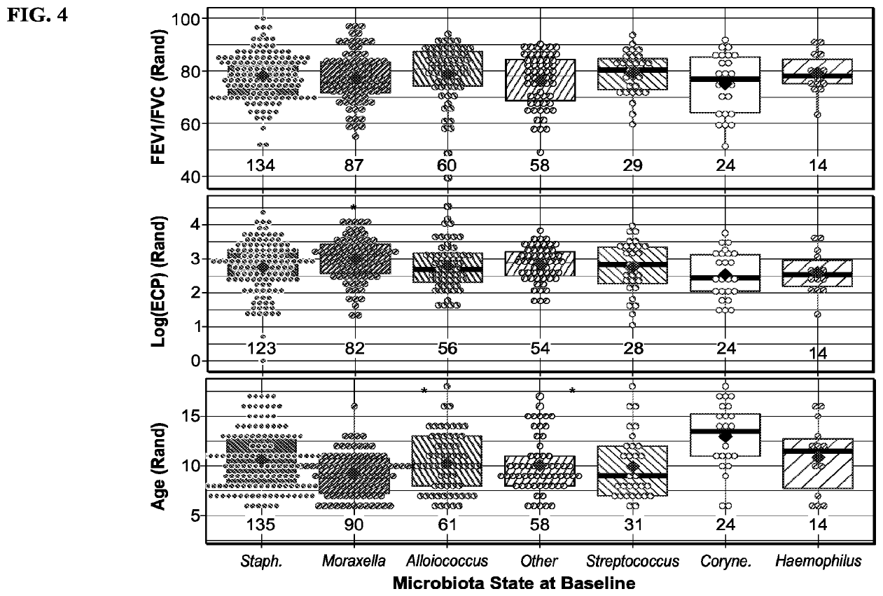

In addition to exacerbation and RV, whether bacterial microbiota states differed with respect to other demographic or clinical features was also assessed. At baseline, children with Moraxella-dominated communities were significantly younger, while those with Corynebacterium-dominated microbiota were significantly older (FIG. 4). Pulmonary function at randomization (FEV1/FVC) did not differ by microbial community state. Interestingly, children with Moraxella-dominated communities had significantly elevated concentrations of eosinophil cationic protein (ECP) in nasal secretions implicating this microbiota state in eosinophil activation in the upper airways (FIG. 4).

Staphylococcaceae- and Moraxella-Dominated Communities Exhibit Temporal Stability.

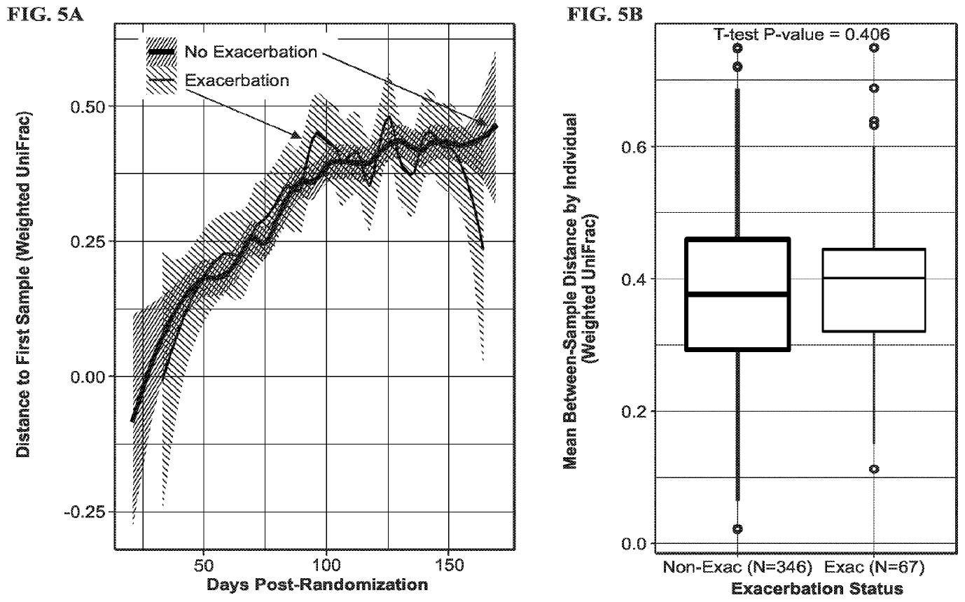

It had been hypothesized that bacterial community stability would be linked to exacerbations and that those children who experienced asthma exacerbations would exhibit decreased community stability compared to those who did not. However, when comparing temporal community composition to the baseline sample, or mean distances between samples in those participants who did or did not experience exacerbations, no significant difference in temporal stability was observed (FIGS. 5A-5B).

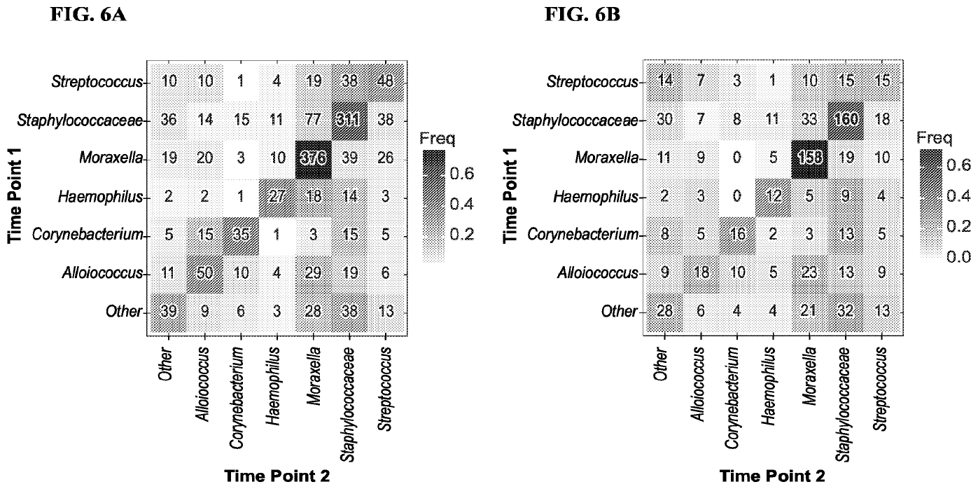

It was next asked whether community stability was related to the specific bacterial community state that colonized the upper airways of children with asthma, and specifically if the Moraxella-dominated community, which was associated with increased exacerbation risk, represented a temporally stable microbiota within the upper respiratory tract. Transitions (n=2,709) between community states across longitudinally collected samples within the cohort were assessed and visualized using a heatmap (FIG. 3). The data indicated that both Moraxella- and Staphylococcaceae-dominated community states were most frequently stably maintained over time. While the less frequently observed community states (Streptococcus-, Haemophilus-, Corynebacterium- and Alloiococcus-dominated) most frequently transitioned to a similarly-dominated state, they also exhibited a high frequency of transition to either a Moraxella- or Staphylococcaceae-dominated state (FIG. 3). These observations were consistent when either only samples collected within a defined period of time (7-13 days between sample acquisition), or the first two transitions for each participant were considered (to account for possible biases due to protracted periods between sample acquisition, or to uneven sampling across participants; FIGS. 6A-6B).

Using available samples collected pre-, peri-, or post-exacerbation (n=498 samples from n=54 participants), we also examined the effect of exacerbation and RV on upper airway nasal microbiota. We noted that a large number of these participants exhibited compositionally stable nasal microbiota dominated by Moraxella (16/54; 30%) or Staphylococcaceae (7/54; 13%) despite experiencing an exacerbation or RV.

Distinct Upper Airway Bacterial Microbiota Differentially Relate to Rhinovirus Infection and Airway Exacerbation in Pediatric Asthma.

The analyses of the upper airway bacterial microbiota of over four hundred children with asthma in the PROSE trial indicate that while omalizumab therapy successfully reduced fall exacerbations within this population(8), it did not exert appreciable effects on the composition of the upper airway bacterial microbiota. Furthermore, inhaled corticosteroid dose at randomization (a measure of disease severity) was not related to the composition of the upper airway microbiota. It is plausible that the highly adherent microbial communities colonizing the upper airway mucosal surface remain unaffected by treatments that target downstream features of asthma-associated immune dysfunction. The persistence of immunostimulatory mucosal microbiota may in part explain why chronic inflammatory disease frequently recurs following cessation of anti-inflammatory treatments, indicating that respiratory mucosal microbiome manipulation may be necessary to induce a more permanent effect on immune activation in the airways of children with asthma.

The taxon-based comparisons identified specific genera associated with exacerbation and RV. Specifically, multiple Moraxella taxa were found to be enriched in participants who experienced at least one exacerbation in the outcome period. Of these taxa, several were also significantly enriched in participants who experienced RV, specifically the more pathogenic RV-A and RV-C. Moraxella, in particular M. catarrhalis, has previously been implicated in both increased risk of asthma development(4) and, when detected in parallel with RV in children with asthma, exacerbation(6). Interest in M. catarrhalis has recently increased, largely because of its emerging role in airway pathogenesis, particularly in children. This species encodes a range of adhesions and ligands related to adherence and invasion of epithelial cells, and its capacity to evade the complement system supports persistent mucosal survival(9). M. catarrhalis can induce innate immune responses via immunoglobulin D and exhibits the capacity to destroy tissue via neutralizing al-antichymotrypsin, which subsequently promotes its adherence and biofilm formation(9). These features, particularly epithelial damage, may contribute to increased likelihood of RV and of exacerbation, particularly since RV cytotoxicity is known to be enhanced in the context of a compromised epithelium(10).

A key finding of this study is that bacterial mucosal microbiota segregate into compositionally distinct community states, each dominated by a different bacterial taxon and comprised of taxa that form discrete co-association networks. While the majority of the dominant bacteria co-associated with phylogenetically distinct organisms, the dominant Moraxella primarily co-associated with other Moraxella, including several implicated in exacerbation or RV. Unsurprisingly, participants who possessed Moraxella-dominated communities were at significantly higher relative risk of exacerbation, and exhibited significantly higher concentrations of ECP, a marker for activated eosinophils that correlates with severity of airway inflammation. Thus the data indicates that rather than one single Moraxella species, a population of distinct Moraxella appear to co-colonize the upper airway mucosal surface of RV and exacerbation-prone children with asthma, suggesting that their combined activities on the airway mucosal surface may contribute to this heightened susceptibility. Moreover, Moraxella-dominated communities were amongst the most temporally stable upper airway microbiota, in some cases persisting with little or no compositional changes despite RV and exacerbation. This observation may explain why some children with asthma are “exacerbation prone”; plausibly, these children possess a highly stable pathogenic Moraxella-dominated upper airway microbiota that promotes mucosal conditions which enhance respiratory viral cytotoxicity.

In contrast to the Moraxella-dominated communities, children with asthma whose upper airway microbiota are dominated by Corynebacterium, Haemophilus or Staphylococcaceae were at significantly reduced risk of exacerbation in the study. These observations are largely consistent with the findings of Teo and colleagues who noted that infants whose upper airways were colonized by bacterial communities dominated by Corynebacterium or Staphylococcaceae were at significantly lower risk of acute respiratory infection (3). Collectively these observations suggest that distinct microbiota colonization patterns in the upper airway may modulate mucosal integrity and immunity in a manner that dictates susceptibility and severity of respiratory infection, particularly those mediated by RV. It should be noted that the CAS study found that while Haemophilus-dominated communities were infrequently detected in healthy infant samples, they tended to arise in parallel with acute respiratory infection (3). In the study of children with asthma, while Haemophilus-dominated communities were also less frequently observed, their presence associated with reduced risk of exacerbation. This discrepancy may be explained by differences in age and airway development, including mucosal microbiome development, that exists between infants and children. Related to this, a significant relationship between community states and age in was noted in the study, with Moraxella-dominated communities associated with significantly younger participants. Though adjustment for age did not alter the relative risk for exacerbation and RV associated with a particular community state, the data indicates that younger children with asthma who have a Moraxella-dominated upper airway microbiota represent a subset of participants at heightened risk for RV and asthma exacerbation.

Despite the inclusion of a large number of samples for analyses of relationships between the nasal microbiota, asthma and RV, there are a few study limitations to consider. Firstly, fungal communities may play a significant role in the nasal microbiota of children with asthma, however they have yet to be characterized in this population. In addition, the V4 region of the 16S rRNA gene was used to determine bacterial taxonomy within the microbiota in each sample. While this approach represents an economical strategy for examination of bacterial co-association networks, it does not provide information on the functional gene pathways or products produced by these microbial communities. In addition, the study does not include pre-randomization samples nor non-asthmatic upper airway samples, limiting the ability to understand whether the microbiota states described exist pre-treatment or in non-asthmatic children.

Nonetheless, this study forms a foundation for more in-depth investigations to determine the microbial mechanisms that promote or prevent exacerbation in children with asthma. Moreover, identification of children with asthma at heightened risk could lead to development of strategies to promote appropriate upper airway mucosal colonization to reduce pulmonary exacerbation in this population.

Materials and Methods

Study Design.

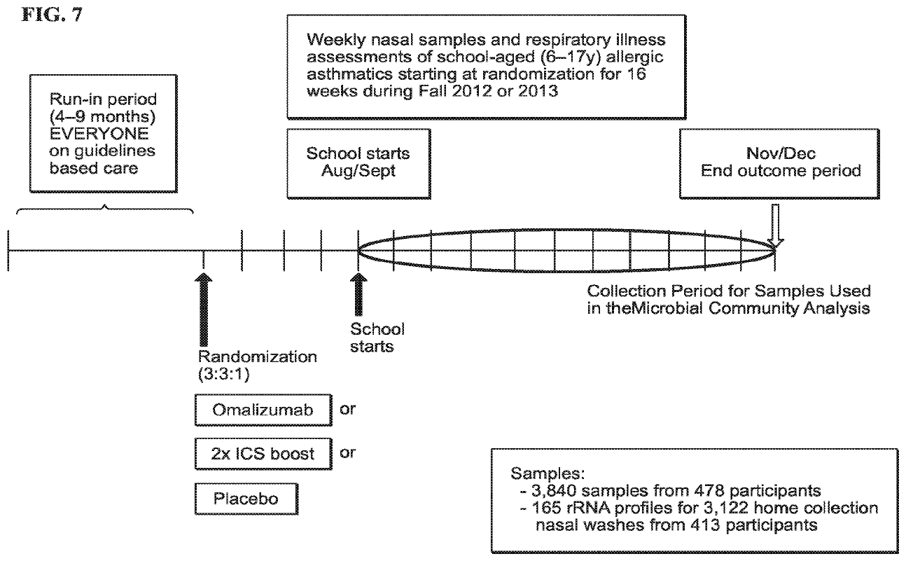

This study leverages nasal wash samples collected post-randomization from children with asthma (ages 6-18 years) participating in the Preventative Omalizumab (Xolair® anti-IgE antibody) or Step-up Therapy for Severe Fall Exacerbations (PROSE) randomized controlled trial (11). Children with asthma from urban communities were enrolled and followed during a run-in period of four to nine months prior to randomization to establish participant standard of care. Randomized treatment began 4-6 weeks prior to participants return to school, and they were followed for 90 days following the start of their school year (outcome period; FIG. 7). Asthma exacerbations were defined by the use of systemic corticosteroids and/or hospitalization for asthma(8). Nasal washes were collected at home throughout the outcome period on weeks without clinic visits.

DNA from nasal wash samples was extracted using a modified cetyltrimethylammonium bromide (CTAB)-polyethylene glycol (PEG) protocol as previously described(12), adjusted for high-throughput. The variable region 4 (V4) of the 16S rRNA gene was amplified and quantified, and subsequently pooled for sequencing on the NextSeq 500 (Illumina, San Diego, Calif.). Once sequenced, paired-end sequences were merged using FLASH 38 v 1.2.7(13) and processed to produce an OTU table using Quantitative Insights into Microbial Ecology (QIIME) pipeline(14) and USEARCH(15). Alpha rarefaction curves of observed species determined the appropriate rarefying depth. At 2,000 sequence reads per sample, sufficient community coverage and retention of the greatest number of samples for downstream analysis was achieved. To find the most representative OTU table of 2,000 reads, the OTU table was rarefied 100 times. The sample profile that was most similar based on the Bray-Curtis algorithm amongst the 100 tables was selected.

De-identified data for participant characteristics were received for analysis following 16S rRNA sequencing and generation of an OTU table used for microbiota analyses. Participants provided between 1 and 13 nasal samples that produced high-quality microbiota profiles, with a mean of 7.15 samples per participant. Participants with fewer than three microbiota profiles were removed from all downstream analyses (n=30 participants and 44 samples), resulting in 3,122 samples and 413 participants for analysis (Table 4).

Statistical Analysis I.

All statistical analyses were completed using R and QIIME(14). Alpha diversity indices (Chaol, Pielou's, and Faith's phylogenetic diversity indices) were calculated in QIIME(14) to evaluate sample richness, evenness, and phylogenetic diversity, respectively. Four distance matrices (Weighted and Unweighted UniFrac(16), Canberra, and Bray-Curtis) were also constructed for downstream analyses in QIIME.

Relationships between community composition and a range of clinical and viral infection variables, considered univariately, were assessed using adonis in the vegan package in R. To confirm PERMANOVA observations made using the initial sample received from each participant, “bootstrapped” PERMANOVA was performed by random resampling of one sample per participant, reiterated 500 times, generating a range of R² values and p-values. Significant differences in taxon relative abundance were assessed as previously described(17), using three different models for count data (Poisson, Negative Binomial, and Zero-Inflated Negative Binomial mixed-effects models).

Samples were grouped based on dominant taxon present. The majority of samples belonged to one of six microbiota states dominated by Moraxella (n=1,058), Staphylococcaceae (n=951), Streptococcus (n=263), Alloiococcus (n=261), Corynebacterium (n=153), or Haemophilus (n=135). All other samples (n=301), which exhibited rarer community states dominated by other genera were categorized as “other”. Longitudinal assessment of community states in subsequent samples was performed for each participant. Community state, based on dominant taxon, was used to classify each sample and the frequency with which the same or distinct community state occurred in the subsequent sample for the individual was determined. From these data we generated a heat map showing the frequency of each state transition. Finally, co-occurrence networks of OTUs were constructed using SparCC(18) and WGCNA(19) packages in R, filtering OTUs to include only those present in at least 25% of the samples.

This study leverages nasal wash samples collected post-randomization from children with asthma (ages 6-18 years) participating in the Preventative Omalizumab (Xolair® anti-IgE antibody) or Step-up Therapy for Severe Fall Exacerbations (PROSE) randomized controlled trial (11). In brief, children with asthma from urban communities were enrolled and followed during a run-in period of four to nine months prior to randomization. During this period a participant-specific standard of care was established based on asthma severity, defined by each participant's level of corticosteroid use, and clinical history of symptoms and exacerbations (8). At randomization, those at treatment steps 2-4 were randomized into (a) their current treatment (“Placebo”), (b) a doubled dose of ICS (“ICS”), or (c) subcutaneous omalizumab. Those with the most severe asthma (step 5) were randomized into (a) placebo, or (b) omalizumab. Treatment began 4-6 weeks prior to participants return to school, and they were followed for 90 days following the start of their school year (outcome period). Asthma exacerbations in the outcome period were defined by the use of systemic corticosteroids and/or hospitalization for asthma(8). Nasal samples examined in this study were collected during weeks without a clinic visit throughout the outcome period. To collect the samples, participants sprayed nasal saline into one nostril, held the saline for 5 seconds, and blew all fluid into a sterile bag; this process was repeated for the second nostril. The sample was then preserved in M4RT transport media (Thermo Fisher Scientific).

Bacterial Microbiota Profiling.

DNA from nasal wash samples was extracted using a modified cetyltrimethylammonium bromide (CTAB)-polyethylene glycol (PEG) protocol as previously described(12), adjusted for high-throughput. Briefly, 0.6 mL of extraction buffer [4% CTAB, 1M NaCl, 200 mM phosphate buffer pH=8) were added to 0.2 mL of nasal wash sample. Cells were lysed in a 96-well bead beating plate (MO BIO) at 20 m/s for 10 min followed by two extractions using 800 μL of phenol:cholorform:isoamyl alcohol (25:24:1) and centrifugation at 4,000×g for 30 min. Following phenol extraction, one volume of chloroform was added to each aqueous supernatant and centrifuged at 4,000×g for 30 min at 4° C. The aqueous layer was transferred to a new 96-well plate and DNA precipitated using two volumes of 20% PEG in 1.5M NaCl. Following 2 h incubation at room temperature, samples were centrifuged at 4,000×g for 60 min. Pelleted DNA was washed twice with ice-cold 70% ethanol and resuspended in 50 μL of molecular-grade H₂O.

The variable region 4 (V4) of the 16S rRNA gene was amplified from purified DNA using the 515F/806R primer combination as previously described (20, 21). Triplicate 25 μL PCR reactions were performed using a final concentration of 0.025 U of Takara ExTaq (Takara Clontech), lx Takara buffer with MgCl₂, 400 nM 515F and barcoded 806R primers, 200 μMdNTP, 0.56 mg·mL⁻¹ BSA (Roche Applied Science) and 10 μL of DNA template. Reaction conditions were as follows: initial denaturation at 98° C. for 3 min followed by 30 cycles of 98° C. for 20 s, annealing at 50° C. for 30 s, and extension at 72° C. 45 s, and a final extension of 72° C. for 10 min. Amplicons were purified using SPRI beads (Beckman Coulter) per the manufacturer's protocol and quantified using the Qubit HS dsDNA kit (Invitrogen). Sample pools consisted of 2 ng of purified amplicon from each sample and 400-500 unique samples per library. A total of n=3,499 samples produced sufficient 16S rRNA amplicon to proceed to sequencing. Libraries were quantified using the KAPA QPCR Illumina Library Quantification kit (KAPA Biosystems), diluted to 2 nM and denatured. Each library was combined with PhiX control at equal molarity, and diluted to 1.5 pM for sequencing on the NextSeq 500 (Illumina, San Diego, Calif.) using a high output cartridge.

Sequence Analysis.

Paired-end sequences were merged using FLASH 38 v 1.2.7(13) and processed to produce an OTU table using Quantitative Insights into Microbial Ecology (QIIME) pipeline(14) and USEARCH(15). Raw sequences were de-multiplexed and quality filtered to remove low quality sequences. Sequences with three or more consecutive bases with a Q score less than 30 were truncated and discarded if their length was less than 75% of the original 250 bp read length. Reads were then de-replicated, removing singletons; Operational taxonomic units (OTUs) were defined by UPARSE-OTU using 97% OTU clustering in USEARCH and chimeras were simultaneously removed(15). Samples (n=3,122) possessing a minimum number (n=2000) of high quality 16S rRNA reads were included in downstream analysis. All quality-filtered reads were then mapped back to the OTU sequences at 97% identity using UCLUST. A phylogenetic tree was constructed using sequences that aligned using PYNAST(22), and any OTUs that failed to align were removed. Taxonomy was assigned using UCHIME(23) and the Greengenes database (13_5)(24). OTUs were additionally filtered to include only those whose total read count over all samples was greater than 0.001% of the total read number of all samples, to remove spurious taxa from the dataset.

Alpha rarefaction curves of observed species determined the appropriate rarefying depth. At 2,000 sequence reads per sample, sufficient community coverage and retention of the greatest number of samples for downstream analysis was achieved. To find the most representative OTU table of 2,000 reads, the OTU table was rarified 100 times. The sample profile that was most representative based on Bray-Curtis distances amongst the 100 tables was selected(25). The resulting table with the representative community for each sample was used for downstream analysis.

De-identified data for participant characteristics were received for analyses following 16S rRNA sequencing and generation of the OTU table to be used for microbiota analyses. Participants provided between 1 and 13 nasal samples that produced a microbiota profile, with a median of 7 samples per participant. Participants with fewer than three samples were removed from all analyses (n=30 participants and 44 samples), resulting in an analysis data set of 3,122 samples from 413 participants. Participants were equally distributed amongst study sites, and were typically non-white and had incomes below the poverty line (4).

Viral Detection.

All nasal wash samples were tested for the presence and quantity of RV by quantitative RT-PCR at the University of Wisconsin using a previously published protocol(26). Samples with detectable RV were considered as an RV infection, regardless of whether participants exhibited respiratory symptoms. Samples designated as RVA, B or C, (RVA, RVB or RVC) only possessed the specific RV strain indicated, whereas those samples designated Multiple RV (M), contained more than one RV strain. RV infection was defined as detection of the virus, in cases where RV was detected in consecutive samples, these were considered as one RV infection event. RV typing was performed by partial sequencing as previously described(26).

Eosinophil Cationic Protein (ECP) Measurement.

ECP was measured by immunoassay (UniCAP, Phadia US Inc, Portage Mich.).

Statistical Analyses II.

All statistical analyses were completed using R(27) and QIIME(14). Alpha diversity indices (Chaol, Pielou's, and Faith's phylogenetic diversity indices) were calculated in QIIME(14) to evaluate sample richness, evenness, and phylogenetic diversity, respectively. Between-group alpha diversity index comparisons were made using linear mixed-effects models (to account for the repeated measures nature of the data); p<0.05 was considered significant. Four distance matrices (Weighted and Unweighted UniFrac(16), Canberra, and Bray-Curtis) were additionally constructed in QIIME. Relationships between beta-diversity and a range of clinical and viral infection variables, considered univariately, were assessed using PERMANOVA in the vegan package in R, using the initial sample received from each patient (baseline). To confirm PERMANOVA observations made using baseline samples, “Bootstrapped” PERMANOVA analysis was performed by random resampling of a single sample per participant, with replacement and reiterated 500 times. This was done to allow for the inclusion of repeated samples within participants, as adonis does not currently support random effects in unbalanced study designs. This bootstrapping method generated R² values and p-values for each iteration, the means of which were calculated and presented as bootstrapped values. In addition, the minimum and maximum value of the R² and p-values are additionally presented.

Significant differences in taxon relative abundance were assessed as previously described(17), using a three-model approach (Poisson, Negative Binomial, and Zero-Inflated Negative Binomial mixed-effects models). To de-noise the OTU table and reduce the number of spurious taxa, a data-driven method was used to further-filter rare OTUs when they were detected in less than 15% of the samples. The Akaike Information Criterion (AIC) was employed to identify the best model fit. Data from models presenting convergence warnings were excluded from consideration in model selection for each OTU. Observations were corrected for false discovery using the Benjamin-Hochberg method, and those with a q-value of less than 0.1 were considered significant. The coefficient from the regression model determined enrichment or depletion.

Samples were grouped into compositionally distinct community states based on dominant taxon present (taxon identity at the highest level of classification was used as a sample classifier). Additional unsupervised clustering methods (Partitioning Around Medoids and Dirichlet Multinomial Mixture models) also supported the observation that the dominant taxon within a community significantly co-varied with beta-diversity. The majority of samples belonged to one of six microbiota-dominated states including Moraxella (n=1,058), Staphylococcaceae (n=951), Streptococcus (n=263), Alloiococcus (n=261), Corynebacterium (n=153), or Haemophilus (n=135). All other samples (n=301), which exhibited rarer community states dominated by other genera were categorized as “other”.

To ask whether or not alpha diversity differed across the seven community states using repeated measures, we used linear mixed effects (LME) models to model the alpha diversity measure against each of the community states as a categorical variable. Analysis of variance tests then asked whether alpha diversity differed across all community states. P-values derived from this method are denoted in the manuscript as “ANOVA (LME)”.

Risk ratios (RRs) were computed using generalized estimating equations (GEE) in the geepack package in R. An exchangable correlation structure for each participant was used to generate the estimates. The outcome of the regression equation was a binary variable comparing each community state versus all other states. Q-values were derived from a Benjamini-Hochberg False Discovery Rate across p-values for each community state within variables of interest; q-values less than 0.10 were considered significant.

Pair-wise assessment of community states in repeated measures samples was performed for each participant; community state based on dominant taxon was used to classify each sample and the frequency with which the same or distinct dominant taxa occurred in the temporally-neighboring sample obtained from an individual was determined(3). A heat map with transition counts was generated for the overall population and for the first three samples provided from each participant. The latter approach was taken to normalize the number of samples assessed per participants.

Co-occurrence networks of OTUs were constructed using the SparCC and WGCNA(19) packages in R, using all samples from all participants. To reduce data complexity, the rarified OTU table was filtered to include only those taxa present in at least 25% of the samples, resulting in 150 OTUs being used for network analysis. SparCC(18) generated the correlation matrix, which was transformed to an adjacency matrix using soft thresholding, and a dissimilarity matrix was generated. The dissimilarity matrix was then hierarchically clustered and the resulting dendrogram was cut using dynamicTreeCut in the stats package in R to generate modules. Closely related modules were combined using transformations of the module eigengenes, which generated a dissimilarity matrix for further hierarchical clustering, and the final network was visualized using WGCNA and phyloseq(28) in R. Hub OTUs are defined as those with a greater number of connections within their module than between modules, while connector OTUs are those with a greater number of connections between modules than within.

### Example 2. Compositionally and Functionally Distinct Sinus Microbiota in Chronic Rhinosinusitis Patients have Immunological and Clinically Divergent Consequences

Abstract.

Chronic rhinosinusitis (CRS) is a heterogeneous disease characterized by persistent sinonasal inflammation and sinus microbiome dysbiosis. The basis of this heterogeneity is poorly understood. Without being bound by any scientific theory, experiments were performed to address the hypothesis that a limited number of compositionally distinct pathogenic bacterial microbiota exist in CRS patients and invoke discrete immune responses and clinical phenotypes in CRS patients.

Sinus brushings from patients with CRS (n=59) and healthy individuals (n=10) collected during endoscopic sinus surgery were analyzed using 16S rRNA gene sequencing, predicted metagenomics, and RNA profiling of the mucosal immune response. Data show that CRS patients cluster into distinct sub-groups (DSI-III), each defined by specific pattern of bacterial co-colonization (PERMANOVA; p=0.001, r²=0.318). Each subgroup was typically dominated by a pathogenic family: Streptococcaceae (DSI), Pseudomonadaceae (DSII), Corynebacteriaceae [DSIII(a)], or Staphylococcaceae [DSIII(b)]. Each pathogenic microbiota was predicted to be functionally distinct (PERMANOVA; p=0.005, r²=0.217) and encode uniquely enriched gene pathways including ansamycin biosynthesis (DSI), tryptophan metabolism (DSII), two-component response [DSIII(b)], and the PPAR-γ signaling pathway [DSIII(a)]. Each is also associated with significantly distinct host immune responses; DSI, II, and III(b) invoked a variety of pro-inflammatory, T_{H}1 responses, while DSIII(a), which exhibited significantly increased incidence of nasal polyps (Fisher's Exact; p=0.034, Relative Risk=2.16), primarily induced IL-5 expression (Kruskal Wallis; FDR p=0.045).

A large proportion of CRS patient heterogeneity may be explained by the composition of their sinus bacterial microbiota and related host immune response—features which may inform strategies for tailored therapy in this patient population.

The field of human microbiome research has profoundly altered the view of the diversity of human-associated microbes and encoded functions, and demonstrated that the microbiome co-varies with host health status [1-3]. Microbes overtly colonize the upper respiratory mucosal surface of healthy subjects [4, 5], with lower bacterial burden and diversity observed in the lower airways [6]. In contrast, patients with chronic inflammatory airway disease exhibit compositionally distinct upper and lower respiratory microbiota, enriched for known or suspected pathogenic species, and related to features of pulmonary disease [1, 5, 7, 8]. Chronic rhinosinusitis (CRS), characterized by persistent inflammation of the sinonasal mucosa lasting at least 12 weeks, is a common and refractory respiratory disease [9, 10], not least because of the immunologic and clinical heterogeneity exhibited by these patients. Until recently, little was known of the microbiome of the sinus mucosa in either healthy subjects or diseased patients. However, several recent culture-independent studies have now demonstrated that loss of sinus microbiota diversity is a common feature of patients with CRS [5, 11, 12], and, independently that greater pre-operative sinus microbiota diversity is associated with improved post-operative outcomes [13]. Respiratory pathogens such as Pseudomonas aeruginosa or Staphylococcus aureus are commonly isolated from CRS patients [14], while pathobionts, such as Corynebacterium tuberculostearicum, also found to be enriched in CRS patients, have demonstrable capacity to induce sinus mucosal infection in murine models [5]. However, these pathogens do not exist in isolation, but in mixed-species mucosal microbiota, the composition and activities of which, it is hypothesized, explain the substantial clinical and immunological heterogeneity observed in CRS patients.

Previous efforts to explain CRS patient heterogeneity have been based on clinical [15, 16], immunologic [17-20], or pathologic [21] endotypes, though these studies have been relatively small and focused on specific immune cell populations or clinical features. More recently airway studies have examined whether subject stratification based on microbiota composition offers an improved approach for understanding immunological or clinical phenotypic variation across populations. A large study (n=234) of the infant nasopharyngeal microbiota identified six compositionally distinct microbiota, each dominated by a common respiratory bacterial genus and associated with significantly different relative risk for acute upper respiratory infection or development of asthma at 5 years [4]. Similarly, three compositionally and functionally distinct pathogenic lung microbiota have been described in HIV-infected pneumonia patients (n=182), each co-associate with a specific host immune response profile and differ in mortality risk [22]. Moreover, the predicted metabolic products of these three distinct pathogenic lower airway communities were found to be enriched in paired serum samples, indicating that the microbiome of the overtly colonized airway may actively contribute both to local and systemic metabolic and immune dysfunction. The capacity for meaningful stratification based on microbiota composition is perhaps most compelling in a recent study of 1 month old infants (n=130), who were divisible into three compositionally distinct gut microbiota states, one of which conferred a 3-fold increased risk of atopy at age 2 years and asthma at age 4 years. The associated products (sterile fecal water) of these compositionally distinct high-risk microbiotas induced CD4+IL4+ cell population expansion and CD4+CD25+FoxP3+ suppression ex vivo; a specific lipid enriched in the stool of high-risk infants, recapitulated the effect of fecal water on the CD4+CD25+FoxP3+ [23]. Hence, several lines of investigation suggest that patient immunological status and clinical outcomes differ significantly based on the specific microbiota composition present. Without being bound by any scientific theory, given these observations, it was hypothesized that CRS patient heterogeneity may be explained by the presence of distinct pathogenic sinus microbiota that invoke discrete host immune responses and relate to clinical phenotypes. To address this hypothesis, the sinus mucosal microbiome and parallel host immune responses of a cohort of CRS and healthy subjects was examined, and these findings were related to clinical outcomes of nasal polyposis. The data demonstrate the presence of distinct pathogenic sinus microbiota in CRS patients each predicted to encode unique functional attributes, which co-associate with specific innate and adaptive immune responses, and significantly different relative risk of nasal polyposis.

Materials and Methods

Study Design.

The UCSF Institutional Review Board (approval number 11-07750) approved this study. All participants were informed of the objectives of this study and signed a written consent form prior to their participation. Adult patients (n=76) undergoing endoscopic sinus surgery (ESS) for CRS were enrolled at the University of California San Francisco. A subset of CRS patients (n=11) was sampled at multiple sites for microbial biogeography analysis. Two patients did not yield PCR product, and five patients were removed from the analysis due to low sequence depth. Therefore, 69 patients (10 healthy, 59 CRS patients) were included (Table 5).

Patient enrollment and sample collection. Disease was clinically diagnosed according to the 2007 Rhinosinusitis Task Force guidelines [24] and severity was radiographically quantified using the Lund-Mackay Computed Tomography (CT) scoring system. All CRS patients had symptoms for more than 12 consecutive weeks and CT evidence of inflammation within a month of sampling for this study. Patient demographics are described in Table 5. Recent clinical history, sino-nasal outcomes test (SNOT-20), and CT sinus review were collected and used to confirm CRS diagnosis. Recent antibiotic use, as well as intraoperative antibiotic administrations were recorded at the time of sample collection. Co-morbidities, including physician-diagnosed asthma or cystic fibrosis (CF) were recorded. Sinus brushings were obtained for 11 control patients undergoing surgery for non-CRS etiologies including oral surgery, trans-sphenoidal pituitary surgery, or endoscopic cerebral spinal fluid leak repair. Endoscopically guided protected brushes (ConMed #149, NY) were used to collect mucosal samples of the diseased sinus by brushing each surface gently while rotating the brush five times. Each sample was immediately placed in 1 ml of RNAlater, transferred to 4° C. for 24-48 hours to permit the nucleic acid preservative to permeate cells prior to storage at −80° C.[25].

TABLE 5
Demographics of CRS patients and healthy subjects included in this study

| | | | | Pre-operative | Lund- | | |
|---|---|---|---|---|---|---|---|
| | | | | antibiotics ≤3 | Mackay | | |
| Sample ID | Age | Gender | Disease | months | score | Polyp | Status |
| 33 | 47 | F | CRS + CF | Multiple (≥3) | 22 | N | Included |
| 36 | 27 | F | CRS + CF | Multiple (≥3) | 18 | Y | Included |
| 39 | 28 | M | CRS + CF | Multiple (≥3) | 13 | N | Included |
| 61 | 20 | M | CRS + CF | Azithromycin | 15 | Y | Included |
| 64 | 71 | M | CRS + CF | Diclozacillin | 16 | Y | Included |
| 83 | 24 | M | CRS + CF | Multiple | 19 | Y | Included |
| 90 | 20 | F | CRS + CF | Ceftrioxone | 19 | Y | Included |
| 91 | 47 | M | CRS + CF | Zithromax/dapsone | 17 | Y | Included |
| 108 | 30 | M | CRS + CF | Multiple (≥3) | 16 | Y | Included |
| 1 | 41 | M | CRS | Augmentin | 5 | N | Included |
| 7 | 39 | F | CRS | Augmentin | 12 | N | Included |
| 8 | 55 | F | CRS + Asthma | Augmentin | 23 | Y | Included |
| 15 | 55 | M | CRS + Asthma | Clarithromycin | 15 | N | Included |
| 16 | 51 | M | CRS | Augmentin | 22 | N | Included |
| 17 | 61 | F | CRS + Asthma | Multiple (≥3) | 12 | N | Included |
| 22 | 58 | M | CRS + Asthma | Bactrim | 12 | Y | Included |
| 32 | 19 | M | CRS | Augmentin | 22 | Y | Included |
| 34 | 51 | M | CRS + Asthma | Levofloxacin | 5 | N | Included |
| 40 | 72 | F | CRS | Tobramycin | 9 | N | Included |
| 43 | 46 | M | CRS | Augmentin | 12 | N | Included |
| 44 | 62 | M | CRS | Levofloxacin | 20 | Y | Included |
| 54 | 71 | M | CRS + Asthma | dicloxacillin | 13 | Y | <10,000 |
| | | | | | | | sequences/sample |
| 55 | 68 | F | CRS + Asthma | Clotrimazole | 21 | Y | Included |
| 56 | 67 | F | CRS | Augmentin/Bactrim | 14 | Y | Included |
| 58 | 85 | M | CRS | Ampicillin/sulbactam | ND^{a} | ND | No PCR product |
| 59 | 42 | F | CRS + Asthma | Clindamycin | 4 | Y | Included |
| 60 | 35 | M | CRS | Multiple (≥3) | 16 | N | Included |
| 63 | 77 | F | CRS | Levofloxacin | 6 | N | Included |
| 80 | 68 | M | CRS | Augmentin | 8 | N | Included |
| 81 | 65 | F | CRS + Asthma | Augmentin | 10 | N | Included |
| 82 | 28 | M | CRS | Augmentin | 9 | Y | Included |
| 85 | 24 | F | CRS + Asthma | Augmentin | 4 | N | Included |
| 86 | 88 | F | CRS | None | 19 | Y | Included |
| 88 | 73 | M | CRS | None | 21 | Y | Included |
| 89 | 52 | M | CRS | Clarithromycin | ND | ND | No PCR product |
| 92 | 59 | M | CRS | Multiple (≥3) | 13 | N | Included |
| 93 | 48 | M | CRS | Azithromycin/ | 11 | Y | Included |
| | | | | augmentin | | | |
| 94 | 72 | F | CRS + Asthma | Augmentin | 8 | N | Included |
| 96 | 54 | M | CRS | Cephalexin | 11 | Y | Included |
| 97 | 52 | M | CRS | Cetirizine | 22 | Y | Included |
| 98 | 39 | M | CRS + Asthma | Multiple (≥3) | 16 | Y | Included |
| 99 | 57 | M | CRS | Levofloxacin | 16 | Y | Included |
| 100 | 62 | F | CRS | Bactrim | 4 | N | Included |
| 101 | 27 | M | CRS + Asthma | Azithromycin | ND | Y | Included |
| 103 | 36 | M | CRS | Augmentin | 10 | Y | Included |
| 104 | 23 | M | CRS | None | 12 | N | Included |
| 105 | 18 | M | CRS | None | 21 | N | Included |
| 107 | 37 | F | CRS | Cephalexin | 7 | Y | Included |
| 109 | 71 | M | CRS + Asthma | Augmentin | 11 | Y | Included |
| 110 | 18 | F | CRS | Multiple (≥3) | 16 | N | Included |
| 111 | 59 | M | CRS | Augmentin | 10 | N | Included |
| 112 | 48 | F | CRS | Augmentin | 9 | N | Included |
| 114 | 37 | F | CRS | Clindamycin | 1 | N | Included |
| 115 | 26 | F | CRS | Augmentin | 3 | N | Included |
| 117 | 33 | M | CRS | Ciprofloxacin | 19 | N | Included |
| 120 | 50 | F | CRS + Asthma | Multiple (≥3) | 15 | Y | Included |
| 121 | 33 | F | CRS | Augmentin | 9 | Y | Included |
| 122 | 45 | F | CRS + Asthma | Augmentin | 21 | Y | Included |
| 123 | 74 | M | CRS + Asthma | Augmentin | 16 | Y | Included |
| 124 | 30 | M | CRS + Asthma | Augmentin | 6 | N | <10,000 |
| | | | | | | | sequences/sample |
| 126 | 43 | F | CRS + Asthma | Augmentin | 11 | Y | <10,000 |
| | | | | | | | sequences/sample |
| 128 | 69 | M | CRS + Asthma | Bactrim | 21 | Y | Included |
| 130 | 59 | M | CRS + Asthma | Augmentin | 17 | Y | Included |
| 132 | 48 | M | CRS | Rifampin | 13 | N | <10,000 |
| | | | | | | | sequences/sample |
| 143 | 61 | M | CRS | Augmentin | ND | N | Included |
| 30 | 38 | M | Healthy | Topical bacitracin | ND | N | <10,000 |
| | | | | | | | sequences/sample |
| 31 | 59 | M | Healthy | Amoxicillin/ | ND | N | Included |
| | | | | azithromycin | | | |
| 131 | 59 | F | Healthy^{b} | None | 1 | N | Included |
| CRS14 | 41 | M | Healthy^{b} | None | ND | N | Included |
| CRS15 | 39 | M | Healthy | None | ND | N | Included |
| CRS16 | 37 | F | Healthy | None | ND | N | Included |
| CRS17 | 46 | F | Healthy | None | ND | N | Included |
| CRS18 | 46 | M | Healthy | None | ND | N | Included |
| CRS19 | 31 | F | Healthy | None | ND | N | Included |
| CRS20 | 18 | F | Healthy | None | ND | N | Included |
| ctrl4 | 22 | M | Healthy | None | ND | N | Included |

^{a}ND not determined
^{b}Allergic rhinitis

DNA Extraction.

Nucleic acids were extracted as previously described using the AllPrep kit (Qiagen, CA), to purify DNA and RNA in parallel[5, 25]. Briefly, brushes were placed in Lysis Matrix B tubes in 600 μl Buffer RLT Plus with β-mercaptoethanol and bead beaten for 30 seconds at 5.5 m sec⁻¹ for nucleic acid extraction per manufacturer's protocol. DNA and RNA were quantified using a NanoDrop2000 (ThermoFisher, CA). DNA concentrations were normalized to 50 ng μl⁻¹ per sample for 16S rRNA gene sequence library preparation, described below.

16S rRNA Gene Library Preparation.

Barcoded primers 515F/806R were used to amplify the V4 region of the 16S rRNA gene as previously described [26, 27]. Since double bands were present, one human mitochondrial band and a microbial 16S band, amplicons of the correct size (384 bp) were gel extracted with a Qiagen Gel Extraction kit per manufacturer protocol. Purified PCR product was analyzed on Bioanalyzer (Aligent), quantified using the Qubit HS dsDNA kit (Invitrogen), and pooled at 25 ng per sample. The pooled library was quantified using the KAPA QPCR Illumina Library Quantification kit (KAPA Biosystems), diluted to 2 nM, denatured, and 5 pM was loaded onto the Illumina Mi Seq cartridge (V2) in combination with a 15% (v/v) of denatured 12.5 pM PhiX spike-in. In addition to negative control extraction blanks, a mock community composed of equal genomic concentration (2 ng each per reaction) of Escherichia coli ATCC25922, Pseudomonas aeruginosa ATCC27853, Corynebacterium tuberculostearicum ATCC35692, Lactobacillus sakei ATCC15521, and L. rhamnosus ATCC53103 was also used to monitor runs.

16S rRNA Gene Sequence Processing.

Sequence analysis of 16S rRNA data was performed using the QIIME version 1.8.0[28] and in the R environment.

Sequence analysis of 16S rRNA data was performed using the QIIME version 1.8.0⁷⁵ and in the R environment. Briefly, raw sequence data were de-multiplexed by barcode, and low-quality bases were discarded. Each 251×151 paired read was assembled using FLASh (Fast Length Adjustment of SHort reads⁷⁶) with parameters: -r 251 -f 300 -s 30 -m 15. Sequences were quality filtered in QIIME 1.8.0 as follows. Phred quality scores of Q30 were retained; if three consecutive bases were <Q30, then the read was truncated before the low-quality bases. The resulting read was retained in the dataset if it was at least 75% of the original length. Operational taxonomic units (OTUs) were picked at 97% sequence identity using uclust against the Greengenes database (13_5)^{77,78}. Reads that failed to hit the reference sequence collection were retained and clustered de novo. Sequences were aligned using PyNAST and taxonomy was assigned using uclust in the qiime environment⁷⁰. PyNAST-aligned sequences were chimera checked using ChimeraSlayer and putative chimeras were removed from the OTU table. Eight OTUs that were present in the negative extraction controls, which corresponded to members of Pseudomonadaceae, Delftia, Mycoplana, Bradyrhizobium, and Neisseriaceae, were removed from the OTU table. A phylogenetic tree was then built using FastTree^{w} and used to compute Faith's Phylogenetic Diversity and UniFrac distances. Since the rarefaction curves approached an asymptote (indicating adequate community coverage) at a sequence depth 10,055 sequences, and all but 5 samples were sequenced at least to this depth, the OTU table was multiply rarefied to 10,055 high-quality, chimera checked sequences per sample for subsequent analyses using a custom script (github.com/alifar76/MicroNorm).

Sequence and Statistical Analyses.

Since the rarefaction curves approached an asymptote (indicating adequate community coverage) at a sequence depth 10,055 sequences, and all but 5 samples were sequenced at least to this depth, the OTU table was multiple rarefied to 10,055 high-quality, chimera checked sequences per sample for subsequent analyses using a custom script (https://github.com/alifar76/MicroNorm). All subsequent analyses were performed on this rarefied table. UniFrac, Canberra and Bray-Curtis dissimilarity matrices were generated in QIIME 1.9.0, and Principal Coordinates Analysis (PCoA) plots were used to visualize ordinations using emperor[29]. Permutational multivariate analysis of variance (PERMANOVA) using the adonis function in the R Vegan package was used to determine significance in dissimilarity matrices across samples by metadata categories (e.g. disease, sinotype, antibiotic use, age, and disease severity [30, 31]). Faith's phylogenetic diversity, number of unique OTUs (richness), and Pielou's Evenness were calculated and a permutational t-test (999 monte carlo permutations) was used to determine changes in alpha-diversity. Multiple comparisons were corrected for false discovery using the Benj amini-Hochberg (BH) method and a corrected p≤0.05 was considered significant. Changes in taxon relative abundance were determined per OTU using a zero-inflated negative binomial (github.com/alifar76/NegBinSig-Test) distribution on a regression model. Multiple comparisons were corrected for false discovery using the BH method and q values are reported [32]. Kruskal-Wallis was used to determine if statistically significant differences in OTU or KEGG pathway abundances existed between more than two groups, such as healthy patients, nonCF-CRS, and CF-CRS patients. To identify clusters, the Dirichlet Multinomial Mixtures probabilistic community modeling was performed using the DirichletMultinomial package [33] in R with family-level taxonomy using absolute abundances of each family. The Laplace approximation was used to calculate model fit and to determine the number of components (clusters). Distinct sample clusters that represented the best model fit were termed Dirichlet states (DS). To determine whether DSIII could be separated into two phylogenetically distinct groups, hierarchical cluster analysis was performed on a weighted UniFrac distance matrix using an edited version of pvclust in R (code presented below). Kruskal-Wallis was used to determine whether host genes were significantly up or down-regulated in disease. Multiple comparisons were corrected for false discovery using the BH method and q values were reported. Statistical analysis was performed using R.

Edited pvclust R code for use with a precomputed distance matrix:

Predicted Metagenomics.

Metagenome prediction from the closed-reference OTUs (greengenes 13_5) of the multiple rarefied OTU table was performed using the PICRUSt [34]. QIIME 1.8.0 was used to analyze the predicted metagenomes. Differential abundances of pathways were tested using a Kruskal Wallis test when comparing more than two groups, or a three-model approach (Negative Binomial, Zero-Inflated Negative Binomial, or Poisson distributions) applied on a regression to test pairwise comparisons. Model fit was determined using AIC values and the associated statistic was reported (github.com/alifar76/NegBinSig-Test). Multiple comparisons were corrected for false discovery using the BH method and q values were reported. NTSI (Nearest Sequenced Taxon Index) scores were calculated using the -a flag in metagenome_predictions.py. These represent the average branch length separating OTUs in a sample from a reference bacterial genome. A heatmap was constructed for KEGG categories that were enriched or depleted in each disease state using Heatmap.3. in R. For visualization, read counts were normalized [log 2(x+1)] and scaled by row.

Metagenome prediction from the closed-reference OTUs (greengenes 13_5) of the multiply rarefied OTU table was performed using the PICRUSt software (picrust.github.io/picrust/)⁸¹. QIIME 1.8.0 was used to analyze the predicted metagenomes. A table of KEGG pathways collapsed from KOs to level 3 was used for subsequent analysis. Since the resulting table had a range of count depths for each pathway, the table was rarefied to 2,000,000 KEGG pathway counts per sample prior to computing between-sample distances (Bray Curtis, Canberra) or testing differential abundances of pathways using a Kruskal wallis for comparing more than two groups, or a three-model approach (Negative Binomial, Zero-Inflated Negative Binomial, or Poisson distributions) to test pairwise comparisons. Model fit was determined using AIC values and the associated statistic was reported (github.com/alifar76/NegBinSig-Test).

Quantitative PCR for Bacterial Burden and Human Gene Expression.

Quantitative PCR (qPCR) was used to quantify bacterial burden as a ratio to human beta-actin. A custom qPCR array was developed (SA Biosciences) and used to quantify host gene expression using RNA extracted in parallel from patient sinus brushes.

QPCR was used to quantify bacterial burden. The universal primers 338F/518-R (338F, 5′-ACTCCTACGGGAGGCAGCAG-3′⁸² (SEQ ID NO: 1) and 518R 5′-ATTACCGCGGCTGCTGG-3′(SEQ ID NO: 2)) were used to amplify the 196 bp region of the V3-V4 rRNA gene for quantification of total 16S rRNA copy number as previously described⁸³. Copy number was normalized to host beta-actin (ACTB-F, 5′-AAGATGACCCAGATCATGTTTGAGACC-3′ (SEQ ID NO: 3), ACTB-R, 5′-AGCCAGTCCAGACGCAGGAT-3′ (SEQ ID NO: 4)). Reaction mixtures (20 μl total) contained 10 μl SYBRgreen MM (2×), 1 μM each primer, 20 ng template DNA, and 4 μl water. Reactions were amplified using the QuantStudio 6 (Life Technologies) per the following conditions: 95° C. for 10 min and 40 cycles of 95° C. for 30 sec, 55° C. for 60 sec, and 72° C. for 30 sec. The data acquisition step was set at 55° C. and a disassociation curve was recorded. Standard curves of known 16S rRNA (Escherichia coli) or human β-actin gene copy number were used to calculate copy number in test samples⁸⁴.

To determine whether sinonasal microbial community composition correlated with aberrant host immune responses, mucin secretion or epithelial barrier function, a custom QPCR array was developed (SA Biosciences) and used to quantify host gene expression using RNA extracted in parallel from patient sinus brushes. Contaminating DNA was removed from 250 ng of total RNA and cDNA was synthesized using the RT² First Strand Synthesis kit (Qiagen). Resulting cDNA was used in a 10 μl SYBR green reaction with custom primers for each gene of interest on a Life Technologies Quant 6 QPCR instrument. PCR conditions were as follows: One cycle at 95° C. for 10 min, 40 cycles of 95° C. for 15 s and 60° C. for 60 seconds, followed by a melt curve. Expression of Occludin, Claudin 2, MUCSAC, IL-4, IL-5, IL-6, IL-8, IL-25, IL-17A, IL-10, IL-1β, IL-33, CCL11 (eotaxin), TSLP (thymic stromal lymphopoietin), TNF-α, ARG1, TGFβ1 (transforming growth factor, beta 1), CLCA1 (chloride channel accessory), and IFN-γ were normalized to β-actin housekeeping gene by the AACt method⁸⁵. Fold change is reported as 2^{-ΔΔCt}.

Results

Sinus Mucosal Microbiome Perturbations Characterize CRS and are Related to Disease Status.

Our cohort consisted of 76 subjects. Sixty-five were CRS patients and 11 were healthy subjects. CRS patients included those with cystic fibrosis (CF-CRS; n=9) or without CF (NonCF-CRS; n=56). Approximately 37% of nonCF-CRS patients were asthmatic (n=21 asthmatic, n=35 non-asthmatic, Table 5). Sinus brushing samples from 2 subjects yielded no 16S rRNA amplicons (both from nonCF CRS patients), and a further 5 samples were removed due to low sequence depth (<10,000 sequences/sample; n=4 NonCF-CRS and n=1 healthy). Thus, 10 healthy individuals and 59 CRS patients were included in the analyses presented.

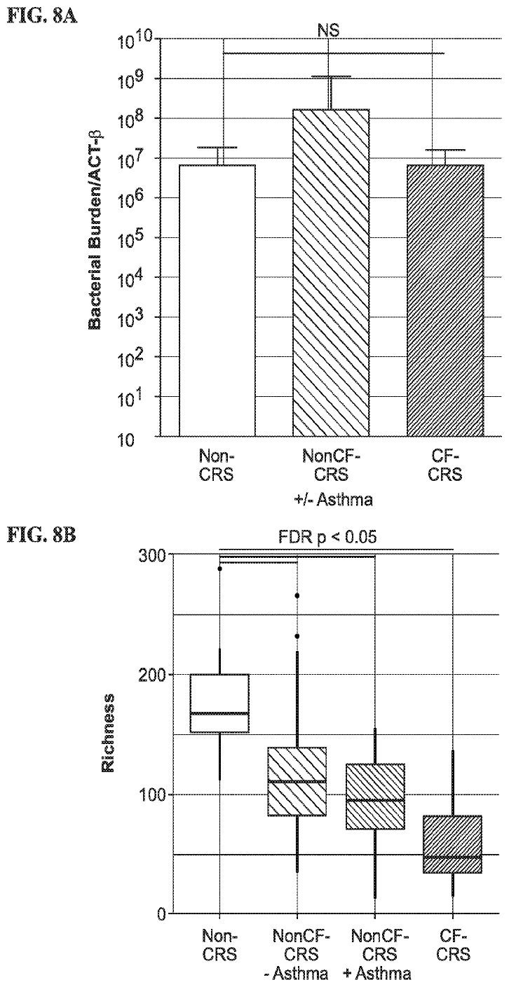

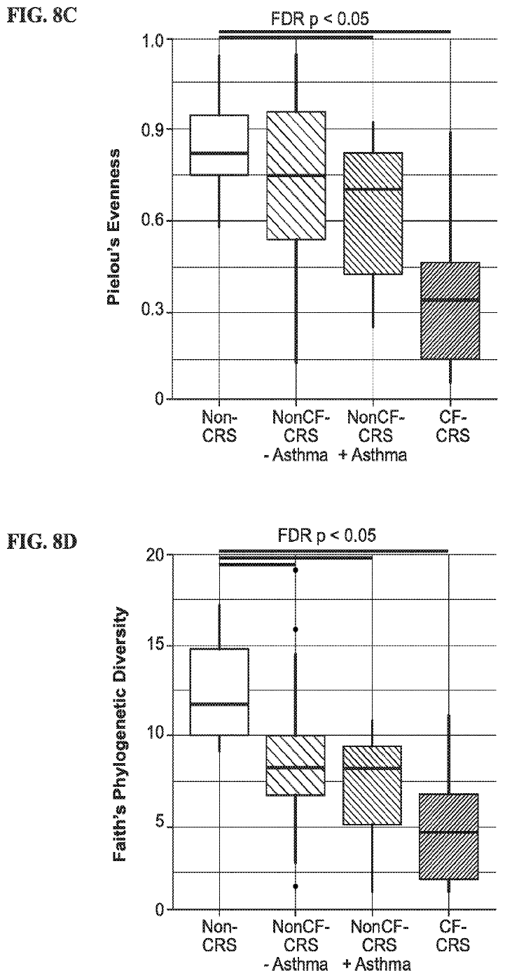

CRS disease severity (as assessed by Lund-Mackay scores) did not differ across CF or asthma patients (Tukey's post hoc comparison p>0.05, FIG. 12A). Mucosal bacterial burden (based on total 16S rRNA copy number), was not significantly different across healthy and CRS patients (ANOVA p=0.781; FIG. 8A), consistent with previous reports[5, 12]. Also consistent with previous findings[5, 12], was the observation that compared with healthy subjects, CRS patients exhibited significantly lower microbiota richness, evenness and diversity. Of note, diminished alpha-diversity in CRS patients was more pronounced in those with concomitant lower airway disease (Permutational t-test, all p<0.01; FIGS. 8B-D). Multivariate analysis (PERMANOVA) of sinus bacterial beta diversity on a weighted UniFrac distance matrix was used to determine whether factors such as age, antimicrobial administration, polyposis, revision surgery (a complete list is provided in Table 6) explained the observed variation in community composition in either all patients (CRS and healthy), or within the CRS patients. Of these, only disease status (healthy, CF-CRS, nonCF-CRS+Asthma, nonCF-CRS−Asthma) was significantly related to beta-diversity, but only explained a small portion of microbiota compositional variance and the effect size was small (PERMANOVA p=0.001; 8.9% of variation explained; Table 6).

TABLE 6
Multivariate analysis (PERMANOVA) of sinus bacterial
beta diversity on a weighted UniFrac distance matrix

| PERMANOVA (Patient Cohort) | r² | p value |
|---|---|---|
| Dirichlet State (disease³) | 0.326 | 0.001 |
| Dirichlet State (all^{b}) | 0.318 | 0.001 |
| Disease (all) | 0.065 | 0.014 |
| Antibiotic use <3 months (disease) | 0.036 | 0.052 |
| Polyp Presence/Absence (disease) | 0.025 | 0.152 |
| Anatomic Location (all) | 0.039 | 0.185 |
| Age bin 10 year (all) | 0.118 | 0.196 |
| Antibiotic class <3 months (all) | 0.260 | 0.24 |
| Age bin 5 year (all) | 0.153 | 0.367 |
| Antibiotic class <3 months (disease) | 0.286 | 0.399 |
| Anatomic Location (disease) | 0.0348 | 0.403 |
| Age bin 10 year (disease) | 0.119 | 0.473 |
| Age bin 5 year (disease) | 0.168 | 0.521 |
| LMS Bin (Low/Medium/High) (disease) | 0.046 | 0.554 |
| Revision surgery (Y/N) (disease) | 0.042 | 0.668 |
| Age (disease) | 0.674 | 0.799 |
| Lund-Mackay Score (LMS) (disease) | 0.327 | 0.906 |
| Age (all) | 0.561 | 0.944 |

^{a}CF-CRS, nonCF-CRS + Asthma, nonCF-CRS − Asthma
^{b}Healthy, CF-CRS, nonCF-CRS + Asthma, nonCF-CRS − Asthma

Discrete Pathogenic Sinus Microbiota Exists in CRS Patients.

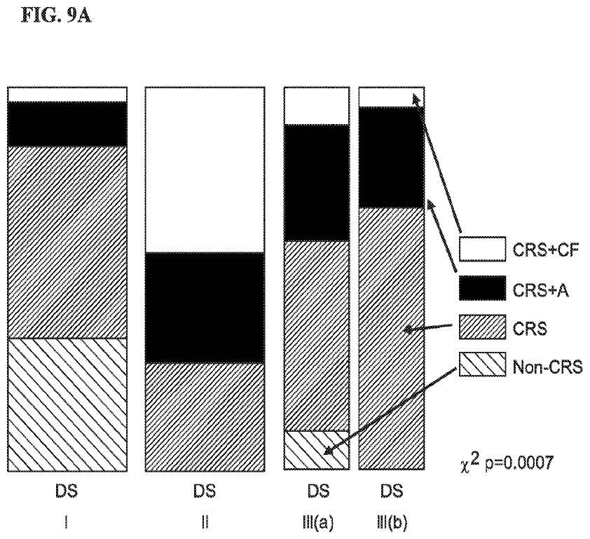

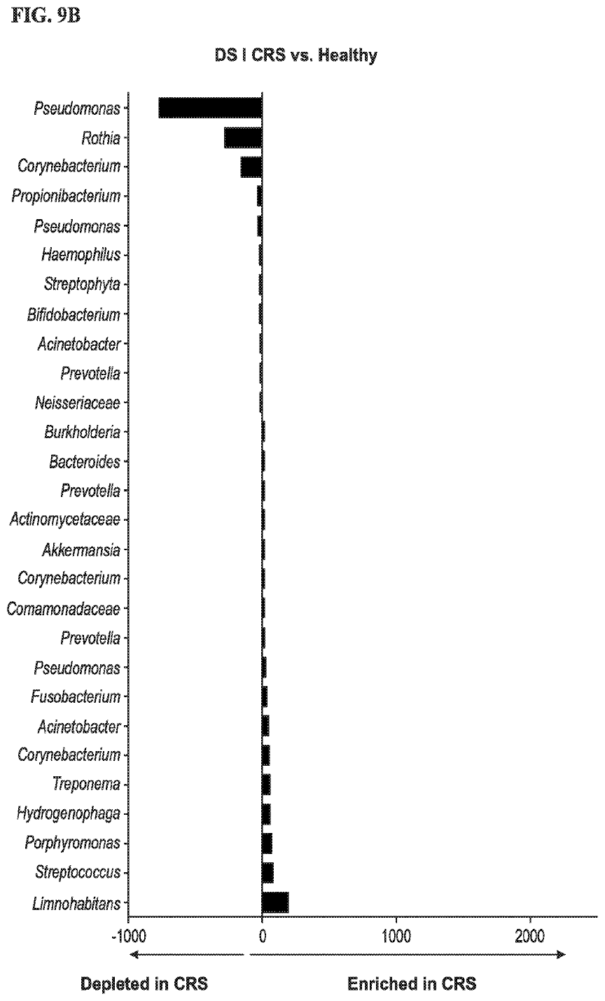

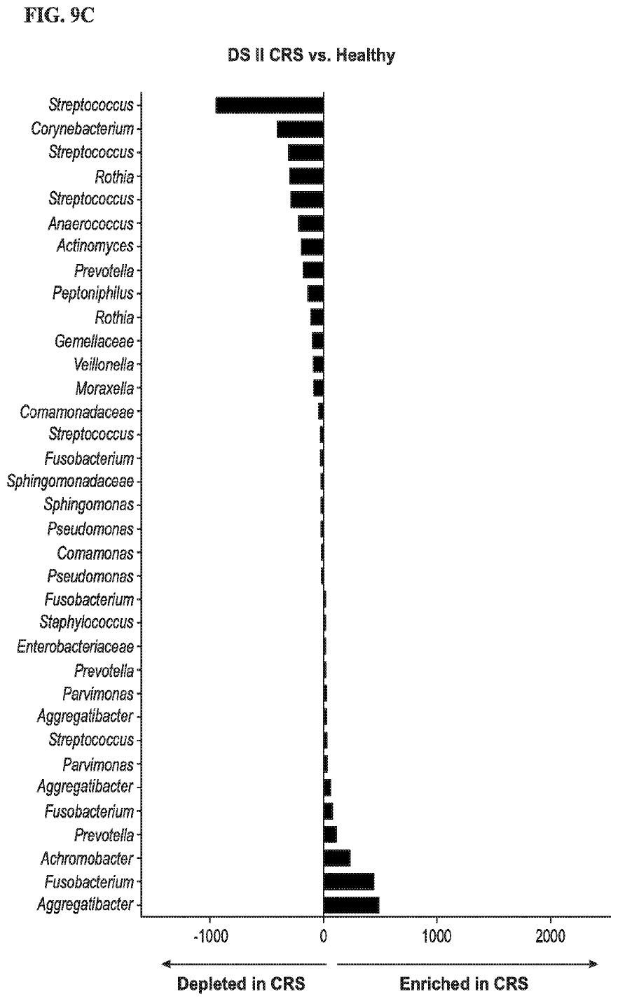

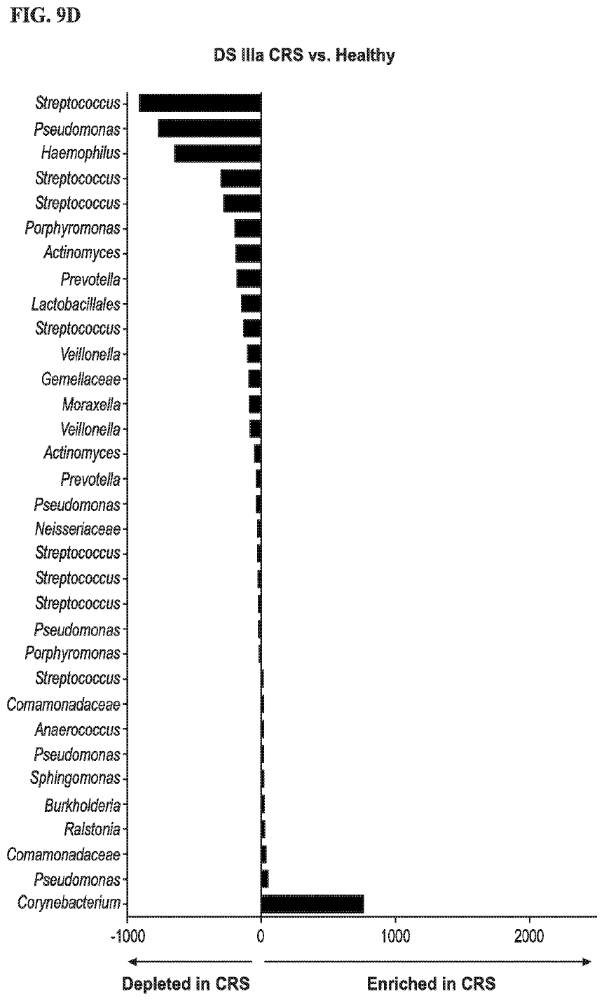

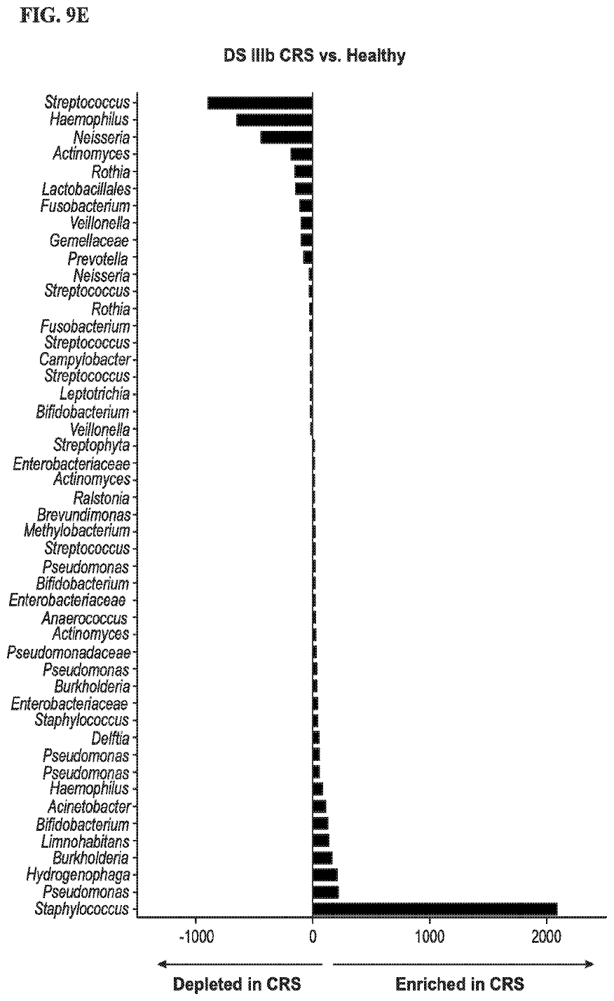

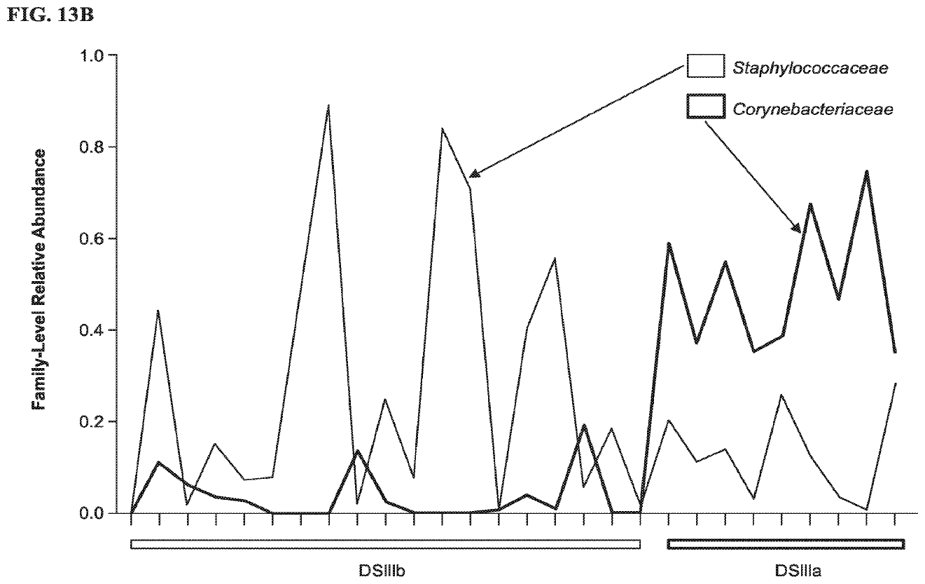

Without being bound by any scientific theory, it was postulated that the microbiota dysbiosis exhibited by CRS patients does not represent a single state, but rather a gradient of dysbioses punctuated by a limited number of distinct pathogenic microbiota compositional states. This hypothesis was addressed through the application of an unbiased probabilistic model, Dirichlet Multinomial Mixtures (DMM)[33] which identifies clusters of samples based on bacterial community composition. Based on a La Place approximation, three distinct sample clusters, termed Dirichlet states (DSI-III) represented the best model fit (FIG. 13A); DSI comprised 26 subjects (n=9/10 healthy, n=17 CRS), DSII comprised 14 CRS patients, and DSIII comprised 28 CRS patients and one non-CRS subject. Upon chart review, it was noted that this subject had allergic rhinitis. DS clusters were confirmed as compositionally distinct by PERMANOVA (weighted UniFrac PERMANOVA p=0.001, 31.8% variation; FIG. 9A), a finding that was robust irrespective of the distance matrix used to analyze the 16S rRNA data (Table 7). Since both a weighted and unweighted UniFrac distance matrix significantly explained DS-defined sample clustering, indicated that both bacterial phylogeny and rarer taxa in these communities discriminated DS groups. To further confirm this, sequence reads associated with the dominant family in each sample were removed and the data reanalyzed. DS classification remained significantly related to community composition (weighted UniFrac; PERMANOVA p=0.001, 18.2% variation), indicating that patterns of co-associated lower-abundance taxa are discrete and relatively conserved within each of the three DS microbiota.

TABLE 7
Weighted and unweighted UniFrac distance matrix

| PERMANOVA (Dirichlet States) | r² | p value |
|---|---|---|
| Weighted Unifrac | 0.326 | 0.001 |
| Unweighted Unifrac | 0.182 | 0.001 |
| Bray-Curtis | 0.195 | 0.001 |
| Canberra | 0.099 | 0.001 |

The proportion of healthy subjects and CRS patients with or without pulmonary co-morbidities varied significantly across DSI-III (Chi Square p=0.0007), with DSI possessing the least and DSII the greatest proportion of asthmatic and CF-CRS patients. This implicates a co-association between specific pathogenic sinus community states and lower airway disease and also provides the first evidence that specific pathogenic sinus microbiota are common to both CF and asthmatic patients. While recent antibiotic use trended toward significance, it only explained a very minor portion of community compositional variance (PERMANOVA p=0.052, r²=0.036, Table 6) and did not differ across DSI-III (Chi Square p=0.149), likely because these microbiota exist in antimicrobial resistant biofilms on the sinus mucosal surface [35]. Disease severity, as measured by Lund-Mackay radiographic scores also did not differ across DSI-III (ANOVA p=0.825), suggesting that distinct pathogenic microbiota may drive equally severe disease symptoms, albeit via distinct mechanisms (it should be noted that these patients were undergoing functional endoscopic sinus surgery at the time of sample collection).

DSIII was the largest group and was comprised of patients whose sinus mucosal microbiota represented a compositional continuum dominated by either Staphylococcaceae (Firmicutes) or Corynebacteriaceae (Actinobacteria). Since these taxa are phylogenetically distinct, are known competitors in the upper airways [36, 37], and elicit unique immune responses [38], Corynebacteriaceae- or Staphylococcaceae-dominated patients within this group were identified as distinct DSIII sub-groups, identified as DSIII(a) (n=9) or III(b) (n=19) respectively. This sub-grouping strategy was statistically supported by hierarchical clustering analysis on a weighted UniFrac distance matrix (au, p<0.05), and the existence of a reciprocal relationship between Corynebacteriaceae [DSIII(a)] and Staphylococcaceae [DSIII(b)] relative abundance across DSIII samples was confirmed (FIG. 13B).

Each pathogenic microbiota state (DSI-III) was characteristically dominated by a distinct bacterial family that co-associated with a relatively unique suite of lower abundance taxa (FIG. 9B). To identify taxonomic differentials characteristic of each CRS microbiota state, each was compared to healthy subjects using zero-inflated negative binomial (ZINB) regression (FIGS. 9C-9E; Tables 13-16). The identity and magnitude of depleted taxa was relatively consistent irrespective of the CRS microbiota state examined and included Streptococcus, Rothia, Haemophilus, and Lactobacillales members (ZINB p<0.05, q<0.10; FIGS. 9C-9E). The magnitude and types of taxa enriched in CRS patients differed by community state (FIG. 9B), and were most pronounced in DSIII(a) and III(b), which exhibited relatively large Corynebacterium or Staphylococcus enrichments respectively. DSI, though most compositionally similar to healthy controls, exhibited relative enrichment of Streptococcus as well as Streptococcus, Porphyromonas, Tannerella, Treponema, Bacteroides, Dialister, and Akkermansia (ZINB; p<0.05; q<0.05). DSII, dominated by Pseudomonadaceae, was also relatively enriched for Fusobacterium, Aggregatibacter, Achromobacter and Prevotella (p<0.05; q<0.05), known airway pathobionts characteristically enriched in CF and asthmatic lungs[1, 7, 39, 40]. Presumably this reflects the increased number of such patients in this sub-group, and indicates that archetypal lower airway microbiome dysbioses in CF and asthmatic patients may also be reflected in their upper airway bacterial community composition. While DSIII(a) and III(b) shared substantial taxonomic overlap, explaining their statistical grouping into a single DMM cluster, DSIII(a) was uniquely enriched for Sphingomonas (ZINB p<0.0001, q<0.0001; FIG. 9E) and DSIII(b) uniquely co-enriched for eight taxa absent in III(a) [Actinobacteria, Bifidobacterium, Haemophilus, Enterobacteriaceae, Pseudomonadaceae, Sphingomonadaceae (unclassified genus), Selenomonas and Streptophyta (ZINB p<0.05, q<0.05].

Because the majority of healthy individuals were classified into DSI, we also compared DSII, III(a), and III(b) individually to DSI. General concordance was observed between taxa enriched in DSII or III when compared to either DSI or healthy subjects (ZINB p<0.05, q<0.05; Tables 8-10). The primary discriminating genera for each DS remained consistent; differences were only observed in a select few low abundance taxa, validating the observation that the DSI microbiota was compositionally similar to that of healthy subjects. When compared to DSI, DSII remained enriched for Aggregatibacter, Achromobacter, Fusobacterium, and Prevotella, but was also relatively enriched for Pseudomonas (ZINB p=0.004 q=0.026). DSIII(a) and III(b) remained highly enriched for Corynebacterium or Staphylococcus although Cloacibacterium were uniquely co-enriched with Corynebacterium and Serratia were uniquely co-enriched with Staphylococcus when these groups were compared with DSI.

TABLE 8
DS I v. DS II

| | ZINB p | ZINB q | Fold | |
|---|---|---|---|---|
| OTU_IDs | value | value | Difference | Taxonomy |
| 274754 | 2.05E−05 | 0.0003 | 611.31 | Enterobacteriaceae; g_; s_{—} |
| 545299 | 0.0003 | 0.0029 | 450.07 | Fusobacteriaceae; g_Fusobacterium; s_{—} |
| 242070 | 0.0035 | 0.0266 | 284.62 | Pseudomonadaceae; g_Pseudomonas; s_{—} |
| 68617 | 5.96E−08 | 1.59E−06 | 230.20 | Alcaligenaceae; g_Achromobacter; s_{—} |
| 4302571 | 1.24E−125 | 5.95E−123 | 113.05 | Prevotellaceae; g_Prevotella; s_{—} |
| 114510 | 6.39E−08 | 1.62E−06 | 78.92 | Enterobacteriaceae; g_; s_{—} |
| 4432431 | 0.0058 | 0.0406 | 52.32 | Pasteurellaceae; g_Aggregatibacter; s_segnis |
| 1147942 | 7.99E−28 | 1.92E−25 | 39.48 | Pasteurellaceae; g_Aggregatibacter; s_{—} |
| 3678349 | 8.79E−05 | 0.0011 | 25.70 | Streptococcaceae; g_Streptococcus; s_anginosus |
| 4466150 | 8.82E−07 | 1.84E−05 | 24.72 | Pasteurellaceae; g_Aggregatibacter; s_segnis |
| 4426163 | 2.56E−10 | 1.03E−08 | 20.59 | Prevotellaceae; g_Prevotella; s_{—} |
| 610111 | 3.37E−20 | 4.05E−18 | 15.48 | Prevotellaceae; g_Prevotella; s_{—} |
| 928538 | 0.0013 | 0.0112 | 15.46 | Staphylococcaceae; g_Staphylococcus; s_{—} |
| 656881 | 8.31E−05 | 0.0011 | 13.37 | Enterobacteriaceae; g_; s_{—} |
| 4448731 | 0.0005 | 0.0052 | 11.14 | Fusobacteriaceae; g_Fusobacterium; s_{—} |
| 3385021 | 0.0001 | 0.0017 | 9.92 | Staphylococcaceae; g_Staphylococcus; s_{—} |
| 269901 | 0.0017 | 0.0135 | 8.71 | Pseudomonadaceae; g_; s_{—} |
| 144814 | 1.68E−06 | 3.37E−05 | 7.42 | Enterobacteriaceae |
| 91557 | 2.08E−09 | 6.66E−08 | 7.23 | Enterobacteriaceae |
| 4415943 | 0.0004 | 0.0044 | 6.93 | Fusobacteriaceae; g_Fusobacterium; s_{—} |
| 141145 | 0.0046 | 0.0337 | 4.96 | Enterobacteriaceae; g_; s_{—} |
| 996487 | 2.83E−07 | 6.80E−06 | 4.07 | Staphylococcaceae; g_Staphylococcus; s_epidermidis |
| 939252 | 3.89E−06 | 7.26E−05 | 3.98 | Staphylococcaceae; g_Staphylococcus; s_{—} |
| 122049 | 4.04E−08 | 1.14E−06 | 3.96 | Enterobacteriaceae; g_; s_{—} |
| 137056 | 2.04E−09 | 6.66E−08 | 3.70 | Planococcaceae; g_; s_{—} |
| 4312969 | 0.0044 | 0.0327 | 2.68 | Staphylococcaceae; g_Staphylococcus |
| 1076316 | 0.0009 | 0.0084 | 2.53 | Staphylococcaceae; g_Staphylococcus; s_{—} |
| New.ReferenceOTU160 | 0.0016 | 0.0130 | 1.75 | Pseudomonadaceae; g_Pseudomonas; s_{—} |
| 960695 | 0.0014 | 0.0122 | 1.14 | Planococcaceae; g_; s_{—} |
| 4466659 | 7.89E−05 | 0.0010 | 1.03 | Fusobacteriaceae; g_Fusobacterium; s_{—} |
| 4415319 | 0.0071 | 0.0485 | 0.96 | Alcaligenaceae; g_Achromobacter; s_{—} |
| 1055132 | 0.0073 | 0.0491 | 0.71 | Staphylococcaceae; g_Staphylococcus |
| 982266 | 0.0030 | 0.0231 | −0.55 | [Chromatiaceae] |
| 4322739 | 0.0003 | 0.0035 | −0.73 | Dermacoccaceae; g_Dermacoccus; s_{—} |
| 159017 | 0.0006 | 0.0056 | −0.97 | Caulobacteraceae; g_; s_{—} |
| 109060 | 0.0053 | 0.0372 | −1.70 | Comamonadaceae; g_Delftia; s_{—} |
| 809192 | 0.0001 | 0.0013 | −2.31 | Dermabacteraceae; g_Brachybacterium; s_conglomeratum |
| 4328567 | 0.0032 | 0.0243 | −2.43 | Comamonadaceae; g_Delftia; s_{—} |
| 4449609 | 2.03E−05 | 0.0003 | −3.62 | Sphingomonadaceae; g_Sphingomonas; s_{—} |
| 4323897 | 0.0028 | 0.0219 | −3.78 | Oxalobacteraceae; g_; s_{—} |
| 823916 | 0.0048 | 0.0342 | −5.49 | Moraxellaceae; g_Enhydrobacter; s_{—} |
| 142419 | 7.70E−06 | 0.0001 | −5.52 | Pseudomonadaceae |
| 4363066 | 0.0002 | 0.0023 | −5.70 | Pasteurellaceae; g_Aggregatibacter; s_{—} |
| 668514 | 0.0006 | 0.0056 | −5.99 | Enterobacteriaceae; g_; s_{—} |
| 400315 | 3.92E−06 | 7.26E−05 | −6.18 | Pseudomonadaceae; g_Pseudomonas; s |
| 1082607 | 8.36E−21 | 1.34E−18 | −9.27 | Corynebacteriaceae; g_Corynebacterium; s_{—} |
| 4331815 | 0.0011 | 0.0097 | −9.98 | Sphingomonadaceae; g_; s_{—} |
| 4344371 | 4.68E−05 | 0.0007 | −12.47 | Sphingomonadaceae; g_Sphingomonas; s_{—} |
| 4456891 | 9.54E−06 | 0.0002 | −12.93 | Pseudomonadaceae; g_Pseudomonas; s_{—} |
| 615020 | 0.0004 | 0.0037 | −13.27 | Mycoplasmataceae; g_Mycoplasma; s_{—} |
| 2685602 | 1.21E−05 | 0.0002 | −13.74 | Comamonadaceae; g_Delftia; s |
| 3384047 | 4.31E−05 | 0.0006 | −14.12 | Streptococcaceae; g_Streptococcus; s_{—} |
| 610486 | 0.0001 | 0.0017 | −21.62 | Comamonadaceae |
| 1053321 | 0.0006 | 0.0055 | −35.16 | Moraxellaceae; g_Moraxella; s_{—} |
| 1981302 | 3.44E−10 | 1.27E−08 | −37.45 | Burkholderiaceae; g_Burkholderia; s_{—} |
| 254888 | 6.48E−05 | 0.0009 | −41.40 | Comamonadaceae; g_; s_{—} |
| 1566691 | 3.32E−07 | 7.58E−06 | −47.23 | Pseudomonadaceae; g_Pseudomonas; s_{—} |
| 4416763 | 0.0003 | 0.0035 | −52.71 | Streptococcaceae; g_Streptococcus; s_{—} |
| 866280 | 4.48E−06 | 7.98E−05 | −55.89 | Micrococcaceae; g_Rothia; s_mucilaginosa |
| 494906 | 0.0002 | 0.0019 | −59.76 | [Tissierellaceae]; g_Peptoniphilus; s_{—} |
| 4458959 | 1.97E−11 | 9.45E−10 | −71.25 | Veillonellaceae; g_Veillonella; s_parvula |
| 4405869 | 3.47E−07 | 7.58E−06 | −85.44 | Fusobacteriaceae; g_Fusobacterium; s_{—} |
| 937813 | 5.73E−05 | 0.0008 | −89.12 | [Tissierellaceae]; g_Anaerococcus; s_{—} |
| 4446902 | 4.15E−11 | 1.82E−09 | −115.01 | Gemellaceae; g_; s_{—} |
| 4411138 | 9.82E−09 | 2.95E−07 | −121.47 | Micrococcaceae; g_Rothia; s_mucilaginosa |
| 495067 | 0.0017 | 0.0140 | −136.15 | Corynebacteriaceae; g_Corynebacterium; s_{—} |
| 12574 | 1.19E−14 | 8.19E−13 | −144.40 | Actinomycetaceae; g_Actinomyces; s_{—} |
| 4465561 | 2.66E−13 | 1.60E−11 | −233.97 | Prevotellaceae; g_Prevotella; s_melaninogenica |
| 4439603 | 3.85E−17 | 3.09E−15 | −234.40 | Streptococcaceae; g_Streptococcus; s_{—} |
| 4425214 | 2.39E−12 | 1.28E−10 | −298.58 | Streptococcaceae; g_Streptococcus; s_{—} |
| 4309301 | 8.28E−18 | 7.96E−16 | −837.31 | Streptococcaceae; g_Streptococcus; s_{—} |

TABLE 9
DS I v. DS IIIa

| | ZINB p | ZINB q | Fold | |
|---|---|---|---|---|
| OTU_IDs | value | value | Difference | Taxonomy |
| 1015518 | 4.51E−07 | 2.02E−05 | 2818.55 | Corynebacteriaceae; g_Corynebacterium; s_{—} |
| 1062051 | 3.33E−13 | 5.48E−11 | 694.68 | Corynebacteriaceae; g_Corynebacterium; s_{—} |
| 4154872 | 3.41E−78 | 1.68E−75 | 102.76 | [Weeksellaceae]; g_Cloacibacterium; s_{—} |
| 504674 | 0.0055 | 0.0450 | 54.20 | [Tissierellaceae]; g_Anaerococcus; s_{—} |
| 71872 | 0.0018 | 0.0180 | 35.38 | Comamonadaceae; g_Comamonas; s_{—} |
| 1116384 | 6.57E−07 | 2.50E−05 | 30.62 | Comamonadaceae; g_; s_{—} |
| 1077373 | 3.49E−08 | 2.16E−06 | 28.27 | Prevotellaceae; g_Prevotella; s_{—} |
| 3393186 | 2.13E−07 | 1.17E−05 | 20.06 | Neisseriaceae; g_; s_{—} |
| 102915 | 4.97E−09 | 4.09E−07 | 19.32 | Sphingomonadaceae; g_Sphingomonas; s_{—} |
| 207936 | 1.82E−06 | 5.62E−05 | 17.16 | [Tissierellaceae]; g_Anaerococcus; s_{—} |
| 259272 | 0.0008 | 0.0100 | 8.27 | Bradyrhizobiaceae; g_; s_{—} |
| 410908 | 6.60E−09 | 4.66E−07 | 8.05 | Corynebacteriaceae; g_Corynebacterium; s_{—} |
| 441265 | 1.85E−05 | 0.0004 | 6.09 | Corynebacteriaceae; g_Corynebacterium; s_{—} |
| 4312969 | 0.0019 | 0.0188 | 4.04 | Staphylococcaceae; g_Staphylococcus |
| 4396717 | 0.0002 | 0.0027 | 4.02 | Methylobacteriaceae; g_Methylobacterium; s_{—} |
| 802064 | 0.0025 | 0.0234 | 3.35 | Burkholderiaceae; g_Burkholderia; s_{—} |
| 4383166 | 0.0010 | 0.0113 | 3.06 | Comamonadaceae |
| 122049 | 0.0056 | 0.0450 | 2.78 | Enterobacteriaceae; g_; s_{—} |
| 1068955 | 0.0012 | 0.0125 | 1.31 | Staphylococcaceae; g_Staphylococcus; s_{—} |
| 979261 | 0.0027 | 0.0242 | 0.92 | Staphylococcaceae; g_Staphylococcus; s_aureus |
| 91557 | 0.0017 | 0.0172 | 0.83 | Enterobacteriaceae |
| 982266 | 0.0058 | 0.0461 | −0.27 | [Chromatiaceae] |
| 159017 | 0.0002 | 0.0039 | −0.72 | Caulobacteraceae; g_; s_{—} |
| 4423410 | 0.0015 | 0.0152 | −0.98 | Sphingomonadaceae; g_; s_{—} |
| New.ReferenceOTU32 | 2.45E−11 | 2.42E−09 | −1.28 | Unassigned |
| 1049188 | 0.0014 | 0.0150 | −1.48 | Corynebacteriaceae; g_Corynebacterium; s_{—} |
| 1100972 | 9.52E−12 | 1.18E−09 | −2.10 | Streptococcaceae; g_Lactococcus; s_{—} |
| 4337755 | 0.0027 | 0.0239 | −2.98 | Gemellaceae; g_; s_{—} |
| 4449609 | 3.34E−07 | 1.65E−05 | −3.49 | Sphingomonadaceae; g_Sphingomonas; s_{—} |
| 4437024 | 0.0005 | 0.0066 | −3.53 | Streptococcaceae; g_Streptococcus; s_{—} |
| 1927234 | 0.0002 | 0.0039 | −3.71 | Leptotrichiaceae; g_Leptotrichia; s_{—} |
| 511378 | 0.0031 | 0.0261 | −4.17 | Veillonellaceae; g_Megasphaera; s_{—} |
| 790466 | 4.43E−06 | 0.0001 | −4.86 | [Mogibacteriaceae]; g_Anaerovorax; s_{—} |
| 4302049 | 0.0004 | 0.0048 | −4.89 | Streptococcaceae; g_Streptococcus; s_{—} |
| 1029036 | 0.0005 | 0.0064 | −7.02 | Porphyromonadaceae; g_Porphyromonas; s_{—} |
| 611110 | 1.43E−30 | 3.52E−28 | −10.13 | Prevotellaceae; g_Prevotella; s_intermedia |
| 4424239 | 0.0006 | 0.0074 | −11.98 | Streptococcaceae; g_Streptococcus; s_{—} |
| 4431355 | 0.0003 | 0.0042 | −14.05 | Neisseriaceae; g_; s_{—} |
| 4456889 | 1.73E−05 | 0.0004 | −15.32 | Pseudomonadaceae; g_Pseudomonas; s_{—} |
| 1042479 | 0.0003 | 0.0042 | −16.17 | Prevotellaceae; g_Prevotella; s_melaninogenica |
| 4306048 | 3.08E−06 | 8.96E−05 | −16.41 | Streptococcaceae; g_Streptococcus; s_{—} |
| 4432431 | 5.09E−05 | 0.0010 | −17.94 | Pasteurellaceae; g_Aggregatibacter; s_segnis |
| 4455767 | 8.38E−05 | 0.0016 | −18.52 | Streptococcaceae; g_Streptococcus; s_{—} |
| 4296424 | 5.51E−06 | 0.0001 | −28.75 | Actinomycetaceae; g_Actinomyces; s_{—} |
| 1053321 | 0.0026 | 0.0239 | −35.13 | Moraxellaceae; g_Moraxella; s_{—} |
| 4469359 | 0.0011 | 0.0120 | −40.45 | Pasteurellaceae; g_Haemophilus; s_{—} |
| 851704 | 0.0011 | 0.0125 | −43.48 | [Tissierellaceae]; g_Parvimonas; s_{—} |
| 4319899 | 0.0003 | 0.0042 | −67.36 | Fusobacteriaceae; g_Fusobacterium; s_{—} |
| 4405869 | 0.0025 | 0.0237 | −83.25 | Fusobacteriaceae; g_Fusobacterium; s_{—} |
| 4446902 | 8.19E−06 | 0.0002 | −111.88 | Gemellaceae; g_; s_{—} |
| 12574 | 1.14E−05 | 0.0003 | −140.94 | Actinomycetaceae; g_Actinomyces; s_{—} |
| 4453501 | 6.10E−07 | 2.50E−05 | −152.38 | Veillonellaceae; g_Veillonella; s_dispar |
| 1059655 | 8.60E−07 | 3.03E−05 | −219.71 | Streptococcaceae; g_Streptococcus; s_{—} |
| 4439603 | 4.65E−05 | 0.0010 | −221.22 | Streptococcaceae; g_Streptococcus; s_{—} |
| 4465561 | 9.67E−05 | 0.0018 | −223.64 | Prevotellaceae; g_Prevotella; s_melaninogenica |
| 4323555 | 0.0003 | 0.0042 | −234.71 | Fusobacteriaceae; g_Fusobacterium; s_{—} |
| 4425214 | 1.14E−06 | 3.75E−05 | −290.55 | Streptococcaceae; g_Streptococcus; s_{—} |
| 225088 | 0.0001 | 0.0019 | −297.29 | Pseudomonadaceae; g_Pseudomonas; s_{—} |
| 4471251 | 0.0029 | 0.0248 | −306.95 | Pasteurellaceae; g_Haemophilus; s_{—} |
| 4477696 | 0.0011 | 0.0120 | −363.78 | Pasteurellaceae; g_Haemophilus |
| 22951 | 0.0029 | 0.0249 | −373.26 | Prevotellaceae; g_Prevotella; s_{—} |
| 4309301 | 0.0004 | 0.0048 | −796.88 | Streptococcaceae; g_Streptococcus; s_{—} |

TABLE 10
DS I v. DS IIIa

| | ZINB P | ZINB q | Fold | |
|---|---|---|---|---|
| OTU_IDs | value | value | Difference | Taxonomy |
| 4345285 | 6.02E−05 | 0.0006 | 2119.55 | Staphylococcaceae; g_Staphylococcus; s_{—} |
| 4416113 | 7.57E−09 | 2.84E−07 | 433.99 | Enterobacteriaceae; g_Serratia; s_marcescens |
| 254888 | 0.0073 | 0.0415 | 139.22 | Comamonadaceae; g_; s_{—} |
| 553611 | 7.83E−14 | 5.89E−12 | 125.39 | Bifidobacteriaceae; g_Bifidobacterium; s_{—} |
| 4428313 | 9.23E−07 | 2.17E−05 | 113.05 | Lactobacillaceae; g_Lactobacillus; s_{—} |
| 4361528 | 0.0010 | 0.0074 | 110.53 | Moraxellaceae; g_Acinetobacter; s_{—} |
| 4386317 | 2.49E−23 | 3.28E−21 | 90.41 | ; g_; s_{—} |
| 4319936 | 3.29E−07 | 8.25E−06 | 65.67 | ; g_; s_{—} |
| 274365 | 0.0001 | 0.0010 | 48.85 | Enterobacteriaceae; g_; s_{—} |
| 928538 | 1.15E−05 | 0.0002 | 41.72 | Staphylococcaceae; g_Staphylococcus; s_{—} |
| 656881 | 9.47E−07 | 2.17E−05 | 38.47 | Enterobacteriaceae; g_; s_{—} |
| 4415684 | 0.0063 | 0.0359 | 30.22 | Micrococcaceae; g_Kocuria; s_rhizophila |
| 4396025 | 1.07E−49 | 2.82E−47 | 24.46 | Sphingomonadaceae; g_; s_{—} |
| 1040220 | 0.0024 | 0.0149 | 20.36 | Staphylococcaceae; g_Staphylococcus |
| 825808 | 7.08E−06 | 0.0001 | 20.24 | Bifidobacteriaceae; g_Bifidobacterium; s_{—} |
| 584109 | 4.38E−91 | 2.30E−88 | 19.84 | Streptococcaceae; g_Streptococcus; s_{—} |
| 240252 | 0.0012 | 0.0087 | 18.40 | Acetobacteraceae; g_Acidocella; s_{—} |
| 4396717 | 7.06E−09 | 2.84E−07 | 18.03 | Methylobacteriaceae; g_Methylobacterium; s_{—} |
| 526682 | 0.0032 | 0.0191 | 14.41 | Actinomycetaceae; g_Actinomyces; s_{—} |
| 3385021 | 0.0020 | 0.0130 | 12.77 | Staphylococcaceae; g_Staphylococcus; s_{—} |
| New.ReferenceOTU66 | 9.21E−12 | 6.05E−10 | 11.71 | Actinomycetaceae; g_Actinomyces; s_{—} |
| 4349519 | 9.49E−28 | 1.66E−25 | 11.57 | [Tissierellaceae]; g_Anaerococcus; s_{—} |
| 244657 | 2.72E−05 | 0.0003 | 11.53 | Bradyrhizobiaceae |
| 119663 | 1.02E−05 | 0.0001 | 11.22 | Alcaligenaceae |
| 1010113 | 2.27E−09 | 1.19E−07 | 10.78 | Enterobacteriaceae; g_; s_{—} |
| 139289 | 1.53E−08 | 5.03E−07 | 10.18 | Pseudomonadaceae; g_Pseudomonas; s_{—} |
| 326163 | 2.15E−06 | 4.34E−05 | 10.12 | Thermaceae; g_Meiothermus; s_{—} |
| 509021 | 0.0002 | 0.0021 | 9.51 | Sphingomonadaceae |
| 114510 | 0.0014 | 0.0096 | 8.50 | Enterobacteriaceae; g_; s_{—} |
| 4423410 | 5.22E−20 | 5.49E−18 | 8.40 | Sphingomonadaceae; g_; s_{—} |
| 809192 | 2.99E−16 | 2.62E−14 | 8.30 | Dermabacteraceae; g_Brachybacterium; s_conglomeratum |
| 996487 | 0.0001 | 0.0011 | 8.05 | Staphylococcaceae; g_Staphylococcus; s_epidermidis |
| 1076316 | 5.98E−06 | 9.05E−05 | 7.67 | Staphylococcaceae; g_Staphylococcus; s_{—} |
| 137056 | 1.86E−05 | 0.0002 | 7.40 | Planococcaceae; g_; s_{—} |
| 219151 | 8.22E−06 | 0.0001 | 7.12 | Moraxellaceae; g_Acinetobacter; s_{—} |
| 939252 | 3.02E−07 | 7.95E−06 | 6.61 | Staphylococcaceae; g_Staphylococcus; s_{—} |
| 4421747 | 0.0022 | 0.0138 | 6.49 | Burkholderiaceae; g_Burkholderia; s_{—} |
| 258707 | 0.0048 | 0.0280 | 6.28 | Methylobacteriaceae; g_Methylobacterium; s_{—} |
| 4312969 | 8.66E−05 | 0.0009 | 5.78 | Staphylococcaceae; g_Staphylococcus |
| 4459414 | 2.02E−05 | 0.0002 | 5.28 | Veillonellaceae; g_Selenomonas; s_noxia |
| 2468881 | 0.0001 | 0.0010 | 5.24 | Pseudomonadaceae; g_Pseudomonas; s_{—} |
| 4374322 | 2.69E−09 | 1.28E−07 | 4.79 | Moraxellaceae; g_Acinetobacter; s_{—} |
| 141145 | 0.0016 | 0.0108 | 4.70 | Enterobacteriaceae; g_; s_{—} |
| 1116384 | 0.0013 | 0.0087 | 3.95 | Comamonadaceae; g_; s_{—} |
| 4473295 | 0.0021 | 0.0136 | 3.63 | Fusobacteriaceae; g_Fusobacterium; s_{—} |
| 4383166 | 9.02E−05 | 0.0009 | 3.59 | Comamonadaceae |
| 1068955 | 5.85E−06 | 9.05E−05 | 3.28 | Staphylococcaceae; g_Staphylococcus; s_{—} |
| 122049 | 0.0079 | 0.0443 | 3.15 | Enterobacteriaceae; g_; s_{—} |
| 1111636 | 0.0003 | 0.0022 | 3.09 | Comamonadaceae |
| 268968 | 2.70E−08 | 8.36E−07 | 3.09 | Alcaligenaceae; g_Achromobacter; s_{—} |
| 678813 | 1.35E−05 | 0.0002 | 3.07 | Xanthomonadaceae; g_; s_{—} |
| 368134 | 0.0083 | 0.0459 | 3.07 | Planococcaceae; g_; s_{—} |
| 1058950 | 0.0032 | 0.0191 | 2.71 | Planococcaceae; g_; s_{—} |
| 960695 | 1.46E−05 | 0.0002 | 2.40 | Planococcaceae; g_; s_{—} |
| 544841 | 1.98E−06 | 4.33E−05 | 1.54 | Sphingomonadaceae; g_; s_{—} |
| 4306773 | 0.0013 | 0.0089 | 1.50 | Leptotrichiaceae; g_Leptotrichia; s_{—} |
| 979261 | 0.0006 | 0.0044 | 1.19 | Staphylococcaceae; g_Staphylococcus; s_aureus |
| 984924 | 0.0050 | 0.0290 | 0.99 | Staphylococcaceae; g_Staphylococcus |
| 4322998 | 0.0020 | 0.0131 | −0.95 | Fusobacteriaceae; g_Fusobacterium; s_{—} |
| 4383953 | 6.02E−06 | 9.05E−05 | −1.31 | Clostridiaceae; g_; s_{—} |
| 4428042 | 0.0006 | 0.0046 | −2.20 | Streptococcaceae; g_Streptococcus; s_{—} |
| 3678349 | 0.0016 | 0.0108 | −2.68 | Streptococcaceae; g_Streptococcus; s_anginosus |
| 4460509 | 1.13E−08 | 3.97E−07 | −2.86 | Dethiosulfovibrionaceae; g_TG5; s_{—} |
| 4337755 | 0.0001 | 0.0010 | −2.92 | Gemellaceae; g_; s_{—} |
| 4404577 | 0.0007 | 0.0053 | −3.06 | Peptostreptococcaceae; g_Peptostreptococcus |
| 1049188 | 2.07E−05 | 0.0002 | −3.83 | Corynebacteriaceae; g_Corynebacterium; s_{—} |
| 109413 | 0.0005 | 0.0042 | −3.99 | Pasteurellaceae; g_Haemophilus; s_parainfluenzae |
| 1079708 | 4.78E−06 | 8.11E−05 | −4.13 | Streptococcaceae; g_Streptococcus; s_{—} |
| 4302049 | 0.0030 | 0.0181 | −4.46 | Streptococcaceae; g_Streptococcus; s_{—} |
| 526804 | 0.0004 | 0.0029 | −5.37 | Streptococcaceae; g_Streptococcus; s_{—} |
| 513646 | 0.0032 | 0.0191 | −5.83 | Streptococcaceae; g_Streptococcus; s_{—} |
| 1082607 | 9.67E−05 | 0.0009 | −7.19 | Corynebacteriaceae; g_Corynebacterium; s_{—} |
| 4307230 | 0.0004 | 0.0031 | −8.45 | Dethiosulfovibrionaceae; g_TG5; s_{—} |
| 4430826 | 6.23E−05 | 0.0007 | −10.22 | Leptotrichiaceae; g_Leptotrichia; s_{—} |
| 4424239 | 2.15E−05 | 0.0002 | −11.87 | Streptococcaceae; g_Streptococcus; s_{—} |
| 3384047 | 4.67E−06 | 8.11E−05 | −14.09 | Streptococcaceae; g_Streptococcus; s_{—} |
| 4306048 | 3.05E−09 | 1.34E−07 | −16.31 | Streptococcaceae; g_Streptococcus; s_{—} |
| 4340162 | 0.0002 | 0.0015 | −16.67 | [Paraprevotellaceae]; g_[Prevotella]; s_{—} |
| 4455767 | 0.0009 | 0.0065 | −17.67 | Streptococcaceae; g_Streptococcus; s_{—} |
| 4318672 | 0.0002 | 0.0016 | −21.13 | Neisseriaceae; g_Neisseria; s_{—} |
| 4326219 | 3.70E−06 | 6.96E−05 | −21.74 | Campylobacteraceae; g_Campylobacter; s_{—} |
| 3801267 | 6.68E−05 | 0.0007 | −30.02 | Veillonellaceae; g_Veillonella; s_parvula |
| 4294457 | 3.91E−06 | 7.10E−05 | −39.96 | Micrococcaceae; g_Rothia; s_mucilaginosa |
| 4307391 | 2.23E−07 | 6.17E−06 | −45.49 | Prevotellaceae; g_Prevotella; s_melaninogenica |
| 2613485 | 5.54E−06 | 9.05E−05 | −53.83 | Porphyromonadaceae; g_Porphyromonas; s_{—} |
| 4387092 | 0.0003 | 0.0022 | −54.78 | Fusobacteriaceae; g_Fusobacterium; s_{—} |
| 4319899 | 3.71E−06 | 6.96E−05 | −67.30 | Fusobacteriaceae; g_Fusobacterium; s_{—} |
| 4405869 | 1.69E−09 | 9.88E−08 | −85.50 | Fusobacteriaceae; g_Fusobacterium; s_{—} |
| 4446902 | 0.0006 | 0.0047 | −112.66 | Gemellaceae; g_; s_{—} |
| 271159 | 1.28E−07 | 3.74E−06 | −113.61 | Lactobacillales |
| 12574 | 0.0003 | 0.0025 | −137.64 | Actinomycetaceae; g_Actinomyces; s_{—} |
| 4453501 | 1.93E−05 | 0.0002 | −150.33 | Veillonellaceae; g_Veillonella; s_dispar |
| 630141 | 0.0007 | 0.0051 | −157.89 | Staphylococcaceae; g_Staphylococcus; s_{—} |
| 4396235 | 1.06E−05 | 0.0001 | −483.39 | Neisseriaceae; g_Neisseria; s_subflava |
| 4309301 | 2.11E−06 | 4.34E−05 | −786.49 | Streptococcaceae; g_Streptococcus; s_{—} |

Predicted Functional Capacity Discriminates Sinus Bacterial Dirichlet States.

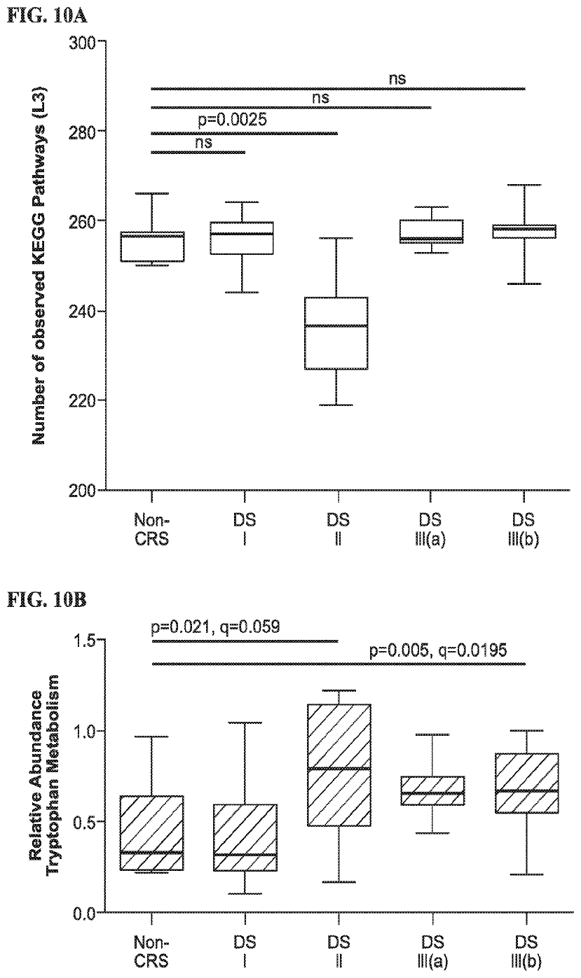

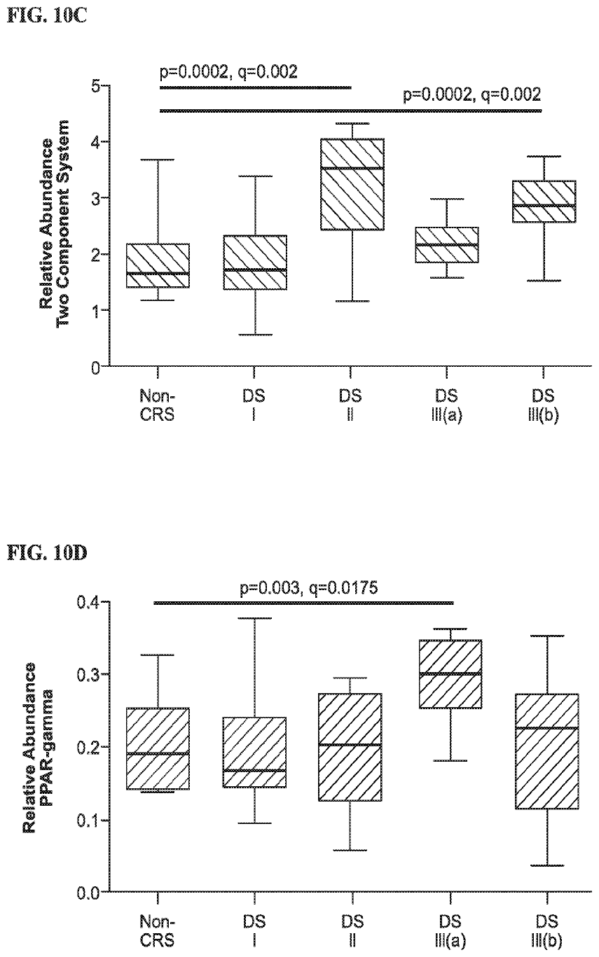

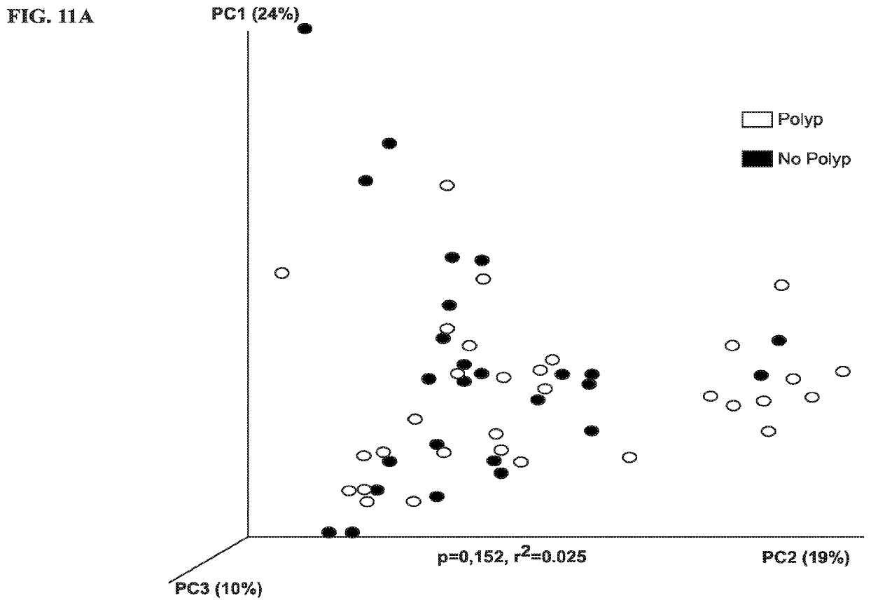

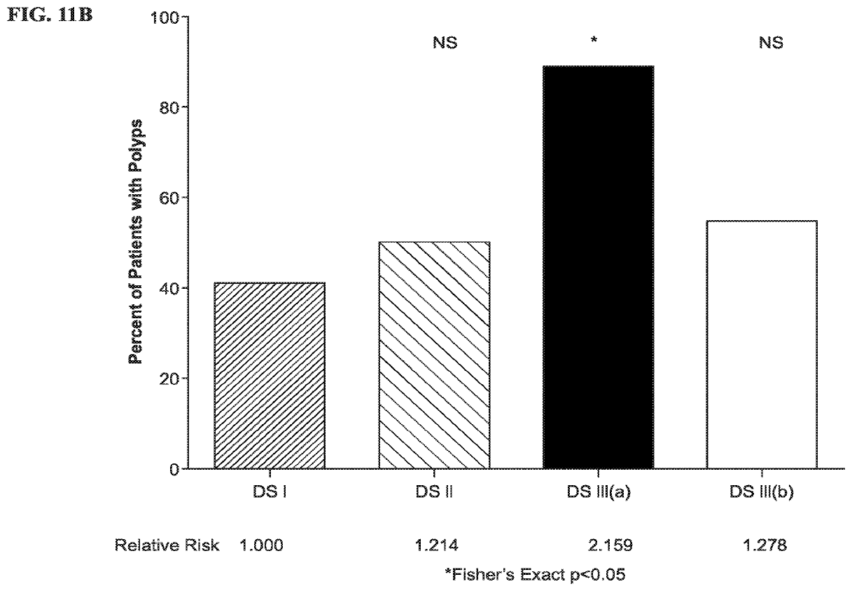

Bacterial metagenomes were predicted in silico for each patient using Phylogenetic Investigation of Communities by Reconstruction of Unobserved States (PICRUSt v. 1.0.0 [41]), an algorithm which uses biomarker gene sequence data i.e. 16S rRNA to infer functional capacity using representative sequenced and predicted ancestral genomes. Associated nearest Sequenced Taxon Index (NTSI) scores which indicate the degree of relatedness between OTUs and sequenced genomes used for PICRUST predictions are detailed in Table 18. Each microbiota state was predicted to encode a distinct metagenome (Bray-Curtis PERMANOVA p=0.001, 23.2% variation explained) and a total of 196 KEGG pathways differentiated pathogenic microbiota states compared control patients (Three-model test; p<0.05 q<0.10). Only 21 KEGG pathways discriminated patients with distinct lower airway co-morbidities (Asthma+/−, CF; Kruskal-Wallis p<0.05, q<0.10, Table 11), indicating substantial overlap in microbial function across distinct airway diseases. Compared to healthy microbiota, the DSII group were the least functionally diverse (permutational t-test, FDR p<0.05, FIG. 10A) and depleted of 67 KEGG pathways for lipid, carbohydrate, terpenoid, and xenobiotic metabolism. DSII and III(b) were both significantly enriched in bacterial virulence pathways, including two-component response systems, and for fatty acid and tryptophan metabolism pathways associated with inflammation (Negative Binomial p<0.05, q<0.05; FIGS. 10B-10C, Table 12), when compared to healthy controls. DSI patients were depleted of polyketide and folate biosynthesis, and enriched for the a pathway responsible for ansamycin biosynthesis, a microbial secondary metabolite with broad range antimicrobial activity (Poisson p<0.0001, q<0.0001; Table 12A)[42]. Corynebacterium-dominated DSIII(a) was characterized by both peroxisome proliferator-activated receptor-γ [(PPAR-γ) Negative Binomial p=0.003, q=0.018; FIG. 3E0, Table 12C] and the retinoic acid-inducible gene-1 (RIG-I) signaling pathways (Negative Binomial p=0.015, q=0.062; Table 12C), both of which are increased in eosinophilic polyp tissue in CRS patients [43, 44].

TABLE 11

| KEGG Pathway; CRS, Asthma-CRS, CF-CRS, | Test- | | |
|---|---|---|---|
| non-CRS | Statistic | P | FDR_P |
| Other glycan degradation | 16.269 | 0.001 | 0.103 |
| Various types of N-glycan biosynthesis | 15.725 | 0.001 | 0.103 |
| D-Glutamine and D-glutamate metabolism | 15.280 | 0.002 | 0.103 |
| Betalain biosynthesis | 14.046 | 0.003 | 0.103 |
| Vasopressin-regulated water reabsorption | 13.874 | 0.003 | 0.103 |
| Insulin signaling pathway | 13.874 | 0.003 | 0.103 |
| Lysosome | 13.690 | 0.003 | 0.103 |
| Melanogenesis | 13.460 | 0.004 | 0.103 |
| Glycosphingolipid biosynthesis - lacto | 13.355 | 0.004 | 0.103 |
| and neolacto series | | | |
| Carbohydrate digestion and absorption | 12.703 | 0.005 | 0.103 |
| Sphingolipid metabolism | 12.694 | 0.005 | 0.103 |
| Glycosphingolipid biosynthesis - globo | 12.498 | 0.006 | 0.103 |
| series | | | |
| Arachidonic acid metabolism | 12.454 | 0.006 | 0.103 |
| Glycosphingolipid biosynthesis - ganglio | 12.385 | 0.006 | 0.103 |
| series | | | |
| Nucleotide metabolism | 12.292 | 0.006 | 0.103 |
| Xylene degradation | 12.191 | 0.007 | 0.103 |
| Cell cycle - Caulobacter | 12.177 | 0.007 | 0.103 |
| Electron transfer carriers | 12.067 | 0.007 | 0.103 |
| Type I diabetes mellitus | 12.064 | 0.007 | 0.103 |
| Glycosaminoglycan degradation | 11.976 | 0.007 | 0.103 |
| Nucleotide excision repair | 11.947 | 0.008 | 0.103 |

TABLE 12A

| KEGG Pathway; DS I v. | Test- | | | Fold |
|---|---|---|---|---|
| non-CRS | Statistic | p value | q value | Difference |
| Biosynthesis of ansamycins | 0.902 | <0.0001 | <0.0001 | 177.259 |
| Vibrio cholerae infection | 7.115 | <0.0001 | <0.0001 | −48.559 |
| Biosynthesis of type II | 8.145 | <0.0001 | <0.0001 | −140.794 |
| polyketide products | | | | |
| DNA repair and recombination | 1.003 | 0.063 | 0.075 | −210.000 |
| proteins | | | | |
| Alzheimer's disease | 1.139 | <0.0001 | <0.0001 | −312.912 |
| Folate biosynthesis | 1.043 | <0.0001 | <0.0001 | −596.441 |

TABLE 12B

| | Test- | | | Fold |
|---|---|---|---|---|
| KEGG Pathway; DII v. non-CRS | Statistic | p value | q value | Difference |
| Two-component system | 0.593 | 0.0002 | 0.002 | 26656.143 |
| Bacterial motility proteins | 0.446 | 0.045 | 0.099 | 22226.071 |
| Secretion system | 0.787 | 0.024 | 0.067 | 11887.614 |
| Other ion-coupled transporters | 0.773 | 0.002 | 0.010 | 10485.914 |
| Flagellar assembly | 0.344 | 0.014 | 0.045 | 9471.471 |
| Bacterial chemotaxis | 0.416 | 0.026 | 0.069 | 8941.343 |
| Valine, leucine and isoleucine degradation | 0.641 | 0.037 | 0.087 | 7554.614 |
| Function unknown | 0.861 | 0.035 | 0.084 | 7455.586 |
| Pores ion channels | 0.688 | 0.021 | 0.059 | 6450.543 |
| Tryptophan metabolism | 0.605 | 0.021 | 0.059 | 5977.957 |
| Arginine and proline metabolism | 0.815 | 0.034 | 0.083 | 5954.214 |
| Membrane and intracellular structural molecules | 0.753 | 0.039 | 0.091 | 5587.429 |
| Fatty acid metabolism | 0.687 | 0.043 | 0.098 | 5373.371 |
| Propanoate metabolism | 0.782 | 0.043 | 0.098 | 5179.286 |
| Porphyrin and chlorophyll metabolism | 0.797 | 0.028 | 0.071 | 4818.071 |
| Lysine degradation | 0.630 | 0.015 | 0.046 | 4521.486 |
| Limonene and pinene degradation | 0.549 | 0.010 | 0.034 | 4142.129 |
| beta-Alanine metabolism | 0.641 | 0.026 | 0.069 | 4141.643 |
| Glyoxylate and dicarboxylate metabolism | 0.763 | 0.036 | 0.085 | 3975.214 |
| Protein kinases | 0.724 | 0.035 | 0.084 | 3647.000 |
| Biosynthesis of unsaturated fatty acids | 0.569 | 0.001 | 0.008 | 3490.229 |
| Pertussis | 0.279 | 0.010 | 0.033 | 2888.500 |
| Lipopolysaccharide biosynthesis | 0.746 | <0.0001 | <0.0001 | 2815.443 |
| Nitrogen metabolism | 0.876 | 0.007 | 0.025 | 2743.957 |
| Tyrosine metabolism | 0.811 | 0.030 | 0.076 | 2458.671 |
| Sulfur relay system | 0.843 | 0.034 | 0.082 | 1658.086 |
| Other transporters | 0.793 | 0.029 | 0.073 | 1594.614 |
| Metabolism of cofactors and vitamins | 0.758 | 0.001 | 0.005 | 1533.614 |
| Atrazine degradation | 0.377 | 0.018 | 0.053 | 1266.214 |
| Amino acid metabolism | 0.761 | 0.026 | 0.069 | 1248.429 |
| Arachidonic acid metabolism | 0.564 | 0.001 | 0.005 | 1107.300 |
| Vibrio cholerae pathogenic cycle | 0.684 | 0.007 | 0.026 | 1043.471 |
| beta-Lactam resistance | 0.415 | 0.012 | 0.039 | 846.514 |
| Glycan biosynthesis and metabolism | 0.644 | <0.0001 | <0.0001 | 805.300 |
| Ethylbenzene degradation | 0.589 | 0.019 | 0.055 | 803.500 |
| Cellular antigens | 0.699 | 0.044 | 0.098 | 582.357 |
| Transcription related proteins | 0.350 | 0.015 | 0.046 | 374.429 |
| Cardiac muscle contraction | 0.796 | <0.0001 | <0.0001 | 193.586 |
| Parkinson's disease | 0.897 | <0.0001 | <0.0001 | 99.971 |
| Melanogenesis | 21.000 | 0.020 | 0.058 | −1.429 |
| Betalain biosynthesis | 23.800 | 0.014 | 0.045 | −1.629 |
| Cytochrome P450 | 29.400 | 0.004 | 0.017 | −6.086 |
| Endocytosis | 97.300 | 0.002 | 0.010 | −13.757 |
| GnRH signaling pathway | 67.200 | 0.0002 | 0.002 | −14.186 |
| Fc gamma R-mediated phagocytosis | 112.000 | 0.001 | 0.004 | −15.857 |
| Bile secretion | 116.900 | <0.0001 | <0.0001 | −16.557 |
| Various types of N-glycan biosynthesis | 35.933 | <0.0001 | 0.001 | −22.457 |
| Caffeine metabolism | 25.900 | 0.0004 | 0.003 | −24.900 |
| Germination | 7.382 | 0.022 | 0.062 | −30.086 |
| Steroid biosynthesis | 11.310 | 0.0003 | 0.002 | −37.557 |
| Flavone and flavonol biosynthesis | 30.200 | <0.0001 | <0.0001 | −102.200 |
| Basal transcription factors | 34.300 | <0.0001 | <0.0001 | −128.443 |
| Type II diabetes mellitus | 1.201 | 0.003 | 0.014 | −224.514 |
| Apoptosis | 12.751 | 0.001 | 0.004 | −389.457 |
| Zeatin biosynthesis | 1.504 | 0.006 | 0.021 | −462.757 |
| Biosynthesis of ansamycins | 1.466 | 0.026 | 0.069 | −520.443 |
| Type I diabetes mellitus | 1.534 | 0.007 | 0.025 | −524.029 |
| Primary immunodeficiency | 1.559 | <0.0001 | <0.0001 | −642.857 |
| Phosphatidylinositol signaling system | 1.281 | 0.005 | 0.018 | −725.829 |
| Vitamin B6 metabolism | 1.184 | <0.0001 | <0.0001 | −807.071 |
| Xylene degradation | 7.310 | 0.0004 | 0.003 | −1006.829 |
| D-Glutamine and D-glutamate metabolism | 1.332 | 0.011 | 0.037 | −1077.986 |
| Dioxin degradation | 2.505 | 0.0003 | 0.002 | −1126.743 |
| Sphingolipid metabolism | 2.374 | 0.033 | 0.081 | −1182.329 |
| Tuberculosis | 1.463 | <0.0001 | <0.0001 | −1267.500 |
| Cytoskeleton proteins | 1.360 | 0.003 | 0.014 | −1451.743 |
| Nicotinate and nicotinamide metabolism | 1.158 | 0.025 | 0.068 | −1557.800 |
| RNA polymerase | 1.488 | 0.003 | 0.014 | −1699.557 |
| Drug metabolism - other enzymes | 1.404 | 0.001 | 0.008 | −2248.686 |
| Prenyltransferases | 1.342 | 0.018 | 0.054 | −2476.229 |
| Streptomycin biosynthesis | 1.512 | 0.001 | 0.004 | −2494.243 |
| Other glycan degradation | 3.993 | 0.014 | 0.045 | −2507.229 |
| Alanine, aspartate and glutamate metabolism | 1.120 | 0.0003 | 0.002 | −2578.200 |
| Methane metabolism | 1.122 | 0.029 | 0.073 | −2763.314 |
| Photosynthesis proteins | 1.414 | 0.008 | 0.027 | −2939.214 |
| Pantothenate and CoA biosynthesis | 1.225 | 0.006 | 0.021 | −2978.400 |
| Photosynthesis | 1.450 | 0.002 | 0.011 | −3005.443 |
| Pentose phosphate pathway | 1.193 | 0.002 | 0.010 | −3140.686 |
| Lysine biosynthesis | 1.240 | <0.0001 | <0.0001 | −3335.657 |
| Base excision repair | 1.338 | <0.0001 | 0.001 | −3395.157 |
| One carbon pool by folate | 1.263 | 0.002 | 0.011 | −3464.086 |
| Nucleotide excision repair | 1.503 | <0.0001 | <0.0001 | −3531.014 |
| Translation factors | 1.329 | 0.002 | 0.010 | −3619.586 |
| Terpenoid backbone biosynthesis | 1.296 | 0.008 | 0.027 | −3710.171 |
| Cell cycle - Caulobacter | 1.381 | 0.0003 | 0.002 | −3786.614 |
| Phenylalanine, tyrosine and tryptophan biosynthesis | 1.218 | 0.0004 | 0.003 | −3823.929 |
| Translation proteins | 1.182 | 0.028 | 0.071 | −3886.000 |
| Valine, leucine and isoleucine biosynthesis | 1.259 | 0.014 | 0.045 | −4277.486 |
| Fructose and mannose metabolism | 1.330 | 0.002 | 0.011 | −4558.657 |
| Starch and sucrose metabolism | 1.385 | 0.001 | 0.007 | −5010.886 |
| DNA replication | 1.379 | 0.004 | 0.017 | −5133.343 |
| Mismatch repair | 1.326 | 0.012 | 0.039 | −5273.243 |
| Peptidoglycan biosynthesis | 1.303 | 0.015 | 0.046 | −5490.286 |
| Protein export | 1.434 | 0.003 | 0.014 | −5519.986 |
| Peptidases | 1.143 | 0.043 | 0.098 | −5621.571 |
| Galactose metabolism | 1.801 | 0.003 | 0.014 | −6532.129 |
| Homologous recombination | 1.358 | 0.005 | 0.019 | −6838.129 |
| Ribosome Biogenesis | 1.197 | 0.017 | 0.050 | −6901.043 |
| Amino acid related enzymes | 1.224 | <0.0001 | 0.001 | −7130.800 |
| DNA replication proteins | 1.336 | 0.004 | 0.017 | −7874.714 |
| Amino sugar and nucleotide sugar metabolism | 1.297 | 0.003 | 0.014 | −7916.729 |
| Aminoacyl-tRNA biosynthesis | 1.463 | 0.0003 | 0.002 | −10834.329 |
| Pyrimidine metabolism | 1.415 | 0.001 | 0.008 | −14619.400 |
| Purine metabolism | 1.285 | 0.001 | 0.003 | −14627.371 |
| DNA repair and recombination proteins | 1.289 | 0.001 | 0.008 | −18016.643 |
| Ribosome | 1.463 | 0.002 | 0.011 | −21194.914 |

TABLE 12C

| | Test- | | | Fold |
|---|---|---|---|---|
| KEGG Pathway; DS IIIa v. non-CRS | Statistic | p value | q value | Difference |
| Other ion-coupled transporters | 0.748 | 0.001 | 0.006 | 12036.478 |
| ABC transporters | 0.932 | 0.019 | 0.074 | 6601.422 |
| Histidine metabolism | 0.701 | <0.0001 | <0.0001 | 5058.233 |
| Tryptophan metabolism | 0.663 | 0.022 | 0.082 | 4649.822 |
| Porphyrin and chlorophyll metabolism | 0.804 | 0.009 | 0.043 | 4609.444 |
| Butanoate metabolism | 0.835 | 0.010 | 0.045 | 4019.600 |
| Limonene and pinene degradation | 0.586 | 0.020 | 0.075 | 3565.756 |
| Ubiquinone and other terpenoid-quinone biosynthesis | 0.703 | <0.0001 | <0.0001 | 3426.178 |
| Glycine, serine and threonine metabolism | 0.887 | <0.0001 | <0.0001 | 2979.222 |
| Alanine, aspartate and glutamate metabolism | 0.892 | 0.003 | 0.017 | 2926.189 |
| Phenylalanine metabolism | 0.642 | 0.017 | 0.070 | 2887.167 |
| Sulfur relay system | 0.784 | <0.0001 | 0.0001 | 2446.800 |
| Ascorbate and aldarate metabolism | 0.598 | <0.0001 | 0.001 | 2157.789 |
| Peroxisome | 0.750 | <0.0001 | 0.001 | 2018.667 |
| PPAR signaling pathway | 0.694 | 0.003 | 0.018 | 1832.922 |
| Sulfur metabolism | 0.819 | 0.0001 | 0.001 | 1813.056 |
| Riboflavin metabolism | 0.832 | 0.003 | 0.015 | 1466.822 |
| Tyrosine metabolism | 0.878 | 0.019 | 0.074 | 1456.544 |
| Adipocytokine signaling pathway | 0.694 | 0.011 | 0.051 | 1081.178 |
| Toluene degradation | 0.813 | 0.0003 | 0.003 | 945.800 |
| Ethylbenzene degradation | 0.551 | <0.0001 | 0.0003 | 941.167 |
| Proximal tubule bicarbonate reclamation | 0.474 | <0.0001 | 0.001 | 809.067 |
| Ubiquitin system | 0.526 | 0.001 | 0.005 | 723.300 |
| Carotenoid biosynthesis | 0.497 | 0.019 | 0.075 | 648.922 |
| Novobiocin biosynthesis | 0.833 | 0.002 | 0.011 | 576.156 |
| Phosphonate and phosphinate metabolism | 0.754 | 0.005 | 0.027 | 567.211 |
| Amyotrophic lateral sclerosis (ALS) | 0.606 | 0.006 | 0.028 | 532.844 |
| Basal transcription factors | 0.201 | 0.0002 | 0.002 | 526.367 |
| Chagas disease (American trypanosomiasis) | 0.452 | 0.0001 | 0.002 | 441.900 |
| Meiosis - yeast | 0.528 | 0.003 | 0.018 | 435.300 |
| African trypanosomiasis | 0.490 | 0.002 | 0.011 | 427.744 |
| Proteasome | 0.705 | 0.020 | 0.075 | 413.822 |
| alpha-Linolenic acid metabolism | 0.520 | 0.014 | 0.059 | 402.567 |
| Lipoic acid metabolism | 0.854 | 0.025 | 0.092 | 333.400 |
| Renal cell carcinoma | 0.705 | <0.0001 | 0.0004 | 313.722 |
| RIG-I-like receptor signaling pathway | 0.410 | 0.015 | 0.062 | 269.478 |
| N-Glycan biosynthesis | 0.695 | 0.029 | 0.099 | 254.311 |
| Steroid biosynthesis | 0.351 | 0.029 | 0.098 | 76.244 |
| Betalain biosynthesis | 0.097 | 0.0005 | 0.004 | 15.744 |
| Melanogenesis | 0.099 | 0.001 | 0.006 | 13.611 |
| Glycosphingolipid biosynthesis - lacto and neolacto series | 0.087 | <0.0001 | <0.0001 | 3.700 |
| Fatty acid elongation in mitochondria | 9.477 | <0.0001 | 0.000 | −0.844 |
| Various types of N-glycan biosynthesis | 25.988 | 0.001 | 0.009 | −22.211 |
| Bacterial invasion of epithelial cells | 7.217 | <0.0001 | <0.0001 | −73.911 |
| Flavone and flavonol biosynthesis | 14.199 | <0.0001 | 0.001 | −98.256 |
| Systemic lupus erythematosus | 6.166 | 0.001 | 0.006 | −126.844 |
| Biosynthesis of type II polyketide products | 6.449 | 0.019 | 0.075 | −135.611 |
| Type II diabetes mellitus | 1.122 | 0.024 | 0.089 | −145.689 |
| Protein digestion and absorption | 3.657 | 0.002 | 0.011 | −214.911 |
| NOD-like receptor signaling pathway | 1.797 | 0.0003 | 0.002 | −222.222 |
| Zeatin biosynthesis | 1.211 | 0.026 | 0.094 | −240.233 |
| Apoptosis | 2.432 | 0.006 | 0.030 | −248.822 |
| Ion channels | 1.626 | 0.005 | 0.025 | −565.578 |
| Glycosphingolipid biosynthesis - ganglio series | 4.224 | <0.0001 | 0.0002 | −598.922 |
| Cyanoamino acid metabolism | 1.122 | 0.027 | 0.096 | −616.089 |
| Xylene degradation | 2.140 | 0.0003 | 0.003 | −621.400 |
| Epithelial cell signaling in Helicobacter pylori infection | 1.709 | 0.008 | 0.038 | −621.722 |
| MAPK signaling pathway - yeast | 2.389 | <0.0001 | <0.0001 | −644.300 |
| Dioxin degradation | 1.814 | 0.002 | 0.015 | −841.822 |
| Glycosaminoglycan degradation | 4.826 | <0.0001 | <0.0001 | −903.878 |
| Glycosphingolipid biosynthesis - globo series | 3.587 | 0.0003 | 0.003 | −926.589 |
| Lysosome | 3.905 | <0.0001 | 0.0004 | −1045.989 |
| Bacterial toxins | 1.448 | <0.0001 | 0.001 | −1103.411 |
| Sphingolipid metabolism | 2.516 | <0.0001 | 0.0003 | −1230.789 |
| Restriction enzyme | 1.493 | 0.014 | 0.060 | −1321.111 |
| Cell motility and secretion | 1.456 | 0.002 | 0.012 | −1656.989 |
| Lipid biosynthesis proteins | 1.103 | 0.009 | 0.044 | −1751.544 |
| Tetracycline biosynthesis | 1.739 | 0.0002 | 0.002 | −1761.867 |
| RNA transport | 2.345 | <0.0001 | <0.0001 | −1849.889 |
| Cell cycle - Caulobacter | 1.156 | 0.005 | 0.028 | −1853.733 |
| Glycerophospholipid metabolism | 1.145 | 0.0001 | 0.001 | −1904.411 |
| Other glycan degradation | 3.850 | 0.0001 | 0.001 | −2476.022 |
| Membrane and intracellular structural molecules | 1.275 | 0.028 | 0.098 | −3668.722 |
| Peptidoglycan biosynthesis | 1.184 | 0.018 | 0.074 | −3675.000 |
| Lipopolysaccharide biosynthesis | 1.873 | 0.006 | 0.030 | −3856.311 |
| Chaperones and folding catalysts | 1.176 | <0.0001 | 0.0002 | −4174.611 |
| Translation proteins | 1.204 | 0.0002 | 0.002 | −4278.556 |
| Fatty acid biosynthesis | 1.444 | <0.0001 | <0.0001 | −4418.856 |
| DNA replication proteins | 1.188 | 0.010 | 0.044 | −4956.611 |
| Lipopolysaccharide biosynthesis proteins | 1.780 | 0.004 | 0.020 | −5051.678 |
| Chromosome | 1.192 | <0.0001 | 0.0004 | −6410.111 |
| Ribosome Biogenesis | 1.204 | 0.0004 | 0.003 | −7118.900 |

TABLE 12D

| KEGG Pathway; DSIIIb vs. non-CRS | Test-Statistic | p value | q value | Fold Difference |
|---|---|---|---|---|
| Two-component system | 0.678 | 0.0002 | 0.002 | 18455.395 |
| Bacterial motility proteins | 0.562 | 0.029 | 0.073 | 13903.395 |
| ABC transporters | 0.869 | 0.0002 | 0.002 | 13623.253 |
| Valine, leucine and isoleucine degradation | 0.668 | 0.008 | 0.026 | 6719.663 |
| Flagellar assembly | 0.445 | 0.013 | 0.038 | 6217.453 |
| Glyoxylate and dicarboxylate metabolism | 0.676 | 0.0001 | 0.002 | 6107.658 |
| Propanoate metabolism | 0.754 | 0.0003 | 0.002 | 6059.579 |
| Butanoate metabolism | 0.781 | 0.0002 | 0.002 | 5704.653 |
| Bacterial chemotaxis | 0.535 | 0.017 | 0.047 | 5548.095 |
| Fatty acid metabolism | 0.684 | 0.008 | 0.026 | 5445.274 |
| Benzoate degradation | 0.640 | 0.007 | 0.023 | 5124.937 |
| Transcription factors | 0.883 | 0.008 | 0.026 | 5122.316 |
| Tryptophan metabolism | 0.649 | 0.005 | 0.020 | 4940.705 |
| Lysine degradation | 0.652 | 0.005 | 0.018 | 4114.253 |
| Limonene and pinene degradation | 0.555 | 0.002 | 0.007 | 4047.095 |
| Aminobenzoate degradation | 0.641 | 0.001 | 0.007 | 3922.553 |
| Pyruvate metabolism | 0.889 | 0.032 | 0.078 | 3681.621 |
| beta-Alanine metabolism | 0.672 | 0.013 | 0.038 | 3603.605 |
| Protein kinases | 0.751 | 0.001 | 0.005 | 3171.447 |
| Pentose and glucuronate interconversions | 0.730 | 0.001 | 0.006 | 2711.979 |
| Phenylalanine metabolism | 0.677 | 0.037 | 0.086 | 2466.132 |
| Chloroalkane and chloroalkene degradation | 0.678 | 0.001 | 0.007 | 2329.974 |
| Drug metabolism - cytochrome P450 | 0.589 | 0.003 | 0.011 | 2202.189 |
| Metabolism of xenobiotics by cytochrome P450 | 0.591 | 0.002 | 0.010 | 2126.374 |
| Naphthalene degradation | 0.709 | 0.001 | 0.006 | 2095.068 |
| Signal transduction mechanisms | 0.852 | <0.0001 | <0.0001 | 2079.884 |
| Ascorbate and aldarate metabolism | 0.638 | 0.0004 | 0.003 | 1819.637 |
| Nitrogen metabolism | 0.921 | 0.033 | 0.080 | 1662.837 |
| Synthesis and degradation of ketone bodies | 0.608 | 0.004 | 0.015 | 1609.400 |
| Biosynthesis of unsaturated fatty acids | 0.749 | 0.016 | 0.045 | 1546.142 |
| Chlorocyclohexane and chlorobenzene degradation | 0.401 | 0.001 | 0.006 | 1542.832 |
| Atrazine degradation | 0.340 | 0.0002 | 0.002 | 1486.789 |
| Tyrosine metabolism | 0.879 | 0.033 | 0.080 | 1446.416 |
| Sulfur metabolism | 0.854 | 0.008 | 0.026 | 1404.342 |
| Sulfur relay system | 0.872 | 0.034 | 0.080 | 1301.379 |
| Carotenoid biosynthesis | 0.343 | 0.001 | 0.003 | 1226.437 |
| Bisphenol degradation | 0.586 | 0.018 | 0.049 | 1138.747 |
| Amino acid metabolism | 0.783 | <0.0001 | 0.001 | 1100.789 |
| beta-Lactam resistance | 0.362 | <0.0001 | 0.001 | 1056.905 |
| Toluene degradation | 0.803 | 0.004 | 0.017 | 1010.695 |
| Ethylbenzene degradation | 0.572 | <0.0001 | 0.0004 | 863.500 |
| Retinol metabolism | 0.707 | 0.022 | 0.059 | 751.342 |
| Arachidonic acid metabolism | 0.676 | 0.002 | 0.007 | 687.432 |
| Nitrotoluene degradation | 0.594 | 0.038 | 0.088 | 621.916 |
| Linoleic acid metabolism | 0.626 | 0.035 | 0.083 | 484.000 |
| Primary bile acid biosynthesis | 0.333 | 0.0002 | 0.002 | 410.700 |
| Amyotrophic lateral sclerosis (ALS) | 0.701 | 0.037 | 0.086 | 349.611 |
| D-Arginine and D-ornithine metabolism | 0.258 | <0.0001 | <0.0001 | 344.616 |
| alpha-Linolenic acid metabolism | 0.582 | 0.024 | 0.062 | 313.216 |
| Secondary bile acid biosynthesis | 0.234 | 0.0002 | 0.002 | 278.211 |
| Ether lipid metabolism | 0.500 | 0.025 | 0.065 | 222.237 |
| Prion diseases | 0.494 | 0.013 | 0.038 | 142.589 |
| Amoebiasis | 0.460 | 0.009 | 0.027 | 130.258 |
| Steroid biosynthesis | 0.273 | 0.003 | 0.011 | 109.537 |
| Betalain biosynthesis | 0.061 | <0.0001 | <0.0001 | 26.089 |
| Melanogenesis | 0.099 | <0.0001 | 0.0001 | 13.605 |
| Various types of N-glycan biosynthesis | 9.975 | 0.029 | 0.073 | −20.784 |
| Systemic lupus erythematosus | 3.288 | 0.002 | 0.009 | −105.347 |
| Protein digestion and absorption | 3.790 | 0.001 | 0.004 | −217.747 |
| Type II diabetes mellitus | 1.232 | 0.0004 | 0.003 | −252.695 |
| Butirosin and neomycin biosynthesis | 1.293 | 0.008 | 0.025 | −264.747 |
| Carbohydrate digestion and absorption | 2.350 | 0.006 | 0.022 | −315.611 |
| Ribosome biogenesis in eukaryotes | 1.242 | 0.004 | 0.016 | −361.974 |
| Primary immunodeficiency | 1.258 | 0.007 | 0.023 | −367.579 |
| Type 1 diabetes mellitus | 1.345 | 0.001 | 0.004 | −386.100 |
| Zeatin biosynthesis | 1.435 | 0.0001 | 0.001 | −418.374 |
| Biosynthesis of vancomycin group antibiotics | 1.555 | 0.013 | 0.038 | −434.495 |
| Phosphatidylinositol signaling system | 1.191 | 0.009 | 0.028 | −531.400 |
| Glutamatergic synapse | 1.307 | 0.003 | 0.012 | −568.811 |
| Ion channels | 1.659 | 0.002 | 0.008 | −583.589 |
| Insulin signaling pathway | 1.424 | 0.029 | 0.073 | −588.405 |
| Vitamin B6 metabolism | 1.131 | 0.002 | 0.007 | −599.684 |
| Glycosphingolipid biosynthesis - ganglio series | 5.296 | <0.0001 | <0.0001 | −636.542 |
| Glycosphingolipid biosynthesis - globo series | 2.531 | 0.006 | 0.021 | −777.174 |
| Glycosaminoglycan degradation | 3.392 | 0.002 | 0.008 | −803.942 |
| D-Glutamine and D-glutamate metabolism | 1.288 | <0.0001 | 0.001 | −966.121 |
| Lysosome | 3.270 | 0.001 | 0.004 | −976.047 |
| Bacterial toxins | 1.382 | 0.001 | 0.004 | −986.721 |
| Sphingolipid metabolism | 2.050 | 0.003 | 0.013 | −1046.163 |
| RNA polymerase | 1.280 | 0.006 | 0.021 | −1133.832 |
| Restriction enzyme | 1.507 | 0.0002 | 0.002 | −1345.263 |
| Carbon fixation in photosynthetic organisms | 1.108 | 0.030 | 0.074 | −1356.037 |
| Prenyltransferases | 1.167 | 0.021 | 0.058 | −1392.537 |
| Glycosyltransferases | 1.148 | 0.016 | 0.045 | −1474.168 |
| RNA degradation | 1.138 | 0.007 | 0.024 | −1546.868 |
| Alanine, aspartate and glutamate metabolism | 1.071 | 0.025 | 0.065 | −1592.121 |
| Drug metabolism - other enzymes | 1.258 | 0.014 | 0.040 | −1600.847 |
| Transcription machinery | 1.096 | 0.029 | 0.073 | −1685.268 |
| Polyketide sugar unit biosynthesis | 1.686 | 0.001 | 0.007 | −1734.495 |
| Nicotinate and nicotinamide metabolism | 1.191 | 0.0001 | 0.001 | −1832.142 |
| Base excision repair | 1.167 | 0.003 | 0.012 | −1920.142 |
| Lysine biosynthesis | 1.146 | 0.0003 | 0.002 | −2192.932 |
| Cell cycle - Caulobacter | 1.192 | 0.001 | 0.003 | −2213.611 |
| Streptomycin biosynthesis | 1.442 | 0.0002 | 0.002 | −2255.837 |
| Other glycan degradation | 3.602 | <0.0001 | 0.0001 | −2416.168 |
| Nucleotide excision repair | 1.329 | <0.0001 | 0.0002 | −2611.642 |
| Terpenoid backbone biosynthesis | 1.201 | 0.0005 | 0.003 | −2716.153 |
| Phenylalanine, tyrosine and tryptophan biosynthesis | 1.146 | 0.001 | 0.003 | −2731.842 |
| Translation factors | 1.310 | <0.0001 | <0.0001 | −3456.800 |
| Protein export | 1.234 | 0.004 | 0.016 | −3459.805 |
| Chaperones and folding catalysts | 1.142 | 0.013 | 0.038 | −3472.658 |
| DNA replication | 1.253 | 0.002 | 0.009 | −3770.884 |
| Peptidoglycan biosynthesis | 1.191 | 0.029 | 0.073 | −3784.474 |
| One carbon pool by folate | 1.316 | <0.0001 | <0.0001 | −4001.958 |
| Chromosome | 1.115 | 0.008 | 0.025 | −4091.316 |
| Mismatch repair | 1.241 | 0.001 | 0.006 | −4157.442 |
| Translation proteins | 1.214 | 0.0001 | 0.001 | −4450.947 |
| Homologous recombination | 1.285 | 0.0004 | 0.003 | −5753.963 |
| Amino acid related enzymes | 1.177 | <0.0001 | 0.0004 | −5857.142 |
| DNA replication proteins | 1.244 | 0.001 | 0.007 | −6140.763 |
| Peptidases | 1.170 | <0.0001 | 0.0002 | −6559.579 |
| Amino sugar and nucleotide sugar metabolism | 1.259 | 0.001 | 0.006 | −7100.958 |
| Aminoacyl-tRNA biosynthesis | 1.297 | 0.0001 | 0.001 | −7845.874 |
| Ribosome Biogenesis | 1.244 | 0.0003 | 0.002 | −8238.532 |
| Purine metabolism | 1.192 | 0.0003 | 0.002 | −10597.247 |
| Pyrimidine metabolism | 1.303 | 0.0001 | 0.002 | −11601.058 |
| DNA repair and recombination proteins | 1.215 | 0.0001 | 0.002 | −14214.526 |
| Ribosome | 1.326 | 0.0002 | 0.002 | −16446.253 |

Sinus Microbial Communities Correlate with Clinical Outcomes.

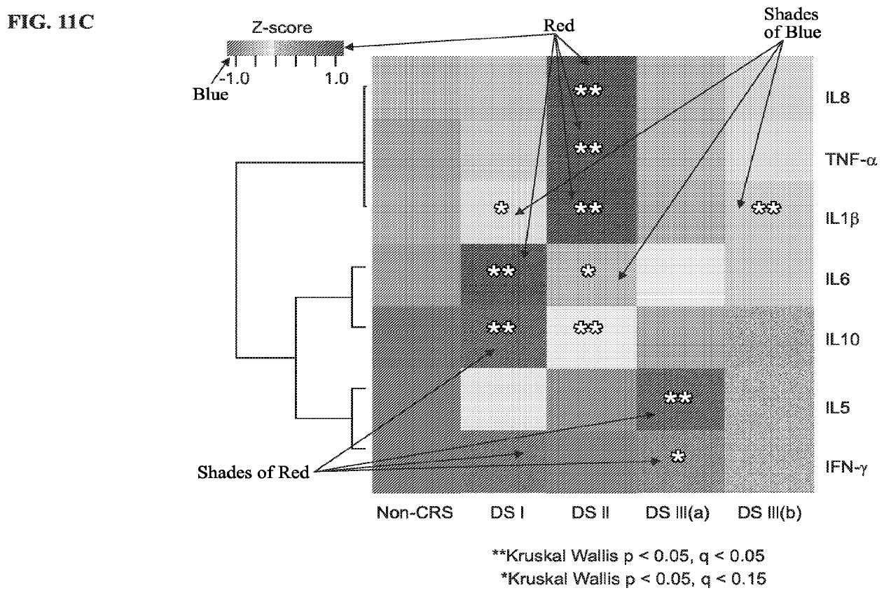

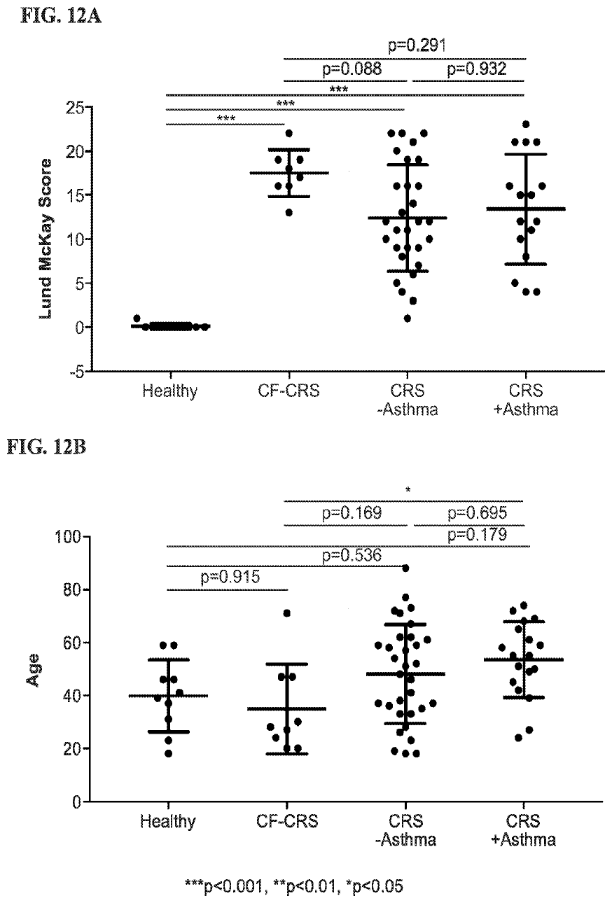

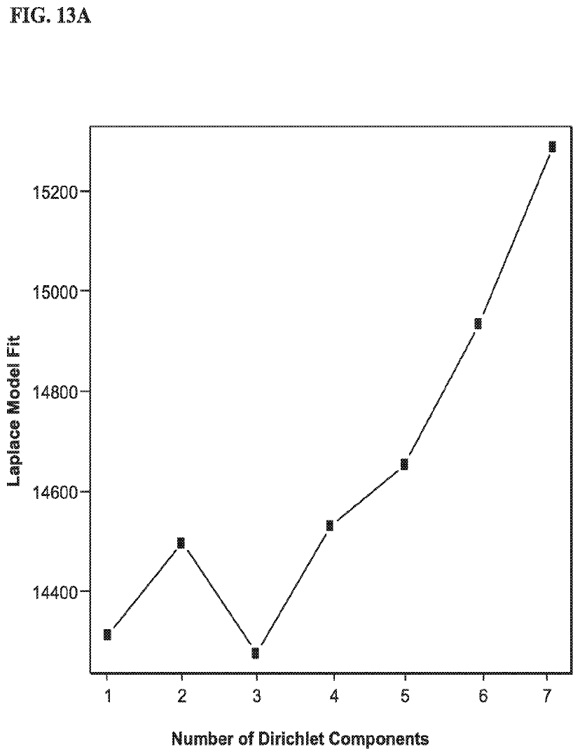

Bacterial community composition within the cohort did not correlate with polyposis (Weighted UniFrac; PERMANOVA p=0.152, 2.5% variation; FIG. 11A). However, based on the microbiota and predicted metagenome data, specifically the enrichment of RIG-I and PPAR-γ signaling pathways (previously associated with polyposis) in the Corynebacterium-dominated DSIII(a) patients, it was predicted that these patients would exhibit significantly increased incidence of polyposis. An assessment of polyp relative-risk was performed across microbiologically discrete CRS patient sub-groups compared to DSII patients, who possessed compositionally similar microbiota to those of healthy subjects and the lowest incidence of polyposis (41%; 7 of 17 patients). As expected, the DSIII(a) sub-group exhibited a significantly higher relative-risk of polyposis compared to all of the other microbiologically-defined patient sub-groups, with 89% (8 of 9 patients) of patients in this group exhibiting polyposis (Fisher's Exact; Relative Risk=2.159; p=0.039, FIG. 11B).

Bacterial co-colonization patterns correlate with patterns of host gene expression. To determine, as we hypothesized, whether distinct DS induced discrete host immune responses, we used quantitative real time PCR (qPCR) to measure expression of innate and adaptive genes previously associated with CRS (Th1, Th2, Th17 and Treg cytokines, mucin, and epithelial barrier genes) using RNA extracted in parallel with DNA used to profile microbial communities from sinus brushings of all subjects. Fold change in gene expression (compared to healthy subjects) was used to generate a multivariate immune response profile for each subject.

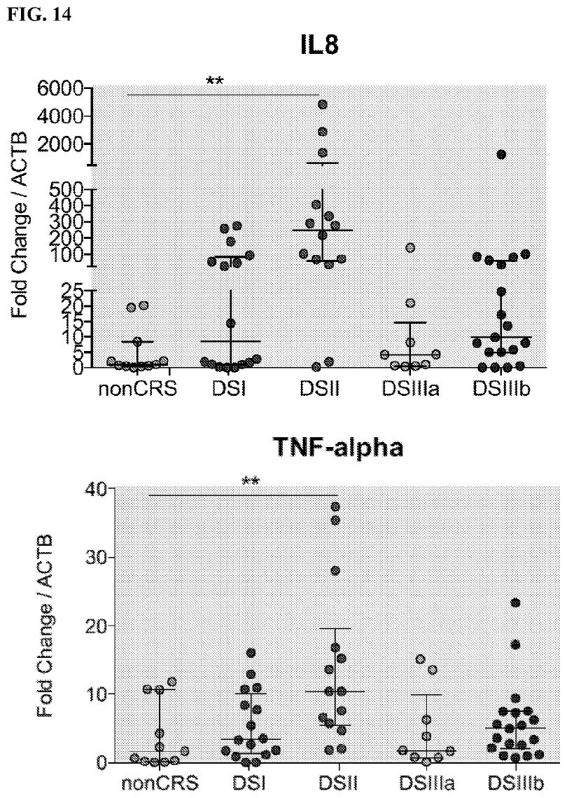

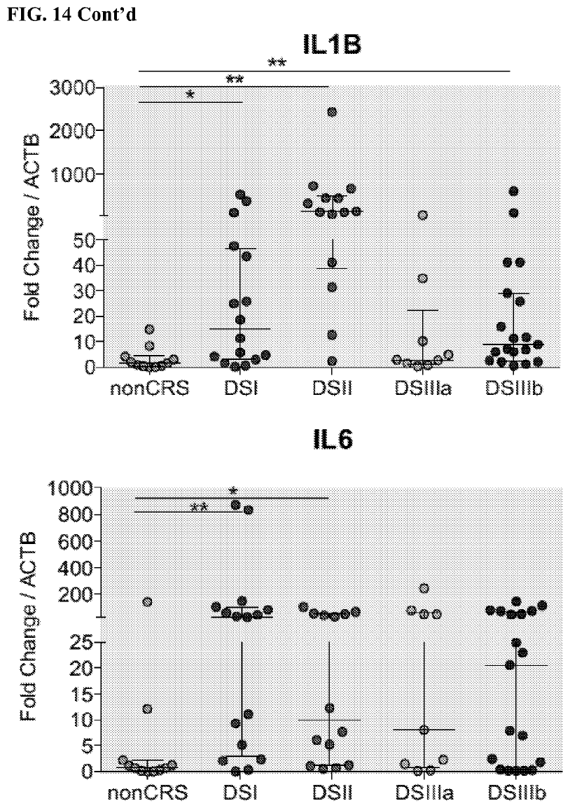

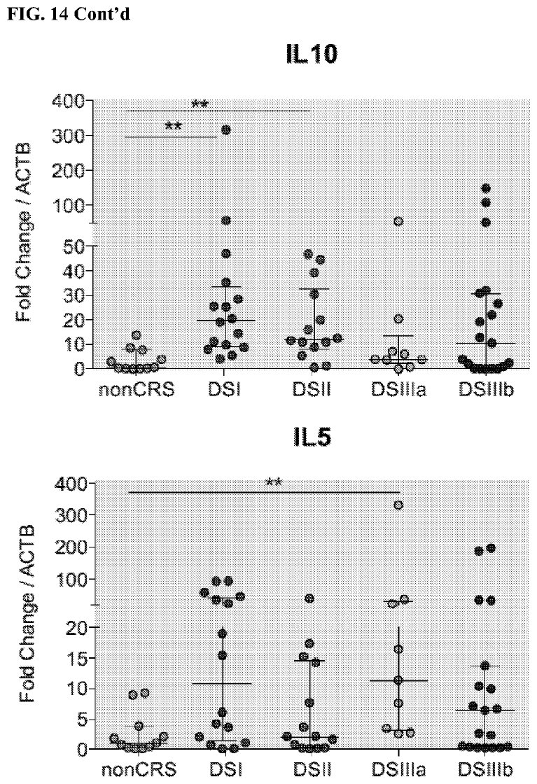

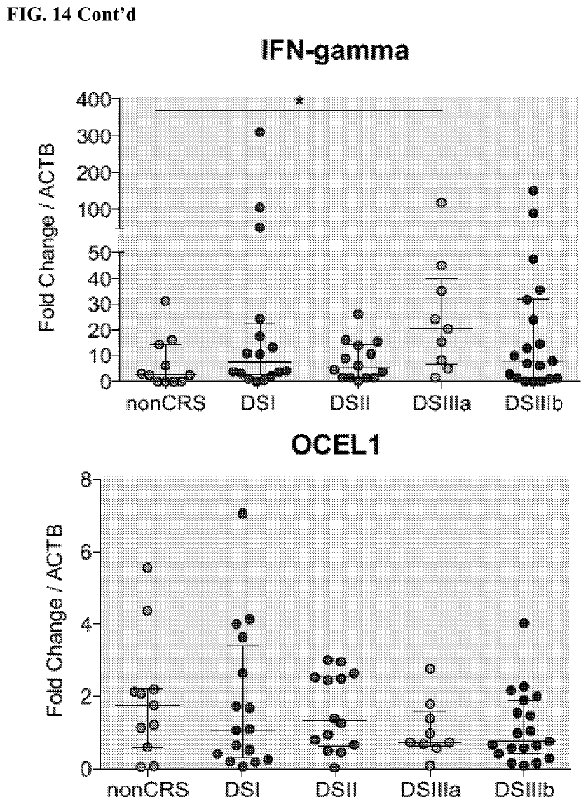

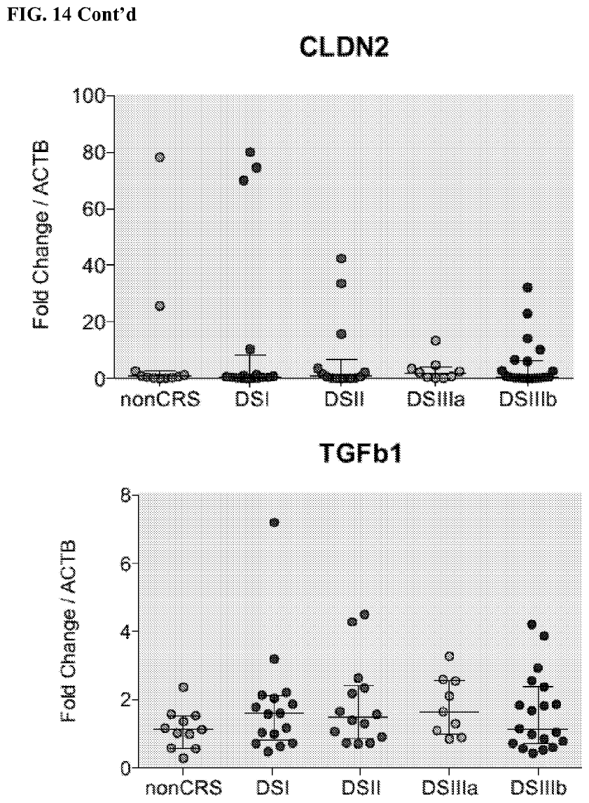

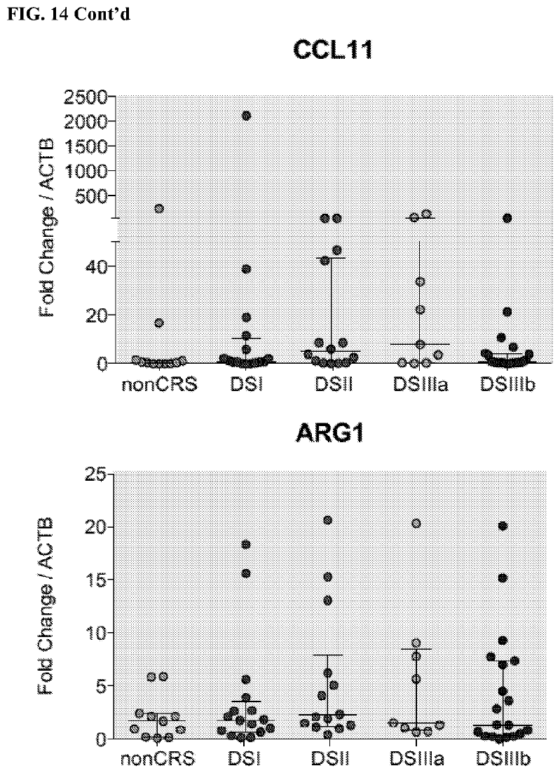

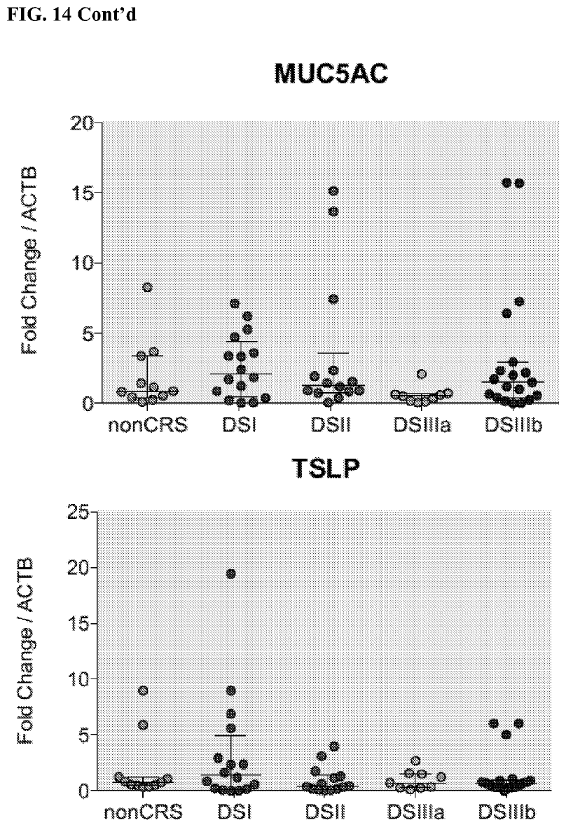

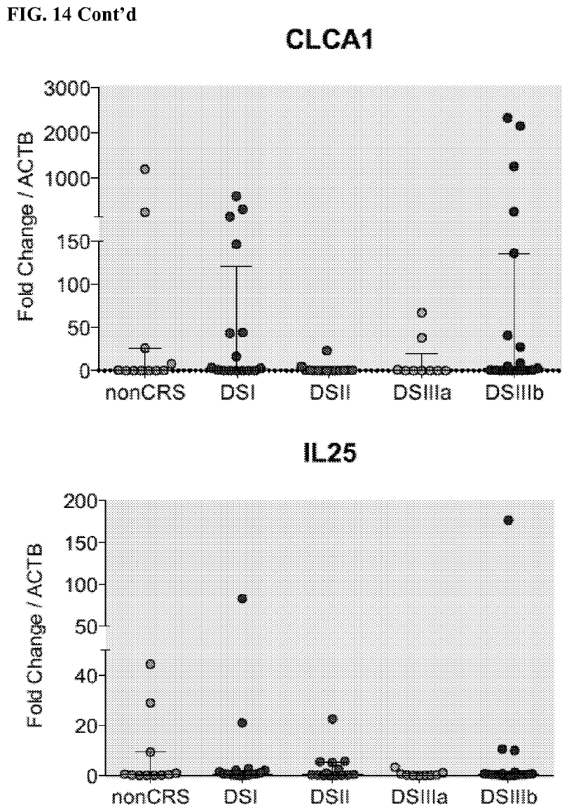

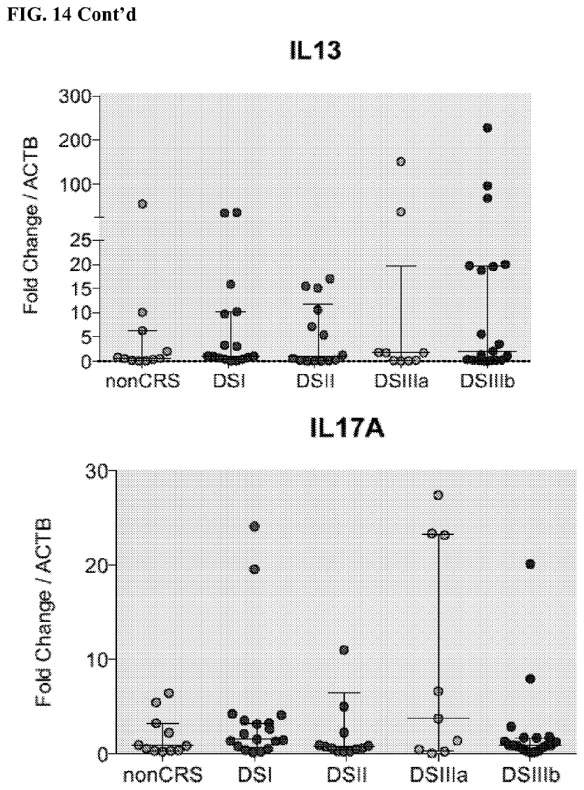

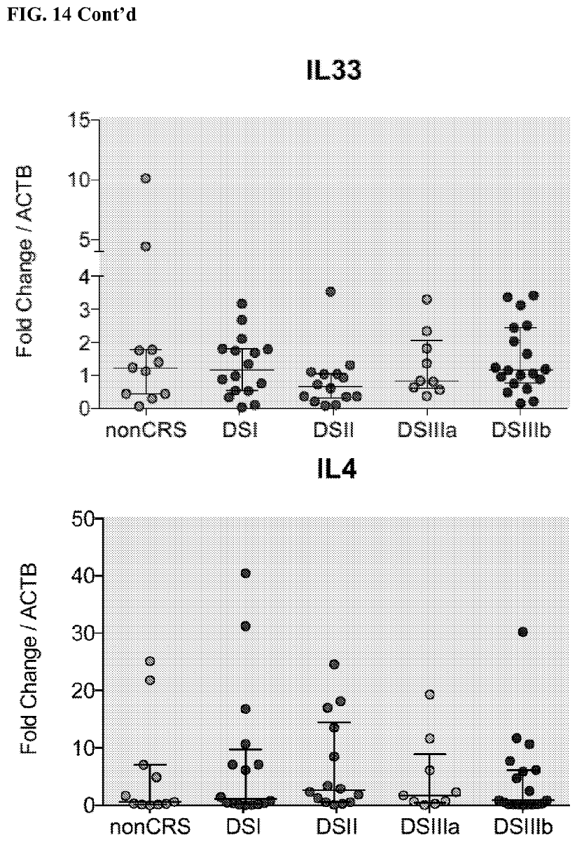

Each CRS DS group exhibited a significantly distinct host immune response, the specifics of which varied across CRS patient sub-groups (FIG. 11C; full array in FIG. 14). DSI, II, and III(b) patients exhibited significantly increased IL-1β, implicating macrophage and inflammasome involvement in these patients. In addition to IL-1β, patients in DSII also exhibited increased IL-6, TNF-α, IL-8, and IL-10 gene expression (p<0.05, q<0.05, Kruskal Wallis), suggestive of an epithelial/endothelial and/or macrophage-driven mucosal inflammatory response. DSI patients, whose microbiota composition differed subtly in taxonomic content from healthy individuals, were immunologically distinct and exhibited significantly increased IL-1β, IL-6 and IL-10 compared to healthy individuals (Kruskal Wallis p<0.05, q<0.05). Thus subtle taxonomic differences may influence the activity of this microbiota, or, alternatively, non-bacterial microbiota members contribute to immune-stimulation in this subset of patients. DSIII(a) patients who are at higher relative-risk for polyposis and whose sinus mucosal microbiome was enriched for Corynebacterium and predicted to encode PPAR-γ and RIG-I signaling pathways, were the only group to exhibit a significant increase in IL-5 expression (Kruskal Wallis p<0.05, q<0.05). IL-5 is a potent activator of eosinophils, the dominant immune cell type in polyp tissue in western populations [45]. Furthermore, these patients also had increased levels of IFN-γ (Kruskal Wallis p=0.017, q=0.107), which has been associated with non-eosinophilic polyposis [46]. Collectively, these findings indicate that distinct dysbiotic pathogenic bacterial microbiota states exist in CRS patient sub-groups that differ in relative risk for polyposis and induce discrete immune responses related to their clinical phenotypes.

Compositionally and Functionally Distinct Sinus Microbiota in Chronic Rhinosinusitis Patients have Immunological and Clinically Divergent Consequences.

Clinical diagnosis of CRS is somewhat subjective and often does not correlate well with patient outcomes[19]. Improved stratification of patients offers the opportunity to better tailor therapeutic regimens and advance towards the ultimate goal of personalized therapy. A previous study of the CRS-associated microbiota demonstrated evidence for mucosal microbiota collapse in patients with severe disease, and enrichment of Corynebacterium tuberculostearicum [5]. That study also noted that though the number of CRS patients was very small, they parsed into two distinct groups based on sinus microbiota composition. In the current study, previous findings are validated and extend, demonstrating that the CRS bacterial microbiota can exist in at least four distinct taxonomic states (one of which is dominated by Corynebacteriaceae). Without being bound by any scientific theory, it is suspected that these represent a gradient of pathogenic microbial co-colonization that are related to patient treatment history and/or disease progression. Previous CRS microbiota studies have described high inter-patient taxonomic variability, and dominance of common respiratory pathogens Corynebacterium, Staphylococcus, Pseudomonas, and anaerobes such as Fusobacterium and members of Prevotellaceae [11-13, 47, 48]. These genera also feature prominently in this study, but these findings were expanded upon to demonstrate that these respiratory pathogens co-associate with distinct and reproducible microbial partners and explain a large proportion of the observed inter-personal microbiota variation in CRS patients. These microbiologically distinct states are predicted to encode different metagenomes, are associated with a characteristic innate and adaptive host immune response, and differ significantly in the incidence of nasal polyposis, an important clinical phenotype of CRS.

Approximately one fifth of CRS patients had a mucosal microbiota characteristically enriched for Corynebacteriaceae and depleted of Streptococcus. Lemon and colleagues recently demonstrated that Corynebacterium accolens, a common skin commensal, metabolizes triacylglycerols in nasal secretions to oleic and linoleic acid, which inhibits Streptococcus pneumoniae growth [49]. Metagenome predictions indicated that the Corynebacteriaceae OTUs in dysbiotic CRS patient microbiota also encode the capacity for linoleic acid biosynthesis, suggesting that this mechanism of Streptococcus inhibition may play a role in deterministically shaping the pattern of co-colonizing species around this dominant respiratory pathogen in the chronically inflamed sinus microbiota. However, further in vitro and in vivo studies are required to determine whether this mechanism plays a role in defining CRS microbiota composition. Independent of this pathway, a recently described phylogenetic-related species, Corynbecterium pyruviciproducens, has been shown to stimulate dendritic cell maturation and proliferation and up-regulate Th2 responses in mice [50]. Additionally, the lipoarabinomannan-based lipoglycans of Corynbecterium glutamicum induce Th17 responses via TLR2 recognition on dendritic cells [51], indicating several discrete pathogenic pathways exist in this genus. In this study, Corynebacteriaceae-defined microbial communities were enriched in Peroxisome Proliferator-Activated Receptor-gamma (PPAR-γ) and Retinoic Acid-Inducible Gene 1 (RIG-I) pathways. PPAR-γ, a lipid-sensing receptor, controls gene expression and metabolism and has recently been shown to regulate eosinophil activation in polyp tissue of CRS patients [43]. It has also been associated with asthma [52] and airway remodeling following allergic inflammation in mice [53]. RIG-I, an intracellular sensor of viral DNA, is elevated in nasal polyp tissue [44] and is induced by INF-γ [54]. Consistent with these observations, patients possessing a Corynebacteriaceae-dominated community state were uniquely associated with increased IL-5 and IFN-γ gene expression and were at a higher risk for developing polyposis. Mounting evidence suggests that members of this family, particularly in the context of a taxonomically and functionally depleted sinus microbiota, represent a group of under-appreciated pathobionts, whose activities induce TH2-skewed immune responses.

Of the remaining DMM-identified microbial states, patients classified into DSII (Pseudomonadaceae-dominated) were the least functionally diverse, the most immunologically active, and housed the greatest proportion of CF and asthma patients, who also commonly exhibit lower airway microbiota dominated by this family. Predicted functional enrichments in DSII included pathways involved in tryptophan metabolism and lipopolysaccharide biosynthesis, both of which induce host inflammatory responses [55]. For example, recent studies have demonstrated that HIV-infected patients with the greatest degree of peripheral immune activation are enriched for Pseudomonas species in their gastrointestinal microbiota, and that isolates of Pseudomonas from these patients exhibit the capacity to catabolize tryptophan to pro-inflammatory kynurenine in vitro [56, 57]. Interestingly, other co-colonizing members of the DSII community are known producers of tryptophan e.g. Achromobacter [58], implicating metabolic cross-feeding between co-colonizers and the dominant respiratory pathogen as a deterministic mechanism that underlies their frequent co-association in Pseudomonas-dominated sinus microbiota. Additionally tryptophan metabolites increase biofilm formation [59, 60] and virulence gene expression[61, 62], indicating that enhanced capacity for the production and metabolism of this crucial amino acid by co-associated members of this community state may be critical to enhanced antimicrobial resistance and pathogenicity. Immunologically, patients in DSII, which had the highest prevalence of CF patients, exhibited increases in genes associated with neutrophil and macrophage activation, including TNF-α and IL-8, which is consistent with CF airway immune responses associated with strains of P. aeruginosa specifically adapted to the lung environment[63, 64]. IL-1β gene expression was increased in DSI, II, and III(b), which may indicate a role for inflammasome activation in CRS patients with T_{H}1-skewed disease. Inflammasomes are multi-meric protein complexes that assemble in cells to control the production of IL-1β and IL-18 following activation by Pathogen Associated Molecular Patterns (PAMPs), such as peptidoglycan[65].

The goal of this study was to better understand CRS patient heterogeneity by leveraging high-resolution microbiota profiles to stratify patients into discrete sub-groups, and to determine whether such a stratification strategy explained immunological and clinical outcomes in these patients as has been demonstrated in other chronic diseases [4, 22, 23, 66]. The data demonstrate the existence of distinct microbiota states and show that they are robust and encode unique functional attributes that correlate with mucosal immune responses and clinical outcomes. Without being bound by any scientific theory, it is recognized that this cross-sectional study cannot address whether these microbiota states are stable or transient, however, it is plausible that they represent a gradient of pathogenic bacterial community successional states associated with disease progression. It will be interesting to determine whether medical management of CRS, such as antimicrobial treatment or surgery, alters a patient's microbiota state and associated inflammatory response to a different conformation and whether the states we have identified are related to disease progression or duration. Antibiotics can rapidly and pervasively shift the composition of the microbiota in the human gut [67, 68], and can influence sinus microbial composition, at least in the short term [69]. Future studies will examine the effects of medical and surgical management of CRS on the stability of the disease microbiota. Although clinical subtypes of CRS patients (asthma or CF) explained a small portion of beta-diversity variation, some asthmatics share a microbiota state with CF patients. This observation suggests that subsets of patients with clinically defined distinct respiratory diseases share the same pathogenic microbiota and host immune response. The concept that discrete respiratory diseases have overlapping pathophysiology has been recently explored in asthma and chronic obstructive pulmonary disease (COPD) [70, 71]. The data suggests this phenomenon may extend to CF and can be explained, at least in part, by overlapping microbiota colonization states. Furthermore, enrichment of Proteobacteria in the lower airways patients with established asthma or CF is well documented [7, 72, 73]; data demonstrate that lower airway bacterial biomarkers of these respiratory diseases exist in the upper respiratory tract, which may represent a source of these lower airway pathogens. Future studies will incorporate more thoroughly characterized asthmatics, since asthma immune subtypes are well described and may plausibly be explained by the microbiota states observed in our study [74].

This initial study did not profile viral or fungal components of these microbiota states, which also likely play a role in driving the observed bacterial heterogeneity or host immune response. The limitations of using a predictive software to infer metagenome content are recognized, particularly since PICRUSt has been most thoroughly studied in the GI tract, although PICRUSt predictions were shown to be robust in nasal airway samples from the Human Microbiome project when compared to shotgun metagenome sequencing [33]. Future studies will use shotgun metagenomics and transcriptomics approaches to confirm PICRUSt-predicted metagenomes as well as to identify viral and fungal taxa in these patients. It is anticipated that metagenomics, in parallel with metabolomics and transcriptomics will substantially improve the capacity to meaningfully stratify patients based on their microbiome. A non-significant trend towards an association between antibiotic usage and microbiota composition was also observed. It is possible that this study was not sufficiently powered to find an association between antibiotic use and microbial composition and that the compositional differences between CRS and non-CRS subjects may, at least in part be antibiotic-mediated. We are continuing to recruit patients and will examine this possibility in larger cohorts of cases and controls. Finally, though the precise cellular source of the cytokines induced by the pathogenic bacterial community states cannot be gleaned from the gene expression studies of human sinus mucosa, significantly upregulated genes associated with each state were identified that warrant further investigation. The microbial and immunological features described herein provide an explanation for CRS patient heterogeneity, and provide a foundation for improved understanding of how distinct pathogenic sinus microbiota may collectively and distinctly drive mucosal disease processes in CRS patients.

Heterogeneity among CRS patients is poorly understood and represents a significant barrier to disease treatment and to the development of more effective therapies. This study validates and extends previous findings that show collapse of mucosal-associated microbiota in CRS patients [5, 12, 48]. Here, the data demonstrate that CRS microbiota can exist in at least four compositional states that are predicted to have distinct functional attributes, correlate with distinct host immune responses, and associate with differential risk for nasal polyps, an important clinical disease phenotype. The presence of Corynebacteriaceae-dominant microbial communities in CRS patients were associated with increased IL-5 gene expression and increased risk for nasal polyps while the remaining three microbial community states were immunologically diverse and were not associated with polyp risk. These findings support prior studies that characterize the immunological heterogeneity of CRS patients using similar clustering approaches [20], but by examining microbial signatures the studies may provide an explanation for these diverse immune profiles that exist within this patient population. The microbial and immunological features described here may inform strategies for tailored therapy in this patient population.

TABLE 13
Control v. DS I

| | | | Fold | |
|---|---|---|---|---|
| OTU_IDs | ZINB_pval | ZINB_qval | Difference | Taxonomy |
| 4466646 | 0.001 | 0.011 | 194.312 | k_Bacteria; p_Proteobacteria; c_Betaproteobacteria; o_Burkholderiales; |
| | | | | f_Comamonadaceae; g_Limnohabitans; s_{—} |
| 4416763 | 0.004 | 0.038 | 78.171 | k_Bacteria; p_Firmicutes; c_Bacilli; o_Lactobacillales; f_Streptococcaceae; |
| | | | | g_Streptococcus; s_{—} |
| 2613485 | 0.004 | 0.036 | 68.753 | k_Bacteria; p_Bacteroidetes; c_Bacteroidia; o_Bacteroidales; f_Porphyromonadaceae; |
| | | | | g_Porphyromonas; s_{—} |
| 339015 | 0.000 | 0.004 | 55.729 | k_Bacteria; p_Proteobacteria; c_Betaproteobacteria; o_Burkholderiales; |
| | | | | f_Comamonadaceae; g_Hydrogenophaga; s_{—} |
| 4447514 | 0.002 | 0.025 | 53.259 | k_Bacteria; p_Spirochaetes; c_Spirochaetes; o_Spirochaetales; f_Spirochaetaceae; |
| | | | | g_Treponema; s_amylovorum |
| 4470078 | 0.001 | 0.014 | 51.165 | k_Bacteria; p_Actinobacteria; c_Actinobacteria; o_Actinomycetales; |
| | | | | f_Corynebacteriaceae; g_Corynebacterium; s_{—} |
| 3453734 | 1.61E−09 | 6.32E−08 | 42.429 | k_Bacteria; p_Proteobacteria; c_Gammaproteobacteria; o_Pseudomonadales; |
| | | | | f_Moraxellaceae; g_Acinetobacter; s_{—} |
| 4452538 | 4.70E−42 | 2.22E−39 | 30.394 | k_Bacteria; p_Fusobacteria; c_Fusobacteriia; o_Fusobacteriales; f_Fusobacteriaceae; |
| | | | | g_Fusobacterium; s_{—} |
| 109263 | 0.000 | 0.003 | 22.559 | k_Bacteria; p_Proteobacteria; c_Gammaproteobacteria; o_Pseudomonadales; |
| | | | | f_Pseudomonadaceae; g_Pseudomonas; s_{—} |
| 611110 | 1.43E−30 | 1.35E−28 | 16.341 | k_Bacteria; p_Bacteroidetes; c_Bacteroidia; o_Bacteroidales; f_Prevotellaceae; |
| | | | | g_Prevotella; s_intermedia |
| 1135830 | 0.000 | 0.008 | 14.741 | k_Bacteria; p_Proteobacteria; c_Betaproteobacteria; o_Burkholderiales; |
| | | | | f_Comamonadaceae; g_; s_{—} |
| 1082607 | 6.49E−22 | 4.37E−20 | 14.141 | k_Bacteria; p_Actinobacteria; c_Actinobacteria; o_Actinomycetales; |
| | | | | f_Corynebacteriaceae; g_Corynebacterium; s_{—} |
| 4306262 | 4.41E−34 | 6.93E−32 | 13.641 | k_Bacteria; p_Verrucomicrobia; c_Verrucomicrobiae; o_Verrucomicrobiales; |
| | | | | f_Verrucomicrobiaceae; g_Akkermansia; s_muciniphila |
| 4472050 | 6.66E−32 | 7.86E−30 | 12.282 | k_Bacteria; p_Actinobacteria; c_Actinobacteria; o_Actinomycetales; f_Actinomycetaceae; |
| | | | | g_; s_{—} |
| 2469654 | 0.006 | 0.051 | 12.135 | k_Bacteria; p_Bacteroidetes; c_Bacteroidia; o_Bacteroidales; f_[Paraprevotellaceae]; |
| | | | | g_[Prevotella]; s_{—} |
| 3600504 | 1.10E−28 | 8.67E−27 | 11.171 | k_Bacteria; p_Bacteroidetes; c_Bacteroidia; o_Bacteroidales; f_Bacteroidaceae; |
| | | | | g_; s_{—} |
| 565936 | 0.003 | 0.035 | 10.000 | k_Bacteria; p_Proteobacteria; c_Betaproteobacteria; o_Burkholderiales; |
| | | | | f_Burkholderiaceae; g_Burkholderia; s_{—} |
| 1790396 | 0.005 | 0.044 | 9.429 | k_Bacteria; p_Proteobacteria; c_Gammaproteobacteria; o_Pseudomonadales; |
| | | | | f_Moraxellaceae; g_Acinetobacter; s_johnsonii |
| 4318284 | 2.11E−39 | 4.98E−37 | 9.365 | k_Bacteria; p_Firmicutes; c_Clostridia; o_Clostridiales; f_Veillonellaceae; |
| | | | | g_Dialister; s_{—} |
| 668514 | 2.57E−07 | 8.68E−06 | 9.029 | k_Bacteria; p_Proteobacteria; c_Gammaproteobacteria; o_Enterobacteriales; |
| | | | | f_Enterobacteriaceae; g_; s_{—} |
| 4419276 | 0.001 | 0.014 | 6.124 | k_Bacteria; p_Proteobacteria; c_Gammaproteobacteria; o_Pseudomonadales; |
| | | | | f_Pseudomonadaceae; g_Pseudomonas; s_{—} |
| 1049188 | 9.34E−12 | 4.90E−10 | 5.241 | k_Bacteria; p_Actinobacteria; c_Actinobacteria; o_Actinomycetales; |
| | | | | f_Corynebacteriaceae; g_Corynebacterium; s_{—} |
| 4406621 | 1.54E−10 | 6.62E−09 | 5.206 | k_Bacteria; p_Bacteroidetes; c_Bacteroidia; o_Bacteroidales; f_Porphyromonadaceae; |
| | | | | g_Tannerella; s_{—} |
| New.Reference | 2.45E−11 | 1.15E−09 | 3.871 | Unassigned |
| OTU32 | | | | |
| 3678349 | 0.003 | 0.031 | 3.382 | k_Bacteria; p_Firmicutes; c_Bacilli; o_Lactobacillales; f_Streptococcaceae; |
| | | | | g_Streptococcus; s_anginosus |
| 4421864 | 0.001 | 0.015 | 3.088 | k_Bacteria; p_Firmicutes; c_Clostridia; o_Clostridiales; f_Veillonellaceae; |
| | | | | g_Selenomonas; s_{—} |
| 3588390 | 1.15E−06 | 3.40E−05 | 2.241 | k_Bacteria; p_Bacteroidetes; c_Bacteroidia; o_Bacteroidales; f_Bacteroidaceae; |
| | | | | g_Bacteroides; s_{—} |
| 4384058 | 0.004 | 0.036 | 1.135 | k_Bacteria; p_Proteobacteria; c_Gammaproteobacteria; o_Enterobacteriales; |
| | | | | f_Enterobacteriaceae |
| 4425648 | 0.000 | 0.003 | 0.147 | k_Bacteria; p_Proteobacteria; c_Deltaproteobacteria; o_Desullovibrionales; |
| | | | | f_Desulfovibrionaceae; g_Desulfovibrio; s_{—} |
| 3385021 | 0.001 | 0.014 | −2.853 | k_Bacteria; p_Firmicutes; c_Bacilli; o_Bacillales; f_Staphylococcaceae; |
| | | | | g_Staphylococcus; s_{—} |
| 28841 | 0.005 | 0.049 | −3.041 | k_Bacteria; p_Proteobacteria; c_Gammaproteobacteria; o_Pseudomonadales; |
| | | | | f_Pseudomonadaceae; g_Pseudomonas; s_{—} |
| 2055565 | 0.000 | 0.004 | −3.294 | k_Bacteria; p_Spirochaetes; c_Spirochaetes; o_Spirochaetales; f_Spirochaetaceae; |
| | | | | g_Treponema; s_{—} |
| 4456252 | 0.001 | 0.013 | −4.576 | k_Bacteria; p_Firmicutes; c_Clostridia; o_Clostridiales; f_Veillonellaceae; |
| | | | | g_Megasphaera; s_{—} |
| 1106060 | 0.003 | 0.034 | −5.724 | k_Bacteria; p_Proteobacteria; c_Betaproteobacteria; o_Neisseriales; f_Neisseriaceae; |
| | | | | g_; s_{—} |
| 1017181 | 3.44E−05 | 0.0008 | −6.124 | k_Bacteria; p_Actinobacteria; c_Actinobacteria; o_Actinomycetales; f_Micrococcaceae; |
| | | | | g_Rothia; s_mucilaginosa |
| 61836 | 0.004 | 0.041 | −6.282 | k_Bacteria; p_Proteobacteria; c_Betaproteobacteria; o_Burkholderiales; |
| | | | | f_Comamonadaceae; g_Roseateles; s_depolymerans |
| 4407979 | 8.32E−05 | 0.002 | −6.541 | k_Bacteria; p_Firmicutes; c_Bacilli; o_Bacillales; f_Planococcaceae; |
| | | | | g_Lysinibacillus; s_boronitolerans |
| 4435065 | 0.006 | 0.050 | −6.741 | k_Bacteria; p_Bacteroidetes; c_Bacteroidia; o_Bacteroidales; f_Prevotellaceae; |
| | | | | g_Prevotella; s_{—} |
| 4213913 | 4.15E−05 | 0.0009 | −7.082 | k_Bacteria; p_SR1; c_; o_; f_; g_; s_{—} |
| 965500 | 9.92E−15 | 5.85E−13 | −7.229 | k_Bacteria; p_Firmicutes; c_Bacilli; o_Lactobacillales; f_Streptococcaceae; |
| | | | | g_Streptococcus; s_{—} |
| 4430581 | 4.06E−07 | 1.28E−05 | −7.824 | k_Bacteria; p_Bacteroidetes; c_Bacteroidia; o_Bacteroidales; f_[Paraprevotellaceae]; |
| | | | | g_[Prevotella]; s_{—} |
| 4377418 | 0.001 | 0.012 | −8.041 | k_Bacteria; p_Firmicutes; c_Clostridia; o_Clostridiales; f_[Tissierellaceae]; |
| | | | | g_Parvimonas; s_{—} |
| 239506 | 1.97E−05 | 0.0005 | −8.141 | k_Bacteria; p_Bacteroidetes; c_Bacteroidia; o_Bacteroidales; f_Prevotellaceae; |
| | | | | g_Prevotella; s_{—} |
| 3393186 | 1.86E−05 | 0.0005 | −10.141 | k_Bacteria; p_Proteobacteria; c_Betaproteobacteria; o_Neisseriales; f_Neisseriaceae; |
| | | | | g_; s_{—} |
| 4454385 | 0.002 | 0.023 | −10.141 | k_Bacteria; p_Bacteroidetes; c_Bacteroidia; o_Bacteroidales; f_Prevotellaceae; |
| | | | | g_Prevotella; s_{—} |
| 161677 | 0.001 | 0.012 | −10.282 | k_Bacteria; p_Proteobacteria; c_Gammaproteobacteria; o_Pseudomonadales; |
| | | | | f_Moraxellaceae; g_Acinetobacter |
| 72820 | 0.001 | 0.013 | −16.324 | k_Bacteria; p_Actinobacteria; c_Actinobacteria; o_Bifidobacteriales; |
| | | | | f_Bifidobacteriaceae; g_Bifidobacterium; s_{—} |
| 4420570 | 5.17E−08 | 1.88E−06 | −18.424 | k_Bacteria; p_Cyanobacteria; c_Chloroplast; o_Streptophyta; f_; g_; s_{—} |
| 956702 | 0.001 | 0.013 | −19.088 | k_Bacteria; p_Proteobacteria; c_Gammaproteobacteria; o_Pasteurellales; |
| | | | | f_Pasteurellaceae; g_Haemophilus; s_influenzae |
| 242070 | 0.001 | 0.010 | −32.229 | k_Bacteria; p_Proteobacteria; c_Gammaproteobacteria; o_Pseudomonadales; |
| | | | | f_Pseudomonadaceae; g_Pseudomonas; s_{—} |
| 4447394 | 0.004 | 0.036 | −33.794 | k_Bacteria; p_Actinobacteria; c_Actinobacteria; o_Actinomycetales; |
| | | | | f_Propionibacteriaceae; g_Propionibacterium; s_acnes |
| 1062051 | 1.55E−05 | 0.0004 | −160.482 | k_Bacteria; p_Actinobacteria; c_Actinobacteria; o_Actinomycetales; |
| | | | | f_Corynebacteriaceae; g_Corynebacterium; s_{—} |
| 4411138 | 0.002 | 0.021 | −275.359 | k_Bacteria; p_Actinobacteria; c_Actinobacteria; o_Actinomycetales; f_Micrococcaceae; |
| | | | | g_Rothia; s_mucilaginosa |
| 225088 | 1.89E−06 | 5.24E−05 | −764.865 | k_Bacteria; p_Proteobacteria; c_Gammaproteobacteria; o_Pseudomonadales; |
| | | | | f_Pseudomonadaceae; g_Pseudomonas; s_{—} |

TABLE 14
Control v. DS II

| | | | Fold | |
|---|---|---|---|---|
| OTU_IDs | ZINB_pval | ZINB_qval | Difference | Taxonomy |
| 891034 | 1.44E−10 | 3.64E−09 | 486.914 | k_Bacteria; p_Proteobacteria; c_Gammaproteobacteria; |
| | | | | o_Pasteurellales; f_Pasteurellaceae; g_Aggregatibacter; s_{—} |
| 219439 | 0.0485 | 0.2867 | 460.657 | k_Bacteria; p_Proteobacteria; c_Gammaproteobacteria; |
| | | | | o_Xanthomonadales; f_Xanthomonadaceae; g_; s_{—} |
| 545299 | 0.0011 | 0.0112 | 440.743 | k_Bacteria; p_Fusobacteria; c_Fusobacteriia; o_Fusobacteriales; |
| | | | | f_Fusobacteriaceae; g_Fusobacterium; s_{—} |
| 242070 | 0.0506 | 0.2900 | 264.800 | k_Bacteria; p_Proteobacteria; c_Gammaproteobacteria; |
| | | | | o_Pseudomonadales; f_Pseudomonadaceae; g_Pseudomonas; s_{—} |
| 68617 | 7.09E−06 | 0.0001 | 230.057 | k_Bacteria; p_Proteobacteria; c_Betaproteobacteria; |
| | | | | o_Burkholderiales; f_Alcaligenaceae; g_Achromobacter; s_{—} |
| 4302571 | 2.00E−86 | 7.57E−84 | 112.486 | k_Bacteria; p_Bacteroidetes; c_Bacteroidia; o_Bacteroidales; |
| | | | | f_Prevotellaceae; g_Prevotella; s_{—} |
| 4319899 | 1.42E−07 | 2.82E−06 | 76.557 | k_Bacteria; p_Fusobacteria; c_Fusobacteriia; o_Fusobacteriales; |
| | | | | f_Fusobacteriaceae; g_Fusobacterium; s_{—} |
| 4432431 | 0.0017 | 0.0167 | 60.357 | k_Bacteria; p_Proteobacteria; c_Gammaproteobacteria; |
| | | | | o_Pasteurellales; f_Pasteurellaceae; g_Aggregatibacter; s_segnis |
| 4377418 | 0.0130 | 0.0943 | 32.257 | k_Bacteria; p_Firmicutes; c_Clostridia; o_Clostridiales; |
| | | | | f_[Tissierellaceae]; g_Parvimonas; s_{—} |
| 3678349 | 1.77E−26 | 2.23E−24 | 27.929 | k_Bacteria; p_Firmicutes; c_Bacilli; o_Lactobacillales; |
| | | | | f_Streptococcaceae; g_Streptococcus; s_anginosus |
| 4466150 | 8.85E−22 | 8.36E−20 | 25.243 | k_Bacteria; p_Proteobacteria; c_Gammaproteobacteria; |
| | | | | o_Pasteurellales; f_Pasteurellaceae; g_Aggregatibacter; s_segnis |
| 851704 | 1.09E−09 | 2.43E−08 | 24.371 | k_Bacteria; p_Firmicutes; c_Clostridia; o_Clostridiales; |
| | | | | f_[Tissierellaceae]; g_Parvimonas; s_{—} |
| 4426163 | 1.06E−33 | 2.00E−31 | 20.657 | k_Bacteria; p_Bacteroidetes; c_Bacteroidia; o_Bacteroidales; |
| | | | | f_Prevotellaceae; g_Prevotella; s_{—} |
| 656881 | 0.0069 | 0.0534 | 13.543 | k_Bacteria; p_Proteobacteria; c_Gammaproteobacteria; |
| | | | | o_Enterobacteriales; f_Enterobacteriaceae; g_; s_{—} |
| 928538 | 0.0023 | 0.0203 | 13.400 | k_Bacteria; p_Firmicutes; c_Bacilli; o_Bacillales; |
| | | | | f_Staphylococcaceae; g_Staphylococcus; s_{—} |
| 4448731 | 0.0029 | 0.0246 | 11.014 | k_Bacteria; p_Fusobacteria; c_Fusobacteriia; o_Fusobacteriales; |
| | | | | f_Fusobacteriaceae; g_Fusobacterium; s_{—} |
| 3385021 | 0.0025 | 0.0217 | 8.071 | k_Bacteria; p_Firmicutes; c_Bacilli; o_Bacillales; |
| | | | | f_Staphylococcaceae; g_Staphylococcus; s_{—} |
| 269901 | 0.0412 | 0.2514 | 7.914 | k_Bacteria; p_Proteobacteria; c_Gammaproteobacteria; |
| | | | | o_Pseudomonadales; f_Pseudomonadaceae; g_; s_{—} |
| 4452538 | 0.0283 | 0.1814 | 6.814 | k_Bacteria; p_Fusobacteria; c_Fusobacteriia; |
| | | | | o_Fusobacteriales; f_Fusobacteriaceae; g_Fusobacterium; s_{—} |
| 4415943 | 0.0186 | 0.1302 | 6.757 | k_Bacteria; p_Fusobacteria; c_Fusobacteriia; |
| | | | | o_Fusobacteriales; f_Fusobacteriaceae; g_Fusobacterium; s_{—} |
| 141145 | 0.0256 | 0.1695 | 4.900 | k_Bacteria; p_Proteobacteria; c_Gammaproteobacteria; |
| | | | | o_Enterobacteriales; f_Enterobacteriaceae; g_; s_{—} |
| 939252 | 0.0498 | 0.2898 | 3.886 | k_Bacteria; p_Firmicutes; c_Bacilli; o_Bacillales; |
| | | | | f_Staphylococcaceae; g_Staphylococcus; s_{—} |
| 996487 | 1.24E−05 | 0.0002 | 3.471 | k_Bacteria; p_Firmicutes; c_Bacilli; o_Bacillales; |
| | | | | f_Staphylococcaceae; g_Staphylococcus; s_epidermidis |
| 137056 | 3.04E−05 | 0.0004 | 3.429 | k_Bacteria; p_Firmicutes; c_Bacilli; o_Bacillales; |
| | | | | f_Planococcaceae; g_; s_{—} |
| 4312969 | 0.0319 | 0.2009 | 2.443 | k_Bacteria; p_Firmicutes; c_Bacilli; o_Bacillales; |
| | | | | f_Staphylococcaceae; g_Staphylococcus |
| 87506 | 0.0052 | 0.0411 | 2.271 | k_Bacteria; p_Proteobacteria; c_Betaproteobacteria; |
| | | | | o_Neisseriales; f_Neisseriaceae; g_Eikenella; s_{—} |
| 368134 | 0.0013 | 0.0134 | 2.057 | k_Bacteria; p_Firmicutes; c_Bacilli; o_Bacillales; |
| | | | | f_Planococcaceae; g_; s_{—} |
| New.ReferenceOTU160 | 0.0022 | 0.0203 | 1.686 | k_Bacteria; p_Proteobacteria; c_Gammaproteobacteria; |
| | | | | o_Pseudomonadales; f_Pseudomonadaceae; g_Pseudomonas; s_{—} |
| 960695 | 0.0282 | 0.1814 | 1.114 | k_Bacteria; p_Firmicutes; c_Bacilli; o_Bacillales; |
| | | | | f_Planococcaceae; g_; s_{—} |
| 1068955 | 0.0209 | 0.1414 | 0.986 | k_Bacteria; p_Firmicutes; c_Bacilli; o_Bacillales; |
| | | | | f_Staphylococcaceae; g_Staphylococcus; s_{—} |
| 2211108 | 0.0002 | 0.0021 | 0.686 | k_Bacteria; p_Proteobacteria; c_Alphaprotcobacteria; o_Caulobacterales; |
| | | | | f_Caulobacteraceae; g_Brevundimonas; s_diminuta |
| 104313 | 0.0167 | 0.1194 | 0.257 | k_Bacteria; p_Proteobacteria; c_Gammaproteobacteria; |
| | | | | o_Pseudomonadales; f_Pseudomonadaceae; g_Pseudomonas; s_{—} |
| 4466659 | 6.72E−05 | 0.0008 | 0.171 | k_Bacteria; p_Fusobacteria; c_Fusobacteriia; o_Fusobacteriales; |
| | | | | f_Fusobacteriaceae; g_Fusobacterium; s_{—} |
| 1135830 | 0.0426 | 0.2559 | 0.157 | k_Bacteria; p_Proteobacteria; c_Betaproteobacteria; |
| | | | | o_Burkholderiales; f_Comamonadaceae; g_; s_{—} |
| 400315 | 0.0036 | 0.0293 | −1.143 | k_Bacteria; p_Proteobacteria; c_Gammaproteobacteria; |
| | | | | o_Pseudomonadales; f_Pseudomonadaceae; g_Pseudomonas; s_{—} |
| 4328567 | 0.0129 | 0.0943 | −1.329 | k_Bacteria; p_Proteobacteria; c_Betaproteobacteria; |
| | | | | o_Burkholderiales; f_Comamonadaceae; g_Delftia; s_{—} |
| 142419 | 0.0004 | 0.0038 | −2.014 | k_Bacteria; p_Proteobacteria; c_Gammaproteobacteria; |
| | | | | o_Pseudomonadales; f_Pseudomonadaceae |
| 4456891 | 0.0011 | 0.0112 | −3.629 | k_Bacteria; p_Proteobacteria; c_Gammaproteobacteria; |
| | | | | o_Pseudomonadales; f_Pseudomonadaceae; g_Pseudomonas; s_{—} |
| 4363066 | 0.0047 | 0.0378 | −3.629 | k_Bacteria; p_Proteobacteria; c_Gammaproteobacteria; |
| | | | | o_Pasteurellales; f_Pasteurellaceae; g_Aggregatibacter; s_{—} |
| 2685602 | 0.0025 | 0.0217 | −5.129 | k_Bacteria; p_Proteobacteria; c_Betaproteobacteria; |
| | | | | o_Burkholderiales; f_Comamonadaceae; g_Delftia; s_{—} |
| 61836 | 0.0193 | 0.1326 | −6.757 | k_Bacteria; p_Proteobacteria; c_Betaproteobacteria; o_Burkholderiales; |
| | | | | f_Comamonadaceae; g_Roseateles; s_depolymerans |
| 1981302 | 1.33E−05 | 0.0002 | −7.014 | k_Bacteria; p_Proteobacteria; c_Betaproteobacteria; |
| | | | | o_Burkholderiales; f_Burkholderiaceae; g_Burkholderia; s_{—} |
| 610486 | 3.65E−15 | 1.97E−13 | −8.200 | k_Bacteria; p_Proteobacteria; c_Betaproteobacteria; |
| | | | | o_Burkholderiales; f_Comamonadaceae |
| 823916 | 8.98E−06 | 0.0001 | −8.443 | k_Bacteria; p_Proteobacteria; c_Gammaproteobacteria; |
| | | | | o_Pseudomonadales; f_Moraxellaceae; g_Enhydrobacter; s_{—} |
| 4456889 | 5.66E−14 | 2.38E−12 | −13.671 | k_Bacteria; p_Proteobacteria; c_Gammaproteobacteria; |
| | | | | o_Pseudomonadales; f_Pseudomonadaceae; g_Pseudomonas; s_{—} |
| 71872 | 4.99E−10 | 1.18E−08 | −15.786 | k_Bacteria; p_Proteobacteria; c_Betaproteobacteria; |
| | | | | o_Burkholderiales; f_Comamonadaceae; g_Comamonas; s_{—} |
| 1566691 | 8.02E−05 | 0.0010 | −17.771 | k_Bacteria; p_Proteobacteria; c_Gammaproteobacteria; |
| | | | | o_Pseudomonadales; f_Pseudomonadaceae; g_Pseudomonas; s_{—} |
| 4344371 | 1.85E−06 | 3.04E−05 | −17.957 | k_Bacteria; p_Proteobacteria; c_Alphaproteobacteria; |
| | | | | o_Sphingomonadales; f_Sphingomonadaceae; g_phingomonas; s_{—} |
| 4331815 | 9.10E−05 | 0.001 | −20.186 | k_Bacteria; p_Proteobacteria; c_Alphaproteobacteria; |
| | | | | o_Sphingomonadales; f_Sphingomonadaceae; g_; s_{—} |
| 495067 | 0.0400 | 0.2482 | −21.200 | k_Bacteria; p_Actinobacteria; c_Actinobacteria; o_Actinomycetales; |
| | | | | f_Corynebacteriaceae; g_Corynebacterium; s_{—} |
| 4405869 | 2.15E−05 | 0.0003 | −24.186 | k_Bacteria; p_Fusobacteria; c_Fusobacteriia; o_Fusobacteriales; |
| | | | | f_Fusobacteriaceae; g_Fusobacterium; s_{—} |
| 3384047 | 5.41E−07 | 1.02E−05 | −27.129 | k_Bacteria; p_Firmicutes; c_Bacilli; o_Lactobacillales; |
| | | | | f_Streptococcaceae; g_Streptococcus; s_{—} |
| 254888 | 0.0021 | 0.0200 | −39.857 | k_Bacteria; p_Proteobacteria; c_Betaproteobacteria; |
| | | | | o_Burkholderiales; f_Comamonadaceae; g_; s_{—} |
| 1053321 | 0.0001 | 0.0012 | −83.929 | k_Bacteria; p_Proteobacteria; c_Gammaproteobacteria; |
| | | | | o_Pseudomonadales; f_Moraxellaceae; g_Moraxella; s_{—} |
| 4458959 | 1.07E−11 | 3.11E−10 | −87.114 | k_Bacteria; p_Firmicutes; c_Clostridia; o_Clostridiales; |
| | | | | f_Veillonellaceae; g_Veillonella; s_parvula |
| 4446902 | 1.37E−10 | 3.64E−09 | −94.829 | k_Bacteria; p_Firmicutes; c_Bacilli; o_Gemellales; |
| | | | | f_Gemellaceae; g_; s_{—} |
| 866280 | 1.81E−06 | 3.04E−05 | −113.729 | k_Bacteria; p_Actinobacteria; c_Actinobacteria; o_Actinomycetales; |
| | | | | f_Micrococcaceae; g_Rothia; s_mucilaginosa |
| 494906 | 1.19E−08 | 2.50E−07 | −134.843 | k_Bacteria; p_Firmicutes; c_Clostridia; o_Clostridiales; |
| | | | | f_[Tissierellaceae]; g_Peptoniphilus; s_{—} |
| 4465561 | 4.44E−13 | 1.53E−11 | −178.229 | k_Bacteria; p_Bacteroidetes; c_Bacteroidia; o_Bacteroidales; |
| | | | | f_Prevotellaceae; g_Prevotella; s_melaninogenica |
| 12574 | 1.15E−14 | 5.45E−13 | −192.757 | k_Bacteria; p_Actinobacteria; c_Actinobacteria; |
| | | | | o_Actinomycetales; f_Actinomycetaceae; g_Actinomyces; s_{—} |
| 937813 | 6.25E−07 | 1.13E−05 | −219.629 | k_Bacteria; p_Firmicutes; c_Clostridia; o_Clostridiales; |
| | | | | f_[Tissierellaceae]; g_Anaerococcus; s_{—} |
| 4439603 | 5.56E−20 | 4.20E−18 | −288.786 | k_Bacteria; p_Firmicutes; c_Bacilli; o_Lactobacillales; |
| | | | | f_Streptococcaceae; g_Streptococcus; s_{—} |
| 4411138 | 3.50E−13 | 1.32E−11 | −294.229 | k_Bacteria; p_Actinobacteria; c_Actinobacteria; o_Actinomycetales; |
| | | | | f_Micrococcaceae; g_Rothia; s_mucilaginosa |
| 4425214 | 3.56E−12 | 1.12E−10 | −304.829 | k_Bacteria; p_Firmicutes; c_Bacilli; o_Lactobacillales; |
| | | | | f_Streptococcaceae; g_Streptococcus; s_{—} |
| 1015518 | 0.0086 | 0.0652 | −406.857 | k_Bacteria; p_Actinobacteria; c_Actinobacteria; o_Actinomycetales; |
| | | | | f_Corynebacteriaceae; g_Corynebacterium; s_{—} |
| 4309301 | 2.17E−18 | 1.37E−16 | −944.129 | k_Bacteria; p_Firmicutes; c_Bacilli; o_Lactobacillales; |
| | | | | f_Streptococcaceae; g_Streptococcus; s_{—} |

TABLE 15
Control v. DS IIIa

| | | | Fold | |
|---|---|---|---|---|
| OTU_IDs | ZINB_pval | ZINB_qval | Difference | Taxonomy |
| 4465561 | 4.28E−18 | 1.67E−15 | −177.522 | k_Bacteria; p_Bacteroidetes; c_Bacteroidia; o_Bacteroidales; f_Prevotellaceae; |
| | | | | g_Prevotella; s_melaninogenica |
| 4456889 | 1.12E−12 | 2.17E−10 | −13.767 | k_Bacteria; p_Proteobacteria; c_Gammaproteobacteria; o_Pseudomonadales; |
| | | | | f_Pseudomonadaceae; g_Pseudomonas; s_{—} |
| 4321559 | 1.15E−09 | 1.49E−07 | −192.589 | k_Bacteria; p_Bacteroidetes; c_Bacteroidia; o_Bacteroidales; f_Porphyromonadaceae; |
| | | | | g_Porphyromonas; s_{—} |
| 102915 | 4.97E−09 | 4.85E−07 | 19.400 | k_Bacteria; p_Proteobacteria; c_Alphaproteobacteria; o_Sphingomonadales; |
| | | | | f_Sphingomonadaceae; g_Sphingomonas; s_{—} |
| 109263 | 1.35E−07 | 1.05E−05 | 48.722 | k_Bacteria; p_Proteobacteria; c_Gammaproteobacteria; o_Pseudomonadales; |
| | | | | f_Pseudomonadaceae; g_Pseudomonas; s_{—} |
| 225088 | 9.53E−07 | 6.20E−05 | −767.211 | k_Bacteria; p_Proteobacteria; c_Gammaproteobacteria; o_Pseudomonadales; |
| | | | | f_Pseudomonadaceae; g_Pseudomonas; s_{—} |
| 4416763 | 1.94E−06 | 9.48E−05 | 10.811 | k_Bacteria; p_Firmicutes; c_Bacilli; o_Lactobacillales; f_Streptococcaceae; |
| | | | | g_Streptococcus; s_{—} |
| 271159 | 1.91E−06 | 9.48E−05 | −146.211 | k_Bacteria; p_Firmicutes; c_Bacilli; o_Lactobacillales |
| 207936 | 2.50E−06 | 0.0001 | 19.011 | k_Bacteria; p_Firmicutes; c_Clostridia; o_Clostridiales; f_[Tissierellaceae]; |
| | | | | g_Anacrococcus; s_{—} |
| 4296424 | 2.89E−06 | 0.0001 | −47.789 | k_Bacteria; p_Actinobacteria; c_Actinobacteria; o_Actinomycetales; f_Actinomycetaceae; |
| | | | | g_Actinomyces; s_{—} |
| 1059655 | 5.08E−06 | 0.0002 | −125.989 | k_Bacteria; p_Firmicutes; c_Bacilli; o_Lactobacillales; f_Streptococcaceae; |
| | | | | g_Streptococcus; s_{—} |
| 4439603 | 1.17E−05 | 0.0004 | −278.667 | k_Bacteria; p_Firmicutes; c_Bacilli; o_Lactobacillales; f_Streptococcaceae; |
| | | | | g_Streptococcus; s_{—} |
| 4425214 | 1.55E−05 | 0.0005 | −296.900 | k_Bacteria; p_Firmicutes; c_Bacilli; o_Lactobacillales; f_Streptococcaceae; |
| | | | | g_Streptococcus; s_{—} |
| 1042479 | 1.98E−05 | 0.0006 | −33.389 | k_Bacteria; p_Bacteroidetes; c_Bacteroidia; o_Bacteroidales; f_Prevotellaceae; |
| | | | | g_Prevotella; s_melaninogenica |
| 242070 | 2.55E−05 | 0.0007 | −32.367 | k_Bacteria; p_Proteobacteria; c_Gammaproteobacteria; o_Pseudomonadales; |
| | | | | f_Pseudomonadaceae; g_Pseudomonas; s_{—} |
| 4396717 | 4.43E−05 | 0.001 | 3.500 | k_Bacteria; p_Proteobacteria; c_Alphaproteobacteria; o_Rhizobiales; |
| | | | | f_Methylobacteriaceae; g_Methylobacterium; s_{—} |
| 1135830 | 5.08E−05 | 0.001 | 14.356 | k_Bacteria; p_Proteobacteria; c_Betaproteobacteria; o_Burkholderiales; |
| | | | | f_Comamonadaceae; g_; s_{—} |
| 410908 | 5.91E−05 | 0.001 | 8.478 | k_Bacteria; p_Actinobacteria; c_Actinobacteria; o_Actinomycetales; |
| | | | | f_Corynebacteriaceae; g_Corynebacterium; s_{—} |
| 4425634 | 5.81E−05 | 0.001 | −7.389 | k_Bacteria; p_Proteobacteria; c_Gammaproteobacteria; o_Pseudomonadales; |
| | | | | f_Moraxellaceae; g_Acinetobacter; s_{—} |
| 4306048 | 5.70E−05 | 0.001 | −19.400 | k_Bacteria; p_Firmicutes; c_Bacilli; o_Lactobacillales; f_Streptococcaceae; |
| | | | | g_Streptococcus; s_{—} |
| 4431355 | 7.92E−05 | 0.001 | −22.089 | k_Bacteria; p_Proteobacteria; c_Betaproteobacteria; o_Neisseriales; f_Neisseriaceae; |
| | | | | g_; s_{—} |
| 4419276 | 9.16E−05 | 0.002 | 19.189 | k_Bacteria; p_Proteobacteria; c_Gammaproteobacteria; o_Pseudomonadales; |
| | | | | f_Pseudomonadaceae; g_Pseudomonas; s_{—} |
| 1116384 | 0.0001 | 0.002 | 34.367 | k_Bacteria; p_Proteobacteria; c_Betaproteobacteria; o_Burkholderiales; |
| | | | | f_Comamonadaceae; g_; s_{—} |
| 1927234 | 0.0001 | 0.002 | −5.989 | k_Bacteria; p_Fusobacteria; c_Fusobacteriia; o_Fusobacteriales; f_Leptotrichiaceae; |
| | | | | g_Leptotrichia; s_{—} |
| 109057 | 0.0002 | 0.003 | 9.456 | k_Bacteria; p_Proteobacteria; c_Deltaproteobacteria; o_Myxococcales; f_0319-6G20; |
| | | | | g_; s_{—} |
| 2211108 | 0.0002 | 0.003 | 9.956 | k_Bacteria; p_Proteobacteria; c_Alphaproteobacteria; o_Caulobacterales; |
| | | | | f_Caulobacteraceae; g_Brevundimonas; s_diminuta |
| 4302049 | 0.0002 | 0.003 | −5.767 | k_Bacteria; p_Firmicutes; c_Bacilli; o_Lactobacillales; f_Streptococcaceae; |
| | | | | g_Streptococcus; s_{—} |
| 368134 | 0.0002 | 0.003 | 2.811 | k_Bacteria; p_Firmicutes; c_Bacilli; o_Bacillales; f Planococcaceae; g_; s_{—} |
| 1029036 | 0.0003 | 0.003 | −11.589 | k_Bacteria; p_Bacteroidetes; c_Bacteroidia; o_Bacteroidales; f_Porphyromonadaccac; |
| | | | | g_Porphyromonas; s_{—} |
| 4455767 | 0.0003 | 0.004 | −21.989 | k_Bacteria; p_Firmicutes; c_Bacilli; o_Lactobacillales; f_Streptococcaceae; |
| | | | | g_Streptococcus; s_{—} |
| 4437024 | 0.0004 | 0.005 | −3.478 | k_Bacteria; p_Firmicutes; c_Bacilli; o_Lactobacillales; f_Streptococcaceae; |
| | | | | g_Streptococcus; s_{—} |
| 1105919 | 0.0004 | 0.005 | 25.344 | k_Bacteria; p_Proteobacteria; c_Betaproteobacteria; o_Burkholderiales; |
| | | | | f_Oxalobacteraceae; g_Ralstonia; s_{—} |
| 4446902 | 0.0005 | 0.005 | −91.344 | k_Bacteria; p_Firmicutes; c_Bacilli; o_Gemellales; f_Gemellaceae; g_; s_{—} |
| 4319899 | 0.001 | 0.006 | −8.689 | k_Bacteria; p_Fusobacteria; c_Fusobacteriia; o_Fusobacteriales; f_Fusobacteriaceae; |
| | | | | g_Fusobacterium; s_{—} |
| 1053321 | 0.001 | 0.007 | −83.889 | k_Bacteria; p_Proteobacteria; c_Gammaproteobacteria; o_Pseudomonadales; |
| | | | | f_Moraxellaceae; g_Moraxella; s_{—} |
| 4453501 | 0.001 | 0.008 | −98.578 | k_Bacteria; p_Firmicutes; c_Clostridia; o_Clostridiales; f_Veillonellaceae; |
| | | | | g_Veillonella; s_dispar |
| 4477696 | 0.001 | 0.008 | −644.311 | k_Bacteria; p_Proteobacteria; c_Gammaproteobacteria; o_Pasteurellales; |
| | | | | f_Pasteurellaceae; g_Haemophilus |
| 4309301 | 0.001 | 0.013 | −907.089 | k_Bacteria; p_Firmicutes; c_Bacilli; o_Lactobacillales; f_Streptococcaceae; |
| | | | | g_Streptococcus; s_{—} |
| 4423410 | 0.002 | 0.015 | −2.689 | k_Bacteria; p_Proteobacteria; c_Alphaproteobacteria; o_Sphingomonadales; |
| | | | | f_Sphingomonadaceae; g_; s_{—} |
| 4432431 | 0.002 | 0.015 | −9.889 | k_Bacteria; p_Proteobacteria; c_Gammaproteobacteria; o_Pasteurellales; |
| | | | | f_Pasteurellaceae; g_Aggregatibacter; s_segnis |
| 565936 | 0.002 | 0.015 | 23.000 | k_Bacteria; p_Proteobacteria; c_Betaproteobacteria; o_Burkholderiales; |
| | | | | f_Burkholderiaceae; g_Burkholderia; s_{—} |
| 4469359 | 0.002 | 0.015 | −9.178 | k_Bacteria; p_Proteobacteria; c_Gammaproteobacteria; o_Pasteurellales; |
| | | | | f_Pasteurellaceae; g_Haemophilus; s_{—} |
| 4424239 | 0.002 | 0.015 | −17.789 | k_Bacteria; p_Firmicutes; c_Bacilli; o_Lactobacillales; f_Streptococcaceae; |
| | | | | g_Streptococcus; s_{—} |
| 4337755 | 0.002 | 0.015 | −3.689 | k_Bacteria; p_Firmicutes; c_Bacilli; o_Gemellales; f_Gemellaceae; g_; s_{—} |
| 12574 | 0.003 | 0.025 | −188.900 | k_Bacteria; p_Actinobacteria; c_Actinobacteria; o_Actinomycetales; f_Actinomycetaceae; |
| | | | | g_Actinomyces; s_{—} |
| 495067 | 0.005 | 0.040 | 782.356 | k_Bacteria; p_Actinobacteria; c_Actinobacteria; o_Actinomycetales; |
| | | | | f_Corynebacteriaceae; g_Corynebacterium; s_{—} |
| 4458959 | 0.006 | 0.047 | −80.844 | k_Bacteria; p_Firmicutes; c_Clostridia; o_Clostridiales; f_Veillonellaceae; |
| | | | | g_Veillonella; s_parvula |
| 2468881 | 0.006 | 0.051 | 2.144 | k_Bacteria; p_Proteobacteria; c_Gammaproteobacteria; o_Pseudomonadales; |
| | | | | f_Pseudomonadaceae; g_Pseudomonas; s_{—} |

TABLE 16
Control v. DS IIIb

| | | | Fold | |
|---|---|---|---|---|
| OTU_IDs | ZINB_pval | ZINB_qval | Difference | Taxonomy |
| 4345285 | 0.002 | 0.013 | 2090.116 | k_Bacteria; p_Firmicutes; c_Bacilli; o_Bacillales; f_Staphylococcaceae; |
| | | | | g_Staphylococcus; s_{—} |
| 1566691 | 0.0002 | 0.002 | 213.958 | k_Bacteria; p_Proteobacteria; c_Gammaproteobacteria; o_Pseudomonadales; |
| | | | | f_Pseudomonadaceae; g_Pseudomonas; s_{—} |
| 339015 | 0.002 | 0.013 | 206.726 | k_Bacteria; p_Proteobacteria; c_Betaproteobacteria; o_Burkholderiales; |
| | | | | f_Comamonadaceae; g_Hydrogenophaga; s_{—} |
| 1981302 | 0.002 | 0.013 | 159.595 | k_Bacteria; p_Proteobacteria; c_Betaproteobacteria; o_Burkholderiales; |
| | | | | f_Burkholderiaceae; g_Burkholderia; s_{—} |
| 4466646 | 3.67E−05 | 0.0005 | 131.163 | k_Bacteria; p_Proteobacteria; c_Betaproteobacteria; o_Burkholderiales; |
| | | | | f_Comamonadaceae; g_Limnohabitans; s_{—} |
| 553611 | 1.37E−08 | 6.64E−07 | 125.195 | k_Bacteria; p_Actinobacteria; c_Actinobacteria; o_Bifidobacteriales; |
| | | | | f_Bifidobacteriaceae; g_Bifidobacterium; s_{—} |
| 4361528 | 0.004 | 0.021 | 110.042 | k_Bacteria; p_Proteobacteria; c_Gammaproteobacteria; o_Pseudomonadales; |
| | | | | f_Moraxellaceae; g_Acinetobacter; s_{—} |
| 956702 | 0.011 | 0.051 | 83.289 | k_Bacteria; p_Proteobacteria; c_Gammaproteobacteria; o_Pasteurellales; |
| | | | | f_Pasteurellaceae; g_Haemophilus; s_influenzae |
| 4456891 | 8.86E−05 | 0.001 | 57.826 | k_Bacteria; p_Proteobacteria; c_Gammaproteobacteria; o_Pseudomonadales; |
| | | | | f_Pseudomonadaceae; g_Pseudomonas; s_{—} |
| 109263 | 1.80E−09 | 9.81E−08 | 56.132 | k_Bacteria; p_Proteobacteria; c_Gammaproteobacteria; o_Pseudomonadales; |
| | | | | f_Pseudomonadaceae; g_Pseudomonas; s_{—} |
| 2685602 | 0.002 | 0.014 | 53.168 | k_Bacteria; p_Proteobacteria; c_Betaproteobacteria; o_Burkholderiales; |
| | | | | f_Comamonadaceae; g_Delftia; s_{—} |
| 928538 | 0.004 | 0.019 | 39.663 | k_Bacteria; p_Firmicutes; c_Bacilli; o_Bacillales; f_Staphylococcaceae; |
| | | | | g_Staphylococcus; s_{—} |
| 656881 | 0.0002 | 0.002 | 38.637 | k_Bacteria; p_Proteobacteria; c_Gammaproteobacteria; o_Enterobacteriales; |
| | | | | f_Enterobacteriaceae; g_; s_{—} |
| 565936 | 1.70E−05 | 0.0003 | 32.053 | k_Bacteria; p_Proteobacteria; c_Betaproteobacteria; o_Burkholderiales; |
| | | | | f_Burkholderiaceae; g_Burkholderia; s_{—} |
| 400315 | 2.27E−06 | 4.97E−05 | 31.658 | k_Bacteria; p_Proteobacteria; c_Gammaproteobacteria; o_Pseudomonadales; |
| | | | | f_Pseudomonadaceae; g_Pseudomonas; s_{—} |
| 142419 | 0.0001 | 0.001 | 27.753 | k_Bacteria; p_Proteobacteria; c_Gammaproteobacteria; o_Pseudomonadales; |
| | | | | f_Pseudomonadaceae |
| 875735 | 0.004 | 0.021 | 21.621 | k_Bacteria; p_Actinobacteria; c_Actinobacteria; o_Actinomycetales; |
| | | | | f_Actinomycetaceae; g_Actinomyces; s_{—} |
| 451449 | 4.75E−06 | 8.64E−05 | 20.853 | k_Bacteria; p_Firmicutes; c_Clostridia; o_Clostridiales; |
| | | | | f_[Tissierellaceae]; g_Anacrococcus; s_{—} |
| 668514 | 3.54E−05 | 0.0005 | 20.447 | k_Bacteria; p_Proteobacteria; c_Gammaproteobacteria; o_Enterobacteriales; |
| | | | | f_Enterobacteriaceae; g_; s_{—} |
| 825808 | 7.05E−07 | 1.71E−05 | 20.116 | k_Bacteria; p_Actinobacteria; c_Actinobacteria; o_Bifidobacteriales; |
| | | | | f_Bifidobacteriaceae ;g_Bifidobacterium; s_{—} |
| 4419276 | 1.45E−07 | 4.89E−06 | 19.932 | k_Bacteria; p_Proteobacteria; c_Gammaproteobacteria; o_Pseudomonadales; |
| | | | | f_Pseudomonadaceae; g_Pseudomonas; s_{—} |
| 584109 | 8.68E−57 | 3.79E−54 | 18.942 | k_Bacteria; p_Firmicutes; c_Bacilli; o_Lactobacillales; f_Streptococcaceae; |
| | | | | g_Streptococcus; s_{—} |
| 4396717 | 0.002 | 0.014 | 17.605 | k_Bacteria; p_Proteobacteria; c_Alphaproteobacteria; o_Rhizobiales; |
| | | | | f_Methylobacteriaceae; g_Methylobacterium; s_{—} |
| 2211108 | 2.91E−07 | 8.48E−06 | 14.242 | k_Bacteria; p_Proteobacteria; c_Alphaproteobacteria; o_Caulobacterales; |
| | | | | f_Caulobacteraceae; g_Brevundimonas; s_diminuta |
| 1105919 | 8.57E−05 | 0.001 | 12.637 | k_Bacteria; p_Proteobacteria; c_Betaproteobacteria; o_Burkholderiales; |
| | | | | f_Oxalobacteraceae; g_Ralstonia; s_{—} |
| New.Reference | 4.91E−07 | 1.26E−05 | 11.689 | k_Bacteria; p_Actinobacteria; c_Actinobacteria; o_Actinomycetales; |
| OTU66 | | | | f_Actinomycetaceae; g_Actinomyces; s_{—} |
| 1010113 | 2.19E−06 | 4.97E−05 | 10.695 | k_Bacteria; p_Proteobacteria; c_Gammaproteobacteria; o_Enterobacteriales; |
| | | | | f_Enterobacteriaceae; g_; s_{—} |
| 4420570 | 0.003 | 0.017 | 10.611 | k_Bacteria; p_Cyanobacteria; c_Chloroplast; o_Streptophyta; f_; g_; s_{—} |
| 509021 | 1.30E−37 | 1.89E−35 | 9.616 | k_Bacteria; p_Proteobacteria; c_Alphaproteobacteria; o_Sphingomonadales; |
| | | | | f_Sphingomonadaceae |
| 109057 | 8.15E−05 | 0.0009 | 9.216 | k_Bacteria; p_Proteobacteria; c_Deltaproteobacteria; o_Myxococcales; |
| | | | | f_0319-6G20; g_; s_{—} |
| 109060 | 0.0002 | 0.002 | 9.137 | k_Bacteria; p_Proteobacteria; c_Betaproteobacteria; o_Burkholderiales; |
| | | | | f_Comamonadaceae; g_Delftia; s_{—} |
| 4328567 | 0.009 | 0.042 | 9.126 | k_Bacteria; p_Proteobacteria; c_Betaproteobacteria; o_Burkholderiales; |
| | | | | f_Comamonadaceae; g_Delftia; s_{—} |
| 526682 | 0.003 | 0.018 | 7.768 | k_Bacteria; p_Actinobacteria; c_Actinobacteria; o_Actinomycetales; |
| | | | | f_Actinomycetaceae; g_Actinomyces; s_{—} |
| 1076316 | 0.004 | 0.019 | 7.511 | k_Bacteria; p_Firmicutes; c_Bacilli; o_Bacillales; f_Staphylococcaceae; |
| | | | | g_Staphylococcus; s_{—} |
| 244657 | 3.10E−05 | 0.0004 | 7.405 | k_Bacteria; p_Proteobacteria; c_Alphaproteobacteria; o_Rhizobiales; |
| | | | | f_Bradyrhizobiaceae |
| 2360704 | 3.21E−05 | 0.0004 | 7.337 | k_Bacteria; p_Proteobacteria; c_Betaproteobacteria; o_Burkholderiales; |
| | | | | f_Comamonadaceae; g_Delftia; s_{—} |
| 137056 | 0.005 | 0.024 | 7.132 | k_Bacteria; p_Firmicutes; c_Bacilli; o_Bacillales; f_Planococcaceae; g_; s_{—} |
| 219151 | 4.51E−06 | 8.57E−05 | 7.058 | k_Bacteria; p_Proteobacteria; c_Gammaproteobacteria; o_Pseudomonadales; |
| | | | | f_Moraxellaceae; g_Acinetobacter; s_{—} |
| 4423410 | 5.22E−20 | 4.56E−18 | 6.674 | k_Bacteria; p_Proteobacteria; c_Alphaproteobacteria; o_Sphingomonadales; |
| | | | | f_Sphingomonadaceae; g_; s_{—} |
| 1790396 | 0.002 | 0.015 | 6.426 | k_Bacteria; p_Proteobacteria; c_Gammaproteobacteria; o_Pseudomonadales; |
| | | | | f_Moraxellaceae; g_Acinetobacter; s_johnsonii |
| 2468881 | 6.72E−05 | 0.0008 | 5.753 | k_Bacteria; p_Proteobacteria; c_Gammaproteobacteria; o_Pseudomonadales; |
| | | | | f_Pseudomonadaceae; g_Pseudomonas; s_{—} |
| 4421747 | 0.001 | 0.010 | 5.747 | k_Bacteria; p_Proteobacteria; c_Betaproteobacteria; o_Burkholderiales; |
| | | | | f_Burkholderiaceae; g_Burkholderia; s_{—} |
| 258707 | 0.003 | 0.019 | 5.637 | k_Bacteria; p_Proteobacteria; c_Alphaproteobacteria; o_Rhizobiales; |
| | | | | f_Methylobacteriaceae; g_Methylobacterium; s_{—} |
| 4312969 | 0.004 | 0.021 | 5.537 | k_Bacteria; p_Firmicutes; c_Bacilli; o_Bacillales; f_Staphylococcaceae; |
| | | | | g_Staphylococcus |
| 4305160 | 0.002 | 0.013 | 4.563 | k_Bacteria; p_Proteobacteria; c_Betaproteobacteria; o_Burkholderiales; |
| | | | | f_Comamonadaceae |
| 2480553 | 6.60E−27 | 7.21E−25 | 4.095 | k_Bacteria; p_Fusobacteria; c_Fusobacteriia; o_Fusobacteriales; |
| | | | | f_Leptotrichiaceae; g_Leptotrichia; s_{—} |
| 4459414 | 0.0005 | 0.004 | 3.695 | k_Bacteria; p_Firmicutes; c_Clostridia; o_Clostridiales; f_Veillonellaceae; |
| | | | | g_Selenomonas; s_noxia |
| 1109251 | 0.002 | 0.013 | 3.395 | k_Bacteria; p_Proteobacteria; c_Gammaproteobacteria; o_Pseudomonadales; |
| | | | | f_Pseudomonadaceae; g_Pseudomonas; s_{—} |
| 368134 | 0.001 | 0.005 | 3.384 | k_Bacteria; p_Firmicutes; c_Bacilli; o_Bacillales; f_Planococcaceae; g_; s_{—} |
| 1068955 | 0.002 | 0.014 | 3.174 | k_Bacteria; p_Firmicutes; c_Bacilli; o_Bacillales; f_Staphylococcaceae; |
| | | | | g_Staphylococcus; s_{—} |
| 1058950 | 0.005 | 0.025 | 2.853 | k_Bacteria; p_Firmicutes; c_Bacilli; o_Bacillales; f_Planococcaceae; g_; s_{—} |
| 960695 | 0.004 | 0.019 | 2.374 | k_Bacteria; p_Firmicutes; c_Bacilli; o_Bacillales; f_Planococcaceae; g_; s_{—} |
| 161677 | 2.69E−38 | 5.87E−36 | 2.126 | k_Bacteria; p_Proteobacteria; c_Gammaproteobacteria; o_Pseudomonadales; |
| | | | | f_Moraxellaceae; g_Acinetobacter |
| 1135830 | 0.002 | 0.012 | 2.011 | k_Bacteria; p_Proteobacteria; c_Betaproteobacteria; o_Burkholderiales; |
| | | | | f_Comamonadaceae; g_; s_{—} |
| 1127804 | 0.007 | 0.032 | 1.968 | k_Bacteria; p_Proteobacteria; c_Betaproteobacteria; o_Burkholderiales; |
| | | | | f_Comamonadaceae; g_Delftia; s_{—} |
| 544841 | 8.43E−17 | 6.14E−15 | 1.700 | k_Bacteria; p_Proteobacteria; c_Alphaproteobacteria; o_Sphingomonadales; |
| | | | | f_Sphingomonadaceae; g_; s_{—} |
| 3530625 | 4.00E−07 | 1.09E−05 | 1.668 | k_Bacteria; p_Proteobacteria; c_Gammaproteobacteria; o_Pseudomonadales; |
| | | | | f_Moraxellaceae; g_Acinetobacter; s_{—} |
| 4473295 | 0.003 | 0.018 | 1.011 | k_Bacteria; p_Fusobacteria; c_Fusobacteriia; o_Fusobacteriales; |
| | | | | f_Fusobacteriaceae; g_Fusobacterium; s_{—} |
| 4353264 | 0.001 | 0.005 | 0.753 | k_Bacteria; p_Proteobacteria; c_Alphaproteobacteria; o_Caulobacterales; |
| | | | | f_Caulobacteraceae; g_; s_{—} |
| 441265 | 0.003 | 0.016 | −0.589 | k_Bacteria; p_Actinobacteria; c_Actinobacteria; o_Actinomycetales; |
| | | | | f_Corynebacteriaceae; g_Corynebacterium; s_{—} |
| 630141 | 0.003 | 0.016 | −0.637 | k_Bacteria; p_Firmicutes; c_Bacilli; o_Bacillales; f_Staphylococcaceae; |
| | | | | g_Staphylococcus; s_{—} |
| 4470078 | 0.001 | 0.007 | −0.811 | k_Bacteria; p_Actinobacteria; c_Actinobacteria; o_Actinomycetales; |
| | | | | f_Corynebacteriaceae; g_Corynebacterium; s_{—} |
| 109413 | 0.005 | 0.026 | −1.047 | k_Bacteria; p_Proteobacteria; c_Gammaproteobacteria; o_Pasteurellales; |
| | | | | f_Pasteurellaceae; g_Haemophilus; s_parainfluenzae |
| 4428042 | 0.0004 | 0.004 | −2.195 | k_Bacteria; p_Firmicutes; c_Bacilli; o_Lactobacillales; f_Streptococcaceae; |
| | | | | g_Streptococcus; s_{—} |
| 4307230 | 0.002 | 0.013 | −2.247 | k_Bacteria; p_Synergistetes; c_Synergistia; o_Synergistales; |
| | | | | f_Dethiosulfovibrionaceae; g_TG5; s_{—} |
| 4337755 | 4.99E−05 | 0.0006 | −3.642 | k_Bacteria; p_Firmicutes; c_Bacilli; o_Gemellales; f_Gemellaceae; g_; s_{—} |
| 1079708 | 3.12E−06 | 6.48E−05 | −5.295 | k_Bacteria; p_Firmicutes; c_Bacilli; o_Lactobacillales; f_Streptococcaceae; |
| | | | | g_Streptococcus; s_{—} |
| 4404577 | 0.0001 | 0.001 | −5.747 | k_Bacteria; p_Firmicutes; c_Clostridia; o_Clostridiales; |
| | | | | f_Peptostreptococcaceae; g_Peptostreptococcus |
| 965500 | 7.81E−08 | 2.85E−06 | −6.647 | k_Bacteria; p_Firmicutes; c_Bacilli; o_Lactobacillales; f_Streptococcaceae; |
| | | | | g_Streptococcus; s_{—} |
| 513646 | 0.001 | 0.007 | −7.847 | k_Bacteria; p_Firmicutes; c_Bacilli; o_Lactobacillales; f_Streptococcaceae; |
| | | | | g_Streptococcus; s_{—} |
| 2613485 | 3.19E−05 | 0.0004 | −8.547 | k_Bacteria; p_Bacteroidetes; c_Bacteroidia; o_Bacteroidales; |
| | | | | f_Porphyromonadaccac; g_Porphyromonas; s_{—} |
| 4319899 | 2.89E−05 | 0.0004 | −8.642 | k_Bacteria; p_Fusobacteria; c_Fusobacteriia; o_Fusobacteriales; |
| | | | | f_Fusobacteriaceae; g_Fusobacterium; s_{—} |
| 526804 | 0.001 | 0.008 | −9.147 | k_Bacteria; p_Firmicutes; c_Bacilli; o_Lactobacillales; f_Streptococcaceae; |
| | | | | g_Streptococcus; s_{—} |
| 3801267 | 0.003 | 0.017 | −15.011 | k_Bacteria; p_Firmicutes; c_Clostridia; o_Clostridiales; f_Veillonellaceae; |
| | | | | g_Veillonella; s_parvula |
| 72820 | 0.001 | 0.006 | −15.974 | k_Bacteria; p_Actinobacteria; c_Actinobacteria; o_Bifidobacteriales; |
| | | | | f_Bifidobacteriaceae; g_Bifidobacterium; s_{—} |
| 4430826 | 1.04E−05 | 0.0002 | −16.747 | k_Bacteria; p_Fusobacteria; c_Fusobacteriia; o_Fusobacteriales; |
| | | | | f_Leptotrichiaceae; g_Leptotrichia; s_{—} |
| 4424239 | 0.0004 | 0.003 | −17.689 | k_Bacteria; p_Firmicutes; c_Bacilli; o_Lactobacillales; f_Streptococcaceae; |
| | | | | g_Streptococcus; s_{—} |
| 4326219 | 1.71E−05 | 0.0003 | −18.537 | k_Bacteria; p_Proteobacteria; c_Epsilonproteobacteria; o_Campylobacterales; |
| | | | | f_Campylobacteraceae; g_Campylobacter; s_{—} |
| 4306048 | 2.31E−07 | 7.20E−06 | −19.400 | k_Bacteria; p_Firmicutes; c_Bacilli; o_Lactobacillales; f_Streptococcaceae; |
| | | | | g_Streptococcus; s_{—} |
| 4405869 | 1.15E−10 | 7.15E−09 | −24.242 | k_Bacteria; p_Fusobacteria; c_Fusobacteriia; o_Fusobacteriales; |
| | | | | f_Fusobacteriaceae; g_Fusobacterium; s_{—} |
| 4294457 | 0.001 | 0.008 | −26.342 | k_Bacteria; p_Actinobacteria; c_Actinobacteria; o_Actinomycetales; |
| | | | | f_Micrococcaceae; g_Rothia; s_mucilaginosa |
| 3384047 | 5.58E−08 | 2.44E−06 | −27.095 | k_Bacteria; p_Firmicutes; c_Bacilli; o_Lactobacillales; f_Streptococcaceae; |
| | | | | g_Streptococcus; s_{—} |
| 4318672 | 0.0002 | 0.002 | −27.474 | k_Bacteria; p_Proteobacteria; c_Betaproteobacteria; o_Neisseriales; |
| | | | | f_Neisseriaceae; g_Neisseria; s_{—} |
| 4307391 | 4.03E−06 | 8.01E−05 | −76.284 | k_Bacteria; p_Bacteroidetes; c_Bacteroidia; o_Bacteroidales; f_Prevotellaceae; |
| | | | | g_Prevotella; s_melaninogenica |
| 4446902 | 0.0003 | 0.003 | −92.479 | k_Bacteria; p_Firmicutes; c_Bacilli; o_Gemellales; f_Gemellaceae; g_; s_{—} |
| 4453501 | 0.009 | 0.042 | −96.747 | k_Bacteria; p_Firmicutes; c_Clostridia; o_Clostridiales; f_Veillonellaceae; |
| | | | | g_Veillonella; s_dispar |
| 4387092 | 0.002 | 0.012 | −108.053 | k_Bacteria; p_Fusobacteria; c_Fusobacteriia; o_Fusobacteriales; |
| | | | | f_Fusobacteriaceae; g_Fusobacterium; s_{—} |
| 271159 | 6.59E−08 | 2.62E−06 | −148.363 | k_Bacteria; p_Firmicutes; c_Bacilli; o_Lactobacillales |
| 4466006 | 5.18E−05 | 0.0006 | −152.784 | k_Bacteria; p_Actinobacteria; c_Actinobacteria; o_Actinomycetales; |
| | | | | f_Micrococcaceae; g_Rothia; s_dentocariosa |
| 12574 | 0.001 | 0.006 | −186.005 | k_Bacteria; p_Actinobacteria; c_Actinobacteria; o_Actinomycetales; |
| | | | | f_Actinomycetaceae; g_Actinomyces; s_{—} |
| 4396235 | 0.0004 | 0.004 | −440.558 | k_Bacteria; p_Proteobacteria; c_Betaproteobacteria; o_Neisseriales; |
| | | | | f_Neisseriaceae; g_Neisseria; s_subflava |
| 4477696 | 0.003 | 0.018 | −649.147 | k_Bacteria; p_Proteobacteria; c_Gammaproteobacteria; o_Pasteurellales; |
| | | | | f_Pasteurellaceae; g_Haemophilus |
| 4309301 | 1.88E−05 | 0.0003 | −893.305 | k_Bacteria; p_Firmicutes; c_Bacilli; o_Lactobacillales; f_Streptococcaceae; |
| | | | | g_Streptococcus; s_{—} |

TABLE 17
DS I v. DS II

| | ZINB p | ZINB q | Fold | |
|---|---|---|---|---|
| OTU_IDs | value | value | Difference | Taxonomy |
| 274754 | 2.05E−05 | 0.0003 | 611.31 | Enterobacteriaceae; g_; s_{—} |
| 545299 | 0.0003 | 0.0029 | 450.07 | Fusobacteriaceae; g_Fusobacterium; s_{—} |
| 242070 | 0.0035 | 0.0266 | 284.62 | Pseudomonadaceae; g_Pseudomonas; s_{—} |
| 68617 | 5.96E−08 | 1.59E−06 | 230.20 | Alcaligenaceae; g_Achromobacter; s_{—} |
| 4302571 | 1.24E−125 | 5.95E−123 | 113.05 | Prevotellaceae; g_Prevotella; s_{—} |
| 114510 | 6.39E−08 | 1.62E−06 | 78.92 | Enterobacteriaceae; g_; s_{—} |
| 4432431 | 0.0058 | 0.0406 | 52.32 | Pasteurellaceae; g_Aggregatibacter; s_segnis |
| 1147942 | 7.99E−28 | 1.92E−25 | 39.48 | Pasteurellaceae; g_Aggregatibacter; s_{—} |
| 3678349 | 8.79E−05 | 0.0011 | 25.70 | Streptococcaceae; g_Streptococcus; |
| | | | | s_anginosus |
| 4466150 | 8.82E−07 | 1.84E−05 | 24.72 | Pasteurellaceae; g_Aggregatibacter; s_segnis |
| 4426163 | 2.56E−10 | 1.03E−08 | 20.59 | Prevotellaceae; g_Prevotella; s_{—} |
| 610111 | 3.37E−20 | 4.05E−18 | 15.48 | Prevotellaceae; g_Prevotella; s_{—} |
| 928538 | 0.0013 | 0.0112 | 15.46 | Staphylococcaceae; g_Staphylococcus; s_{—} |
| 656881 | 8.31 E−05 | 0.0011 | 13.37 | Enterobacteriaceae; g_; s_{—} |
| 4448731 | 0.0005 | 0.0052 | 11.14 | Fusobacteriaceae; g_Fusobacterium; s_{—} |
| 3385021 | 0.0001 | 0.0017 | 9.92 | Staphylococcaceae; g_Staphylococcus; s_{—} |
| 269901 | 0.0017 | 0.0135 | 8.71 | Pseudomonadaceae; g_; s_{—} |
| 144814 | 1.68E−06 | 3.37E−05 | 7.42 | Enterobacteriaceae |
| 91557 | 2.08E−09 | 6.66E−08 | 7.23 | Enterobacteriaceae |
| 4415943 | 0.0004 | 0.0044 | 6.93 | Fusobacteriaceae; g_Fusobacterium; s_{—} |
| 141145 | 0.0046 | 0.0337 | 4.96 | Enterobacteriaceae; g_; s_{—} |
| 996487 | 2.83E−07 | 6.80E−06 | 4.07 | Staphylococcaceae; g_Staphylococcus; |
| | | | | s_epidermidis |
| 939252 | 3.89E−06 | 7.26E−05 | 3.98 | Staphylococcaceae; g_Staphylococcus; s_{—} |
| 122049 | 4.04E−08 | 1.14E−06 | 3.96 | Enterobacteriaceae; g_; s_{—} |
| 137056 | 2.04E−09 | 6.66E−08 | 3.70 | Planococcaceae; g_; s_{—} |
| 4312969 | 0.0044 | 0.0327 | 2.68 | Staphylococcaceae; g_Staphylococcus |
| 1076316 | 0.0009 | 0.0084 | 2.53 | Staphylococcaceae; g_Staphylococcus; s_{—} |
| New.ReferenceOTU160 | 0.0016 | 0.0130 | 1.75 | Pseudomonadaceae; g_Pseudomonas; s_{—} |
| 960695 | 0.0014 | 0.0122 | 1.14 | Planococcaceae; g_; s_{—} |
| 4466659 | 7.89E−05 | 0.0010 | 1.03 | Fusobacteriaceae; g_Fusobacterium; s_{—} |
| 4415319 | 0.0071 | 0.0485 | 0.96 | Alcaligenaceae; g_Achromobacter; s_{—} |
| 1055132 | 0.0073 | 0.0491 | 0.71 | Staphylococcaceae; g_Staphylococcus |
| 982266 | 0.0030 | 0.0231 | −0.55 | [Chromatiaceae] |
| 4322739 | 0.0003 | 0.0035 | −0.73 | Dermacoccaceae; g_Dermacoccus; s_{—} |
| 159017 | 0.0006 | 0.0056 | −0.97 | Caulobacteraceae; g_; s_{—} |
| 109060 | 0.0053 | 0.0372 | −1.70 | Comamonadaceae; g_Delftia; s_{—} |
| 809192 | 0.0001 | 0.0013 | −2.31 | Dermabacteraceae; g_Brachybacterium; |
| | | | | s_conglomeratum |
| 4328567 | 0.0032 | 0.0243 | −2.43 | Comamonadaceae; g_Delftia; s_{—} |
| 4449609 | 2.03E−05 | 0.0003 | −3.62 | Sphingomonadaceae; g_phingomonas; s_{—} |
| 4323897 | 0.0028 | 0.0219 | −3.78 | Oxalobacteraceae; g_; s_{—} |
| 823916 | 0.0048 | 0.0342 | −5.49 | Moraxellaceae; g_Enhydrobacter; s_{—} |
| 142419 | 7.70E−06 | 0.0001 | −5.52 | Pseudomonadaceae |
| 4363066 | 0.0002 | 0.0023 | −5.70 | Pasteurellaceae; g_Aggregatibacter; s_{—} |
| 668514 | 0.0006 | 0.0056 | −5.99 | Enterobacteriaceae; g_; s_{—} |
| 400315 | 3.92E−06 | 7.26E−05 | −6.18 | Pseudomonadaceae; g_Pseudomonas; s_{—} |
| 1082607 | 8.36E−21 | 1.34E−18 | −9.27 | Corynebacteriaceae; g_Corynebacterium; s_{—} |
| 4331815 | 0.0011 | 0.0097 | −9.98 | Sphingomonadaceae; g_; s_{—} |
| 4344371 | 4.68E−05 | 0.0007 | −12.47 | Sphingomonadaceae; g_phingomonas; s_{—} |
| 4456891 | 9.54E−06 | 0.0002 | −12.93 | Pseudomonadaceae; g_Pseudomonas; s_{—} |
| 615020 | 0.0004 | 0.0037 | −13.27 | Mycoplasmataceae; g Mycoplasma; s_{—} |
| 2685602 | 1.21E−05 | 0.0002 | −13.74 | Comamonadaceae; g_Delftia; s_{—} |
| 3384047 | 4.31E−05 | 0.0006 | −14.12 | Streptococcaceae; g_Streptococcus; s_{—} |
| 610486 | 0.0001 | 0.0017 | −21.62 | Comamonadaceae |
| 1053321 | 0.0006 | 0.0055 | −35.16 | Moraxellaceae; g_Moraxella; s_{—} |
| 1981302 | 3.44E−10 | 1.27E−08 | −37.45 | Burkholderiaceae; g_Burkholderia; s_{—} |
| 254888 | 6.48E−05 | 0.0009 | −41.40 | Comamonadaceae; g_; s_{—} |
| 1566691 | 3.32E−07 | 7.58E−06 | −47.23 | Pseudomonadaceae; g_Pseudomonas; s_{—} |
| 4416763 | 0.0003 | 0.0035 | −52.71 | Streptococcaceae; g_Streptococcus; s_{—} |
| 866280 | 4.48E−06 | 7.98E−05 | −55.89 | Micrococcaceae; g_Rothia; s_mucilaginosa |
| 494906 | 0.0002 | 0.0019 | −59.76 | [Tissierellaceae]; g_Peptoniphilus; s_{—} |
| 4458959 | 1.97E−11 | 9.45E−10 | −71.25 | Veillonellaceae; g_Veillonella; s_parvula |
| 4405869 | 3.47E−07 | 7.58E−06 | −85.44 | Fusobacteriaceae; g_Fusobacterium; s_{—} |
| 937813 | 5.73E−05 | 0.0008 | −89.12 | [Tissierellaceae]; g_Anaerococcus; s_{—} |
| 4446902 | 4.15E−11 | 1.82E−09 | −115.01 | Gemellaceae; g_; s_{—} |
| 4411138 | 9.82E−09 | 2.95E−07 | −121.47 | Micrococcaceae; g_Rothia; s_mucilaginosa |
| 495067 | 0.0017 | 0.0140 | −136.15 | Corynebacteriaceae; g_Corynebacterium; s_{—} |
| 12574 | 1.19E−14 | 8.19E−13 | −144.40 | Actinomycetaceae; g_Actinomyces; s_{—} |
| 4465561 | 2.66E−13 | 1.60E−11 | −233.97 | Prevotellaceae; g_Prevotella; |
| | | | | s_melaninogenica |
| 4439603 | 3.85E−17 | 3.09E−15 | −234.40 | Streptococcaceae; g_Streptococcus; s_{—} |
| 4425214 | 2.39E−12 | 1.28E−10 | −298.58 | Streptococcaceae; g_Streptococcus; s_{—} |
| 4309301 | 8.28E−18 | 7.96E−16 | −837.31 | Streptococcaceae; g_Streptococcus; s_{—} |

TABLE 18

| #Sample | Metric | Value |
|---|---|---|
| 111re | Weighted NSTI | 0.07804709 |
| 103re | Weighted NSTI | 0.03736167 |
| 110le | Weighted NSTI | 0.08932045 |
| 98le | Weighted NSTI | 0.09345745 |
| 96LE | Weighted NSTI | 0.04207512 |
| 99lm | Weighted NSTI | 0.03690656 |
| 93re | Weighted NSTI | 0.10173422 |
| 88le | Weighted NSTI | 0.02533562 |
| 109le | Weighted NSTI | 0.04475136 |
| 56LM | Weighted NSTI | 0.02350493 |
| CRS18 | Weighted NSTI | 0.03933224 |
| 128lm | Weighted NSTI | 0.01137079 |
| 40RM | Weighted NSTI | 0.04207741 |
| 83re | Weighted NSTI | 0.02567592 |
| 60re | Weighted NSTI | 0.15454807 |
| 80rm | Weighted NSTI | 0.02526999 |
| 114lit | Weighted NSTI | 0.02551737 |
| 31IT | Weighted NSTI | 0.03056151 |
| 32LE | Weighted NSTI | 0.02412145 |
| 64LE | Weighted NSTI | 0.02208247 |
| CRS20 | Weighted NSTI | 0.03030393 |
| 7LM | Weighted NSTI | 0.0318742 |
| 39RE | Weighted NSTI | 0.0249914 |
| CRS15 | Weighted NSTI | 0.02066796 |
| ctrl4 | Weighted NSTI | 0.07845484 |
| 15RM | Weighted NSTI | 0.04695093 |
| 81lm | Weighted NSTI | 0.06910842 |
| 85lm | Weighted NSTI | 0.01767781 |
| CRS14 | Weighted NSTI | 0.03160402 |
| 90le | Weighted NSTI | 0.01676486 |
| 33LE | Weighted NSTI | 0.02358757 |
| 82re | Weighted NSTI | 0.05254296 |
| 131RM | Weighted NSTI | 0.03148185 |
| 123le | Weighted NSTI | 0.02967039 |
| 105le | Weighted NSTI | 0.0391358 |
| 61RE | Weighted NSTI | 0.03439586 |
| 44LE | Weighted NSTI | 0.0375247 |
| 16RM | Weighted NSTI | 0.02807576 |
| 143LM | Weighted NSTI | 0.02634699 |
| 107le | Weighted NSTI | 0.03886195 |
| 100RM | Weighted NSTI | 0.0220393 |
| 92le | Weighted NSTI | 0.06680343 |
| 91le | Weighted NSTI | 0.02611467 |
| CRS19 | Weighted NSTI | 0.04162983 |
| CRS17 | Weighted NSTI | 0.02058987 |
| 43LM | Weighted NSTI | 0.13773745 |
| 101lm | Weighted NSTI | 0.06001752 |
| 86le | Weighted NSTI | 0.03391079 |
| 97le | Weighted NSTI | 0.05725306 |
| 59RE | Weighted NSTI | 0.04945922 |
| 104re | Weighted NSTI | 0.03647425 |
| 117re | Weighted NSTI | 0.03578929 |
| 8RM | Weighted NSTI | 0.08071753 |
| 108re | Weighted NSTI | 0.02371532 |
| 36LM | Weighted NSTI | 0.0205843 |
| 63lm | Weighted NSTI | 0.03186186 |
| 55re | Weighted NSTI | 0.03759735 |
| 115re | Weighted NSTI | 0.00559684 |
| 130lm | Weighted NSTI | 0.02824808 |
| CRS16 | Weighted NSTI | 0.0406482 |
| 1LM | Weighted NSTI | 0.01632011 |
| 17RM | Weighted NSTI | 0.13313886 |
| 122le | Weighted NSTI | 0.0487135 |
| 112lm | Weighted NSTI | 0.04522731 |
| 22LE | Weighted NSTI | 0.02516208 |
| 94lm | Weighted NSTI | 0.04761255 |
| 121lm | Weighted NSTI | 0.02740169 |
| 120le | Weighted NSTI | 0.02443964 |
| 34LM | Weighted NSTI | 0.02985559 |
| | Average | 0.04257585 |
| | Min | 0.00559684 |
| | Max | 0.15454807 |

### REFERENCES

### References for Example 1

1. K. Biswas, M. Hoggard, R. Jain, M. W. Taylor, R. G. Douglas, The nasal microbiota in health and disease: variation within and between subjects, Front. Microbiol. 6 (2015), doi:10.3389/fmicb.2015.00134.
2. N. A. Abreu, N. A. Nagalingam, Y. Song, F. C. Roediger, S. D. Pletcher, A. N.

Goldberg, S. V. Lynch, Sinus Microbiome Diversity Depletion and Corynebacterium tuberculostearicum Enrichment Mediates Rhinosinusitis, Sci. Transl. Med. 4, 1-9 (2012).
3. S. M. Teo, D. Mok, K. Pham, M. Kusel, M. Serralha, N. Troy, B. J. Holt, B. J. Hales, M. L. Walker, E. Hollams, Y. A. Bochkov, K. Grindle, S. L. Johnston, J. E. Gem, P. D. Sly, P. G. Holt, K. E. Holt, M. Inouye, The Infant Nasopharyngeal Microbiome Impacts Severity of Lower Respiratory Infection and Risk of Asthma Development, Cell Host Microbe 17, 704-715 (2015).
4. H. Bisgaard, M. N. Hermansen, F. Buchvald, L. Loland, L. B. Halkjaer, K. Bønnelykke, M. Brasholt, A. Heltberg, N. H. Vissing, S. V. Thorsen, M. Stage, C. B. Pipper, Childhood Asthma after Bacterial Colonization of the Airway in Neonates, N. Engl. J. Med. 357, 1487-1495 (2007).
5. C. A. Santee, N. A. Nagalingam, A. A. Faruqi, G. P. DeMuri, J. E. Gem, E. R. Wald, S. V. Lynch, Nasopharyngeal microbiota composition of children is related to the frequency of upper respiratory infection and acute sinusitis, Microbiome 4, 34 (2016).
6. K. M. Kloepfer, W. M. Lee, T. E. Pappas, T. Kang, R. F. Vrtis, M. D. Evans, R. E. Gangnon, Y. A. Bochkov, D. J. Jackson, R. F. Lemanske, J. E. Gem, Detection of Pathogenic Bacteria During Rhinovirus Infection is Associated with Increased Respiratory Symptoms and Exacerbations of Asthma, J. Allergy Clin. Immunol. 133, 1301-1307.e3 (2014).
7. W. W. Busse, R. F. Lemanske, J. E. Gem, The Role of Viral Respiratory Infections in Asthma and Asthma Exacerbations, Lancet 376, 826-834 (2010).
8. S. J. Teach, M. A. Gill, A. Togias, C. A. Sorkness, S. J. Arbes, A. Calatroni, J. J. Wildfire, P. J. Gergen, R. T. Cohen, J. A. Pongracic, C. M. Kercsmar, G. K. K. Hershey, R. S. Gruchalla, A. H. Liu, E. M. Zoratti, M. Kattan, K. A. Grindle, J. E. Gem, W. W. Busse, S. J. Szefler, Preseasonal treatment with either omalizumab or an inhaled corticosteroid boost to prevent fall asthma exacerbations, J. Allergy Clin. Immunol. 136, 1476-1485 (2015).
9. M. L. Perez Vidakovics, K. Riesbeck, Virulence mechanisms of Moraxella in the pathogenesis of infection, Curr. Opin. Infect. Dis. 22, 279-285 (2009).
10. A. Bossios, S. Psarras, D. Gourgiotis, C. L. Skevaki, A. G. Constantopoulos, P. Saxoni-Papageorgiou, N. G. Papadopoulos, Rhinovirus infection induces cytotoxicity and delays wound healing in bronchial epithelial cells, Respir. Res. 6, 114 (2005).
11. W. W. Busse, W. J. Morgan, P. J. Gergen, H. E. Mitchell, J. E. Gem, A. H. Liu, R. S. Gruchalla, M. Kattan, S. J. Teach, J. A. Pongracic, J. F. Chmiel, S. F. Steinbach, A. Calatroni, A. Togias, K. M. Thompson, S. J. Szefler, C. A. Sorkness, Randomized Trial of Omalizumab (Anti-IgE) for Asthma in Inner-City Children, N. Engl. J. Med. 364, 1005-1015 (2011).
12. K. M. DeAngelis, E. L. Brodie, T. Z. DeSantis, G. L. Andersen, S. E. Lindow, M. K. Firestone, Selective progressive response of soil microbial community to wild oat roots, ISMS J. 3, 168-178 (2008).
13. T. Magoč, S. L. Salzberg, FLASH: fast length adjustment of short reads to improve genome assemblies, Bioinforma. Oxf. Engl. 27, 2957-2963 (2011).
14. J. G. Caporaso, J. Kuczynski, J. Stombaugh, K. Bittinger, F. D. Bushman, E. K. Costello, N. Fierer, A. G. Peña, J. K. Goodrich, J. I. Gordon, G. A. Huttley, S. T. Kelley, D. Knights, J. E. Koenig, R. E. Ley, C. A. Lozupone, D. McDonald, B. D. Muegge, M. Pirrung, J. Reeder, J. R. Sevinsky, P. J. Turnbaugh, W. A. Walters, J. Widmann, T. Yatsunenko, J. Zaneveld, R. Knight, QIIME allows analysis of high-throughput community sequencing data, Nat. Methods 7, 335-336 (2010).
15. R. C. Edgar, Search and clustering orders of magnitude faster than BLAST, Bioinformatics 26, 2460-2461 (2010).
16. C. Lozupone, M. E. Lladser, D. Knights, J. Stombaugh, R. Knight, UniFrac: an effective distance metric for microbial community comparison, ISMS J. 5, 169-172 (2011).
17. R. Romero, S. S. Hassan, P. Gajer, A. L. Tarca, D. W. Fadrosh, L. Nikita, M. Galuppi, R. F. Lamont, P. Chaemsaithong, J. Miranda, T. Chaiworapongsa, J. Ravel, The composition and stability of the vaginal microbiota of normal pregnant women is different from that of non-pregnant women, Microbiome 2, 4 (2014).
18. J. Friedman, E. J. Alm, Inferring Correlation Networks from Genomic Survey Data, PLOS Comput. Biol. 8, e1002687 (2012).
19. P. Langfelder, S. Horvath, WGCNA: an R package for weighted correlation network analysis, BMC Bioinformatics 9, 559 (2008).
20. J. G. Caporaso, C. L. Lauber, W. A. Walters, D. Berg-Lyons, J. Huntley, N. Fierer, S. M. Owens, J. Betley, L. Fraser, M. Bauer, N. Gormley, J. A. Gilbert, G. Smith, R. Knight, Ultra-high-throughput microbial community analysis on the Illumina HiSeq and MiSeq platforms, ISMS J. 6, 1621-1624 (2012).
21. J. G. Caporaso, C. L. Lauber, W. A. Walters, D. Berg-Lyons, C. A. Lozupone, P. J. Turnbaugh, N. Fierer, R. Knight, Global patterns of 16S rRNA diversity at a depth of millions of sequences per sample, Proc. Natl. Acad. Sci. U.S.A. 108 Suppl 1, 4516-4522 (2011).
22. J. G. Caporaso, K. Bittinger, F. D. Bushman, T. Z. DeSantis, G. L. Andersen, R. Knight, PyNAST: a flexible tool for aligning sequences to a template alignment, Bioinforma. Oxf. Engl. 26, 266-267 (2010).
23. R. C. Edgar, B. J. Haas, J. C. Clemente, C. Quince, R. Knight, UCHIME improves sensitivity and speed of chimera detection, Bioinforma. Oxf. Engl. 27, 2194-2200 (2011).
24. T. Z. DeSantis, P. Hugenholtz, N. Larsen, M. Rojas, E. L. Brodie, K. Keller, T. Huber, D. Dalevi, P. Hu, G. L. Andersen, Greengenes, a chimera-checked 16S rRNA gene database and workbench compatible with ARB, Appl. Environ. Microbiol. 72, 5069-5072 (2006).
25. K. E. Fujimura, A. R. Sitarik, S. Haystad, D. L. Lin, S. Levan, D. Fadrosh, A. R. Panzer, B. LaMere, E. Rackaityte, N. W. Lukacs, G. Wegienka, H. A. Boushey, D. R. Ownby, E. M. Zoratti, A. M. Levin, C. C. Johnson, S. V. Lynch, Neonatal gut microbiota associates with childhood multisensitized atopy and T cell differentiation, Nat. Med. 22, 1187-1191 (2016).
26. Y. A. Bochkov, K. Grindle, F. Vang, M. D. Evans, J. E. Gem, Improved Molecular Typing Assay for Rhinovirus Species A, B, and C, J. Clin. Microbiol. 52, 2461-2471 (2014).
27. R Core Team, R: A language and environment for statistical computing (R Foundation for Statistical Computing, Vienna, Austria, 2015; https://www.R-project.org).
28. P. J. McMurdie, S. Holmes, phyloseq: An R Package for Reproducible Interactive Analysis and Graphics of Microbiome Census Data, PLOS ONE 8, e61217 (2013).

### References for Example 2

1. Huang Y J, Nelson C E, Brodie E L, Desantis T Z, Baek M S, Liu J, Woyke T, Allgaier M, Bristow J, Wiener-Kronish J P et al: Airway microbiota and bronchial hyperresponsiveness in patients with suboptimally controlled asthma. J Allergy Clin Immunol 2011, 127(2):372-381 e371-373.
2. Morgan X C, Tickle T L, Sokol H, Gevers D, Devaney K L, Ward D V, Reyes J A, Shah S A, LeLeiko N, Snapper S B et al: Dysfunction of the intestinal microbiome in inflammatory bowel disease and treatment. Genome biology 2012, 13(9):R79.
3. Yatsunenko T, Rey F E, Manary M J, Trehan I, Dominguez-Bello M G, Contreras M, Magris M, Hidalgo G, Baldassano R N, Anokhin A P et al: Human gut microbiome viewed across age and geography. Nature 2012, 486(7402):222-227.
4. Teo S M, Mok D, Pham K, Kusel M, Serralha M, Troy N, Holt B J, Hales B J, Walker M L, Hollams E et al: The infant nasopharyngeal microbiome impacts severity of lower respiratory infection and risk of asthma development. Cell host & microbe 2015, 17(5):704-715.
5. Abreu N A, Nagalingam N A, Song Y, Roediger F C, Pletcher S D, Goldberg A N, Lynch S V: Sinus microbiome diversity depletion and Corynebacterium tuberculostearicum enrichment mediates rhinosinusitis. Science translational medicine 2012, 4(151):151ra124.
6. Charlson E S, Bittinger K, Haas A R, Fitzgerald A S, Frank I, Yadav A, Bushman F D, Collman R G: Topographical continuity of bacterial populations in the healthy human respiratory tract. Am J Respir Crit Care Med 2011, 184(8):957-963.
7. Huang Y J, Nariya S, Harris J M, Lynch S V, Choy D F, Arron J R, Boushey H: The airway microbiome in patients with severe asthma: Associations with disease features and severity. J Allergy Clin Immunol 2015, 136(4): 874-884.
8. Simpson J L, Daly J, Baines K J, Yang I A, Upham J W, Reynolds P N, Hodge S, James A L, Hugenholtz P, Willner D et al: Airway dysbiosis: Haemophilus influenzae and Tropheryma in poorly controlled asthma. Eur Respir J 2016, 47(3):792-800.
9. Fokkens W J, Lund V J, Mullol J, Bachert C, Alobid I, Baroody F, Cohen N, Cervin A, Douglas R, Gevaert P et al: European Position Paper on Rhinosinusitis and Nasal Polyps 2012. Rhinology Supplement 2012(23):3 p preceding table of contents, 1-298.
10. Caulley L, Thavorn K, Rudmik L, Cameron C, Kilty S J: Direct costs of adult chronic rhinosinusitis by using 4 methods of estimation: Results of the U S Medical Expenditure Panel Survey. JAllergy Clin Immunol 2015, 136(6):1517-1522.
11. Kim R J, Biswas K, Hoggard M, Taylor M W, Douglas R G: Paired analysis of the microbiota of surface mucus and whole-tissue specimens in patients with chronic rhinosinusitis. Int Forum Allergy Rhinol 2015, 5(10):877-883.
12. Biswas K, Hoggard M, Jain R, Taylor M W, Douglas R G: The nasal microbiota in health and disease: variation within and between subjects. Frontiers in microbiology 2015, 9:134.
13. Ramakrishnan V R, Hauser L J, Feazel L M, Ir D, Robertson C E, Frank D N: Sinus microbiota varies among chronic rhinosinusitis phenotypes and predicts surgical outcome. J Allergy Clin Immunol 2015, 136(2):334-342 e331.
14. Bendouah Z, Barbeau J, Hamad W A, Desrosiers M: Biofilm formation by Staphylococcus aureus and Pseudomonas aeruginosa is associated with an unfavorable evolution after surgery for chronic sinusitis and nasal polyposis. Otolaryngology—head and neck surgery: official journal of American Academy of Otolaryngology-Head and Neck Surgery 2006, 134(6):991-996.
15. Divekar R, Patel N, Jin J, Hagan J, Rank M, Lal D, Kita H, O'Brien E: Symptom-Based Clustering in Chronic Rhinosinusitis Relates to History of Aspirin Sensitivity and Postsurgical Outcomes. J Allergy Clin Immunol Pract 2015, 3(6):934-940 e933.
16. Lal D, Rounds A B, Rank M A, Divekar R: Clinical and 22-item Sino-Nasal Outcome Test symptom patterns in primary headache disorder patients presenting to otolaryngologists with “sinus” headaches, pain or pressure. Int Forum Allergy Rhinol 2015, 5(5):408-416.
17. Derycke L, Eyerich S, Van Crombruggen K, Perez-Novo C, Holtappels G, Deruyck N, Gevaert P, Bachert C: Mixed T helper cell signatures in chronic rhinosinusitis with and without polyps. PLoS One 2014, 9(6):e97581.
18. Yang Y, Zhang N, Crombruggen K V, Lan F, Hu G, Hong S, Bachert C: Differential Expression and Release of Activin A and Follistatin in Chronic Rhinosinusitis with and without Nasal Polyps. PLoS One 2015, 10(6):e0128564.
19. Akdis C A, Bachert C, Cingi C, Dykewicz M S, Hellings P W, Naclerio R M, Schleimer R P, Ledford D: Endotypes and phenotypes of chronic rhinosinusitis: a PRACTALL document of the European Academy of Allergy and Clinical Immunology and the American Academy of Allergy, Asthma & Immunology. J Allergy Clin Immunol 2013, 131(6):1479-1490.
20. Tomassen P, Vandeplas G, Van Zele T, Cardell L O, Arebro J, Olze H, Forster-Ruhrmann U, Kowalski M L, Olszewska-Ziaber A, Holtappels G et al: Inflammatory endotypes of chronic rhinosinusitis based on cluster analysis of biomarkers. J Allergy Clin Immunol 2016, 137(5):1449-1456 e1444.
21. Tan N C, Cooksley C M, Roscioli E, Drilling A J, Douglas R, Wormald P J, Vreugde S: Small-colony variants and phenotype switching of intracellular Staphylococcus aureus in chronic rhinosinusitis. Allergy 2014, 69(10):1364-1371.
22. Shenoy M K, Iwai S, Lin D L, Worodria W, Ayakaka I, Byanyima P, Kaswabuli S, Fong S, Stone S, Chang E et al: Immune Response and Mortality Risk Relate to Distinct Lung Microbiomes in HIV-Pneumonia Patients. Am J Respir Crit Care Med 2016.
23. Fujimura K E, Sitarik A R, Haystad S, Lin D L, Levan S, Fadrosh D, Panzer A R, LaMere B, Rackaityte E, Lukacs N W et al: Neonatal gut microbiota associates with childhood multisensitized atopy and T cell differentiation. Nature medicine 2016, 22(10):1187-1191.
24. Rosenfeld R M: Clinical practice guideline on adult sinusitis. Otolaryngol Head Neck Surg 2007, 137(3):365-377.
25. Roediger F C, Slusher N A, Allgaier S, Cox M J, Pletcher S D, Goldberg A N, Lynch S V: Nucleic acid extraction efficiency and bacterial recovery from maxillary sinus mucosal samples obtained by brushing or biopsy. Am J Rhinol Allergy 2010, 24(4):263-265.
26. Caporaso J G, Lauber C L, Walters W A, Berg-Lyons D, Huntley J, Fierer N, Owens S M, Betley J, Fraser L, Bauer M et al: Ultra-high-throughput microbial community analysis on the Illumina HiSeq and MiSeq platforms. ISME J 2012, 6(8): 1621-1624.
27. Caporaso J G, Lauber C L, Walters W A, Berg-Lyons D, Lozupone C A, Turnbaugh P J, Fierer N, Knight R: Global patterns of 16S rRNA diversity at a depth of millions of sequences per sample. Proc Natl Acad Sci USA 2011, 108 Suppl 1:4516-4522.
28. Caporaso J G, Kuczynski J, Stombaugh J, Bittinger K, Bushman F D, Costello E K, Fierer N, Pena A G, Goodrich J K, Gordon J I et al: QIIME allows analysis of high-throughput community sequencing data. Nat Methods 2010, 7(5):335-336.
29. Vazquez-Baeza Y, Pirrung M, Gonzalez A, Knight R: EMPeror: a tool for visualizing high-throughput microbial community data. Gigascience 2013, 2(1):16.
30. Oksanen J, Blanchet, F. G., Kindt, R., Legendre, P., Minchin, P. R., O'Hara, R. B., Simpson, G. L., Solymos, P., Stevens, H. H., Wagner, H.: vegan: Community Ecology Package. R package version 20-10 2013.
31. Anderson M J: A new method for non-parametric multivariate analysis of variance. Austral Ecology 2001, 26:32-46.
32. Storey J D: A direct approach to false discovery rates. J Roy Stat Soc B 2002, 64:479-498.
33. Holmes I, Harris K, Quince C: Dirichlet multinomial mixtures: generative models for microbial metagenomics. PLoS One 2012, 7(2):e30126.
34. Langille M G, Zaneveld J, Caporaso J G, McDonald D, Knights D, Reyes J A, Clemente J C, Burkepile D E, Vega Thurber R L, Knight R et al: Predictive functional profiling of microbial communities using 16S rRNA marker gene sequences. Nat Biotechnol 2013.
35. Foreman A, Psaltis A J, Tan L W, Wormald P J: Characterization of bacterial and fungal biofilms in chronic rhinosinusitis. American journal of rhinology & allergy 2009, 23(6):556-561.
36. Lina G, Boutite F, Tristan A, Bes M, Etienne J, Vandenesch F: Bacterial competition for human nasal cavity colonization: role of Staphylococcal agr alleles. Appl Environ Microbiol 2003, 69(1):18-23.
37. Yan M, Pamp S J, Fukuyama J, Hwang P H, Cho D Y, Holmes S, Relman D A: Nasal microenvironments and interspecific interactions influence nasal microbiota complexity and S. aureus carriage. Cell host & microbe 2013, 14(6):631-640.
38. Kobayashi T, Glatz M, Horiuchi K, Kawasaki H, Akiyama H, Kaplan D H, Kong H H, Amagai M, Nagao K: Dysbiosis and Staphylococcus aureus Colonization Drives Inflammation in Atopic Dermatitis. Immunity 2015, 42(4):756-766.
39. Coburn B, Wang P W, Diaz Caballero J, Clark S T, Brahma V, Donaldson S, Zhang Y, Surendra A, Gong Y, Elizabeth Tullis D et al: Lung microbiota across age and disease stage in cystic fibrosis. Sci Rep 2015, 5:10241.
40. Madan J C, Koestler D C, Stanton B A, Davidson L, Moulton L A, Housman M L, Moore J H, Guill M F, Morrison H G, Sogin M L et al: Serial analysis of the gut and respiratory microbiome in cystic fibrosis in infancy: interaction between intestinal and respiratory tracts and impact of nutritional exposures. mBio 2012, 3(4).
41. Langille M G, Zaneveld J, Caporaso J G, McDonald D, Knights D, Reyes J A, Clemente J C, Burkepile D E, Vega Thurber R L, Knight R et al: Predictive functional profiling of microbial communities using 16S rRNA marker gene sequences. Nat Biotechnol 2013, 31(9):814-821.
42. Evans S E, Goult B T, Fairall L, Jamieson A G, Ko Ferrigno P, Ford R, Schwabe J W, Wagner S D: The ansamycin antibiotic, rifamycin S V, inhibits BCL6 transcriptional repression and forms a complex with the BCL6-BTB/POZ domain. PLoS One 2014, 9(3):e90889.
43. Asaka C, Honda K, Ito E, Fukui N, Chihara J, Ishikawa K: Peroxisome proliferator-activated receptor-gamma is expressed in eosinophils in nasal polyps. Int Arch Allergy Immunol 2011, 155 Suppl 1:57-63.
44. Bogefors J, Kvarnhammar A M, Latif L, Petterson T, Uddman R, Cardell L O: Retinoic acid-inducible gene 1-like receptors in the upper respiratory tract. American journal of rhinology & allergy 2011, 25(6):e262-267.
45. Bachert C, Wagenmann M, Hauser U, Rudack C: IL-5 synthesis is upregulated in human nasal polyp tissue. J Allergy Clin Immunol 1997, 99(6 Pt 1):837-842.
46. Cho K S, Kim C S, Lee H S, Seo S K, Park H Y, Roh H J: Role of interferon-gamma-producing t cells in the pathogenesis of chronic rhinosinusitis with nasal polyps associated with staphylococcal superantigen. J Otolaryngol Head Neck Surg 2010, 39(5):600-605.
47. Joss T V, Burke C M, Hudson B J, Darling A E, Forer M, Alber D G, Charles I G, Stow N W: Bacterial Communities Vary between Sinuses in Chronic Rhinosinusitis Patients. Front Microbiol 2015, 6:1532.
48. Hoggard M, Biswas K, Zoing M, Wagner Mackenzie B, Taylor M W, Douglas R G: Evidence of microbiota dysbiosis in chronic rhinosinusitis. Int Forum Allergy Rhinol 2016.
49. Bomar L, Brugger S D, Yost B H, Davies S S, Lemon K P: Corynebacterium accolens Releases Antipneumococcal Free Fatty Acids from Human Nostril and Skin Surface Triacylglycerols. MBio 2016, 7(1).
50. Qingzhen H, Jia T, Shengjun W, Yang Z, Yanfang L, Pei S, Essien B S, Zhaoliang S, Sheng X, Qixiang S et al: Corynebacterium pyruviciproducens promotes the production of ovalbumin specific antibody via stimulating dendritic cell differentiation and up-regulating Th2 biased immune response. Vaccine 2012, 30(6):1115-1123.
51. Mishra A K, Alves J E, Krumbach K, Nigou J, Castro A G, Geurtsen J, Eggeling L, Saraiva M, Besra G S: Differential arabinan capping of lipoarabinomannan modulates innate immune responses and impacts T helper cell differentiation. J Biol Chem 2012, 287(53):44173-44183.
52. Lee K S, Park S J, Hwang P H, Yi H K, Song C H, Chai O H, Kim J S, Lee M K, Lee Y C: PPAR-gamma modulates allergic inflammation through up-regulation of PTEN. FASEB J 2005, 19(8):1033-1035.
53. Honda K, Marquillies P, Capron M, Dombrowicz D: Peroxisome proliferator-activated receptor gamma is expressed in airways and inhibits features of airway remodeling in a mouse asthma model. J Allergy Clin Immunol 2004, 113(5):882-888.
54. Imaizumi T, Tanaka H, Tajima A, Tsuruga K, Oki E, Sashinami H, Matsumiya T, Yoshida H, Inoue I, Ito E: Retinoic acid-inducible gene-I (RIG-I) is induced by IFN-{gamma} in human mesangial cells in culture: possible involvement of RIG-I in the inflammation in lupus nephritis. Lupus 2010, 19(7):830-836.
55. Harden J L, Lewis S M, Lish S R, Suarez-Farinas M, Gareau D, Lentini T, Johnson-Huang L M, Krueger J G, Lowes M A: The tryptophan metabolism enzyme L-kynureninase is a novel inflammatory factor in psoriasis and other inflammatory diseases. J Allergy Clin Immunol 2015.
56. Favre D, Mold J, Hunt P W, Kanwar B, Loke P, Seu L, Barbour J D, Lowe M M, Jayawardene A, Aweeka F et al: Tryptophan catabolism by indoleamine 2,3-dioxygenase 1 alters the balance of TH17 to regulatory T cells in HIV disease. Science translational medicine 2010, 2(32):32ra36.
57. Vujkovic-Cvijin I, Dunham R M, Iwai S, Maher M C, Albright R G, Broadhurst M J, Hernandez R D, Lederman M M, Huang Y, Somsouk M et al: Dysbiosis of the gut microbiota is associated with HIV disease progression and tryptophan catabolism. Science translational medicine 2013, 5(193):193ra191.
58. Ujimaru T, Kakimoto T, Chibata I: 1-Tryptophan Production by Achromobacter liquidum. Applied and environmental microbiology 1983, 46(1):1-5.
59. Sasaki-Imamura T, Yano A, Yoshida Y: Production of indole from L-tryptophan and effects of these compounds on biofilm formation by Fusobacterium nucleatum ATCC 25586. Applied and environmental microbiology 2010, 76(13):4260-4268.
60. Lee J, Attila C, Cirillo S L, Cirillo J D, Wood T K: Indole and 7-hydroxyindole diminish Pseudomonas aeruginosa virulence. Microb Biotechnol 2009, 2(1):75-90.
61. Anyanful A, Dolan-Livengood J M, Lewis T, Sheth S, Dezalia M N, Sherman M A, Kalman L V, Benian G M, Kalman D: Paralysis and killing of Caenorhabditis elegans by enteropathogenic Escherichia coli requires the bacterial tryptophanase gene. Mol Microbiol 2005, 57(4):988-1007.
62. Hirakawa H, Kodama T, Takumi-Kobayashi A, Honda T, Yamaguchi A: Secreted indole serves as a signal for expression of type III secretion system translocators in enterohaemorrhagic Escherichia coli I157:H7. Microbiology 2009, 155(Pt 2):541-550.
63. Lubamba B A, Jones L C, O'Neal W K, Boucher R C, Ribeiro C M: X-Box-Binding Protein 1 and Innate Immune Responses of Human Cystic Fibrosis Alveolar Macrophages. Am J Respir Crit Care Med 2015, 192(12):1449-1461.
64. LaFayette S L, Houle D, Beaudoin T, Wojewodka G, Radzioch D, Hoffman L R, Burns J L, Dandekar A A, Smalley N E, Chandler J R et al: Cystic fibrosis-adapted quorum sensing mutants cause hyperinflammatory responses. Sci Adv 2015, 1(6).
65. Jones C L, Weiss D S: TLR2 signaling contributes to rapid inflammasome activation during F. novicida infection. PLoS One 2011, 6(6):e20609.
66. Makowska J S, Burney P, Jarvis D, Keil T, Tomassen P, Bislimovska J, Brozek G, Bachert C, Baelum J, Bindslev-Jensen C et al: Respiratory Hypersensitivity Reactions to NSAIDs in Europe: The Global Allergy and Asthma Network (GA2 LEN) Survey. Allergy 2016.
67. Dethlefsen L, Relman D A: Incomplete recovery and individualized responses of the human distal gut microbiota to repeated antibiotic perturbation. Proc Natl Acad Sci USA 2011, 108 Suppl 1:4554-4561.
68. Theriot C M, Koenigsknecht M J, Carlson P E, Jr., Hatton G E, Nelson A M, Li B, Huffnagle G B, J Z L, Young V B: Antibiotic-induced shifts in the mouse gut microbiome and metabolome increase susceptibility to Clostridium difficile infection. Nat Commun 2014, 5:3114.
69. Liu C M, Soldanova K, Nordstrom L, Dwan M G, Moss O L, Contente-Cuomo T L, Keim P, Price L B, Lane A P: Medical therapy reduces microbiota diversity and evenness in surgically recalcitrant chronic rhinosinusitis. Int Forum Allergy Rhinol 2013, 3(10):775-781.
70. Postma D S, Rabe K F: The Asthma-COPD Overlap Syndrome. N Engl J Med 2015, 373(13):1241-1249.
71. Christenson S A, Steiling K, van den Berge M, Hijazi K, Hiemstra P S, Postma D S, Lenburg M E, Spira A, Woodruff P G: Asthma-COPD overlap. Clinical relevance of genomic signatures of type 2 inflammation in chronic obstructive pulmonary disease. Am J Respir Crit Care Med 2015, 191(7):758-766.
72. Marri P R, Stern D A, Wright A L, Billheimer D, Martinez F D: Asthma-associated differences in microbial composition of induced sputum. J Allergy Clin Immunol 2013, 131(2):346-352 e341-343.
73. Bacci G, Paganin P, Lopez L, Vanni C, Dalmastri C, Cantale C, Daddiego L, Perrotta G, Dolce D, Morelli P et al: Pyrosequencing Unveils Cystic Fibrosis Lung Microbiome Differences Associated with a Severe Lung Function Decline. PLoS One 2016, 11(6):e0156807.
74. Woodruff P G, Modrek B, Choy D F, Jia G, Abbas A R, Ellwanger A, Koth L L, Anon J R, Fahy J V: T-helper type 2-driven inflammation defines major subphenotypes of asthma. Am J Respir Crit Care Med 2009, 180(5):388-395.
75. Caporaso, J. G. et al. QIIME allows analysis of high-throughput community sequencing data. Nat Methods 7, 335-336, doi:nmeth.f.303 [pii]10.1038/nmeth.f.303 (2010).
76. Magoč, T. & Salzberg, S. L. FLASH: fast length adjustment of short reads to improve genome assemblies. Bioinformatics 27, 2957-2963, doi:10.1093/bioinformatics/btr507 (2011).
77. DeSantis, T. Z. et al. Greengenes, a chimera-checked 16S rRNA gene database and workbench compatible with ARB. Appl Environ Microbiol 72, 5069-5072, doi:72/7/5069 [pii]10.1128/AEM.03006-05 (2006).
78. Edgar, R. C. Search and clustering orders of magnitude faster than BLAST. Bioinformatics 26, 2460-2461, doi:btq461 [pii]10.1093/bioinformatics/btq461 (2010).
79. Caporaso, J. G. et al. PyNAST: a flexible tool for aligning sequences to a template alignment. Bioinformatics 26, 266-267, doi:btp636 [pii]10.1093/bioinformatics/btp636 (2010).
80. Price, M. N., Dehal, P. S. & Arkin, A. P. FastTree: computing large minimum evolution trees with profiles instead of a distance matrix. Mol Biol Evol 26, 1641-1650, doi:msp077 [pii]10.1093/molbev/msp077 (2009).
81. Langille, M. G. et al. Predictive functional profiling of microbial communities using 16S rRNA marker gene sequences. Nat Biotechnol, doi:nbt.2676 [pii]10.1038/nbt.2676 (2013).
82. Lane, D. 16S/23S rRNA sequencing., pp 115-175. (John Wiley & Sons., 1991).
83. Roediger, F. C. et al. Nucleic acid extraction efficiency and bacterial recovery from maxillary sinus mucosal samples obtained by brushing or biopsy. Am J Rhinol Allergy 24, 263-265, doi:10.2500/ajra.2010.24.3472 (2010).
84. Einen, J., Thorseth, I. H. & Ovreas, L. Enumeration of Archaea and Bacteria in seafloor basalt using real-time quantitative PCR and fluorescence microscopy. FEMS Microbiol Lett 282, 182-187, doi:10.1111/j.1574-6968.2008.01119.x (2008).
85. Livak, K. J. & Schmittgen, T. D. Analysis of relative gene expression data using real-time quantitative PCR and the 2(-Delta Delta C(T)) Method. Methods 25, 402-408, doi:10.1006/meth.2001.1262S1046-2023(01)91262-9 [pii] (2001).

1. A method of detecting a nasal or sinus microbiome in a subject who has asthma, the method comprising detecting bacteria, or a proportion of bacteria, in a biological sample from the subject that are in 1 of or any combination of the following taxa: Moraxella, Staphylococcaceae, Corynebacterium, Corynebacteriaceae, Staphylococcus, Staphylococcaceae, Streptococcus, Streptococcaceae, Pseudomonadaceae, Haemophilus, Fusobacteriaceae, Pasteruellaceae, Prevotellaceae, Fusobacteriaceae, Neisseriaceae, and/or Enterobacteriaceae.

2. The method of claim 1, wherein
(a) detecting the nasal or sinus microbiome comprises amplifying and sequencing 16S rRNA genes, or portions thereof, of microorganisms in the biological sample;
(b) detecting nasal or sinus microbiome comprises amplifying and sequencing the V4 region of 16S rRNA genes of microorganisms in the biological sample;
(c) the method comprises detecting the proportion of bacteria in the biological sample that are within 1 of or any combination of 2, 3, 4, 5, 6, 7, 8, 9, or 10 of the following taxa; Moraxella, Staphylococcaceae, Corynebacterium, Staphylococcus, Strepococcus, and/or Haemophilus;
(d) the method comprises detecting the proportion of bacteria in the biological sample that are within 1 of or any combination of 2, 3, 4, 5, 6, 7, 8, 9, or 10 of the following families: Corynebacteriaceae, Staphylococcaceae, Pseudomonadaceae, Streptococcaceae, Fusobacteriaceae, Pasteruellaceae, Prevotellaceae, Fusobacteriaceae, Neisseriaceae, and/or Enterobacteriaceae;
(e) the method comprises determining the expression level of at least one gene in the biological sample;
(f) the method comprises determining the gene expression level of 1, 2, 3, 4, 5, 6, 7, 8, 9, 10, 11, 12, 13, 14, 15, 16, 17, 18, or 19 of Occludin, Claudin 2, MUCSAC, IL-4, IL-5, IL-6, IL-8, IL-25, IL-17A, IL-10, IL-1β, IL-33, CCL11, TSLP, TNF-α, ARG1, TGFβ1, CLCA1, or IFN-γ;
(g) the biological sample is a nasal saline wash, a bodily fluid, nasal mucus, nasal discharge, sinus mucus, or sinus brushing comprising mucus from the surface of a sinus;
(h) the subject is 6-18 years old;
(i) omalizumab was previously administered to the subject;
(j) the biological sample is obtained in June, July, August, or September.

3.-5. (canceled)

6. The method of claim 1, comprising detecting whether:
(a) the biological sample has an increased proportion of bacteria in the Moraxella genus of bacteria compared to a general population of subjects who have asthma;
(b) the biological sample has an increased proportion of bacteria in the Neisseria genus of bacteria compared to a general population of subjects who have asthma;
(c) the biological sample has a decreased proportion of bacteria in the Staphylococcaceae family or Corynebacterium genus of bacteria compared to a general population of subjects who have asthma;
(d) the biological sample has a decreased proportion of bacteria in the Corynebacterium genus of bacteria compared to a general population of subjects who have asthma;
(e) the biological sample has decreased proportion of bacteria in the Haemophilus genus of bacteria compared to a general population of subjects who have asthma;
(f) the biological sample has decreased proportion of bacteria in the Staphylococcaceae family of bacteria compared to a general population of subjects who have asthma;
(g) the biological sample has an increased proportion of bacteria in the Staphylococcus genus or Corynebacterium genus of bacteria compared to a general population of subjects who have asthma;
(h) the biological sample has an increased proportion of bacteria in the Streptococcus genus of bacteria compared to a general population of subjects who have asthma; and/or
(i) the biological sample has an increased proportion of bacteria in the Haemophilus genus of bacteria compared to a general population of subjects who have asthma.

7. The method of claim 1, comprising detecting whether the subject:
(a) has an increased proportion of nasal microbiome bacteria in the Moraxella genus of bacteria compared to a general population of subjects who have asthma;
(b) has an increased proportion of nasal microbiome bacteria in the Neisseria genus of bacteria compared to a general population of subjects who have asthma;
(c) has an increased proportion of nasal microbiome bacteria in the Staphylococcaceae family or Corynebacterium genus of bacteria compared to a general population of subjects who have asthma;
(d) has an increased proportion of nasal microbiome bacteria in the Corynebacterium genus of bacteria compared to a general population of subjects who have asthma;
(e) has an increased proportion of nasal microbiome bacteria in the Haemophilus genus of bacteria compared to a general population of subjects who have asthma;
(f) has an increased proportion of nasal microbiome bacteria in the Staphylococcaceae family of bacteria compared to a general population of subjects who have asthma; and/or
(g) has an increased proportion of nasal microbiome bacteria in the Staphylococcus genus or Corynebacterium genus of bacteria compared to a general population of subjects who have asthma.

8. The method of claim 1, comprising detecting whether the subject:
(a) has an increased proportion of nasal microbiome bacteria in the Haemophilus genus of bacteria compared to a general population of subjects who have asthma;
(b) has an increased proportion of nasal microbiome bacteria in the Streptococcus genus of bacteria compared to a general population of subjects who have asthma;
(c) has an increased proportion of nasal microbiome bacteria in the Moraxella genus of bacteria compared to a general population of subjects who have asthma;
(d) has an increased proportion of nasal microbiome bacteria in the Neisseria genus of bacteria compared to a general population of subjects who have asthma; and/or
(e) has a decreased proportion of nasal microbiome bacteria in the Staphyloccocaceae family of bacteria compared to a general population of subjects who have asthma.

9. The method of claim 1, comprising detecting whether:
(a) the biological sample has an increased proportion of bacteria in the Prevotellaceae and Fusobacteriaceae families of bacteria compared to a general or healthy population of subjects;
(b) the biological sample has an increased proportion of bacteria in the Pseudomonas genus of bacteria compared to a general or healthy population of subjects;
(c) the biological sample has an increased proportion of bacteria in the Streptococcus, Porphyromonas, Tannerella, Treponema, Bacteroides, Dialister, and Akkermansia taxa of bacteria compared to a general or healthy population of subjects;
(d) the biological sample has an increased proportion of bacteria in the Prevotellaceae family of bacteria compared to a general or healthy population of subjects, wherein the increased proportion is a proportion of at least about 10% or 20%;
(e) the biological sample has an increased proportion of bacteria in the Fusobacteriaceae family of bacteria compared to a general or healthy population of subjects, wherein the increased proportion is a proportion of at least about 5% or 10%;
(f) at least 10% of the bacteria in the biological sample are in the Prevotellaceae family of bacteria, and at least 5% of the bacteria in the biological sample are in the Fusobacteriaceae family of bacteria;
(g) the biological sample has an increased proportion of bacteria in the Pseudomonadaceae, Fusobacteriaceae, Staphylococcaceae, and Enterobacteriaceae families of bacteria compared to a general or healthy population of subjects;
(h) the biological sample has an increased proportion of bacteria in the Pseudomonadaceae family of bacteria compared to a general or healthy population of subjects, wherein the increased proportion is a proportion of at least about 20%, 30%, 40%, or 50%;
(i) the biological sample has an increased proportion of bacteria in the Fusobacteriaceae family of bacteria compared to a general or healthy population of subjects, wherein the increased proportion is a proportion of at least about 5% or 10%;
(j) the biological sample has an increased proportion of bacteria in the Staphylococcaceae family of bacteria compared to a general or healthy population of subjects, wherein the increased proportion is a proportion of at least about 10% or 20%;
(k) the biological sample has an increased proportion of bacteria in the Enterobacteriaceae family of bacteria compared to a general or healthy population of subjects, wherein the increased proportion is a proportion of at least about 5%;
(l) at least 20% of the bacteria in the biological sample are in the Pseuydomonadaceae family of bacteria, at least 5% of the bacteria in the biological sample are in the Fusobacteriaceae family of bacteria, at least 10% of the bacteria in the biological sample are in the Staphylococcaceae family of bacteria, and at least 5% of the bacteria in the biological sample are in the Enterobacteriaceae family of bacteria;
(m) the biological sample has an increased proportion of bacteria in the Sphingomonas genus of bacteria compared to a general or healthy population of subjects;
(n) the biological sample has an increased proportion of bacteria in the Corynebacteriaceae and Staphylococcaceae families of bacteria compared to a general or healthy population of subjects;
(o) the biological sample has an increased proportion of bacteria in the Corynebacteriaceae family of bacteria compared to a general or healthy population of subjects, wherein the increased proportion is a proportion of at least about 50%;
(p) the biological sample has an increased proportion of bacteria in the Staphylococcaceae family of bacteria compared to a general or healthy population of subjects, wherein the increased proportion is a proportion of at least about 10%;
(q) at least 50% of the bacteria in the biological sample are in the Corynebacteriaceae family of bacteria, and at least 10% of the bacteria in the biological sample are in the Staphylococcaceae family of bacteria;
(r) the biological sample has an increased proportion of bacteria in the Enterobacteriaceae and Staphylococcaceae families of bacteria compared to a general or healthy population of subjects;
(s) the biological sample has an increased proportion of bacteria in the Actinobacteria, Bfidobacterium, Haemophilus, Enterobacteriaceae, Pseudomonadaceae, Sphingomonadaceae, Selenomonas and Streptophyta taxa of bacteria compared to a general or healthy population of subjects;
(t) the biological sample has an increased proportion of bacteria in the Enterobacteriaceae family of bacteria compared to a general or healthy population of subjects, wherein the increased proportion is a proportion of at least about 5%;
(u) the biological sample has an increased proportion of bacteria in the Staphylococcaceae family of bacteria compared to a general or healthy population of subjects, wherein the increased proportion is a proportion of at least about 10%, 20%, 30%, 40%, or 50%; and/or
(v) at least 5% of the bacteria in the biological sample are in the Enterobacteriaceae family of bacteria, and at least 30% of the bacteria in the biological sample are in the Staphylococcaceae family of bacteria.

10. (canceled)

11. (canceled)

12. The method of claim 1, comprising detecting whether the subject:
(a) has increased expression of any 1 of or 2, 3, 4, or 5 of any combination of IL-1β, IL-6, IL-10, IL-5, and IFN-γ compared to a general or healthy population of subjects;
(b) the biological sample has an increased proportion of bacteria in the Pseudomonas genus of bacteria compared to a general or healthy population of subjects;
(c) has an increased proportion of sinus microbiome bacteria in the Prevotellaceae and Fusobacteriaceae families of bacteria compared to a general or healthy population of subjects;
(d) has an increased proportion of sinus microbiome bacteria in the Streptococcus, Porphyromonas, Tannerella, Treponema, Bacteroides, Dialister, and Akkermansia taxa of bacteria compared to a general or healthy population of subjects;
(e) has an increased proportion of sinus microbiome bacteria in the Prevotellaceae family of bacteria compared to a general or healthy population of subjects, wherein the increased proportion is a proportion of at least about 10% or 20%;
(f) has an increased proportion of sinus microbiome bacteria in the Fusobacteriaceae family of bacteria compared to a general or healthy population of subjects, wherein the increased proportion is a proportion of at least about 5% or 10%;
(g) has an increased proportion of sinus microbiome bacteria in the Pseudomonadaceae, Fusobacteriaceae, Staphylococcaceae, and Enterobacteriaceae families of bacteria compared to a general or healthy population of subjects;
(h) has an increased proportion of sinus microbiome bacteria in the Pseudomonadaceae family of bacteria compared to a general or healthy population of subjects, wherein the increased proportion is a proportion of at least about 20%, 30%, 40%, or 50%;
(i) has an increased proportion of sinus microbiome bacteria in the Fusobacteriaceae family of bacteria compared to a general or healthy population of subjects, wherein the increased proportion is a proportion of at least about 5% or 10%;
(j) has an increased proportion of sinus microbiome bacteria in the Staphylococcaceae family of bacteria compared to a general or healthy population of subjects, wherein the increased proportion is a proportion of at least about 10% or 20%;
(k) has an increased proportion of sinus microbiome bacteria in the Enterobacteriaceae family of bacteria compared to a general or healthy population of subjects, wherein the increased proportion is a proportion of at least about 5%;
(l) has an increased proportion of sinus microbiome bacteria in the Sphingomonas genus of bacteria compared to a general or healthy population of subjects;
(m) has an increased proportion of sinus microbiome bacteria in the Corynebacteriaceae and Staphylococcaceae families of bacteria compared to a general or healthy population of subjects;
(n) has an increased proportion of sinus microbiome bacteria in the Corynebacteriaceae family of bacteria compared to a general or healthy population of subjects, wherein the increased proportion is a proportion of at least about 50%;
(o) has an increased proportion of sinus microbiome bacteria in the Staphylococcaceae family of bacteria compared to a general or healthy population of subjects, wherein the increased proportion is a proportion of at least about 10%;
(p) has an increased proportion of sinus microbiome bacteria in the Enterobacteriaceae and Staphylococcaceae families of bacteria compared to a general or healthy population of subjects;
(q) has an increased proportion of sinus microbiome bacteria in the Actinobacteria, Bfidobacterium, Haemophilus, Enterobacteriaceae, Pseudomonadaceae, Sphingomonadaceae, Selenomonas and Streptophyta taxa of bacteria compared to a general or healthy population of subjects;
(r) has an increased proportion of sinus microbiome bacteria in the Enterobacteriaceae family of bacteria compared to a general or healthy population of subjects, wherein the increased proportion is a proportion of at least about 5%; and/or
(s) has an increased proportion of sinus microbiome bacteria in the Staphylococcaceae family of bacteria compared to a general or healthy population of subjects, wherein the increased proportion is a proportion of at least about 10%, 20%, 30%, 40%, or 50%.

13. The method of claim 1, comprising detecting whether the subject:
(a) has increased IL-5 or IFN-γ expression compared to a general or healthy population of subjects;
(b) the biological sample has an increased proportion of bacteria in the Pseudomonas genus of bacteria compared to a general or healthy population of subjects;
(c) has an increased proportion of sinus microbiome bacteria in the Enterobacteriaceae and Staphylococcaceae families of bacteria compared to a general or healthy population of subjects;
(d) has an increased proportion of sinus microbiome bacteria in the Actinobacteria, Bfidobacterium, Haemophilus, Enterobacteriaceae, Pseudomonadaceae, Sphingomonadaceae, Selenomonas and Streptophyta taxa of bacteria compared to a general or healthy population of subjects;
(e) has an increased proportion of sinus microbiome bacteria in the Enterobacteriaceae family of bacteria compared to a general or healthy population of subjects, wherein the increased proportion is a proportion of at least about 5%; and/or
(f) has an increased proportion of sinus microbiome bacteria in the Staphylococaceae family of bacteria compared to a general or healthy population of subjects, wherein the increased proportion is a proportion of at least about 10%, 20%, 30%, 40%, or 50%.

14.-23. (canceled)

24. A method of treating or preventing a condition in a subject in need thereof, comprising administering to the subject an effective amount of an antibiotic compound, wherein:
(a) the condition is asthma, an asthma exacerbation, or a rhinovirus infection;
(b) the condition is asthma, an asthma exacerbation, or a rhinovirus infection, and the method comprises detecting nasal dysbiosis in the subject;
(c) the condition is asthma, an asthma exacerbation, or a rhinovirus infection, and the subject has been identified as having an increased risk of asthma exacerbation or rhinovirus infection compared to a general population of subjects who have asthma;
(d) the condition is chronic rhinosinusitis or nasal polyposis;
(e) the condition is chronic rhinosinusitis or nasal polyposis, and the method comprises detecting sinus dysbiosis in the subject;
(f) the condition is chronic rhinosinusitis or nasal polyposis, and the subject has been identified as having or being at risk of having chronic rhinosinusitis or nasal polyposis.

25.-27. (canceled)

28. The method of claim 24, wherein
(a) the subject has nasal dysbiosis or sinus dysbiosis;
(b) the antibiotic compound is a B-lactam, a cephalosporin, a lincosamide, a macrolide, a tetracycline, a sulfa compound, or mupirocin;
(c) the antibiotic compound is erythromycin, penicillin G. clarithromycin, bactrim DS, ciprofloxacin, vancomycin, daptomycin, or linezolid;
(d) the antibiotic compound is a penicillin, a +/−beta-lactam inhibitor, a cephalosporin, a monobactam, a fluoroquinolone, a carbapenem, an aminoglycoside, or a polymixin;
(e) the antibiotic is a penicillin or a cephalosporin; or
(f) the antibiotic is metronidazole amoxicillin, clavulanate, amoxicillin and clavulanate, a ureidopenicilin, a carbapenem, a cephalosporin, clindamycin, or chloramphenicol.

29. The method of claim 28, wherein the subject has nasal dysbiosis or sinus dysbiosis, and further wherein:
(a) the nasal dysbiosis or sinus dysbiosis comprises an increased proportion or amount of Staphylococcus sp. bacteria compared to a standard control;
(b) the nasal dysbiosis or sinus dysbiosis comprises an increased proportion or amount of Corynebacterium sp. bacteria compared to a standard control
(c) the nasal dysbiosis or sinus dysbiosis comprises an increased proportion or amount of Pseudomonas sp. bacteria compared to a standard control;
(d) the nasal dysbiosis or sinus dysbiosis comprises an increased proportion or amount of Streptococcus sp. bacteria compared to a standard control; or
(e) the nasal dysbiosis or sinus dysbiosis comprises an increased proportion or amount of Prevotella sp. bacteria compared to a standard control.

30.-34. (canceled)

35. The method of claim 28, wherein
(a) the antibiotic compound is oxacillin, flucloxacillin, cefazolin, cephalothin, cephalexin, erythromycin, doxycycline, or minocycline;
(b) the antibiotic is a mupirocin administered in a cream;
(c) the subject has nasal dysbiosis comprising an increased proportion or amount of Staphylococcus sp. bacteria compared to a standard control, and further wherein the antibiotic compound is a B-lactam, a cephalosporin, a lincosamide, a macrolide, a tetracycline, a sulfa compound, or mupirocin.

36.-46. (canceled)

47. The method of claim 24, further comprising administering at least one bacterium or an anti-IL-5 compound to the subject, optionally wherein the at least one bacterium comprises a Lactobacillus sakei bacterium.

48. (canceled)

49. (canceled)

50. A method of treating or preventing chronic rhinosinusitis or nasal polyposis in a subject in need thereof, comprising administering to the subject an effective amount of an anti-IL-5 compound.

51. The method of claim 50, wherein
(a) the anti-L-5 compound is an anti-L-5 antibody;
(b) the anti-IL-5 compound is reslizumab or mepolizumab;
(c) the anti-IL-5 compound is an anti-IL-5 receptor antibody;
(d) the anti-IL-5 compound is benralizumab;
(e) the method further comprises administering at least one bacterium to the subject, optionally wherein the at least one bacterium comprises a Lactobacillus sakei bacterium.

52.-58. (canceled)

59. A method of treating or preventing a condition in a subject in need thereof, comprising administering to the subject an effective amount of at least one bacterium, wherein:
(a) the condition is asthma, an asthma exacerbation, or a rhinovirus infection;
(b) the condition is asthma, an asthma exacerbation, or a rhinovirus infection, and the method further comprises detecting nasal dysbiosis in the subject;
(c) the condition is asthma, an asthma exacerbation, or a rhinovirus infection, and the subject has been identified as having an increased risk of asthma exacerbation or rhinovirus infection compared to a general population of subjects who have asthma;
(d) the condition is chronic rhinosinusitis or nasal polyposis;
(e) the condition is chronic rhinosinusitis or nasal polyposis, and the method further comprises detecting sinus dysbiosis in the subject; or
(f) the condition is chronic rhinosinusitis or nasal polyposis, and the subject has been identified as having or being at risk of having chronic rhinosinusitis or nasal polyposis.

60.-62. (canceled)

63. The method of claim 59, wherein
(a) the subject has nasal dysbiosis or sinus dysbiosis;
(b) the subject has an increased proportion or amount of Corynebacterium sp. bacteria compared to a standard control; or
(c) the subject has an increased proportion or amount of Pseudomonas sp. bacteria compared to a standard control.

64. (canceled)

65. (canceled)

66. The method of claim 59, wherein the at least one bacterium is Lactobacillus sakei.

67.-70. (canceled)

71. A composition comprising an isolated Lactobacillus sakei bacterium and a pharmaceutically acceptable excipient.

72. The composition of claim 71, wherein
(a) the pharmaceutically acceptable excipient is suitable for nasal administration;
(b) the composition is a capsule, a tablet, a suspension, a suppository, a powder, a cream, an oil, an oil-in-water emulsion, a water-in-oil emulsion, or an aqueous solution;
(c) the composition is in the form of a powder, a solid, a semi-solid, or a liquid;
(d) the composition further comprises an anti-IL-5 compound.

73.-75. (canceled)
